SEQUENCE LISTING (270 sequences)

SEQ ID NO: 1 - DNA, 21 bp, Artificial sequence
  Synthetic construct
ggggacgacgtcgtggggggg

SEQ ID NO: 2 - DNA, 20 bp, Artificial sequence
  Synthetic construct
gggggagcatgctggggggg

SEQ ID NO: 3 - protein, 17 aa, Artificial sequence
  Synthetic construct
CCTGCXXXXXXXGCAGG

SEQ ID NO: 4 - protein, 8 aa, Artificial sequence
  Synthetic construct
GGCDGRCG

SEQ ID NO: 5 - protein, 9 aa, Artificial sequence
  Synthetic construct
CRRETAWAC

SEQ ID NO: 6 - protein, 9 aa, Artificial sequence
  Synthetic construct
CDCRGDCFC

SEQ ID NO: 7 - protein, 6 aa, Artificial sequence
  Synthetic construct
RGDWXE

SEQ ID NO: 8 - protein, 6 aa, Artificial sequence
  Synthetic construct
TRGDTF

SEQ ID NO: 9 - protein, 7 aa, Artificial sequence
  Synthetic construct
RGDLXXL

SEQ ID NO: 10 - protein, 7 aa, Artificial sequence
  Synthetic construct
XXDLXXL

SEQ ID NO: 11 - protein, 5 aa, Artificial sequence
  Synthetic construct
SRGDM

SEQ ID NO: 12 - protein, 9 aa, Artificial sequence
  Synthetic construct
VVISYSMPD

SEQ ID NO: 13 - protein, 7 aa, Artificial sequence
  Synthetic construct
IELLQAR

SEQ ID NO: 14 - protein, 21 aa, Artificial sequence
  Synthetic construct
CNGRCGGKLAKLAKKLAKLAK

SEQ ID NO: 15 - protein, 9 aa, Artificial sequence
  Synthetic construct
CVSNPRWKC

SEQ ID NO: 16 - protein, 9 aa, Artificial sequence
  Synthetic construct
CHVLWSTRC

SEQ ID NO: 17 - protein, 8 aa, Artificial sequence
  Synthetic construct
CWDDGWLC

SEQ ID NO: 18 - protein, 9 aa, Artificial sequence
  Synthetic construct
CPCFLLGCC

SEQ ID NO: 19 - protein, 8 aa, Artificial sequence
  Synthetic construct
DFKLFAVY

SEQ ID NO: 20 - protein, 5 aa, Artificial sequence
  Synthetic construct
EWVDV

SEQ ID NO: 21 - protein, 4 aa, Artificial sequence
  Synthetic construct
XXXW

SEQ ID NO: 22 - protein, 10 aa, Artificial sequence
  Synthetic construct
CTTHWGFTLC

SEQ ID NO: 23 - protein, 17 aa, Artificial sequence
  Synthetic construct
NXXEIEXYXXWXXXXXY

SEQ ID NO: 24 - protein, 12 aa, Artificial sequence
  Synthetic construct
HTMYYHHYQHHL

SEQ ID NO: 25 - protein, 7 aa, Artificial sequence
  Synthetic construct
ATWLPPR

SEQ ID NO: 26 - protein, 12 aa, Artificial sequence
  Synthetic construct
WHSDMEWWYLLG

SEQ ID NO: 27 - protein, 6 aa, Artificial sequence
  Synthetic construct
RRKRRR

SEQ ID NO: 28 - protein, 10 aa, Artificial sequence
  Synthetic construct
TAASGVRSMH

SEQ ID NO: 29 - protein, 10 aa, Artificial sequence
  Synthetic construct
LTLRWVGLMS

SEQ ID NO: 30 - protein, 7 aa, Artificial sequence
  Synthetic construct
LMLPRAD

SEQ ID NO: 31 - protein, 9 aa, Artificial sequence
  Synthetic construct
CKGGRAKDC

SEQ ID NO: 32 - protein, 9 aa, Artificial sequence
  Synthetic construct
CLSSRLDAC

SEQ ID NO: 33 - protein, 8 aa, Artificial sequence
  Synthetic construct
VGLPEHTQ

SEQ ID NO: 34 - protein, 12 aa, Artificial sequence
  Synthetic construct
VPWMEPAYQRFL

SEQ ID NO: 35 - protein, 7 aa, Artificial sequence
  Synthetic construct
LTVXPWX

SEQ ID NO: 36 - protein, 7 aa, Artificial sequence
  Synthetic construct
LTVXPWY

SEQ ID NO: 37 - protein, 12 aa, Artificial sequence
  Synthetic construct
LLGPYELWELSH

SEQ ID NO: 38 - protein, 4 aa, Artificial sequence
  Synthetic construct
RPMC

SEQ ID NO: 39 - protein, 7 aa, Artificial sequence
  Synthetic construct
YSGKWGW

SEQ ID NO: 40 - protein, 12 aa, Artificial sequence
  Synthetic construct
TSPLNIHNGQKL

SEQ ID NO: 41 - protein, 8 aa, Artificial sequence
  Synthetic construct
CGFELETC

SEQ ID NO: 42 - protein, 7 aa, Artificial sequence
  Synthetic construct
NSVRDLX

SEQ ID NO: 43 - protein, 7 aa, Artificial sequence
  Synthetic construct
NSVSSXX

SEQ ID NO: 44 - protein, 9 aa, Artificial sequence
  Synthetic construct
CGNKRTRGC

SEQ ID NO: 45 - protein, 7 aa, Artificial sequence
  Synthetic construct
GVLEGRX

SEQ ID NO: 46 - protein, 4 aa, Artificial sequence
  Synthetic construct
XFGX

SEQ ID NO: 47 - protein, 10 aa, Artificial sequence
  Synthetic construct
CVSSNPRWKC

SEQ ID NO: 48 - protein, 9 aa, Artificial sequence
  Synthetic construct
CHVLWSTRC

SEQ ID NO: 49 - protein, 9 aa, Artificial sequence
  Synthetic construct
SWCEPGWCR

SEQ ID NO: 50 - protein, 11 aa, Artificial sequence
  Synthetic construct
AGGDPRATPGS

SEQ ID NO: 51 - protein, 7 aa, Artificial sequence
  Synthetic construct
SMSIARL

SEQ ID NO: 52 - protein, 9 aa, Artificial sequence
  Synthetic construct
CGRRAGGSC

SEQ ID NO: 53 - protein, 12 aa, Artificial sequence
  Synthetic construct
RDVCSCFRDVCC

SEQ ID NO: 54 - protein, 7 aa, Artificial sequence
  Synthetic construct
TPKTSVT

SEQ ID NO: 55 - protein, 7 aa, Artificial sequence
  Synthetic construct
GLSGGRS

SEQ ID NO: 56 - RNA, 21 nt, Artificial sequence
  Synthetic construct
uggauccggcuuugagaucuu

SEQ ID NO: 57 - RNA, 31 nt, Artificial sequence
  Synthetic construct
gggagacagggguguccgccauuuccagguu

SEQ ID NO: 58 - RNA, 31 nt, Artificial sequence
  Synthetic construct
gggagacaggcuauaacucacauaauguauu

SEQ ID NO: 59 - RNA, 18 nt, Artificial sequence
  Synthetic construct
uuuuuuuuuuuuuuuuuu

SEQ ID NO: 60 - RNA, 5 nt, Artificial sequence
  Synthetic construct
ugugu

SEQ ID NO: 61 - RNA, 3 nt, Artificial sequence
  Synthetic construct
ugu

SEQ ID NO: 62 - RNA, 19 nt, Artificial sequence
  Synthetic construct
cuacacaaaucagcgauuu

SEQ ID NO: 63 - RNA, 19 nt, Artificial sequence
  Synthetic construct
aaaucgcugauuuguguag

SEQ ID NO: 64 - RNA, 19 nt, Artificial sequence
  Synthetic construct
uugauguguuuagucgcua

SEQ ID NO: 65 - RNA, 19 nt, Artificial sequence
  Synthetic construct
uagcgacuaaacacaucaa

SEQ ID NO: 66 - RNA, 19 nt, Artificial sequence
  Synthetic construct
gauuauguccgguuaugua

SEQ ID NO: 67 - RNA, 19 nt, Artificial sequence
  Synthetic construct
uacauaaccggacauaauc

SEQ ID NO: 68 - RNA, 19 nt, Artificial sequence
  Synthetic construct
auguauuggccuguauuag

SEQ ID NO: 69 - RNA, 19 nt, Artificial sequence
  Synthetic construct
cuaauacaggccaauacau

SEQ ID NO: 70 - RNA, 19 nt, Artificial sequence
  Synthetic construct
ggucggaaucgaagguuua

SEQ ID NO: 71 - RNA, 19 nt, Artificial sequence
  Synthetic construct
uaaaccuucgauuccgacc

SEQ ID NO: 72 - RNA, 19 nt, Artificial sequence
  Synthetic construct
ggucggagcuaaagguuua

SEQ ID NO: 73 - RNA, 19 nt, Artificial sequence
  Synthetic construct
uaaaccuuuagcuccgacc

SEQ ID NO: 74 - RNA, 19 nt, Artificial sequence
  Synthetic construct
cagcuuugugugagcguau

SEQ ID NO: 75 - RNA, 19 nt, Artificial sequence
  Synthetic construct
auacgcucacacaaagcug

SEQ ID NO: 76 - RNA, 9 nt, Artificial sequence
  Synthetic construct
guccuucaa

SEQ ID NO: 77 - DNA, 12 bp, Artificial Sequence
  Synthetic construct
tctctctctttt

SEQ ID NO: 78 - DNA, 21 bp, Artificial sequence
  Synthetic construct
agcuuaaccuguccuucaatt

SEQ ID NO: 79 - DNA, 21 bp, Artificial sequence
  Synthetic construct
uugaaggacagguuaagcutt

SEQ ID NO: 80 - DNA, 21 bp, Artificial sequence
  Synthetic construct
accuguccuucaauuaccatt

SEQ ID NO: 81 - DNA, 21 bp, Artificial sequence
  Synthetic construct
ugguaauugaaggacaggutt

SEQ ID NO: 82 - RNA, 19 nt, Artificial sequence
  Synthetic construct
aaaaaaaacuguccuucaa

SEQ ID NO: 83 - RNA, 19 nt, Artificial sequence
  Synthetic construct
aaaaaaaaauguccuucaa

SEQ ID NO: 84 - RNA, 19 nt, Artificial sequence
  Synthetic construct
aaaaaaaaaaguccuucaa

SEQ ID NO: 85 - RNA, 20 nt, Artificial sequence
  Synthetic construct
uguccuucaauguccuucaa

SEQ ID NO: 86 - RNA, 19 nt, Artificial sequence
  Synthetic construct
agcuuaaccuguccuucaa

SEQ ID NO: 87 - RNA, 47 nt, Artificial sequence
  Synthetic construct
agcuuaaccuguccuucaacuacacaaauugaaggacagguuaagcu

SEQ ID NO: 88 - RNA, 20 nt, Artificial sequence
  Synthetic construct
ggacugcguucgcgcuuucc

SEQ ID NO: 89 - RNA, 22 nt, Artificial sequence
  Synthetic construct
ggcuuauccauugcacuccgga

SEQ ID NO: 90 - RNA, 19 nt, Artificial sequence
  Synthetic construct
acgaaggugguuuucccag

SEQ ID NO: 91 - RNA, 20 nt, Artificial sequence
  Synthetic construct
uuugugguagugggggacug

SEQ ID NO: 92 - RNA, 20 nt, Artificial sequence
  Synthetic construct
guaguguuugugggggacug

SEQ ID NO: 93 - RNA, 20 nt, Artificial sequence
  Synthetic construct
guagugggggacuguuugug

SEQ ID NO: 94 - RNA, 20 nt, Artificial sequence
  Synthetic construct
ggacugcguuguggcuuucc

SEQ ID NO: 95 - RNA, 12 nt, Artificial sequence
  Synthetic construct
gauacuuaccug

SEQ ID NO: 96 - RNA, 9 nt, Artificial sequence
  Synthetic construct
aauuugugg

SEQ ID NO: 97 - RNA, 9 nt, Artificial sequence
  Synthetic construct
aauuuuuga

SEQ ID NO: 98 - RNA, 11 nt, Artificial sequence
  Synthetic construct
raunnnnnngr

SEQ ID NO: 99 - RNA, 16 nt, Artificial sequence
  Synthetic construct
gacuagcuugcuguuu

SEQ ID NO: 100 - RNA, 11 nt, Artificial sequence
  Synthetic construct
gacuagccuuu

SEQ ID NO: 101 - DNA, 20 bp, Artificial sequence
  Synthetic construct
ggtgcatcgatgcagggggg

SEQ ID NO: 102 - DNA, 21 bp, Artificial sequence
  Synthetic construct
tcgtcgtttttcggtcgtttt

SEQ ID NO: 103 - DNA, 22 bp, Artificial sequence
  Synthetic construct
tcgtcgttttcggcgcgcgccg

SEQ ID NO: 104 - DNA, 7 bp, Artificial sequence
  Synthetic construct
tcgncgn

SEQ ID NO: 105 - DNA, 7 bp, Artificial sequence
  Synthetic construct
tcgntcg

SEQ ID NO: 106 - DNA, 7 bp, Artificial sequence
  Synthetic construct
tcgacgt

SEQ ID NO: 107 - DNA, 7 bp, Artificial sequence
  Synthetic construct
tcgatcg

SEQ ID NO: 108 - DNA, 24 bp, Artificial sequence
  Synthetic construct
tgctgcttttgtgcttttgtgctt

SEQ ID NO: 109 - DNA, 24 bp, Artificial sequence
  Synthetic construct
tcctccttttgtccttttgtcctt

SEQ ID NO: 110 - RNA, 19 nt, Artificial sequence
  Synthetic construct
agcuuaaccuguccuucaa

SEQ ID NO: 111 - RNA, 24 nt, Artificial sequence
  Synthetic construct
ggggcugacccugaaguucaucuu

SEQ ID NO: 112 - RNA, 24 nt, Artificial sequence
  Synthetic construct
ggggaugaacuucagggucagcuu

SEQ ID NO: 113 - RNA, 24 nt, Artificial sequence
  Synthetic construct
ggggcugacccugaaguucaucuu

SEQ ID NO: 114 - RNA, 24 nt, Artificial sequence
  Synthetic construct
ggggaugaacuucagggucagcuu

SEQ ID NO: 115 - RNA, 20 nt, Artificial sequence
  Synthetic construct
ggugcaucgaugcagggggg

SEQ ID NO: 116 - RNA, 20 nt, Artificial sequence
  Synthetic construct
ggugcuucguugcagggggg

SEQ ID NO: 117 - RNA, 20 nt, Artificial sequence
  Synthetic construct
ggugcuucgaugcagggggg

SEQ ID NO: 118 - RNA, 20 nt, Artificial sequence
  Synthetic construct
ggugcuacguugcagggggg

SEQ ID NO: 119 - DNA, 24 bp, Artificial sequence
  Synthetic construct
tcatcattttgtcattttgtcatt

SEQ ID NO: 120 - DNA, 24 bp, Artificial sequence
  Synthetic construct
tcattattttgttattttgtcatt

SEQ ID NO: 121 - DNA, 24 bp, Artificial sequence
  Synthetic construct
tcatccttttgtccttttgtcatt

SEQ ID NO: 122 - DNA, 24 bp, Artificial sequence
  Synthetic construct
tcatctttttgtctttttgtcatt

SEQ ID NO: 123 - DNA, 24 bp, Artificial sequence
  Synthetic construct
tcatcaatttgtcaatttgtcatt

SEQ ID NO: 124 - DNA, 24 bp, Artificial sequence
  Synthetic construct
tcatcatcttgtcatcttgtcatt

SEQ ID NO: 125 - DNA, 24 bp, Artificial sequence
  Synthetic construct
tcatcatgttgtcatgttgtcatt

SEQ ID NO: 126 - DNA, 24 bp, Artificial sequence
  Synthetic construct
tcatcattctgtcattctgtcatt

SEQ ID NO: 127 - DNA, 24 bp, Artificial sequence
  Synthetic construct
tcatcattgtgtcattgtgtcatt

SEQ ID NO: 128 - DNA, 24 bp, Artificial sequence
  Synthetic construct
tcatcatttggtcatttggtcatt

SEQ ID NO: 129 - DNA, 24 bp, Artificial sequence
  Synthetic construct
tcatttttttgtttttttgtcatt

SEQ ID NO: 130 - DNA, 24 bp, Artificial sequence
  Synthetic construct
tcattgttttgttgttttgtcatt

SEQ ID NO: 131 - DNA, 24 bp, Artificial sequence
  Synthetic construct
tcattcttttgttcttttgtcatt

SEQ ID NO: 132 - DNA, 36 bp, Artificial sequence
  Synthetic construct
aagaggtggtggaggaggtggtggaggaggtggagg

SEQ ID NO: 133 - DNA, 27 bp, Artificial sequence
  Synthetic construct
ttgaattcctagtttcccagatacagt

SEQ ID NO: 134 - DNA, 11 bp, Artificial sequence
  Synthetic construct
tcggtaacggg

SEQ ID NO: 135 - DNA, 18 bp, Artificial sequence
  Synthetic construct
ttagggttagggttaggg

SEQ ID NO: 136 - DNA, 5 bp, Artificial sequence
  Synthetic construct
cgtta

SEQ ID NO: 137 - DNA, 8 bp, Artificial sequence
  Synthetic construct
gccactgc

SEQ ID NO: 138 - DNA, 8 bp, Artificial sequence
  Synthetic construct
gcagtggc

SEQ ID NO: 139 - DNA, 2295 bp, Bacillus anthracis
atgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtcaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataa

SEQ ID NO: 140 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 141 - DNA, 564 bp, Bacillus anthracis
atgttattaatcggcacagaagtaaaaccgtttaaagctaatgcttaccataatggagaatttatccaagttactgacgaaagtttaaaaggaaaatggagtgtagtttgtttctacccagctgacttcacattcgtttgcccaactgaacttgaagacttacaaaaccaatatgcaactcttaaagagttaggcgttgaagtatactctgtatctacagacactcacttcactcacaaagcatggcatgatagctcagaaactatcggtaaaatcgagtacatcatgattggtgacccaactcgcacaatcactacaaacttcaacgttttaatggaagaagaaggtcttgctgctcgtggtacattcatcatcgatccagacggtgttatccaatctatggaaatcaatgctgacggtatcggccgtgacgcaagcattcttgttaacaaaattaaagcagctcaatacgtacgtaacaacccaggtgaagtttgcccagctaaatggcaagagggttctgctacacttaaaccaagccttgaccttgtaggcaaaatctaa

SEQ ID NO: 142 - protein, 187 aa, Bacillus anthracis
MLLIGTEVKPFKANAYHNGEFIQVTDESLKGKWSVVCFYPADFTFVCPTELEDLQNQYATLKELGVEVYSVSTDTHFTHKAWHDSSETIGKIEYIMIGDPTRTITTNFNVLMEEEGLAARGTFIIDPDGVIQSMEINADGIGRDASILVNKIKAAQYVRNNPGEVCPAKWQEGSATLKPSLDLVGKI

SEQ ID NO: 143 - DNA, 1170 bp, Bacillus anthracis
atggaagaagcaccattttatcgtgacacttgggtggaagtggatttagatgccatttataacaacgttacacatattaaagaatttatcccgagtgatgtagaaatttttgccgttgttaaagggaatgcatatgggcacgattatgtaccggtggctaaaatagcattagaagcgggggcgacaaggttagcagttgcgttcttagatgaagctttagtgcttcgaagagctggtattactgcgccaattttggtgttaggtccttctcctcctcgtgatataaatgtagctgctgaaaatgatgtagcattaactgtttttcaaaaggaatgggtagatgaagcaatcaaactttgggatggttcgtctacgatgaaataccatattaatttcgatagtggtatggggagaattggaatacgtgaacgtaaagaattaaaaggatttttaaaaagcttagaaggtgcaccattcttagagttggaaggagtttatacgcattttgcaacagcagatgaggtggagacttcttactttgataagcaatataacacatttttggagcagttaagttggttgaaagaattcggagtggatcctaagtttgttcatacagctaatagtgctgcaacgctacgttttcaagggattacatttaatgcagtacgaattggcattgcgatgtatgggttatctccatctgtagaaatacgcccttttttaccgtttaaattagaaccagcgctatcattgcatacgaaagttgctcatattaaacaggtgattaaaggggatggaattagttataacgtcacttatcgaacgaaaactgaagaatggattgcgaccgttgcaattggttatgcagatggctggcttagaagattacaaggatttgaagtacttgtaaatggtaaaagggtaccgattgtagggcgagtaacgatggatcaattcatgattcaccttccttgtgaagtgcctcttggtacgaaagttacactcattggaaggcaaggagatgaatatattagcgctacagaggttgcggaatattcagggactattaattatgaaattattacgacgatcagttttcgtgtgccgagaatatttatacggaatggaaaagttgtggaagtaattaattatttgaacgatatatag

SEQ ID NO: 144 - protein, 389 aa, Bacillus anthracis
MEEAPFYRDTWVEVDLDAIYNNVTHIKEFIPSDVEIFAVVKGNAYGHDYVPVAKIALEAGATRLAVAFLDEALVLRRAGITAPILVLGPSPPRDINVAAENDVALTVFQKEWVDEAIKLWDGSSTMKYHINFDSGMGRIGIRERKELKGFLKSLEGAPFLELEGVYTHFATADEVETSYFDKQYNTFLEQLSWLKEFGVDPKFVHTANSAATLRFQGITFNAVRIGIAMYGLSPSVEIRPFLPFKLEPALSLHTKVAHIKQVIKGDGISYNVTYRTKTEEWIATVAIGYADGWLRRLQGFEVLVNGKRVPIVGRVTMDQFMIHLPCEVPLGTKVTLIGRQGDEYISATEVAEYSGTINYEIITTISFRVPRIFIRNGKVVEVINYLNDI

SEQ ID NO: 145 - DNA, 360 bp, Bacillus anthracis
atgactaaagaacaaatcattgaagcagttaaatctatgactgtattagaacttaacgacttagtaaaagctatcgaggaagaattcggcgtaactgctgctgctcctgtagctgttgctggtggcgctggagaagctgctgctgagaaaactgaatttgatgtggaactaactagcgctggtgcacaaaaaatcaaagttatcaaagttgttcgtgaaatcactggtcttggcttaaaagaagctaaagaattagttgacaacactccaaaagtaatcaaagaagctgctgctaaagaagaagctgaagaaatcaaagctaaacttgaagaagttggcgctgctgtagaagttaagtaa

SEQ ID NO: 146 - protein, 119 aa, Bacillus anthracis
MTKEQIIEAVKSMTVLELNDLVKAIEEEFGVTAAAPVAVAGGAGEAAAEKTEFDVELTSAGAQKIKVIKVVREITGLGLKEAKELVDNTPKVIKEAAAKEEAEEIKAKLEEVGAAVEVK

SEQ ID NO: 147 - DNA, 1707 bp, Bacillus anthracis
atgaagaaaaagatgaagaagttcacggcagttgtagcgcctgttttagcgatgagtgtggcgttgacagcttgttctggatctggtggggagaagaaatcaactacgacgtctagtggtggtggggaagagaaaaagtctgaaattaaatacgcagcgaaacaagtgttaaatcgtacagagaatcaagagattccgacgatggatgtttcaaaatctaccgatacattaggttctcaaattttagggaacacgatggaaggtttatatcgattagataaagataataagccaatcccagctgcagcagaatctagtacgaaaagcgaggatggcaaaaaatatacatttaaattacgtaaagatgcaaaatggtcaaatggtgatcctgtaacagcgaaagatttcgtatatgcatggcagcgcttacttgataaaaatacagcggcagaatatgcatttattgcttactatattaaaaacgcagaggcaattaataaaggtgaaaaaccactaacagatttaggagcaaaagcggtagatgattatacgctagaagtagaattagagaaaccagtaccatatttcttgaatttaatggcattcccatcttactatcctttaaatgaaaagttcgtaaaagaaaaaggagataaattcggtttagaagcagatacaacgttgtataacggaccgttcgttatggcttcatggaaacatgaacaaggatggcagctaaagaaaaatgataagtactgggataataagactgtaaaattagaagaaattaactatagtgtagtaaaagaagttgcgacgaaagtaaacttatatgatacaggatcaattgatttcacgttattatcaggagaattcgttgataaatataaatcgaacaaagaagagtacggcgagtattcggaagcaagtacattcttcttacgtttaaatcaaaagcgtaacggacaagatacaccgttaaagagcaaaaaacttcgtgaagcgatcgcattatcaattgataaaaaaggattagcaaccgttattttaaataacggttcaaaagcaacagatcaattagtaccaaaagggcttgcgacaggaccagacggtaaagactaccaagatacgtttaaaaatggtctaaaatatgatccgaaaaaaggtgcagcagcttgggaagaagcgaaaaaagaacttggaaaagatcaagtgacaattgaattactaagctatgatgatggaactgcgaaaaaaattgctgactactttaaagatcaaattgagaaaaacttaaaaggtgtaacggttaacacgaaaattcaaccgttcaaacaaaaactaaaattagagtcagcacaagattatgaagtttcgtttgcaggttggagtccagattattcggatccaatgacatttattgatatgtttgaatcgaagagcccatataaccaaatgagttattcgaatccaaaatatgatgaaatggtagcgaaagcaggtaatgaattactgtctgatccgaagaagcgttgggaaacgttaggaaaagcagagaaattattccttgaagaagatgcaggattagttcctttatatcaaacaggaagagcgtatgtaatgaaaccgaatgtaaaaggaattgtgaaacataacattagtccagaatatagctttaagtgggcttatgtaacggaaggtaaataa

SEQ ID NO: 148 - protein, 568 aa, Bacillus anthracis
MKKKMKKFTAVVAPVLAMSVALTACSGSGGEKKSTTTSSGGGEEKKSEIKYAAKQVLNRTENQEIPTMDVSKSTDTLGSQILGNTMEGLYRLDKDNKPIPAAAESSTKSEDGKKYTFKLRKDAKWSNGDPVTAKDFVYAWQRLLDKNTAAEYAFIAYYIKNAEAINKGEKPLTDLGAKAVDDYTLEVELEKPVPYFLNLMAFPSYYPLNEKFVKEKGDKFGLEADTTLYNGPFVMASWKHEQGWQLKKNDKYWDNKTVKLEEINYSVVKEVATKVNLYDTGSIDFTLLSGEFVDKYKSNKEEYGEYSEASTFFLRLNQKRNGQDTPLKSKKLREAIALSIDKKGLATVILNNGSKATDQLVPKGLATGPDGKDYQDTFKNGLKYDPKKGAAAWEEAKKELGKDQVTIELLSYDDGTAKKIADYFKDQIEKNLKGVTVNTKIQPFKQKLKLESAQDYEVSFAGWSPDYSDPMTFIDMFESKSPYNQMSYSNPKYDEMVAKAGNELLSDPKKRWETLGKAEKLFLEEDAGLVPLYQTGRAYVMKPNVKGIVKHNISPEYSFKWAYVTEGK

SEQ ID NO: 149 - DNA, 1485 bp, Bacillus anthracis
atgagtcaactagctgtaaatcttcatgaaaaggtagaaaagtttcttcaaggtacgaaaaagttatatgtgaatggatcattcattgaaagcgcttccggtaagacgtttaatacacctaatccagcaactggcgaaacacttgccgtcgtttctgaagccggtcgcgaagatattcataaagctgtagttgcagctcgcatggcttttgacgaaggtccttggtctcgcatgagcactgcggagcgaagccgtcttatgtacaagttagctgatttaatggaagaacataaagaagagcttgcacagctcgagacgttagataacggaaagccaatccgtgaaacaatggcagcagacataccacttgcaattgagcacatgcgctattatgctggctgggcgacgaaaatcgttggtcaaacaatccctgtttccggtgatttctttaactatacacgccatgaagctgttggtgtcgttggtcaaattatcccttggaacttcccgcttcttatggccatgtggaaaatgggagcagcgcttgctacaggatgtacaatcgttttaaaacctgcagaacaaactccactatctgctctatacttagctgaattaattgaagaagctggattcccgaaaggcgttattaatatcgttcctggattcggtgaatcagctggacaagctctcgttaatcatccactcgttgataaaattgcatttaccggttctactccagtcggtaaacaaattatgcgacaagcatctgaatccttgaaacgtgttactttagagcttggtggtaaatcaccgaacattattttaccagacgctgatttatctcgcgcaattcctggtgcactttctggtgttatgtttaaccaagggcaagtatgctctgctggatcacgcctatttgttccgaagaaaatgtatgataatgtcatggctgatctcgtcctctattctaaaaaactaaatcaaggtgtcggtcttgaccctgaaacgacaattggtcctctcgtttccgaagaacaacaaaaacgtgtaatgggctacattgaaaaagggattgaagaaggcgctgaagtactttgcggaggaaataatccattcgatcaaggctacttcatttctcctacagtattcgctgacgtaaatgacgaaatgacaatcgcaaaagaagaaattttcggtccagttatttctgcaataccttttaacgatattgatgaagtaattgaacgagcaaataaatcacaattcggcttagcggctggtgtgtggacagaaaatgttaaaacagcacactatgttgcaagtaaagtacgtgcaggtacagtatgggttaactgttacaacgtctttgatgcagcatctccatttggaggatttaaacaatctggtctcggccgtgaaatgggatcttacgcattaaataactatacagaagtgaagagcgtttggcttaacttaaattaa

SEQ ID NO: 150 - protein, 494 aa, Bacillus anthracis
MSQLAVNLHEKVEKFLQGTKKLYVNGSFIESASGKTFNTPNPATGETLAVVSEAGREDIHKAVVAARMAFDEGPWSRMSTAERSRLMYKLADLMEEHKEELAQLETLDNGKPIRETMAADIPLAIEHMRYYAGWATKIVGQTIPVSGDFFNYTRHEAVGVVGQIIPWNFPLLMAMWKMGAALATGCTIVLKPAEQTPLSALYLAELIEEAGFPKGVINIVPGFGESAGQALVNHPLVDKIAFTGSTPVGKQIMRQASESLKRVTLELGGKSPNIILPDADLSRAIPGALSGVMFNQGQVCSAGSRLFVPKKMYDNVMADLVLYSKKLNQGVGLDPETTIGPLVSEEQQKRVMGYIEKGIEEGAEVLCGGNNPFDQGYFISPTVFADVNDEMTIAKEEIFGPVISAIPFNDIDEVIERANKSQFGLAAGVWTENVKTAHYVASKVRAGTVWVNCYNVFDAASPFGGFKQSGLGREMGSYALNNYTEVKSVWLNLN

SEQ ID NO: 151 - DNA, 1410 bp, Bacillus anthracis
atgaataaagggcgcgttacgcaaatcatgggtccggttgtagacgttaagtttgatggcggaaagctaccagaaatctacaacgcccttacggtaaaacagagcaacgaaaacggaacaagcattaacttaacatttgaagttgcacttcatttaggtgatgacacagttcgtacagttgcaatgtcttccacagatggacttgttcgtggcacagaagtagaagatactggtaaagcaatctctgtaccagttggtgatgcaacacttggtcgtgtatttaacgtattaggtgatgcaattgacttagatggtgaggttcctgcggatgtacgtcgtgatccaattcaccgtcaagcacctgcattcgaagaattatctactaaagtagaaattcttgaaactggtattaaagtagtagacttacttgctccttacattaagggtggtaagatcggtctattcggtggtgccggtgtaggtaaaacggtattaattcaggaattaatcaataacatcgcacaagaacacggtggtatctctgtattcgctggtgtaggtgagcgtactcgtgagggtaatgacttataccacgaaatgagcgattctggcgtaattaagaaaactgcgatggtattcggacaaatgaacgagccacctggagcacgtcaacgtgttgcgttaacaggtttaacaatggctgagcatttccgtgatgagcaaggacaagatgtacttctgttcatcgataatatcttccgtttcacgcaagcaggttctgaagtatctgcccttcttggccgtatgccatctgcggtaggttaccaaccaacacttgcaacagaaatgggtcaattacaagagcgtattacatctacaaataaagggtctatcacgtctatccaagcggtatatgtaccagccgatgactatactgacccagcaccagctacaacgttcgctcacttagatgcaacaacaaacttagagcgtcgtttaacacaaatgggtatttacccagccgtagatccattagcatctacatctcgtgcactttctccagaaatcgtaggagaagagcattatgaagtggctcgtcaagtacagcaaactttacaacgctacaaagagcttcaagatatcatcgctatcttaggtatggatgagttatctgaagaagataagttagttgtacatcgtgctcgtcgtattcaattcttcttatctcaaaacttccacgtagcggagcagtttacaggtcaaaaaggttcttatgtacctgtaaaagaaacagttcgtggtttcaaagaaattctagaaggaaaatatgatgaccttccagaagatgcattccgcttagttggtggcattgaagaagttattgaaaacgcgaagaaaatgatggcgtaa

SEQ ID NO: 152 - protein, 469 aa, Bacillus anthracis
MNKGRVTQIMGPVVDVKFDGGKLPEIYNALTVKQSNENGTSINLTFEVALHLGDDTVRTVAMSSTDGLVRGTEVEDTGKAISVPVGDATLGRVFNVLGDAIDLDGEVPADVRRDPIHRQAPAFEELSTKVEILETGIKVVDLLAPYIKGGKIGLFGGAGVGKTVLIQELINNIAQEHGGISVFAGVGERTREGNDLYHEMSDSGVIKKTAMVFGQMNEPPGARQRVALTGLTMAEHFRDEQGQDVLLFIDNIFRFTQAGSEVSALLGRMPSAVGYQPTLATEMGQLQERITSTNKGSITSIQAVYVPADDYTDPAPATTFAHLDATTNLERRLTQMGIYPAVDPLASTSRALSPEIVGEEHYEVARQVQQTLQRYKELQDIIAILGMDELSEEDKLVVHRARRIQFFLSQNFHVAEQFTGQKGSYVPVKETVRGFKEILEGKYDDLPEDAFRLVGGIEEVIENAKKMMA

SEQ ID NO: 153 - DNA, 582 bp, Bacillus anthracis
atgaatttaattcctacagtaattgaacaaacaaatcgtggagaacgcgcttacgatatttactctcgactattaaaagaccgtatcattatgcttggtagtgcaattgatgacaacgtagctaactcaatcgtttcccagcttttattcttggaatctcaagatcctgaaaaagatattcatatctacatcaacagccctggtggttctatcacagcaggtatggcaatttacgatacaatgcagtttattaaaccgcaagtatcaacaatctgtatcggtatggctgcatctatgggtgcattcttacttgcagcaggtgaaaaaggaaaacgttatgcacttccaaacagtgaagcaatgattcaccaaccacttggtggggcacaaggtcaagcgactgaaatcgaaatcgctgctaaacgtatcctattcttacgtgaaaaactaaaccaaattcttgctgaccgcacaggtcaaccacttgaagtactacaacgcgacacagaccgcgacaacttcatgacagcagaaaaagctttagaatacggtttaatcgataagatctttacaaatcgttaa

SEQ ID NO: 154 - protein, 193 aa, Bacillus anthracis
MNLIPTVIEQTNRGERAYDIYSRLLKDRIIMLGSAIDDNVANSIVSQLLFLESQDPEKDIHIYINSPGGSITAGMAIYDTMQFIKPQVSTICIGMAASMGAFLLAAGEKGKRYALPNSEAMIHQPLGGAQGQATEIEIAAKRILFLREKLNQILADRTGQPLEVLQRDTDRDNFMTAEKALEYGLIDKIFTNR

SEQ ID NO: 155 - DNA, 570 bp, Bacillus anthracis
atgtggatttatgaaaaaaaattacaataccctgttaaagtaggaacttgtaatccagcacttgcaaaattattaattgagcaatacggtggtgcagatggagaattagctgctgcactacgttacttaaatcagcgttatacaatcccggataaagtcattggcctccttaccgatattggtacagaagaatttgcgcatcttgaaatgattgctacgatggtttataagctgacaaaagatgcgactcctgaacagatgaaggcagctggtctcgaccctcattacgtcgatcatgacagcgcacttcattaccataacgcagctggtgttccatttactgcaacctatatacaagctaaaggtgatccaattgccgacctatacgaagatattgcggctgaagaaaaagcgcgtgccacatatcaatggcttatcaaccaatctgacgatcccgacataaatgacagtttacgctttttacgcgaacgagaaattgtccattcacaacgtttccgagaagcggttgaaattttaaaagaagaacgcgatagaaaaatatatttttaa

SEQ ID NO: 156 - protein, 189 aa, Bacillus anthracis
MWIYEKKLQYPVKVGTCNPALAKLLIEQYGGADGELAAALRYLNQRYTIPDKVIGLLTDIGTEEFAHLEMIATMVYKLTKDATPEQMKAAGLDPHYVDHDSALHYHNAAGVPFTATYIQAKGDPIADLYEDIAAEEKARATYQWLINQSDDPDINDSLRFLREREIVHSQRFREAVEILKEERDRKIYF

SEQ ID NO: 157 - DNA, 1077 bp, Bacillus anthracis
atggcaaatcatgaattagatcaattacgtaaacaggtagatgaaattaacttacaactattacaccttttaaacaaacgcggtgaaatcgttcaaaaaattggggaacaaaagcaagtacaaggtacaaaacgttttgatccagtacgtgagcgtgaagtgcttgatatgattgcagagaataacgaaggaccattcgaaacatcaacagttcaacatattttcaaaacaatcttcaaagctagcttagaattacaagaagatgataaccgtaaagcattactagtatcacgtaaaaagaaacaagaaaacacaatcgttgatgtaaaaggtgaattgattggtaacggcacacaaacgttcatcatgggaccttgcgcggtagaaagcttagagcaagttcgccaagtagggcaagcgatgaaagaccaaggcttaaaattaatgcgcggtggtgctttcaaaccgagaacatctccatacgatttccaaggtttaggagtagaagggctacaaattttacgtcaagtagcagatgagttcgacttagcgatcattagtgagattttaaatccaaacgatgttgaaatggcattagactacgttgatgtaattcaagttggtgcacgtaacatgcaaaacttcgatttactacgagctgtaggtaaagttaacaagccagtattattaaaacgtggattagcagcaacaattgatgagttcattaatgcagcggaatacatcattgcacaaggtaatgaccaaattattctatgtgagcgcggtattcgcacatacgaaagagcaacacgtaacacattagacatttcagcagtaccgatcttaaagaaagaaacacatttaccagttgttgttgacgtaacgcattcaactggacgtagagatttattattaccaacagcgaaagcggctcttgcaattggtgcagatgcagtaatggctgaagtacatccagacccagcagttgcattatcagattctgcacaacaaatggatattccggaattccatagattcatggaagagttaaaaggtttcaaaaataaattatcttaa

SEQ ID NO: 158 - protein, 358 aa, Bacillus anthracis
MANHELDQLRKQVDEINLQLLHLLNKRGEIVQKIGEQKQVQGTKRFDPVREREVLDMIAENNEGPFETSTVQHIFKTIFKASLELQEDDNRKALLVSRKKKQENTIVDVKGELIGNGTQTFIMGPCAVESLEQVRQVGQAMKDQGLKLMRGGAFKPRTSPYDFQGLGVEGLQILRQVADEFDLAIISEILNPNDVEMALDYVDVIQVGARNMQNFDLLRAVGKVNKPVLLKRGLAATIDEFINAAEYIIAQGNDQIILCERGIRTYERATRNTLDISAVPILKKETHLPVVVDVTHSTGRRDLLLPTAKAALAIGADAVMAEVHPDPAVALSDSAQQMDIPEFHRFMEELKGFKNKLS

SEQ ID NO: 159 - DNA, 504 bp, Bacillus anthracis
atgttctcttctgattgcgaatttactaaaattgattgcgaggcaaaaccagctagtacactacctgccttcggttttgctttcaacgcgtctgcacctcagttcgcttcattatttacaccactactattacctagcgtaagtccaaacccaaatattactgttcctgtaataaatgatacagtaagtgtcggagatggcattcgaattctacgagctggtatttatcaaatcagttatacattaacaattagtcttgataactcacctgttgcaccagaagctggtcgtttcttcttatcattaggtacaccagctaacattattcctggatcaggtacagcggttcgttctaacgttattggtactggtgaagtagacgtatccagcggtgttattcttattaacttaaaccctggtgacttaatcagaatcgtaccagttgaattgattggaactgtagacatccgtgcagcagcattaacagttgcacaaattagctag

SEQ ID NO: 160 - protein, 167 aa, Bacillus anthracis
MFSSDCEFTKIDCEAKPASTLPAFGFAFNASAPQFASLFTPLLLPSVSPNPNITVPVINDTVSVGDGIRILRAGIYQISYTLTISLDNSPVAPEAGRFFLSLGTPANIIPGSGTAVRSNVIGTGEVDVSSGVILINLNPGDLIRIVPVELIGTVDIRAAALTVAQIS

SEQ ID NO: 161 - DNA, 504 bp, Bacillus anthracis
atgcgatcatctagtcgtaagctcacaaactttaattgtagagcacaagcccccagtacactaccagctctcggttttgcttttaatgctacttcacctcaatttgcaacactatttacaccactactactacctagtacaggcccaaatccaaacattactgtccctgtaatcaatgatacaattagtacaggaactggtattagaattcaagtagctggtatttatcaaatcagttatacattaacaatcagcctcgataatgttccagtaaccccggaagcagcgcgctttttcttaacactaaactcatcaactaatattattgcaggatctggaaccgcagtccgttctaatatcattggcactggtgaagtagatgtatccagcggtgtcattctaataaacttaaaccctggtgatttaattcaaattgtacccgttgaagtaattggtacagtagatattcgttctgccgctttaacagttgcacaaattcgttaa

SEQ ID NO: 162 - protein, 167 aa, Bacillus anthracis
MRSSSRKLTNFNCRAQAPSTLPALGFAFNATSPQFATLFTPLLLPSTGPNPNITVPVINDTISTGTGIRIQVAGIYQISYTLTISLDNVPVTPEAARFFLTLNSSTNIIAGSGTAVRSNIIGTGEVDVSSGVILINLNPGDLIQIVPVEVIGTVDIRSAALTVAQIR

SEQ ID NO: 163 - DNA, 966 bp, Bacillus anthracis
gtggaaagaagtttatctatggagttagtacgtgtaacagaggctgcagctttatcatcagcgcgttggatgggacgcgggaaaaaggatgaggcagacggtgcagcaacatcagctatgcgtgatgtatttgatacaattccgatgaaaggtacagttgtaattggtgaaggtgaaatggatgaagcaccaatgctatatatcggagaaaaattaggtacaggatatggtccacgtgtagacgttgcagttgatcctttagaagggacaaacattgtagcagctggtggatggaatgctcttgctgttattgcaattgcagatcacggtaatttgttacatgctcctgacatgtacatggataaaatcgcggttggcccagaagcggttggggcggtcgatattgatgcgcctattatcgataacttacgtgcagttgcgaaagcgaaaaacaaggatattgaagatgttgtagcgacagttttaaaccgtccacgtcatcaagcgattattgaagaaattcgtaaagctggtgctcgtattaaattgattaatgatggagacgtagcaggtgcaattaatactgcatttgatcgtacaggtgtagatattttattcggatctggtggtgcgcctgagggtgtattagcagcagttgcattaaaatgtttaggtggcgaaattcacggaaagctattaccacaaaacgaagctgaattggcgcgttgcaaaaagatgggcatagaagacatcaaccgcatccttcgcatggaggacttagtaaaaggtgacgatgcaatctttgcagcaacaggtgtaacagacggagaactattacgcggcgttcaatttaaaggtagcgtaggaacaacacaatctcttgttatgcgtgcaaaatcaggcacagtacgcttcgtagacggacgtcatagcttaaataaaaaaccgaacttggttattaaataa

SEQ ID NO: 164 - protein, 321 aa, Bacillus anthracis
VERSLSMELVRVTEAAALSSARWMGRGKKDEADGAATSAMRDVFDTIPMKGTVVIGEGEMDEAPMLYIGEKLGTGYGPRVDVAVDPLEGTNIVAAGGWNALAVIAIADHGNLLHAPDMYMDKIAVGPEAVGAVDIDAPIIDNLRAVAKAKNKDIEDVVATVLNRPRHQAIIEEIRKAGARIKLINDGDVAGAINTAFDRTGVDILFGSGGAPEGVLAAVALKCLGGEIHGKLLPQNEAELARCKKMGIEDINRILRMEDLVKGDDAIFAATGVTDGELLRGVQFKGSVGTTQSLVMRAKSGTVRFVDGRHSLNKKPNLVIK

SEQ ID NO: 165 - DNA, 786 bp, Bacillus anthracis
atgtttagcttaaaagggactgttatgaaaaccgcacttcttgcatccgtcgcaatgttgttcacaagctcggctatggctgccgacatcatcgttgctgaaccggcacccgttgcagtcgacacgttctcttggactggcggctatattggtatcaatgctggttacgctggcggcaagttcaagcatccgttctcaggcatcgagcaggatggggcccaagatttttcaggttcgctcgacgtcacggccagcggctttgttggcggcgttcaggccggttataactggcagcttgccaacggcctcgtgcttggtggcgaagctgacttccagggctcgacggttaagagcaagcttgttgacaacggtgacctctccgatatcggcgttgcaggcaacctcagcggcgacgaaagcttcgtcctcgagaccaaggttcagtggtttggaacggtgcgtgcgcgcctcggcttcaccccgactgaacgcctgatggtctatggtaccggtggtttggcctatggtaaggtcaagacgtcgcttagcgcctatgacgatggtgaatcgttcagcgccggaaactctaagaccaaggctggctggacgcttggtgcaggtgtagaatacgccgtcaccaacaattggaccctgaagtcggaatacctctacaccgacctcggcaagcgttccttcaattacattgatgaagaaaacgtcaatattaacatggaaaacaaggtgaacttccacaccgtccgcctcggtctgaactacaagttctaa

SEQ ID NO: 166 - protein, 261 aa, Bacillus anthracis
MFSLKGTVMKTALLASVAMLFTSSAMAADIIVAEPAPVAVDTFSWTGGYIGINAGYAGGKFKHPFSGIEQDGAQDFSGSLDVTASGFVGGVQAGYNWQLANGLVLGGEADFQGSTVKSKLVDNGDLSDIGVAGNLSGDESFVLETKVQWFGTVRARLGFTPTERLMVYGTGGLAYGKVKTSLSAYDDGESFSAGNSKTKAGWTLGAGVEYAVTNNWTLKSEYLYTDLGKRSFNYIDEENVNINMENKVNFHTVRLGLNYKF

SEQ ID NO: 167 - DNA, 1371 bp, Bacillus anthracis
gtcgttcttggtttaccagttgatccaaaagcaaagccatcatttaaagatgcacaaaaccattgggcagctccgtacattgctgcagtggaaaaagcaggtgtaattaatggggatggtactggtaaattcaatccatcaagccaaattaaccgtgcatctatggcatctatgttagtacaagcatactcattagataagaaaattattggagaacttccaacacagtttaaagatttggaacctcattggggtaagaaacaagctaatattttagtagctttagagatttctaaaggtacgggaaatggctggaatcctgaaggaactgtaactcgtgcagaagcagctcagtttattgcgatggctgatcaaaataaaacaagtacatcaaaaagaatgtatatgaacagaaacgttattacatatcatcaaccatcattatcctctggtattactgatgttcaacataagccacaaatggttgaagtgacagagcaaagagcagacggctggttgaaaattgtaacaagtaaaggtgagaagtggacacctctaacagaaaaaacagaaacgattaatgaagaatttactacttatgaaacagcttcacatagttctaaagtgctaggtacatataatgcacaaacagtaacggttatggaagagagtggtagctggattcgtatccgcgtaggcgctggtttccagtgggttgataaaaatcaattaaatccagtaaaacaagagaactttttagaaggtaaagcaattattattgatccaggtcatggtggaattgactcaggtaatgttggttattacgagaaagaaagtgaaactgtattagatgtatcattacgattaaagaaaatatttgagcaaaaagcaccatttactgttatgttcactcgtacagataatacacgtccaggagtaaactcaacagattcattgaaaaaacgagtagagtttgctcaggaacataatggagatatctttgtaagtatccatgctaatggttctgcagagaaaaatggacaaggtacagaaacattatattatcagtcagcaagagcaaaagtaacgaatccgcatgtagaagacagtaagttattagcacaaaaaattcaagaccgtcttgtagcagcacttggaacaaaagatcgtggtgtgaaacatcaggacttatacgttactagagaaaatacaatgccagctgtattaacagaattagcatttgtagataataaaagtgatgcagataaaattgctacaccaaaacagagacaagctgcagcagaagcgatttatcaaggtattttagattattacgaagcaaagggtaataacgtatcttctttccgttaa

SEQ ID NO: 168 - protein, 456 aa, Bacillus anthracis
VVLGLPVDPKAKPSFKDAQNHWAAPYIAAVEKAGVINGDGTGKFNPSSQINRASMASMLVQAYSLDKKIIGELPTQFKDLEPHWGKKQANILVALEISKGTGNGWNPEGTVTRAEAAQFIAMADQNKTSTSKRMYMNRNVITYHQPSLSSGITDVQHKPQMVEVTEQRADGWLKIVTSKGEKWTPLTEKTETINEEFTTYETASHSSKVLGTYNAQTVTVMEESGSWIRIRVGAGFQWVDKNQLNPVKQENFLEGKAIIIDPGHGGIDSGNVGYYEKESETVLDVSLRLKKIFEQKAPFTVMFTRTDNTRPGVNSTDSLKKRVEFAQEHNGDIFVSIHANGSAEKNGQGTETLYYQSARAKVTNPHVEDSKLLAQKIQDRLVAALGTKDRGVKHQDLYVTRENTMPAVLTELAFVDNKSDADKIATPKQRQAAAEAIYQGILDYYEAKGNNVSSFR

SEQ ID NO: 169 - DNA, 660 bp, Bacillus anthracis
atgaagaagaacatgttacgtataatggcaacggtaactattatgggcggcttgtttgtaagtacgaatgttccaaacgtaaaagcagaagaatatccagaaatgattgtatttgatgatgttccagtaaaccactgggcatatgacgatataatggacgtagtatacaataaagtaatgttaggctatggaaatggtaagtttggtgtaggagataatgtaacacgagaacaagtagctgcagtactctatcgtacattgaatttgaaaaaagaaggacctttaaaaaatccatacaaagatatttcagagagggctacattctttttagatgaaattttagtattaacaaagcatggtatttttgaaggcgatgaaaaaggaaattttagaccagccgcaccagtaacacgtgcagaaacggcgcaaattcttacgaaggcatttacatttgaagtgaagaagaaccatacatttaaagatgtaccaaataatcattgggcaaaaaatgcgattagtgcactgcagtctaatcatgtcatagtaggaacagggaatgggaaatttgaaccgaataaagttgtaacacgtgagcaatatgcaacgtttttaaataaagctgttttttattttccagtaaaagatgagaattatgaatga

SEQ ID NO: 170 - protein, 219 aa, Bacillus anthracis
MKKNMLRIMATVTIMGGLFVSTNVPNVKAEEYPEMIVFDDVPVNHWAYDDIMDVVYNKVMLGYGNGKFGVGDNVTREQVAAVLYRTLNLKKEGPLKNPYKDISERATFFLDEILVLTKHGIFEGDEKGNFRPAAPVTRAETAQILTKAFTFEVKKNHTFKDVPNNHWAKNAISALQSNHVIVGTGNGKFEPNKVVTREQYATFLNKAVFYFPVKDENYE

SEQ ID NO: 171 - DNA, 1437 bp, Bacillus anthracis
atgattaaaacaaatgaaattaaacaaaaagatgcaatattagaagagattacggattatgtattaaataaagaggtaacaagtgcagaagcattcagtactgctcgttacgtattatttgatacacttggatgcggaattttagcattacaatatccagagtgtacgaaattattaggaccagttgtaccaggaacaatcgtgccaaatggaacacgagtgccaggtacgtcttatgtattagatccagtgaaaggtgcatttaatatcggatgtatgatccgttggttagactataacgatacttggcttgcagcagaatggggacatccatctgataaccttggcggcattttagcagttgcagattatattagccgtgttcgtatatcagaaggaaaagaaccgttaaaagtacgtgaagtattagaaatgatgattaaagcacatgaaattcaaggtgtattagctttagaaaacagcttaaaccgggttggtcttgaccacgtattatacgtaaaagtagcaacaactgctgtagttgcgaaaatgcttggcggaacacgtgaagaaatctttaatgcattatcacatgcatggattgataattctagtcttcgtacatatcgtcacgctccaaatactggatcacgtaaatcatgggcagcaggtgatgcaacaagtcgcggtgttcaccttgcaatgactgctttaaaaggtgaaatgggttatccaacagcattatctgcaccgggttggggattccaagatgtattatttaataaacaagaattaaagttagctagaccattagagtcttatgtaatggaaaatgtattatttaaagtttcatatccagcagaattccatgcacaaacagctgcagaatgtgctgtaaaattacatccggaaattaaagaaagattagatgaaattgaccgtattacaattacaactcatgaatcagcaattcgtattattgataaagaaggtccattaaataacccagctgatcgtgatcattgtttacaatatattacggcaattggtttattaaagggagatatcgttgcggatgattatgaggatgcagtagcaaatgatccacgtgtagatgaattacgtaataagatggttgttgttgaaaacaaacagtacagtttagattaccttgacccgaacaagcgctcaatcgccaacgctgttcaagttcatttcaaggatggaactgtaacagaaaacgtggaatgtgaatatccacttggtcaccgtttccgtagagacgaagcaattccaaaagttgttcaaaaattcactgcaagtatggcaggtcattattctagtaaacagcaagaacaaattcatgaagtttgtttaaatgaagagaaactagaaaatatgaatgtaaacgaatttgtagatctattcttaatttaa

SEQ ID NO: 172 - protein, 478 aa, Bacillus anthracis
MIKTNEIKQKDAILEEITDYVLNKEVTSAEAFSTARYVLFDTLGCGILALQYPECTKLLGPVVPGTIVPNGTRVPGTSYVLDPVKGAFNIGCMIRWLDYNDTWLAAEWGHPSDNLGGILAVADYISRVRISEGKEPLKVREVLEMMIKAHEIQGVLALENSLNRVGLDHVLYVKVATTAVVAKMLGGTREEIFNALSHAWIDNSSLRTYRHAPNTGSRKSWAAGDATSRGVHLAMTALKGEMGYPTALSAPGWGFQDVLFNKQELKLARPLESYVMENVLFKVSYPAEFHAQTAAECAVKLHPEIKERLDEIDRITITTHESAIRIIDKEGPLNNPADRDHCLQYITAIGLLKGDIVADDYEDAVANDPRVDELRNKMVVVENKQYSLDYLDPNKRSIANAVQVHFKDGTVTENVECEYPLGHRFRRDEAIPKVVQKFTASMAGHYSSKQQEQIHEVCLNEEKLENMNVNEFVDLFLI

SEQ ID NO: 173 - DNA, 1278 bp, Bacillus anthracis
atggctgcaaaatgggaaaaattagaaggtaacgtaggcgttttaacaatcgaagttgatgctaaagaagtaaacaactctatcgacgctgcgttcaaaaaagtagtaaaaacaatcaacgtaccaggtttccgtaaaggaaaaatgcctcgtccgttattcgaacaacgctttggtatcgaatctttataccaagatgctttagatatcatcttaccaaaagcatacggtgaagcgatcgatgaagctggtatcttcccagttgctcatcctgaaatcgacatcgagaagttcgaaaaaaatgctaaccttatcttcactgcaaaagttacagtgaaacctgaagttaaattaggtgagtacaaaggtttagcagtagaaaaagttgaaacaactgtaactgacgaagatgtagagaacgaattaaaatctttacaagagcgtcaagctgaactagttgttaaagaagaaggaactgttgaaaacggtgatacagctgtaatcgacttcgaaggtttcgttgatggcgaagcatttgaaggcggaaaaggcgaaaactactctctagcaatcggttctggtacattcatcccaggtttcgaagagcaagtaattggtcttaaatctggtgagtctaaagacgttgaagtatcattcccagaagagtaccatgctgctgaattagctggcaaaccagcaacattcaaagtaacagttcacgaaatcaaaacaaaagaacttcctgagttaaacgacgagttcgctaaagaagctgacgaagcggttgcaactcttgatgaattaaaagcaaaacttcgcacaaacttagaagaaggcaaaaagcacgaagctgagcacaaagtacgtgatgaagtagtagaattagctgctgctaacgctgaaatcgacattccagaagctatgatcgacactgagttagatcgtatggttcgtgaattcgagcaacgtttaagccaacaaggtatgaaccttgagctttactaccaattcacaggtactgatgctgacaagttaaaagagcaaatgaaagaagacgctcaaaaacgcgtaagaatcaaccttgttcttgaagctatcattgaagctgaaaacatcgaagttactgaagaagaagtaactgcagaagttgaaaaaatggctgaaatgtacggtatgccagtagacgctatcaagcaagctcttggaagcgtagacgctttagctgaagatcttaaagttcgtaaagctgtagacttcttagtagaaaacgctgcataa

SEQ ID NO: 174 - protein, 425 aa, Bacillus anthracis
MAAKWEKLEGNVGVLTIEVDAKEVNNSIDAAFKKVVKTINVPGFRKGKMPRPLFEQRFGIESLYQDALDIILPKAYGEAIDEAGIFPVAHPEIDIEKFEKNANLIFTAKVTVKPEVKLGEYKGLAVEKVETTVTDEDVENELKSLQERQAELVVKEEGTVENGDTAVIDFEGFVDGEAFEGGKGENYSLAIGSGTFIPGFEEQVIGLKSGESKDVEVSFPEEYHAAELAGKPATFKVTVHEIKTKELPELNDEFAKEADEAVATLDELKAKLRTNLEEGKKHEAEHKVRDEVVELAAANAEIDIPEAMIDTELDRMVREFEQRLSQQGMNLELYYQFTGTDADKLKEQMKEDAQKRVRINLVLEAIIEAENIEVTEEEVTAEVEKMAEMYGMPVDAIKQALGSVDALAEDLKVRKAVDFLVENAA

SEQ ID NO: 175 - DNA, 714 bp, Bacillus anthracis
atgaaaattgcatttacaaagatggtaggtatattaactattagttcaatgttagtgttagtaggctgtcagacttcaggttcatctaaaaagcaagagcaaacatctgaaagtcatacacacgaaaatgaacacgatcacagtcatgatcatagtcatgctcatgatgaatcaacagaaaaaatttatgaagggtatttcgaagacaaccaagtgaaggatcgatcactctccgattggaaaggagactggcaatcggtatatccatatttacaagatggaacgcttgatgaggtatttgcttacaaagcgaaacataaaggtaaaatgtcagccaaagaatataaggagtattataatgaaggatatcaaacagatgtcaaccgtatcgtgattcaaggagatactgtaacattctacaaaaacaaagaagaatattctggtaaatatatctatgatgggtacaaaattttgacatatgatgcagggaatagaggtgtaagatacatatttaaactagcagaaaaaacagaaggagttcctcagtatattcaatttagtgatcatggtatttatccgaataaagctaatcactaccacttgtattggggtgacaatcgtgaagctttattcgatgaagtcatacactggcctacctactacccatcggatatgaatggacatgatattgcgcacgagatgatggcgcattaa

SEQ ID NO: 176 - protein, 237 aa, Bacillus anthracis
MKIAFTKMVGILTISSMLVLVGCQTSGSSKKQEQTSESHTHENEHDHSHDHSHAHDESTEKIYEGYFEDNQVKDRSLSDWKGDWQSVYPYLQDGTLDEVFAYKAKHKGKMSAKEYKEYYNEGYQTDVNRIVIQGDTVTFYKNKEEYSGKYIYDGYKILTYDAGNRGVRYIFKLAEKTEGVPQYIQFSDHGIYPNKANHYHLYWGDNREALFDEVIHWPTYYPSDMNGHDIAHEMMAH

SEQ ID NO: 177 - DNA, 1548 bp, Bacillus anthracis
atggtagtagcatacaaacatgagccatttacagatttttcagtagaggctaacaaattagcgtttgaagaaggtttaaagaaagtagaatcttatcttggacaagactatccattaattattgggggagaaaaaatcactacagaagacaaaattgtttctgtaaaccctgcaaataaagaggaacttgttggtcgcgtttcaaaagcaagccgtgagttagctgaaaaagcaatgcaagtagcggatgaaacattccaaacttggagaaagtcaaaaccagaaatgcgtgcagacattttattccgtgctgcagcgatcgttcgtcgtagaaaacatgaattctctgctattcttgtaaaagaagcaggtaaaccgtggaatgaggcagatgctgatacagcagaagcaatcgactttatggaatattatggtcgccaaatgttgaaattaaaagacggaattccagtagaaagccgtccaattgaatataatcgtttctcttacattccattaggagtaggtgttatcatttctccttggaacttcccattcgcaattatggcaggtatgacaacagctgctttagtttctggtaacacagtattactaaaaccagctagtacaactcctgtagtagcagcgaaattcatggaagtattagaagaagctggcttaccagctggcgtagtaaacttcgtaccaggtaatggttctgaagttggtgactacttagtagatcaccctcgtacacgcttcattagcttcactggatctcgtgatgtaggtatccgtatttatgagcgcgcagcgaaagtaaacccaggccaaatctggttaaaacgcgttatcgctgaaatgggtggtaaagatacaattgttgttgataaagaagcagatcttgaattagcagctaaatctatcgttgcatcagcattcggattctcaggacaaaaatgttctgcatgttctcgtgcagtaatccacgaagatgtatacgatcacgtattaaatcgtgctgttgaattaacgaaagaattaacagttgctaacccagctgtattaggtacaaacatgggtcctgttaatgaccaagctgcattcgataaagtaatgagctatgttgcaattggtaaagaagaaggtagaattttagcaggtggcgaaggagacgactctaaaggctggttcatccaaccaacaatcgttgctgacgttgcagaagatgctcgcctaatgaaagaagaaatcttcggaccagtagtagcattctgtaaagcaaaagactttgatcatgcacttgcaattgcaaacaatacagaatacggtttaacaggagcagttatctctaacaaccgtgatcatattgaaaaagcacgtgaagacttccacgtaggtaacttatacttcaaccgtggatgtactggtgcaatcgtaggataccaaccattcggtggctttaacatgtctggtacagactctaaagctggtggtcctgactacttagcgcttcacatgcaagcaaaaactacttctgaaactttataa

SEQ ID NO: 178 - protein, 515 aa, Bacillus anthracis
MVVAYKHEPFTDFSVEANKLAFEEGLKKVESYLGQDYPLIIGGEKITTEDKIVSVNPANKEELVGRVSKASRELAEKAMQVADETFQTWRKSKPEMRADILFRAAAIVRRRKHEFSAILVKEAGKPWNEADADTAEAIDFMEYYGRQMLKLKDGIPVESRPIEYNRFSYIPLGVGVIISPWNFPFAIMAGMTTAALVSGNTVLLKPASTTPVVAAKFMEVLEEAGLPAGVVNFVPGNGSEVGDYLVDHPRTRFISFTGSRDVGIRIYERAAKVNPGQIWLKRVIAEMGGKDTIVVDKEADLELAAKSIVASAFGFSGQKCSACSRAVIHEDVYDHVLNRAVELTKELTVANPAVLGTNMGPVNDQAAFDKVMSYVAIGKEEGRILAGGEGDDSKGWFIQPTIVADVAEDARLMKEEIFGPVVAFCKAKDFDHALAIANNTEYGLTGAVISNNRDHIEKAREDFHVGNLYFNRGCTGAIVGYQPFGGFNMSGTDSKAGGPDYLALHMQAKTTSETL

SEQ ID NO: 179 - DNA, 2445 bp, Bacillus anthracis
atggcaaagactaactcttacaaaaaagtaatcgctggtacaatgacagcagcaatggtagcaggtgttgtttctccagtagcagcagcaggtaaaacattcccagacgttcctgctgatcactggggaattgattctattaactacttagtagaaaaaggcgcagttaaaggtaacgacaaaggaatgttcgagcctggaaaagaattaactcgtgcagaagcagctacaatgatggctcaaatcttaaacttaccaatcgataaagatgctaaaccatctttcgctgactctcaaggccaatggtacactccattcatcgcagctgtagaaaaagctggcgttattaaaggtacaggaaacggctttgagccaaacggaaaaatcgaccgcgtttctatggcatctcttcttgtagaagcttacaaattagatactaaagtaaacggtactccagcaactaaattcaaagatttagaaacattaaactggggtaaagaaaaagctaacatcttagttgaattaggaatctctgttggtactggtgatcaatgggagcctaagaaaactgtaactaaagcagaagctgctcaattcattgctaagactgacaagcagttcggtacagaagcagcaaaagttgaatctgcaaaagctgttacaactcaaaaagtagaagttaaattcagcaaagctgttgaaaaattaactaaagaagatatcaaagtaactaacaaagctaacaacgataaagtactagttaaagaggtaactttatcagaagataaaaaatctgctacagttgaattatatagtaacttagcagctaaacaaacttacactgtagatgtaaacaaagttggtaaaacagaagtagctgtaggttctttagaagcaaaaacaatcgaaatggctgaccaaacagttgtagctgatgagccaacagcattacaattcacagttaaagatgaaaacggtactgaagttgtttcaccagagggtattgaatttgtaacgccagctgcagaaaaaattaatgcaaaaggtgaaatcactttagcaaaaggtacttcaactactgtaaaagctgtttataaaaaagacggtaaagtagtagctgaaagtaaagaagtaaaagtttctgctgaaggtgctgcagtagcttcaatctctaactggacagttgcagaacaaaataaagctgactttacttctaaagatttcaaacaaaacaataaagtttacgaaggcgacaacgcttacgttcaagtagaattgaaagatcaatttaacgcagtaacaactggaaaagttgaatatgagtcgttaaacacagaagttgctgtagtagataaagctactggtaaagtaactgtattatctgcaggaaaagcaccagtaaaagtaactgtaaaagattcaaaaggtaaagaacttgtttcaaaaacagttgaaattgaagctttcgctcaaaaagcaatgaaagaaattaaattagaaaaaactaacgtagcgctttctacaaaagatgtaacagatttaaaagtaaaagctccagtactagatcaatacggtaaagagtttacagctcctgtaacagtgaaagtacttgataaagatggtaaagaattaaaagaacaaaaattagaagctaaatatgtgaacaaagaattagttctgaatgcagcaggtcaagaagctggtaattatacagttgtattaactgcaaaatctggtgaaaaagaagcaaaagctacattagctctagaattaaaagctccaggtgcattctctaaatttgaagttcgtggtttagaaaaagaattagataaatatgttactgaggaaaaccaaaagaatgcaatgactgtttcagttcttcctgtagatgcaaatggattagtattaaaaggtgcagaagcagctgaactaaaagtaacaacaacaaacaaagaaggtaaagaagtagacgcaactgatgcacaagttactgtacaaaataacagtgtaattactgttggtcaaggtgcaaaagctggtgaaacttataaagtaacagttgtactagatggtaaattaatcacaactcattcattcaaagttgttgatacagcaccaactgctaaaggattagcagtagaatttacaagcacatctcttaaagaagtagctccaaatgctgatttaaaagctgcacttttaaatatcttatctgttgatggtgtacctgcgactacagcaaaagcaacagtttctaatgtagaatttgtttctgctgacacaaatgttgtagctgaaaatggtacagttggtgcaaaaggtgcaacatctatctatgtgaaaaacctgacagttgtaaaagatggaaaagagcaaaaagtagaatttgataaagctgtacaagttgcagtttctattaaagaagcaaaacctgcaacaaaataa

SEQ ID NO: 180 - protein, 814 aa, Bacillus anthracis
MAKTNSYKKVIAGTMTAAMVAGVVSPVAAAGKTFPDVPADHWGIDSINYLVEKGAVKGNDKGMFEPGKELTRAEAATMMAQILNLPIDKDAKPSFADSQGQWYTPFIAAVEKAGVIKGTGNGFEPNGKIDRVSMASLLVEAYKLDTKVNGTPATKFKDLETLNWGKEKANILVELGISVGTGDQWEPKKTVTKAEAAQFIAKTDKQFGTEAAKVESAKAVTTQKVEVKFSKAVEKLTKEDIKVTNKANNDKVLVKEVTLSEDKKSATVELYSNLAAKQTYTVDVNKVGKTEVAVGSLEAKTIEMADQTVVADEPTALQFTVKDENGTEVVSPEGIEFVTPAAEKINAKGEITLAKGTSTTVKAVYKKDGKVVAESKEVKVSAEGAAVASISNWTVAEQNKADFTSKDFKQNNKVYEGDNAYVQVELKDQFNAVTTGKVEYESLNTEVAVVDKATGKVTVLSAGKAPVKVTVKDSKGKELVSKTVEIEAFAQKAMKEIKLEKTNVALSTKDVTDLKVKAPVLDQYGKEFTAPVTVKVLDKDGKELKEQKLEAKYVNKELVLNAAGQEAGNYTVVLTAKSGEKEAKATLALELKAPGAFSKFEVRGLEKELDKYVTEENQKNAMTVSVLPVDANGLVLKGAEAAELKVTTTNKEGKEVDATDAQVTVQNNSVITVGQGAKAGETYKVTVVLDGKLITTHSFKVVDTAPTAKGLAVEFTSTSLKEVAPNADLKAALLNILSVDGVPATTAKATVSNVEFVSADTNVVAENGTVGAKGATSIYVKNLTVVKDGKEQKVEFDKAVQVAVSIKEAKPATK

SEQ ID NO: 181 - DNA, 537 bp, Bacillus anthracis
atgaaagcaactggaatcgtacgtcgaattgatgatttaggtagggtagtaatcccaaaggaaattcgtagaactttacgtattcgagaaggggacccattagaaatatttgttgatcgcgatggagaagtaattttaaagaaatattctccaattagcgaactaggtgattttgcaaaagaatatgcagaggctttatatgatagcttaggacataatgtgcttgtatgcgatcgagattctattatcgcagtatcaggcgtatcaaaaaaagaatacttaaataaaagcgttggcgatttaattgaaaaaacgatggaagaaagaaagtctgttattatgacggacgaaagtgatgtttccattattgatggtgtaacagaaaaggttcattcttatacagttggaccgattgttgcaaatggagacccaattggggctgtcattattttttcaaaagaagcgattataagcgaaatagagcacaaagcggtcaatactgctgccagtttcttagcgaaacaaatggaacagtaa

SEQ ID NO: 182 - protein, 178 aa, Bacillus anthracis
MKATGIVRRIDDLGRVVIPKEIRRTLRIREGDPLEIFVDRDGEVILKKYSPISELGDFAKEYAEALYDSLGHNVLVCDRDSIIAVSGVSKKEYLNKSVGDLIEKTMEERKSVIMTDESDVSIIDGVTEKVHSYTVGPIVANGDPIGAVIIFSKEAIISEIEHKAVNTAASFLAKQMEQ

SEQ ID NO: 183 - DNA, 1701 bp, Bacillus anthracis
atgaaaaagaaaagtttagcgttagtgttagcgacaggaatggcagttacaacgtttggagggacaggctctgcttttgcagattctaaaaatgtgctctctacgaagaagtacaatgagacagtacagtcaccggagtttatttctggggatttaactgaagcaactggtaagaaagcagaatctgttgtgtttgattacttaaatgcagcaaaaggtgattataagttaggggaaaagagtgcgcaagattctttcaaagtgaaacaagcgaagaaagatgctgtaactgattcaacagtattacgtttgcaacaagtttacgaaggagtacctgtatggggttctacgcaagtagctcacgtaagtaaagatggttcattaaaagtattgtctggaacagttgcacctgatttagacaaaaaagaaaagttgaaaaataaaaataagatcgaaggcgcaaaagcaattgaaattgcgcaaaaagatttaggtgttacacctaaatatgaggtagaaccaaaagcggacttatatgtatatcaaaatggtgaagaaacaacatatgcatacgttgtaaatttaaacttcttagagccaagcccaggaaactactactatttcattgaagcggacagcggtaaagtattaaataaatataataaattggatcatgtagcaaatgaagataagtcaccagttaagcaagaggcacctaaacaagaagcgaaaccggctgtaaagcctgtaacaggcacaaatgcagtgggtactggtaaaggtgtattaggagatacgaagtcacttaatacaacgttatctgcatcatcttactatttacaagataatacgcgcggagcaacgattttcacatatgatgcgaaaaaccgctcaacattaccaggaacgttatgggtagatgcggataatgttttcaatgcagcgtatgatgcagcggcagtagatgctcactactatgctggtagaacatatgattactataaagcgacatttaatagaaactctattaatgatgcaggagcaccattaaaatcaacagttcattatggaagtagatataataatgcgttctggaatggctctcaaatggtatacggagatggtgatggtgtaacattcacttcattgtctggtggaattgatgtaattggccatgaattaacgcatgctgttacagagtatagctcagatttaatttatcaaaatgaatcaggagcattaaatgaagctatttcagatgtatttggtacattagtagagtattatgataaccgtaaccctgattgggaaattggtgaagatatttacacgcctggtaaagctggagatgcacttcgctctatgagtgatccaacgaaatatggtgatccagatcattattctaagcgttacacaggtactggtgataacggtggcgttcatacaaatagcggtattattaacaaagcggcttacttactagcgaatggtggtacgcattacggtgttactgtaaacggtattggtaaagataaagtaggagcgatttattaccgtgcaaatacgcaatatttcacacaatctactacgtttagtcaagctcgtgctggattagtacaagctgcagctgacttatatggtgctagctctgcagaagtagcagcagttaagcaatcatatagtgctgttggcgtaaactaa

SEQ ID NO: 184 - protein, 566 aa, Bacillus anthracis
MKKKSLALVLATGMAVTTFGGTGSAFADSKNVLSTKKYNETVQSPEFISGDLTEATGKKAESVVFDYLNAAKGDYKLGEKSAQDSFKVKQAKKDAVTDSTVLRLQQVYEGVPVWGSTQVAHVSKDGSLKVLSGTVAPDLDKKEKLKNKNKIEGAKAIEIAQKDLGVTPKYEVEPKADLYVYQNGEETTYAYVVNLNFLEPSPGNYYYFIEADSGKVLNKYNKLDHVANEDKSPVKQEAPKQEAKPAVKPVTGTNAVGTGKGVLGDTKSLNTTLSASSYYLQDNTRGATIFTYDAKNRSTLPGTLWVDADNVFNAAYDAAAVDAHYYAGRTYDYYKATFNRNSINDAGAPLKSTVHYGSRYNNAFWNGSQMVYGDGDGVTFTSLSGGIDVIGHELTHAVTEYSSDLIYQNESGALNEAISDVFGTLVEYYDNRNPDWEIGEDIYTPGKAGDALRSMSDPTKYGDPDHYSKRYTGTGDNGGVHTNSGIINKAAYLLANGGTHYGVTVNGIGKDKVGAIYYRANTQYFTQSTTFSQARAGLVQAAADLYGASSAEVAAVKQSYSAVGVN

SEQ ID NO: 185 - DNA, 1260 bp, Bacillus anthracis
gtggcatttgaatttaaactaccagatatcggtgaaggtatccacgaaggtgaaatcgtaaaatggtttattaaaccaggcgacgaagtaaacgaagacgacgtacttcttgaagtacaaaatgataaagcagtagtagaaattccttctcctgttaaaggtaaagtacttgaagtacttgtagaagaaggtacggttgcagtagttggagatacattaattaaatttgatgctccaggatacgaaaaccttaaatttaaaggcgacgatcatgacgaagctcctaaagctgaagctactccagcagcaactgcagaagtagtaaatgagcgcgtaatcgctatgccatctgttcgtaaatatgctcgtgaaaacggcgtagacattcataaagtagctggttctggtaagaacggtcgtatcgtaaaagctgacatcgatgcatttgcaaatggtggacaagcagtagcagcaactgaggctccagcagcagtagaagctactccagcagcagcgaaagaagaagcaccaaaagcacaaccaatcccagctggtgaatatccagaaactcgtgagaaaatgagtggtatccgtaaagcaattgcgaaagcaatggttaactctaaacatacagctcctcacgtaacattaatggatgaagtagatgtaactgaacttgttgctcaccgtaagaagttcaaagctgtggcagctgacaaaggtattaaattaacttaccttccatacgttgttaaagctttaacatctgcattacgtgaatacccaatgttaaacacttctttagatgatgcttctcaagaagtagttcataaacattacttcaacatcggtatcgcagctgatacagacaaaggtctattagtaccagttgttaaagatacagatcgcaagtctatcttcacaatttctaacgagatcaatgatcttgctggtaaagcacgtgaaggtcgtttagctcctgctgaaatgaaaggcgcttcttgcacaattacaaacattggttctgcaggtggacaatggttcactccagttatcaaccacccagaagtagcaatccttggtatcggccgtatcgctgagaaaccagttgtgaaaaacggtgagatcgttgcagctccagtattagcattatctctaagctttgaccatcgtttaattgacggcgcaactgctcaaaaagcattaaaccaaattaaacgtctattgaatgacccacaattattagtaatggaggcgtaa

SEQ ID NO: 186 - protein, 419 aa, Bacillus anthracis
VAFEFKLPDIGEGIHEGEIVKWFIKPGDEVNEDDVLLEVQNDKAVVEIPSPVKGKVLEVLVEEGTVAVVGDTLIKFDAPGYENLKFKGDDHDEAPKAEATPAATAEVVNERVIAMPSVRKYARENGVDIHKVAGSGKNGRIVKADIDAFANGGQAVAATEAPAAVEATPAAAKEEAPKAQPIPAGEYPETREKMSGIRKAIAKAMVNSKHTAPHVTLMDEVDVTELVAHRKKFKAVAADKGIKLTYLPYVVKALTSALREYPMLNTSLDDASQEVVHKHYFNIGIAADTDKGLLVPVVKDTDRKSIFTISNEINDLAGKAREGRLAPAEMKGASCTITNIGSAGGQWFTPVINHPEVAILGIGRIAEKPVVKNGEIVAAPVLALSLSFDHRLIDGATAQKALNQIKRLLNDPQLLVMEA

SEQ ID NO: 187 - DNA, 2289 bp, Bacillus anthracis
atggcaattcaaacaagtaacttaggttatccacgtatcggattacaacgagagtggaaaaaaacattggaagctttttggtccaataaaatcaatgaagaacaatttttaacaacaatgaaagaaattcgccttcaacacgtaaaagtacagcaagaaaaagggattgaactcattccaattggcgactttacatattacgatcacgttttggatactgcttatatgctaggatttatcccatcacgtttttctgagtttacatcttacctagatgtatattttgcaatggcgcgtggctctaaagatcacgtagcttccgaaatgacaaaatggtttaacacaaactatcattatatcgttcctgaatatgaagagggattacaaatctctttaaaagataatcgtccacttcgcttatacgaagaggcaaaacaagaattgggtgtagatggaaaacctgttattttaggaccatatactttcttgaaattagctaaaggctatacacaagagcaatttgctactattttaaaacagttagttgcaccttacgtacaactgctttcagaactacatgcagctggtgcacaaatcattcmagttgatgaaccgattttcgcttctttaacgaaagaagaagttcaacaagcaaaagaaatttatgaagctattcgtaaagaggttccaaatgcgactcttcttttacaaacatactttgatagtgtagaagaaaactatgaagaaattattacattcccagtatcaagtattggattagatttcgttcatggtaaagaaggtaatttaaatgctatttcaaaatatggattcccagctgataaaactttagctgttggttgtatagatggccgtaacatttggagagctgaccttgatgaagttcttacgttatttacaacgttacaaaaacaagtccaaacgaaagatctcatcgttcaaccttcttgtagcttattgcatacaccaatcgataaaacagaagaaactcacttatcaactgagctatttgatgcgttggcatttgcaaatcaaaaattagaagagttagttcttattcattccgctctgactcaaggtacagaaagcattagtaatgaactggaaacatatcgaaacgtacatcatacaattcgttcatctgctgcacgtaaccgagaagatgtcaaagcagcacgaacagcactaaaagaagaagatttttcacgtcctcttccatttgaaaaacgatacgaattacaacaagttgccctaaagttaccgttgttaccaacaacgactatcggtagcttccctcaaacaactgaagttcgccaaacgcgaaaagaatggcgtaatggtattatttcaaatgaacaatatgaacaatttattgaaaaagagacagaaaaatggattcgttaccaagaagaaattggtcttgatgttcttgttcatggcgagtttgaaagaactgacatggtcgaatattttggtgagcgccttgctggcttctcattcactaaaaacggttgggtacaatcatacggttctcgttgcgtaaaaccacctgttatttatggtgatgtagcctttattaacggcatgactattaaggaaacggtttatgcacaaagcttaacagagaaagttgtaaaaggaatgttaactggacctgttacgattttaaattggtccttcgttcgaaatgacattccaagaaaagaagtttcgtatcaaattgcattagctcttcgtcatgaaattgaactacttgaatcttctggaattcgagtgatccaagtcgatgagccagcacttcgtgaaggaatgccactgaaagaaaaagattgggacgcttatattacatgggcagtacaatccttccttttagcaacttcttctgtagcaaatgaaacacaaattcatacgcatatgtgttacagtaacttcgaagatattgttgacgcgattcgcgcattagatgcagatgtgatttctatcgaaacatcaagaagtcacggagaatttattgatacattaaaacatacaacatacgaaaagggcatcggtctaggtgtatatgatattcatagcccacgtgtaccaagtaaagatgaaatgtataaaatcgtagaacaatctttacaagtatgcgatcctaaatatttctggattaatcctgattgtggtttaaaaacgcgaagaacagaagaagttattccagctctagaacatatggtgcaagcagcaaaagatgctcgttccctactaaaaacaaacgcataa

SEQ ID NO: 188 - protein, 762 aa, Bacillus anthracis
  misc_feature (201)..(201): Xaa can be any naturally occurring amino acid
MAIQTSNLGYPRIGLQREWKKTLEAFWSNKINEEQFLTTMKEIRLQHVKVQQEKGIELIPIGDFTYYDHVLDTAYMLGFIPSRFSEFTSYLDVYFAMARGSKDHVASEMTKWFNTNYHYIVPEYEEGLQISLKDNRPLRLYEEAKQELGVDGKPVILGPYTFLKLAKGYTQEQFATILKQLVAPYVQLLSELHAAGAQIIXVDEPIFASLTKEEVQQAKEIYEAIRKEVPNATLLLQTYFDSVEENYEEIITFPVSSIGLDFVHGKEGNLNAISKYGFPADKTLAVGCIDGRNIWRADLDEVLTLFTTLQKQVQTKDLIVQPSCSLLHTPIDKTEETHLSTELFDALAFANQKLEELVLIHSALTQGTESISNELETYRNVHHTIRSSAARNREDVKAARTALKEEDFSRPLPFEKRYELQQVALKLPLLPTTTIGSFPQTTEVRQTRKEWRNGIISNEQYEQFIEKETEKWIRYQEEIGLDVLVHGEFERTDMVEYFGERLAGFSFTKNGWVQSYGSRCVKPPVIYGDVAFINGMTIKETVYAQSLTEKVVKGMLTGPVTILNWSFVRNDIPRKEVSYQIALALRHEIELLESSGIRVIQVDEPALREGMPLKEKDWDAYITWAVQSFLLATSSVANETQIHTHMCYSNFEDIVDAIRALDADVISIETSRSHGEFIDTLKHTTYEKGIGLGVYDIHSPRVPSKDEMYKIVEQSLQVCDPKYFWINPDCGLKTRRTEEVIPALEHMVQAAKDARSLLKTNA

SEQ ID NO: 189 - DNA, 1413 bp, Bacillus anthracis
atggtagtaggagatttcccaattgaattagatacagtcgttgttggtgcaggtcctggtggatacgttgcggcaattcgtgcagcacaattaggtcaaaaggtagcaattattgaaaaagctaaccttggtggcgtatgcttaaacgttggatgtattccttcaaaagcgttaatcaatgcaggtcatcgttatgagaatgcaatgcattctgatgacatgggtatcactgcagagaacgtaaaagttgactttacaaaagttcaagaatggaaaaacggcgtagttaagaaattaactggcggtgttgaaggccttcttaaaggtaacaaagttgaaatcattcgcggtgaagcttacttcgtagatgctaatacattacgcgttatgactgaagaggcagctcaaacttatacgtttaaaaatgctgttcttgcaactggttctacaccaatcgaaattccaggattcaaatactctaaacgtgttatcaactctacaggcgctttaagcttacctgaaattcctaaaaaacttgttgtaatcggcggcggttacatcggtatggaattaggtactgcatatgctaacttcggtacagaagttactgtagtagaagctggcgacgaaatcttagctggtttcgaaaaagctatgagctctgttgttaaacgtgctctacagaaaaaaggtaacgtaaatatccatacaaaagctatggctaaaggcgttgaagaaacagaaactggcgtaaaagttagctttgaagttaaaggtgaaatccaaactgtagaagcagattacgtattagtaactgtaggtcgtcgtccaaacactcaagaaatcggtcttgagcaagttggagttaaaatgactgaccgcggcatcatcgaaatcgatgagcaatgtcgtacaaacgtaccaaacatctatgcaatcggtgatatcgttcctggaccaccattagctcacaaagcttcttacgaaggtaaagtagctgtagaagcaattagtggccatgcatcagctatcgattacatcggaattcctgcagtatgcttcactgatccagaattagcatctgttggttacactaagaaacaagctgaagaagctggaatgactgtaactgtatctaagttcccattcgctgctaacggtcgtgcattatcattaaacagcactgacggtttcttacaacttgtaacacgtaaagaagatggtcttcttgtaggtgctcaagttgcaggtgcaggcgcttctgatattatttctgagattggtttagctatcgaagctggaatgacagcagaagatatcgctcaaacaatccacgctcacccaacattaggtgaaatcacaatggaagcagctgaagttgctcttggaatgccaattcacattgtaaaataa

SEQ ID NO: 190 - protein, 470 aa, Bacillus anthracis
MVVGDFPIELDTVVVGAGPGGYVAAIRAAQLGQKVAIIEKANLGGVCLNVGCIPSKALINAGHRYENAMHSDDMGITAENVKVDFTKVQEWKNGVVKKLTGGVEGLLKGNKVEIIRGEAYFVDANTLRVMTEEAAQTYTFKNAVLATGSTPIEIPGFKYSKRVINSTGALSLPEIPKKLVVIGGGYIGMELGTAYANFGTEVTVVEAGDEILAGFEKAMSSVVKRALQKKGNVNIHTKAMAKGVEETETGVKVSFEVKGEIQTVEADYVLVTVGRRPNTQEIGLEQVGVKMTDRGIIEIDEQCRTNVPNIYAIGDIVPGPPLAHKASYEGKVAVEAISGHASAIDYIGIPAVCFTDPELASVGYTKKQAEEAGMTVTVSKFPFAANGRALSLNSTDGFLQLVTRKEDGLLVGAQVAGAGASDIISEIGLAIEAGMTAEDIAQTIHAHPTLGEITMEAAEVALGMPIHIVK

SEQ ID NO: 191 - DNA, 375 bp, Brucella
atggctgatctcgcaaagatcgttgaagacctttcggccctgaccgttctggaagccgctgagctgtccaagcttctcgaagagaagtggggcgtttcggctgctgctccggtcgctgttgctgctgccggtggcgctgcccctgctgctgccgcagaagaaaagaccgaattcgacgtcgttctcgctgacggcggcgctaacaagatcaacgtgatcaaggaagtgcgcgcactcaccggtctcggcctcaaggaagccaaggacctggtcgaaggcgctccgaaggctgtcaaggaaggcgcctcgaaggacgaagctgagaagatcaaggcacagctcgaagctgctggcgccaaggttgaactcaagtaa

SEQ ID NO: 192 - protein, 124 aa, Brucella
MADLAKIVEDLSALTVLEAAELSKLLEEKWGVSAAAPVAVAAAGGAAPAAAAEEKTEFDVVLADGGANKINVIKEVRALTGLGLKEAKDLVEGAPKAVKEGASKDEAEKIKAQLEAAGAKVELK

SEQ ID NO: 193 - DNA, 570 bp, Brucella
atggaagtcattcttctggaacgcattggccgcctcggccagatgggcgacaccgtcaaggtcaaggacggctatgcccgcaacttcctgctgccgcagggcaaggctcttcgtgccaacgaagccaacaagaagaagtttgaaggccagcgcgcacagcttgaagcccagaacctggaacgcaagaacgaagcccaggctgttgccgacaagctcaatggcgaaagcttcatcgtcgtgcgttcggcaggtgaaaccggccagctctacggttccgtttcgacccgcgacatcgccgaaatcatcacggccaacggcttcacgctgcaccgcaaccaggttgagctgaaccacccgatcaagacgatcggcctgcacgaagtttcggtttcgctgcacccggaagtccaggtcaaggtcatggtcaacatcgcgcgctcgaccgaagaagccgaatgtcaggccaagggtgaagacctcacctcgatcgaagccatctacggcatcgaagagcagccgctttcggaagaagtcttcgacgacgaagacgaagctgaagatcaggcttga

SEQ ID NO: 194 - protein, 189 aa, Brucella
MEVILLERIGRLGQMGDTVKVKDGYARNFLLPQGKALRANEANKKKFEGQRAQLEAQNLERKNEAQAVADKLNGESFIVVRSAGETGQLYGSVSTRDIAEIITANGFTLHRNQVELNHPIKTIGLHEVSVSLHPEVQVKVMVNIARSTEEAECQAKGEDLTSIEAIYGIEEQPLSEEVFDDEDEAEDQA

SEQ ID NO: 195 - DNA, 642 bp, Brucella
atgcgcactcttaagtctctcgtaatcgtctcggctgcgttgctgccgttctctgcgaccgcttttgctgccgacgccatccaggaacagcctccggttccggctccggttgaagtagctccccagtatagctgggctggtggctataccggtctttaccttggctacggctggaacaaggccaagaccagcaccgttggcagcatcaagcctgacgattggaaggctggcgcctttgctggctggaacttccagcaggaccagatcgtatacggtgttgaaggtgatgcaggttattcctgggccaagaagtccaaggacggcctggaagtcaagcagggctttgaaggctcgctgcgtgcccgcgttggctacgacctgaacccggttatgccgtacctcacggctggtattgccggttcgcagatcaagcttaacaacggcttggacgacgaaagcaagttccgcgtgggttggacggctggtgccggtctcgaagccaagctgacggacaacatcctcggccgcgttgagtaccgttacacccagtacggcaacaagaactatgatctggccggtacgactgttcgcaacaagctggacacgcaggatttccgcgtcggcatcggctacaagttctaa

SEQ ID NO: 196 - protein, 213 aa, Brucella
MRTLKSLVIVSAALLPFSATAFAADAIQEQPPVPAPVEVAPQYSWAGGYTGLYLGYGWNKAKTSTVGSIKPDDWKAGAFAGWNFQQDQIVYGVEGDAGYSWAKKSKDGLEVKQGFEGSLRARVGYDLNPVMPYLTAGIAGSQIKLNNGLDDESKFRVGWTAGAGLEAKLTDNILGRVEYRYTQYGNKNYDLAGTTVRNKLDTQDFRVGIGYKF

SEQ ID NO: 197 - DNA, 786 bp, Brucella; identical to SEQ ID NO: 165

SEQ ID NO: 198 - protein, 261 aa, Brucella; identical to SEQ ID NO: 166

SEQ ID NO: 199 - DNA, 1128 bp, Brucella
atgcccagacccatttttaactttgactggaggtcagaaatgaacatcaagagccttctccttggctccgctgcagctctggttgcagcttccggcgctcaggctgccgacgcaatcgtcgcgccagagcccgaagccgttgaatatgtccgcgtttgcgacgcttacggcgctggctacttctacattccgggcaccgaaatctgcctgcgcgtccatggttacgtccgttacgacgtaaagggcggcgatgacgtttactccggtaccgaccgcaatggctgggacaagggcgctcgtttcgcactccgcgtttccaccggttcggaaaccgaactcggcaccctcaagaccttcaccgaactgcgcttcaactatgctgcgaacaattcgggcgtagatggtaaatatggtaatgaaaccagcagcggcaccgtcatggagttcgcgtatatccagctcggtggtctgcgcgttggtatcgatgaatcggaattccataccttcaccggttacctcggcgatgtcatcaacgatgacgtgatctcggctggctcctaccgcaccggcaagatctcgtacaccttcactggcggaaacggcttctcggctgtgatcgctctcgaacagggtggcgacaacgacggtggttacactggcacgaccaactaccacatcgacggctacatgcctgacgttgttggcggcctgaagtatgctggcggctggggttcgatcgctggtgttgttgcctatgactcggtcatcgaagaatgggctgccaaggttcgtggcgacgtcaacatcaccgaccagttctcggtttggttgcagggcgcatattcgtccgctgctacgccggatcagaactacggccagtggggcggcgattgggctgtctggggtggtctgaagtatcaggctacgcagaaggctgccttcaacctgcaggctgcgcatgacgactggggcaagacggcagttacggctaacgttgcttacgaactggttcctggcttcaccgttacgccggaagtttcctacaccaagtttggtggcgagtggaagaacactgttgctgaagacaatgcttggggcggtatcgttcgcttccagcgttcgttctaa

SEQ ID NO: 200 - protein, 375 aa, Brucella
MPRPIFNFDWRSEMNIKSLLLGSAAALVAASGAQAADAIVAPEPEAVEYVRVCDAYGAGYFYIPGTEICLRVHGYVRYDVKGGDDVYSGTDRNGWDKGARFALRVSTGSETELGTLKTFTELRFNYAANNSGVDGKYGNETSSGTVMEFAYIQLGGLRVGIDESEFHTFTGYLGDVINDDVISAGSYRTGKISYTFTGGNGFSAVIALEQGGDNDGGYTGTTNYHIDGYMPDVVGGLKYAGGWGSIAGVVAYDSVIEEWAAKVRGDVNITDQFSVWLQGAYSSAATPDQNYGQWGGDWAVWGGLKYQATQKAAFNLQAAHDDWGKTAVTANVAYELVPGFTVTPEVSYTKFGGEWKNTVAEDNAWGGIVRFQRSF

SEQ ID NO: 201 - DNA, 786 bp, Brucella; identical to SEQ ID NO: 165

SEQ ID NO: 202 - protein, 261 aa, Brucella; identical to SEQ ID NO: 166

SEQ ID NO: 203 - DNA, 522 bp, Brucella
atgaagtccttatttattgcatcgacaatggtgcttatggcttttccggctttcgcagaaagcacgacggtaaaaatgtatgaggcgctgccgaccggaccgggtaaagaagttggcaccgtggtcatttccgaagccccgggcgggctgcacttcaaggtgaatatggaaaagctgacgccgggctatcatggctttcatgttcacgaaaatccaagctgcgctccgggagaaaaagacggcaagatcgtaccggctcttgctgccggcgggcattatgatccgggtaatacccatcaccatttagggcctgaaggtgatggacatatgggcgatttgccacgcctgagcgccaatgctgacggcaaggtgagtgaaaccgttgtcgctccacatctcaagaaattggcggaaatcaagcagcgttctttgatggtccatgtcggaggggataattattccgataagcctgagccgcttggtggcggtggtgcccgttttgcctgcggcgtgatcgaataa

SEQ ID NO: 204 - protein, 173 aa, Brucella
MKSLFIASTMVLMAFPAFAESTTVKMYEALPTGPGKEVGTVVISEAPGGLHFKVNMEKLTPGYHGFHVHENPSCAPGEKDGKIVPALAAGGHYDPGNTHHHLGPEGDGHMGDLPRLSANADGKVSETVVAPHLKKLAEIKQRSLMVHVGGDNYSDKPEPLGGGGARFACGVIE

SEQ ID NO: 205 - DNA, 1731 bp, Brucella
atggctattccggatgcaccaggagtatacatgtctcaatccaaccctacccgcgcagatttcgagtccctgctggcagaatcctttgcggaacatgatcttgctgaaggctatgtcgtcaagggccgcatcgtcgccatcgaaaaggacatggcgatcatcgacgccggtctgaaggtcgaaggtcgcgtgccgttgaaggaatttggcgcaaagggcaaagacggcacgctgaagccgggcgacgaagtggaagtttacgtcgagcgtatcgaaaacgctctgggcgaagctgtcctgtcgcgcgaaaaagcacgccgcgaagaaagctgggtcaagctcgagcagaagtttgccaatggcgagcgcgtcgatggtgtcatcttcaatcaggtcaagggtggtttcaccgtcgacctcgatggtgctgttgccttcctgccgcgcagccaggtcgatatccgtccgatccgcgacgtcaccccactcatgcacgtcccgcaaccgtttgaaatcctcaagatggacaagcgccgcggcaacatcgttgtctcgcgccgtaccgttcttgaagaaagccgtgcggaacagcgttcggaaatcgtccagaaccttgaagaaggtcaggtcgttgaaggcgtcgtcaagaacatcaccgattacggtgcgttcgtcgacctcggcggcattgacggtctcctgcacgtgaccgacatggcatggcgccgcgtcaaccatccgtcggaaatcctcaccatcggccagacggtcaaggtgcagatcatccgcatcaaccaggaaacccatcgtatctcgctcggcatgaagcagcttgagagcgatccttgggatggtatcggcgcgaagtacccgatcggcaaaaagatcaccggcaccgtcacgaacatcaccgattacggtgcgttcgtcgaaatcgagccgggcatcgaaggcctcatccacgtttccgaaatgtcgtggaccaagaagaatgtccatccgggcaagattctgtccaccacgcaggaagtcgaagtcgttgtgctcgaagttgatccggtcaagcgccgtatctcgctcggcctcaagcagaccctcgacaatccgtggacgacctttgcccagaagtaccctgtcggtaccgtcgttgaaggcgaagtcaagaacaagaccgaattcggcctgttcatcggcctcgacggcgacgttgacggcatggttcacctctccgacctcgactggaaccgtccgggcgaacaggtcatcgaagagtacaacaagggtgaagtggtcaaggctgtcgttctcgacgttgatgtcgagaaggaacgcatctcgctcggcatcaagcagctttccggcgacaaggtcggcgaagcagcagcttccggcgaactgcgcaagaatgccgtcgtcacctgcgaagtgaccgccgttaccgatggtggccttgaggtccgtctggtcgatcacgacctcgacagcttcatccgccgttcggatctgtcgcgtgaccgcgacgaacagcgtccggaacgcttcacggtcggtcagaaggttgacgcccgcgtcatcgccttcgacaagaagacccgcaagttgcaggtctcgatcaaggcgctcgaaatcgctgaagaaaaggaagcagtcgctcagtacggttcgtccgactccggcgcttcgctcggcgacattctcggcgctgccctgaagaagcaggaaaagaactga

SEQ ID NO: 206 - protein, 576 aa, Brucella
MAIPDAPGVYMSQSNPTRADFESLLAESFAEHDLAEGYVVKGRIVAIEKDMAIIDAGLKVEGRVPLKEFGAKGKDGTLKPGDEVEVYVERIENALGEAVLSREKARREESWVKLEQKFANGERVDGVIFNQVKGGFTVDLDGAVAFLPRSQVDIRPIRDVTPLMHVPQPFEILKMDKRRGNIVVSRRTVLEESRAEQRSEIVQNLEEGQVVEGVVKNITDYGAFVDLGGIDGLLHVTDMAWRRVNHPSEILTIGQTVKVQIIRINQETHRISLGMKQLESDPWDGIGAKYPIGKKITGTVTNITDYGAFVEIEPGIEGLIHVSEMSWTKKNVHPGKILSTTQEVEVVVLEVDPVKRRISLGLKQTLDNPWTTFAQKYPVGTVVEGEVKNKTEFGLFIGLDGDVDGMVHLSDLDWNRPGEQVIEEYNKGEVVKAVVLDVDVEKERISLGIKQLSGDKVGEAAASGELRKNAVVTCEVTAVTDGGLEVRLVDHDLDSFIRRSDLSRDRDEQRPERFTVGQKVDARVIAFDKKTRKLQVSIKALEIAEEKEAVAQYGSSDSGASLGDILGAALKKQEKN

SEQ ID NO: 207 - DNA, 1017 bp, Avian influenza virus
ataatggagaaaattgtactgctgttcgcgattgttagcctggtgaagtccgatcagatctgcatcggttatcatgctaacaacagcaccgaacaagttgataccatcatggagaaaaacgtaaccgtcacccacgctcaggacatcctggaaaaaaagcacaacggtaaactgtgcgatctggatggtgtgaaaccgctgatcctgcgtgactgctccgtagcaggttggctgctgggtaacccgatgtgcgacgagttcatcaacgttccagaatggtcctacattgtcgaaaaagctaacccggttaacgacctgtgttatccgggtgatttcaacgattatgaagaactgaagcacctgctgtctcgcatcaaccactttgaaaagatccagattatcccaaaatcctcttggtcttcccacgaagcgtctctgggtgtgagcagcgcttgtccgtaccagggtaaatcctctttcttccgtaacgttgtttggctgatcaagaaaaattctacctacccaaccatcaaacgttcttacaacaacaccaatcaggaggatctgctggttctgtggggtatccaccacccgaacgacgcagcagaacagactaagctgtaccagaacccgaccacctacatcagcgttggcacttctactctgaaccagcgtctggtgccgcgcatcgcgacccgttctaaggtaaatggtcagtctggtcgtatggaatttttctggaccatcctgaaaccgaacgacgcgatcaactttgagtccaacggtaacttcatcgctccagaatacgcgtacaaaatcgttaaaaagggcgattctactattatgaagtctgaactggaatacggtaactgcaatactaaatgccagacgccgatgggtgctattaacagcagcatgccatttcacaacattcaccctctgactatcggcgagtgcccgaaatacgtaaaaagcaaccgtctggttctggcgaccggcctgcgtaactctccgata

SEQ ID NO: 208 - protein, 339 aa, Avian influenza virus
IMEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPI

SEQ ID NO: 209 - DNA, 984 bp, Avian influenza virus
atggtaccggcaccagcgatggaaaaaaatgttaccgttactcatgctcaagacattctggaaaaaaagcataatggtaaagcgcctgctgacctggacggtgtaaaaccactgattctgcgtgattgttccgtagctggcgctcctgctccggttaacgatctgtgttatccaggcgatttcaacgactacgaggaactggcaccggcgattcagatcatcccgaaatcttcctggtctagccacgaagcgtccctgggcgtttcctccgcttgcccttaccaaggcaaaagctctgcaccggcacgcaacgttgtatggctgatcaagaaaaactccacctatccgaccatcaaacgcagctacaataacaccaaccaggaggacgctccggctcaccatccgaatgacgccgcagaacagacgaagctgtaccagaacccgaccaccgctccagccgttaaaaagggtgacagcacgattatgaaaagcgagctggaatacggcaactgcaacactaaatgccagactccaatgggcgctattaacagctccatgccgtttgctccggccattcaaatcattccaaaatctagctggtccgaccatgaagcatccagcggcgtgtcctctgcctgcccatatcagggcaccccgagcgcaccggctgttccacgcatcgctacgcgttctaaggtgaacggtcagtctggtcgtgctccggctgttaagaaaggcgatagcgccattgttaagtctgaagtggaatacggtaactgtaacactaagtgtcaaactcctatcggtgccatcaactcttccatgccgttcgcaccggcaggtgttagcagcgcatgcccgtaccagggccgcagctctgcgccggctggtgttagctccgcttgtccgtatctgggttctccgagcgcaccagcgggcgttagctctgcctgtccgtacctgggtcgttccagcgctccggca

SEQ ID NO: 210 - protein, 328 aa, Avian influenza virus
MVPAPAMEKNVTVTHAQDILEKKHNGKAPADLDGVKPLILRDCSVAGAPAPVNDLCYPGDFNDYEELAPAIQIIPKSSWSSHEASLGVSSACPYQGKSSAPARNVVWLIKKNSTYPTIKRSYNNTNQEDAPAHHPNDAAEQTKLYQNPTTAPAVKKGDSTIMKSELEYGNCNTKCQTPMGAINSSMPFAPAIQIIPKSSWSDHEASSGVSSACPYQGTPSAPAVPRIATRSKVNGQSGRAPAVKKGDSAIVKSEVEYGNCNTKCQTPIGAINSSMPFAPAGVSSACPYQGRSSAPAGVSSACPYLGSPSAPAGVSSACPYLGRSSAPA

SEQ ID NO: 211 - DNA, 1056 bp, Avian influenza virus
atgtctctgctgaccgaagtagaaactccaactcgtaatgaatgggaatgccgctgctctgactctagcgaccctatcgttgtggcggcaaacattatcggcatcctgcacctgattctgtggattctggaccgcctgtttttcaaatgtatctaccgccgtctgaaatacggtctgaaacgcggtccggctacggcaggcgttccggagtctatgcgcgaagaataccgtcaggagcaacagtctgccgtggatgttgatgacggccacttcgtaaacattgaactggaaggtggtatgtccctgctgactgaagtagaaacctatgtcctgtccatcattccgtctggcccgctgaaagctgagattgctcaaaaactggaagacgttttcgctggtaaaaataccgatctggaggctctgatggagtggctgaaaacccgcccgatcctgtccccactgaccaaaggtatcctgggtttcgttttcaccctgactgtaccgtccgaacgtggtctgcaacgccgtcgctttgtgcaaaacgctctgaacggcaatggtgacccgaacaatatggaccgtgctgtgaaactgtataaaaagctgaagcgtgaaatcaccttccacggcgccaaagaagttgctctgtcctacagcaccggtgcactggcttcctgcatgggtctgatctacaaccgtatgggcactgtaacgacggaagttgccttcggtctggtctgtgccacctgtgaacaaatcgcggattctcagcaccgctcccaccgtcagatggcgactatcactaacccgctgattcgtcacgaaaaccgtatggttctggcgtccactaccgcgaaagcaatggaacagatggctggttcctccgaacaggccgcagaggctatggaaatcgctaaccaagctcgtcagatggttcaggctatgcgcactattggtacccatccgaactcttccgccggtctgcgtgataacctgctggaaaacctgcaagcctaccagaaacgtatgggtgtgcaaatgcagcgtttcaaataa

SEQ ID NO: 212 - protein, 351 aa, Avian influenza virus
MSLLTEVETPTRNEWECRCSDSSDPIVVAANIIGILHLILWILDRLFFKCIYRRLKYGLKRGPATAGVPESMREEYRQEQQSAVDVDDGHFVNIELEGGMSLLTEVETYVLSIIPSGPLKAEIAQKLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVAFGLVCATCEQIADSQHRSHRQMATITNPLIRHENRMVLASTTAKAMEQMAGSSEQAAEAMEIANQARQMVQAMRTIGTHPNSSAGLRDNLLENLQAYQKRMGVQMQRFK

SEQ ID NO: 213 - DNA, 1626 bp, Norovirus
aaaatggcttctaatgatgctgctccttctactgatggtgctgctggtctggtgccagaatccaacaacgaagtcatggccctggagccggttgcgggtgcagcgctggcagcgccggtaaccggtcagaccaatatcatcgatccgtggattcgtgctaatttcgtgcaggccccgaacggcgagtttaccgtgtccccgcgtaacgcaccgggtgaggttctgctgaacctggaactgggcccggaactgaacccgtatctggcacacctggcgcgtatgtacaacggttatgccggtggtatggaggttcaggttatgctggcaggtaacgcgtttaccgcgggcaagctggtatttgcggccgtgcctcctcatttcccagtggagaacctgagcccgcagcagatcaccatgttcccacatgtgattattgatgttcgtactctggaacctgtgctgctgccgctgccggatgttcgcaacaatttcttccactataaccagaaagacgacccgaaaatgcgcatcgtcgctatgctgtatacgccgctgcgttccaatggttctggtgatgacgttttcactgtaagctgccgtgtactgactcgtccatctccggatttcgactttacttacctggtgccgccgactgtagagtctaaaaccaaaccgttcactctgccgatcctgaccctgggtgaactgtctaactctcgtttcccggtatctatcgaccagatgtatacctctcctaatgaagttatctccgtccagtgccagaacggtcgctgcaccctggacggtgaactgcaaggcaccacccaactgcaagtttccggcatctgcgctttcaaaggcgaagtaaccgctcacctgcaagataacgatcacctgtataacatcacgatcaccaacctgaacggcagcccgttcgacccgtctgaggacattccggccccactgggtgtaccggatttccagggccgtgtgtttggtgttatcacccaacgtgacaaacagaacgcggcaggtcagagccagccggcgaaccgtggtcatgacgcagttgttcctacttacacggcgcagtacaccccaaaactgggccaggtacaaatcggtacttggcagactgatgatctgaaggttaaccagccagtgaaattcaccccggttggtctgaacgacactgagcactttaaccagtgggttgtaccgcgttatgcgggtgctctgaatctgaacaccaacctggcgcctagcgtggctccggtattcccgggcgaacgcctgctgttctttcgttcctacctgccgctgaaaggtggttatggtaacccggctattgattgcctgctgccgcaggagtgggtgcagcacttctatcaggaggcggctccgtccatgtctgaagttgcgctggttcgttacatcaacccggacaccggccgtgcgctgttcgaagcgaaactgcaccgcgcaggcttcatgaccgtgtcctctaatacttccgcaccggttgttgtacctgccaatggttacttccgcttcgattcttgggtaaaccagttttactctctggcaccgatgggtactggcaacggccgtcgccgtatccagtaa

SEQ ID NO: 214 - protein, 541 aa, Norovirus
KMASNDAAPSTDGAAGLVPESNNEVMALEPVAGAALAAPVTGQTNIIDPWIRANFVQAPNGEFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTVESKTKPFTLPILTLGELSNSRFPVSIDQMYTSPNEVISVQCQNGRCTLDGELQGTTQLQVSGICAFKGEVTAHLQDNDHLYNITITNLNGSPFDPSEDIPAPLGVPDFQGRVFGVITQRDKQNAAGQSQPANRGHDAVVPTYTAQYTPKLGQVQIGTWQTDDLKVNQPVKFTPVGLNDTEHFNQWVVPRYAGALNLNTNLAPSVAPVFPGERLLFFRSYLPLKGGYGNPAIDCLLPQEWVQHFYQEAAPSMSEVALVRYINPDTGRALFEAKLHRAGFMTVSSNTSAPVVVPANGYFRFDSWVNQFYSLAPMGTGNGRRRIQ

SEQ ID NO: 215 - DNA, 1488 bp, Salmonella
atggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatatcgatctgaagcagatcaactctcagaccctgggtctggatacgctgaatgtgcaacaaaaatataaggtcagcgatacggctgcaactgttacaggatatgccgatactacgattgctttagacaatagtacttttaaagcctcggctactggtcttggtggtactgaccagaaaattgatggcgatttaaaatttgatgatacgactggaaaatattacgccaaagttaccgttacggggggaactggtaaagatggctattatgaagtttccgttgataagacgaacggtgaggtgactcttgctggcggtgcgacttccccgcttacaggtggactacctgcgacagcaactgaggatgtgaaaaatgtacaagttgcaaatgctgatttgacagaggctaaagccgcattgacagcagcaggtgttaccggcacagcatctgttgttaagatgtcttatactgataataacggtaaaactattgatggtggtttagcagttaaggtaggcgatgattactattctgcaactcaaaataaagatggttccataagtattaatactacgaaatacactgcagatgacggtacatccaaaactgcactaaacaaactgggtggcgcagacggcaaaaccgaagttgtttctattggtggtaaaacttacgctgcaagtaaagccgaaggtcacaactttaaagcacagcctgatctggcggaagcggctgctacaaccaccgaaaacccgctgcagaaaattgatgctgctttggcacaggttgacacgttacgttctgacctgggtgcggtacagaaccgtttcaactccgctattaccaacctgggcaacaccgtaaacaacctgacttctgcccgtagccgtatcgaagattccgactacgcgaccgaagtttccaacatgtctcgcgcgcagattctgcagcaggccggtacctccgttctggcgcaggcgaaccaggttccgcaaaacgtcctctctttactgcgttaa

SEQ ID NO: 216 - protein, 495 aa, Salmonella [sequence omitted]

SEQ ID NO: 217 - DNA, 498 bp, Salmonella
atgcgtaaatcagcatctgcagtagcagttcttgctttaattgcatgtggcagtgcccacgcagctggctttgttggtaacaaagcagaggttcaggcagcggttactattgcagctcagaatacaacatcagccaactggagtcaggatcctggctttacagggcctgctgttgctgctggtcagaaagttggtactctcagcattactgctactggtccacataactcagtatctattgcaggtaaaggggcttcggtatctggtggtgtagccactgtcccgttcgttgatggacaaggacagcctgttttccgtgggcgtattcagggagccaatattaatgaccaagcaaatactggaattgacgggcttgcaggttggcgagttgccagctctcaagaaacgctaaatgtccctgtcacaacctttggtaaatcgaccctgccagcagggactttcactgcgaccttctacgttcagcagtatcaaaactaa

SEQ ID NO: 218 - protein, 165 aa, Salmonella
MRKSASAVAVLALIACGSAHAAGFVGNKAEVQAAVTIAAQNTTSANWSQDPGFTGPAVAAGQKVGTLSITATGPHNSVSIAGKGASVSGGVATVPFVDGQGQPVFRGRIQGANINDQANTGIDGLAGWRVASSQETLNVPVTTFGKSTLPAGTFTATFYVQQYQN

SEQ ID NO: 219 - DNA, 543 bp, Salmonella
atgacctctactattgcgagtctgatgtttgtcgctggcgcagcggttgcggctgatcctactccggtgagcgtgagtggcggtactattcatttcgaaggtaaactggttaatgcagcctgtgccgtcagcactaaatccgccgatcaaacggtgacgctgggtcaataccgtaccgccagctttacggcgattggtaatacgactgcgcaggtgcctttctccatcgtcctgaatgactgcgatccgaaagtggcggccaacgctgccgtggctttctctggtcaggcagataacaccaaccctaatttgctggctgtctcctctgcggacaatagcactaccgcaaccggcgtcgggattgagattcttgataatacctcttcaccgttgaagccggacggcgcgaccttctcggcgaagcagtcgctggttgaaggcaccaatacgctgcgttttaccgcacgctataaggcaaccgccgccgccacgacgccaggccaggctaatgccgacgccacctttatcatgaaatacgaataa

SEQ ID NO: 220 - protein, 180 aa, Salmonella
MTSTIASLMFVAGAAVAADPTPVSVSGGTIHFEGKLVNAACAVSTKSADQTVTLGQYRTASFTAIGNTTAQVPFSIVLNDCDPKVAANAAVAFSGQADNTNPNLLAVSSADNSTTATGVGIEILDNTSSPLKPDGATFSAKQSLVEGTNTLRFTARYKATAAATTPGQANADATFIMKYE

SEQ ID NO: 221 - DNA, 243 bp, Salmonella
atggcaacaccttggtcaggctatctggatgacgtctcagcaaaatttgatacgggcgttgataatctacaaacgcaggtaacagaggcgctggataaattagcagcaaaaccctccgatccggcgctactggcggcgtatcagagtaagctctcggaatataacttgtaccgtaacgcgcaatcgaacacggtaaaagtctttaaggatattgatgctgccattattcagaacttccgttaa

SEQ ID NO: 222 - protein, 80 aa, Salmonella
MATPWSGYLDDVSAKFDTGVDNLQTQVTEALDKLAAKPSDPALLAAYQSKLSEYNLYRNAQSNTVKVFKDIDAAIIQNFR

SEQ ID NO: 223 - DNA, 1740 bp, Shigella
cataatgtaagcaccacaaccactggttttcctcttgccaaaatattggcttccactgagcttggagacaatactatccaagctgcaaatgatgcagctaacaaattattttctcttacaattgctgatcttactgctaaccaaaatattaatacaactaatgcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtcccagcaacaggcaagacagcaaaaaaacctagaattctccgataaaattaacactcttctatctgaaactgaaggactaaccagagactatgaaaaacaaattaataaactaaaaaacgcagattctaaaataaaagacctagaaaataaaattaaccaaattcaaacaagattatccgaactcgacccagagtcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctagttgcagcagtcgttctcgtagccactgttggtaaacaggcagcagcaaaacttgcagaaaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtcgacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttga

SEQ ID NO: 224 - protein, 579 aa, Shigella
HNVSTTTTGFPLAKILASTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSELDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 225 - DNA, 1089 bp, Shigella
gaaattcaaaacacaaaaccaacccagactttatatacagatatatccacaaaacaaactcaaagttcttccgaaacacaaaaatcacaaaattatcagcagattgcagcgcatattccacttaatgtcggtaaaaatcccgtattaacaaccacattaaatgatgatcaacttttaaagttatcagagcaggttcagcatgattcagaaatcattgctcgccttactgacaaaaagatgaaagatctttcagagatgagtcacacccttactccagagaacactctggatatttccagtctttcttctaatgctgtttctttaattattagtgtagccgttctactttctgctctccgcactgcagaaactaaattgggctctcaattgtcattgattgcgttcgatgctacaaaatcagctgcagagaacattgttcggcaaggcctggcagccctatcatcaagcattactggagcagtcacacaagtaggtataacgggtatcggtgccaaaaaaacgcattcagggattagcgaccaaaaaggagccttaagaaagaaccttgccactgctcaatctcttgaaaaagagcttgcaggttctaaattagggttaaataaacaaatagatacaaatatcacctcaccacaaactaactctagcacaaaatttttaggtaaaaataaactggcgccagataatatatccctgtcaactgaacataaaacttctcttagttctcccgatatttctttgcaggataaaattgacacccagagaagaacttacgagctcaataccctttctgcgcagcaaaaacaaaacattggccgtgcaacaatggaaacatcagccgttgctggtaatatatccacatcaggagggcgttatgcatctgctcttgaagaagaagaacaactaatcagtcaggccagcagtaaacaagcagaggaagcatcccaagtatctaaagaagcatcccaagcgacaaatcaattaatacaaaaattattgaatataattgacagcatcaaccaatcaaagaattcgacagccagtcagattgctggtaacattcgagcttaa

SEQ ID NO: 226 - protein, 362 aa, Shigella
EIQNTKPTQTLYTDISTKQTQSSSETQKSQNYQQIAAHIPLNVGKNPVLTTTLNDDQLLKLSEQVQHDSEIIARLTDKKMKDLSEMSHTLTPENTLDISSLSSNAVSLIISVAVLLSALRTAETKLGSQLSLIAFDATKSAAENIVRQGLAALSSSITGAVTQVGITGIGAKKTHSGISDQKGALRKNLATAQSLEKELAGSKLGLNKQIDTNITSPQTNSSTKFLGKNKLAPDNISLSTEHKTSLSSPDISLQDKIDTQRRTYELNTLSAQQKQNIGRATMETSAVAGNISTSGGRYASALEEEEQLISQASSKQAEEASQVSKEASQATNQLIQKLLNIIDSINQSKNSTASQIAGNIRA

SEQ ID NO: 227 - DNA, 249 bp, Shigella
agtgttacagtaccgaatgatgattggacattgagttcattatctgaaacttttgatgatggaactcaaacattacaaggtgaactaacattggcactagataaattagctaaaaatccttcgaatccacagttgctggctgaataccaaagtaaattatctgaatatacattatataggaacgcgcaatccaatacagtgaaagtgattaaggatgttgatgctgcaattattcaaaacttcagataa

SEQ ID NO: 228 - protein, 82 aa, Shigella
SVTVPNDDWTLSSLSETFDDGTQTLQGELTLALDKLAKNPSNPQLLAEYQSKLSEYTLYRNAQSNTVKVIKDVDAAIIQNFR

SEQ ID NO: 229 - RNA, 21 nt, Artificial sequence
  Synthetic construct
ggggacgacgucguggggggg

SEQ ID NO: 230 - RNA, 20 nt, Artificial sequence
  Synthetic construct
aauuguguaauguccuucaa

SEQ ID NO: 231 - protein, 5 aa, Artificial sequence
  Synthetic construct
CDGRC

SEQ ID NO: 232 - protein, 37 aa, Artificial sequence
  Synthetic construct
LLGDFFRKSKEKIGKEFKRIVQRIKDFLRNLVPRTES

SEQ ID NO: 233 - protein, 26 aa, Artificial sequence
  Synthetic construct
KKALLALALHHLAHLALHLALALKKA

SEQ ID NO: 234 - protein, 26 aa, Artificial sequence
  Synthetic construct
KKALLALALHHLAHLAHHLALALKKA

SEQ ID NO: 235 - protein, 26 aa, Artificial sequence
  Synthetic construct
KKALLALALHHLALLAHHLALALKKA

SEQ ID NO: 236 - protein, 15 aa, Human immunodeficiency virus
YGRKKRRQRRRPPQC

SEQ ID NO: 237 - protein, 16 aa, Drosophila
RQIKIWFQNRRMKWKK

SEQ ID NO: 238 - DNA, 17 bp, Artificial sequence
  Synthetic construct
gaggctctctctctctc

SEQ ID NO: 239 - DNA, 38 bp, Artificial sequence
  Synthetic construct
tccatgacgttcctgacgtttctctctctctctcggag

SEQ ID NO: 240 - DNA, 52 bp, Artificial sequence
  Synthetic construct
cggcggataaccgcgagcggttattcgccctacggctctctctctctcggag

SEQ ID NO: 241 - DNA, 36 bp, Artificial sequence
  Synthetic construct
gggggacgatcgtcgggggctctctctctctcggag

SEQ ID NO: 242 - protein, 4 aa, Artificial sequence
  Synthetic construct
GFLG

SEQ ID NO: 243 - DNA, 15 bp, Artificial sequence
  Synthetic construct
ctctctctctctctc

SEQ ID NO: 244 - DNA, 14 bp, Artificial sequence
  Synthetic construct
ccttccttccttcc

SEQ ID NO: 245 - DNA, 18 bp, Artificial sequence
  Synthetic construct
cttcttcttcttcttctt

SEQ ID NO: 246 - DNA, 18 bp, Artificial sequence
  Synthetic construct
cctcctcctcctcctcct

SEQ ID NO: 247 - DNA, 11 bp, Artificial sequence
  Synthetic construct
tctcctccttt

SEQ ID NO: 248 - RNA, 15 nt, Artificial sequence
  Synthetic construct
cucucucucucucuc

SEQ ID NO: 249 - RNA, 14 nt, Artificial sequence
  Synthetic construct
ccuuccuuccuucc

SEQ ID NO: 250 - RNA, 18 nt, Artificial sequence
  Synthetic construct
cuucuucuucuucuucuu

SEQ ID NO: 251 - RNA, 18 nt, Artificial sequence
  Synthetic construct
ccuccuccuccuccuccu

SEQ ID NO: 252 - RNA, 11 nt, Artificial sequenced
  Synthetic construct
ucuccuccuuu

SEQ ID NO: 253 - DNA, 13 bp, Artificial sequence
  Synthetic construct
ctctctctctctc

SEQ ID NO: 254 - DNA, 13 bp, Artificial sequence
  Synthetic construct
gagagagagagag

SEQ ID NO: 255 - DNA, 17 bp, Artificial sequence
  Synthetic construct
ctctctctctctctctc

SEQ ID NO: 256 - DNA, 14 bp, Artificial sequence
  Synthetic construct
ccttccttccttcc

SEQ ID NO: 257 - DNA, 14 bp, Artificial sequence
  Synthetic construct
ggaaggaaggaagg

SEQ ID NO: 258 - DNA, 18 bp, Artificial sequence
  Synthetic construct
ctctctctcctctctctc

SEQ ID NO: 259 - DNA, 34 bp, Artificial sequence
  Synthetic construct
tccatgacgttcctgacgtttgagagagagagag

SEQ ID NO: 260 - DNA, 34 bp, Artificial sequence
  Synthetic construct
tccatgagcttcctgagtcttgagagagagagag

SEQ ID NO: 261 - DNA, 26 bp, Artificial sequence
  Synthetic construct
ctctctctctctcctctctctctctc

SEQ ID NO: 262 - DNA, 26 bp, Artificial sequence
  Synthetic construct
ctctctctctctcctctctctctctc

SEQ ID NO: 263 - DNA, 13 bp, Artificial sequence
  Synthetic construct
ctctctctctctc

SEQ ID NO: 264 - DNA, 52 bp, Artificial sequence; identical to SEQ ID NO: 240
  Synthetic construct

SEQ ID NO: 265 - DNA, 36 bp, Artificial sequence; identical to SEQ ID NO: 241
  Synthetic construct

SEQ ID NO: 266 - DNA, 15 bp, Artificial sequence
  Synthetic construct
gaggggaggggaggg

SEQ ID NO: 267 - DNA, 10 bp, Artificial sequence
  Synthetic construct
gctcggatcc

SEQ ID NO: 268 - DNA, 10 bp, Artificial sequence
  Synthetic construct
cgtacggtcg

SEQ ID NO: 269 - DNA, 20 bp, Artificial sequence
  Synthetic construct
cgaccgtacgggatccgagc

SEQ ID NO: 270 - DNA, 22 bp, Artificial sequence
  Primer
ggaaggaagttaggaaggaagg

(19) United States
(12) Patent Application Publication
(10) Pub. No.: US 2009/0123467 A1
(43) Pub. Date: May 14, 2009

(54) Polypeptide-Nucleic Acid Conjugate for Immunoprophylaxis or Immunotherapy for Neoplastic or Infectious Disorders

(75) Inventors: Atul Bedi, Timonium, MD (US); Rajani Ravi, Ruxton, MD (US); Shulin Li, Baton Rouge, LA (US)

Correspondence Address: DLA PIPER LLP (US), 4365 EXECUTIVE DRIVE, SUITE 1100, SAN DIEGO, CA, 92121-2133, US

(73) Assignee: The Johns Hopkins University, Baltimore, MD (US)

(21) Appl. No.: 12/184,246

(22) Filed: Jul. 31, 2008

Related U.S. Application Data
(60) Provisional application No. 61/007,895, filed on Jul. 31, 2007.
(60) Provisional application No. 61/022,173, filed on Jan. 18, 2008.

(51) Int. Cl. A61K 39/39; C07K 2/00; A61P 37/02; C07K 16/00

(52) U.S. Cl. 424/134.1; 530/403; 530/387.3; 424/193.1

(57) ABSTRACT

The present invention discloses compositions which induce cross-activation of immune mediated and direct death signaling in targeted cells by exploiting the properties of a antibody/peptide-nucleic acid conjugate. The conjugate is able to simultaneously activate multiple death signaling mechanisms. Methods of using the conjugate of the present invention as an immunotherapeutic modality for the treatment or prevention of infectious disease, neoplastic diseases or other disorders.

### CROSS-REFERENCE

This application claims the benefit of priority under 35 U.S.C. § 119(e) to U.S. provisional applications No. 61/007,895, filed Jul. 31, 2007 and 61/022,173, filed Jan. 18, 2008, which are in-corporated herein by reference in their entirety. In addition, this application is related to U.S. utility application Ser. No. 11/701,092, filed Jan. 31, 2007 and U.S. provisional applications No. 60/764,223, filed Feb. 1, 2006 and 60/833,100, filed Jul. 25, 2006, each of which is incorporated herein by reference in its entirety.

### FIELD OF THE INVENTION

The present invention relates generally to immunostimulatory therapeutic modalities and, more specifically to antibody/peptide-nucleic acid conjugates for the prevention or treatment of neoplastic, infectious and/or other disorders.

### BACKGROUND INFORMATION

The immune system provides the human body with a means to recognize and defend itself against microorganisms and substances recognized as foreign or potentially harmful. Preventative vaccination against infectious organisms have had a major benefit in protecting populations from infection. However, effective immunoprophylaxis and immunotherapy are still needed for many prevalent infectious diseases and persistent infections. While passive immunotherapy of cancer with monoclonal antibodies and passive transfer of T cells to attack tumor cells have demonstrated clinical efficacy, the goal of active therapeutic vaccination to induce these immune effectors and establish immunological memory against tumor cells has remained challenging. Several tumor-specific and tumor-associated antigens have been identified, yet these antigens are generally weakly immunogenic and tumors employ diverse mechanisms to create a tolerogenic environment that allows them to evade immunologic attack. Strategies to overcome such immune tolerance and activating robust levels of antibody and/or T cell responses hold the key to effective cancer immunotherapy.

Dendritic cells (DCs) are specialized antigen presenting cells (APCs) which play a central role in the initiation and regulation of primary immune responses. (i) Antigen uptake and presentation: DCs capture pathogens (bacteria, viruses), dead or dying cells, proteins, and immune complexes through phagocytosis, endocytosis, and pinocytosis. They have an array of cell surface receptors for antigen uptake, which may also function in signaling and cell-cell interactions (Table 1). DCs process captured proteins into peptides that are loaded on to major histocompatibility complex class I and II (MHC I and II) molecules, and these peptide-MHC complexes are transported to the cell surface for recognition by antigen-specific CD8+ T cells (by MHC I) and CD4+ T cells (by MHC II). Antigens synthesized endogenously within the DC cytosol are typically processed through a proteasome-mediated pathway into the endoplasmic reticuluma and loaded on to MHC I, whereas antigens acquired exogenously from the extracellular environment are typically degraded in endosomes/lysosomes and loaded on to MHC II. An alternative route, linked to specific DC antigen uptake receptors (Table 2), also enables DCs to process exogenous antigens on to MHC I (cross-presentation). Cross-presentation allows DCs to elicit CD8+ as well as CD4+ T cell responses to exogenous antigens such as tumor cells, pathogen-infected cells, and immune complexes. (ii) DC maturation—Role of TLRs: Maturation of DCs is a process of terminal differentiation which transforms DCs from specialized antigen capture cells into cells that can stimulate T cells. DC maturation is induced by recognition of pathogen-derived components or by endogenous host molecules associated with inflammation or tissue damage (termed “danger signals”). These maturation signals engage receptors expressed on DC that trigger intracellular signaling pathways. The recognition of pathogen-associated molecular patterns (PAMPs) expressed by diverse infectious microorganisms (bacteria, fungi, protozoa, viruses) and molecules released by damaged host tissues (damage associated molecular patterns/alarmins) is mediated by pattern recognition receptors (PRRs) such as members of the Toll-like receptor (TLR) family expressed on DCs. TLRs are type I membrane glycoprotein's. In humans, the 10 known functional TLRs with specific expression patterns, subcellular localization, and recognition ability for different molecules. In humans, myeloid DCs express TLRs 1-5,7 and/or 8, while plasmacytoid DCs express TLRs 1,7, and 9. Whereas some TLRs operate at the cell surface (TLR1,2,4,5,6,10), TLRs 3,7,8, and 9 are expressed in intracellular compartments (principally endosomes and endoplasmic reticulum) with the ligand binding domains sampling the lumen of the vesicle. TLR recognition of pathogen-encoded TLR ligands fall into three broad categories of structurally similar molecules: lipids and lipopeptides (TLR2/TLR1; TLR2/TLR6; TLR4), proteins (TLR5) and nucleic acids (TLR3,7,8, and 9). Of the TLRs which recognize immunostimulatory nucleic acids, TLR3 engages ds RNA, TLR7/8 engage ss RNA, and TLR9 engages DNA. In addition to microbial ligands, endogenous ligands have been identified for most TLRs (mRNA for TLR3, ss RNA immune complexes for TLR7/8, and DNA immune complexes for TLR9). Synthetic ligands have also been described for most of the TLRs, including immunostimulatory nucleic acid sequences (INAS) that can activate TLR3, 7, 8 (ds RNA, ss RNA) and TLR9 (oligodeoxynucleotides containing unmethylated CpG motifs)(Table 3). Ligand binding of TLR leads to recruitment of different adaptor proteins leading to the activation of cell-type specific signaling pathways and responses. However, differential patterns of TLR expression among subsets of DCs/APCs (human PDC, but not MDC express TLR9 and respond to DNA; PDC and MDC respond differently to ss RNA) and differences in the cellular distribution of APC at different anatomical sites can result in diverse responses to different TLR ligands (natural or synthetic) or varying routes of administration of the same ligand. Maturation of DCs in response to TLR agonists or other stimuli (cytokines, immune complexes, adhesion molecules) is attended with reduced phagocytic function, migration to lymphoid tissues, and enhanced ability to activate T cells. Maturation of DCs enhances their ability to form MHC I and II molecules, induces cross-presentation, increases expression of adhesion and costimulatory molecules involved in immunologic synapses required for T cell activation (CD40, CD80, CD86), induces secretion of cytokines (IFN-γ, IFN-α, IL-12) that guide T cell differentiation to either CD4+ T helper type (T_{H}1) or CD8+ cytotoxic lymphocytes (CTL), and chemokines that recruit monocytes, DCs, and T cells to the local mileu. Mature DCs also become capable of migration to T cell zones of lymph nodes. In addition to their ability to prime antigen-specific T cell immune responses, DCs engage in a complex bidirectional crosstalk with NK cells to facilitate immune surveillance and elimination of pathogens and tumors. Activated DCs also induce B cell proliferation, isotype switching, and differentiation of plasma cells to produce antibodies. Since DCs plays a crucial role in the coordinated activation of innate and adaptive immune responses, strategies to stimulate DC-mediated activation of antigen-specific T cells and NK cells may not only harness the direct anti-tumor or anti-pathogen effects of the innate immune system, but also facilitate the generation of long-lasting adaptive tumor-specific or pathogen-specific immune responses.

Classical immune responses are initiated when antigen-presenting cells present an antigen to “prime” T cells in secondary lymphoid tissues, resulting in T cell activation, proliferation, and differentiation into effector T lymphocytes and memory cells. The nature of the T cell response is dependent on the concentration of antigen on the DC, the affinity of the T cell receptor for the corresponding pMHC, and the state of DC maturation. Immature DCs abort initial proliferation with activation-induced cell death of antigen-specific T cells, and can also induce tolerance via induction of regulatory T cells. However, stimulation by mature DCs results in long-term T cell survival and differentiation into memory and effector cells, with concurrent inhibition of naturally occurring Tr cells. Following exposure to antigens, such as that which results from infection, naive T cells may differentiate into T_{H}1 and T_{H}2 cells with differing functions, or into T_{H}3 cells, Tr1 cells, T_{H}17 cells, or regulatory T cells (T_{regs}). CD4+ T helper (T_{H}) cells are vital for the induction and maintenance of immune responses and memory. This effect is mediated by ligand/receptor interactions between the T_{H} cells and DCs, such as via CD40L engagement of CD40 expressed on DCs. T_{H} cell help at the time of priming is critically required for priming and secondary expansion of CD8+ T cells and providing help to B cells for antibody production. Once induced, CD8+ memory T cells no longer rely on continued antigen-specific T_{H} support. Since autologous tumor antigens are usually incapable of inducing significant T_{H} responses, the endogenous CD8+ effector T cell response against tumor cells is impaired. Tumors may also evade immunity via loss of antigen or MHC expression or immunosuppressive mechanisms, such as secretion of TGF-β. In addition to interfering with the afferent arm of the immune response, tumor cells may also harbor genetic aberrations or enhanced growth factor receptor-mediated survival pathways which reduce their susceptibility to apoptosis in response to the efferent death signaling pathways entrained by cytotoxic T cells.

### SUMMARY OF THE INVENTION

The present invention describes multifunctional targeted immunoconjugate moieties which enable the effective generation of innate and adaptive immune responses against tumors or pathogens. These immunoconjugates are capable of simultaneously satisfying multiple key requirements for mounting effective antibody- and/or cell-mediated immune responses against the targeted tumor or pathogen: (i) Induce or augment uptake and cross-presentation of tumor- or pathogen antigen(s) or antigenic determinant(s) by antigen presenting cells (APC)/dendritic cells (DC); (ii) Promote the maturation of dendritic cells (DCs) in the target cell milieu; (iii) provide CD4+ T cell help to generate CD8+ T cell memory and antibodies against the tumor or pathogen; (iv) sensitize the targeted tumor cell to antibody dependent cell cytotoxicity (ADCC) and T-cell mediated death. Further, the present invention can be used for targeted immunotherapy or immunoprophylaxis of neoplastic diseases, infectious diseases, and other disorders.

In general, compositions and methods of the invention involve a therapeutic or diagnostic compound comprising a targeting moiety that can bind a target molecule or cell component and one or more active agent(s) which enhance(s) an immune response against a desired antigen or cell. As further described herein, targeting moieties are specific for molecules or components of a cancer or tumor, of a normal cell (such as a dendritic cell or keratinocyte), or of an infectious agent or pathogen. Furthermore, an active agent includes nucleic acids, peptides, polypeptides, lipopeptides, or combinations thereof.

In a first aspect of the invention, products and processes of the invention are directed to a composition comprising a targeting moiety (T) and one, two, three or more active agents (A).

In one embodiment, a composition of the invention comprises a targeting moiety coupled to an active agent. In another embodiment, a composition comprises a targeting moiety, and at least two active agents, which include a non-coding or coding nucleic acid molecule and a peptide or polypeptide or lipopeptide. In a further embodiment, the at least two active agents include a non-coding nucleic acid molecule and a coding nucleic acid molecule (e.g., plasmid or minicircle). In yet a further embodiment, the at least two active agents include a non-coding or coding nucleic acid molecule, and an antigenic peptide or polypeptide. For simplified illustration, compositions of the invention can be covered by the following formula: T-A₁ or T-A₁-A₂, where T=targeting moiety; A₁ is either a nucleic acid molecule or peptide or polypeptide or lipopeptide; and A₂ is either a nucleic acid molecule or peptide or polypeptide or lipopeptide. Furthermore, the nucleic acid molecule can be a coding or non-coding sequence as further described herein. In further embodiments, A₁ can be coupled (directly or indirectly) to an additional component including a nucleic acid molecule, a peptide, a polypeptide, or lipopeptide. Alternatively, in further embodiments an active agent is a component for packaging and/or delivery of a nucleic acid molecule.

As used herein, “targeting moiety” (or moieties) refers to a molecule(s) that has the ability to localize to and bind a target molecule present on a normal cell/tissue and/or cancer cell/tumor or other molecule. In other words, compositions of the invention comprising such a targeting moiety can bind to a targeted cell or molecule (directly or indirectly). The targeting moieties of the invention contemplated for use with the biologically active agents include antibody, polypeptides, peptides, aptamers, other ligands, or any combination thereof, that can bind a component of the target cell or molecule.

As disclosed herein, a nucleic acid molecule comprises one or more of the following: double strand DNA (ds DNA), single strand DNA (ssDNA), multistrand DNA, double strand RNA (ds RNA), single strand RNA (ssRNA), multistrand RNA, DNA-RNA hybrid (single strand or multistrand), peptide nucleic acid (PNA), PNA-DNA hybrid (single or multistrand), PNA-RNA hybrid (single or multistrand), locked nucleic acids (LNA), LNA-DNA hybrid (single or multistrand), LNA-RNA hybrid (single or multistrand). In one embodiment, the nucleic acid molecule encodes one or more products (e.g. nucleic acids such as RNA, peptides, polypeptides, fusion peptides). In one embodiment, the nucleic acid molecule includes one or more immunostimulatory nucleic acid sequences (INAS) that can activate immune cells.

In one embodiment, a composition of the invention comprises one or more targeting moiety (T) which binds a target molecules or component of a cancer or tumor (tumor-targeting moiety). The targeted molecule may be a component of a tumor cells, tumor vasculature, or tumor microenvironment.

In one embodiment, the invention comprises a conjugate of a tumor-targeting moiety, such as an antibody, and a nucleic acid molecule, wherein the nucleic acid molecule encodes one or more products (e.g. nucleic acids such as RNA, peptides, polypeptides, fusion peptides) and is capable of stimulating an immune response. In one embodiment, the nucleic acid molecule includes one or more pathogen associated molecular pattern (PAMP) or other immunostimulatory motif. In another embodiment, the nucleic acid molecule encodes one or more products that stimulate an immune response. In a related embodiment, the nucleic acid molecule includes one or more pathogen associated molecular pattern (PAMP) or other immunostimulatory motif, and encodes one or more products that stimulates an immune response.

In a related embodiment, the nucleic acid molecule of the tumor-targeted conjugate encodes one or more antigens or antigenic determinants which can be processed and presented for recognition by T cells and/or B cells. The encoded antigenic determinants include one or more of each of the following: CD4⁺T cell epitopes, CD8⁺ T cell epitopes, B cell epitopes. In one embodiment, the nucleic acid molecule encodes one or more antigens or antigenic determinants derived from one or more pathogen(s), microorganism(s) or virus(es). For example, the nucleic acid encodes sequences derived from tetanus toxin to provide CD4⁺ T-cell help [e.g. Tetanus derived T_{H} activating sequences: fragment C (FrC), FrC domain DOM1, or the promiscuous MHC class II-binding peptide p30]. In a related embodiment, the nucleic acid encodes one or more antigens or antigenic determinants derived from a microbial vaccine or other non-self source (e.g. Pseudomonas aeruginosa exotoxin, green fluorescent protein, plant viral coat proteins).

In a related embodiment, the invention comprises a conjugate of a tumor-targeting moiety, such as an antibody, one or more pathogen associated molecular pattern (PAMP) and/or nucleic acid molecule(s) encoding one or more antigens or antigenic determinants derived from one or more pathogen(s), microorganism(s) or virus(es)(T or B cell epitopes). In a related embodiment, the conjugate comprises a tumor targeting moiety and one or more PAMP(s). In another related embodiment, the conjugate comprises a tumor targeting moiety and one or more nucleic acid molecule(s) encoding one or more antigens or antigenic determinants derived from one or more pathogen(s), microorganism(s) or virus(es)(T or B cell epitopes). In another related embodiment, the conjugate comprises a tumor targeting moiety, one or more PAMP(s), and one or more nucleic acid molecule(s) encoding one or more antigens or antigenic determinants derived from one or more pathogen(s), microorganism(s) or virus(es)(T or B cell epitopes).

In one embodiment, the invention comprises a conjugate of a tumor-targeting moiety, such as an antibody, one or more damage associated molecular pattern (DAMP) or alarmin(s), and one or more nucleic acid molecule(s) encoding one or more antigens or antigenic determinants derived from one or more pathogen(s), microorganism(s) or virus(es)(T or B cell epitopes). In a related embodiment, the conjugate comprises a tumor targeting moiety and one or more DAMP/Alarmin(s). In another related embodiment, the conjugate comprises a tumor targeting moiety and one or more nucleic acid molecule(s) encoding one or more antigens or antigenic determinants derived from one or more pathogen(s), microorganism(s) or virus(es)(T or B cell epitopes). In another related embodiment, the conjugate comprises a tumor targeting moiety, one or more DAMP/Alarmin(s), and one or more nucleic acid molecule(s) encoding one or more antigens or antigenic determinants derived from one or more pathogen(s), microorganism(s) or virus(es)(T or B cell epitopes).

In one embodiment, the invention comprises a conjugate of a tumor-targeting moiety, such as an antibody, and one or more nucleic acid molecule(s) encoding one or more of the following: (i) one or more antigens or antigenic determinants derived from one or more pathogen(s), microorganism(s) or virus(es)(T or B cell epitopes), (ii) one or more pathogen associated molecular pattern (PAMP), (iii) one or more damage associated molecular patterns (DAMP)/alarmin(s), (iv) one or more immunostimulatory molecules, including molecules that recruit, bind, activate, mature and/or proliferate an antigen presenting cell or dendritic cell or other immune cell (such as T cells, B cells, NK cells) and molecules that counteract immune suppression (e.g. ligands/antibodies for DC uptake receptors, immunostimulatory cytokines, chemokines, costimulatory molecules, growth factors). In a related embodiment, the nucleic acid molecule additionally encodes one or more tumor antigens/antigenic determinants or tumor antigen-containing fusion proteins. In one aspect, the fusion partner of the tumor antigen facilitates antigen uptake by DCs, immune recognition, and/or immune activation. In another example, the fusion partner includes a molecule targeting a DC uptake receptor. In another example, the fusion partner is an antigen or antigenic determinant derived from one or more pathogen(s), microorganism(s) or virus(es). In another example, the fusion partner is an alarmin. In a related embodiment, the targeting moiety-nucleic acid conjugate(s) described herein further comprises one or more PAMP and/or one or more DAMP/Alarmin(s).

In one embodiment, the invention comprises a conjugate of a tumor-targeting moiety, such as an antibody, and one or more nucleic acid molecule(s) encoding one or more RNA molecules that can interfere with expression of one or more target cell genes [e.g. short interfering RNA (siRNA), short hairpin RNA (shRNA)]. In another embodiment, the nucleic acid molecule of the conjugate encodes one or more immunostimulatory RNA molecules.

In one embodiment, the invention comprises a conjugate of a tumor-targeting moiety, such as an antibody, and one or more nucleic acid molecule(s) encoding a molecule that induces death of the target cell.

In each of the targeting moiety-nucleic acid conjugates described herein, the nucleic acid molecule encodes one or more gene of interest under control of a transcription promoter that is functionally active in the desired cell. In one embodiment, tissue or tumor cell selective promoters are used for targeted expression in the desired cell type.

In one embodiment, each of the tumor targeting moiety-nucleic acid conjugates described herein is linked to one or more components for packaging and/or delivery of a nucleic acid molecule or conjugate. For example, these molecules include cationic peptide, cell permeabilizing peptide, DC targeting peptide, nucleic acid binding molecule, nuclear localization peptide, cationic liposome, lipophilic moiety, nanoparticle.

In one embodiment, the invention comprises a conjugate of a tumor-targeting moiety, such as an antibody, one or more nucleic acid molecule(s), and one or more peptide/polypeptide/lipopeptide(s). In one embodiment, the nucleic acid molecule incorporates one or more pathogen associated molecular pattern (PAMP) or other immunostimulatory motif, and/or encodes one or more products that stimulate an immune response, as described herein. In various related embodiments, the peptide/polypeptide/lipopeptide(s) include one or more of the following: (i) one or more antigens or antigenic determinants derived from one or more pathogen(s), microorganism(s) or virus(es)(e.g. CD4+ T cell epitopes), (ii) alarmins, (iii) DC binding molecules (e.g. ligands of DC uptake receptors). In one aspect, the peptide/polypeptides of the conjugate described herein may be fused/linked to each other and/or to a nucleic acid binding peptide or cell permeabilizing peptide (e.g. cationic peptides, protamine, HIV-tat, Arginine- or Histidine-rich sequence, LL-37).

In one embodiment, the invention comprises a conjugate of a tumor-targeting moiety, such as an antibody or aptamer, and one or more of the following: (a) one or more pathogen associated molecular pattern (PAMP), (b) one or more of the following peptide/polypeptide/lipopeptide(s):(i) one or more antigens or antigenic determinants derived from one or more pathogen(s), microorganism(s) or virus(es)(e.g. CD4+ T cell epitopes), (ii) alarmins, (iii) DC binding molecules (e.g. ligands of DC uptake receptors). In one aspect, the peptide/polypeptides of the conjugate described herein may be fused/linked to each other and/or to a nucleic acid binding peptide (e.g. cationic peptides, protamine, HIV-tat, Arginine- or Histidine-rich sequence, LL-37). In one aspect, the conjugate includes an immunostimulatory nucleic acid.

In one embodiment, the invention comprises a conjugate of a targeting moiety, such as an antibody, and a nucleic acid molecule which is an aptamer. In one embodiment the antibody and nucleic acid aptamer bind to different targets on the same cell type or different cell types. In one embodiment, the conjugate comprises an antibody targeting a tumor cell surface receptor (EGFR) and an aptamer targeting prostate specific membrane antigen (PSMA), thereby targeting both proteins in prostate cancer cells. In one embodiment, the nucleic acid molecule comprises the aptamer and one or more of the following: (i) PAMP or other immunostimulatory nucleic acid, (ii) DNA encoding one or more products that stimulate an immune response, as described herein.

In one embodiment, a composition of the invention comprises one or more targeting moiety (T) which binds a target molecules or component of a normal cell or tissue, such as keratinocytes in skin (tissue-targeting moiety). In one embodiment, the targeting moiety binds a cell surface molecule or receptor on keratinocytes, such as the epidermal growth factor receptor (EGFR).

In one embodiment, the invention comprises a conjugate of a tissue-targeting moiety, such as an antibody to EGFR, and a nucleic acid molecule, wherein the nucleic acid molecule encodes one or more products (e.g. nucleic acids such as RNA, peptides, polypeptides, fusion peptides) and is capable of stimulating an immune response. In one embodiment, the nucleic acid molecule includes one or more pathogen associated molecular pattern (PAMP) or other immunostimulatory motif. In another embodiment, the nucleic acid molecule encodes one or more products that stimulate an immune response. In a related embodiment, the nucleic acid molecule includes one or more pathogen associated molecular pattern (PAMP) or other immunostimulatory motif, and encodes one or more products that stimulates an immune response.

In one embodiment, the invention comprises a conjugate of a tissue-targeting moiety, such as an antibody to EGFR, and a nucleic acid molecule, wherein the nucleic acid molecule includes one or more pathogen associated molecular pattern (PAMP) and encodes one or more antigens or antigenic determinants derived from one or more pathogen(s), microorganism(s) or virus(es)(T or B cell epitopes).

In one embodiment, the invention comprises a conjugate of a tissue-targeting moiety, such as an antibody to EGFR, one or more pathogen associated molecular pattern (PAMP), and nucleic acid molecule encoding one or more antigens or antigenic determinants derived from one or more pathogen(s), microorganism(s) or virus(es)(T or B cell epitopes).

In one embodiment, the invention comprises a conjugate of a tissue-targeting moiety, such as an antibody to EGFR, one or more damage associated molecular pattern (DAMP) or alarmin, and a nucleic acid molecule encoding one or more antigens or antigenic determinants derived from one or more pathogen(s), microorganism(s) or virus(es)(T or B cell epitopes).

In one embodiment, the invention comprises a conjugate of a a tissue-targeting moiety, such as an antibody to EGFR, one or more nucleic acid molecule(s) encoding one or more antigens or antigenic determinants derived from one or more pathogen(s), microorganism(s) or virus(es)(T or B cell epitopes), and encoding none, one, or more of the following: (i) one or more pathogen associated molecular pattern (PAMP), (ii) one or more damage associated molecular patterns (DAMP)/alarmin(s), (iii) one or more immunostimulatory molecules, including molecules that recruit, bind, activate, mature and/or proliferate an antigen presenting cell or dendritic cell or other immune cell (such as T cells, B cells, NK cells) and molecules that counteract immune suppression (e.g. ligands/antibodies for DC uptake receptors, immunostimulatory cytokines, chemokines, costimulatory molecules, growth factors). In a related embodiment, the nucleic acid molecule encodes one or more pathogen antigens/antigenic determinants as fusion proteins. In one aspect, the fusion partner of the antigen facilitates antigen uptake by DCs, immune recognition, and/or immune activation. In another aspect, the fusion partner includes a molecule targeting a DC uptake receptor. In another aspect, the fusion partner is an alarmin. In a related embodiment, the targeting moiety-nucleic acid conjugate(s) described herein further comprises one or more PAMP and/or one or more DAMP/Alarm in(s).

In one embodiment, the invention comprises a conjugate of a tissue-targeting moiety, such as an antibody to EGFR, one or more nucleic acid molecule(s) encoding one or more tumor antigens/antigenic determinants and encoding one or more of the following: (i) one or more antigens or antigenic determinants derived from one or more pathogen(s), microorganism(s) or virus(es)(e.g. CD4+ T cell epitopes), (ii) one or more pathogen associated molecular pattern (PAMP), (ii) one or more damage associated molecular patterns (DAMP)/alarmin(s), (iii) one or more immunostimulatory molecules, including molecules that recruit, bind, activate, mature and/or proliferate an antigen presenting cell or dendritic cell or other immune cell (such as T cells, B cells, NK cells) and molecules that counteract immune suppression (e.g. ligands/antibodies for DC uptake receptors, immunostimulatory cytokines, chemokines, costimulatory molecules, growth factors). In a related embodiment, the nucleic acid molecule encodes one or more tumor antigen-containing fusion proteins. In one aspect, the fusion partner of the tumor antigen facilitates antigen uptake by DCs, immune recognition, and/or immune activation. In another example, the fusion partner includes a molecule targeting a DC uptake receptor. In another example, the fusion partner is an antigen or antigenic determinant derived from one or more pathogen(s), microorganism(s) or virus(es)(CD4+ T cell epitope). In another example, the fusion partner is an alarmin. In a related embodiment, the targeting moiety-nucleic acid conjugate(s) described herein further comprises one or more PAMP and/or one or more DAMP/Alarmin(s).

In one embodiment, the invention comprises a conjugate of a tissue-targeting moiety, such as an antibody to EGFR, one or more pathogen associated molecular pattern (PAMP) and/or alarmin, and an antigenic peptide/polypeptide that includes one or more of the following: (i) one or more antigens or antigenic determinants derived from one or more pathogen(s), microorganism(s) or virus(es), (ii) one or more tumor antigens or antigenic determinants. In one aspect of the conjugate, the tumor or pathogen-derived antigen or antigenic determinant is linked or fused to an alarmin (e.g. LL 37).

In one embodiment, the invention comprises a conjugate of a tissue-targeting moiety, such as an antibody to EGFR, one or more a nucleic acid molecule(s), and one or more peptide/polypeptide. In one embodiment, the nucleic acid molecule incorporates one or more pathogen associated molecular pattern (PAMP) or other immunostimulatory motif, and/or encodes one or more products that stimulate an antigen-specific immune response, as described herein. In various embodiments of the conjugate, the peptide/polypeptide includes one or more of the following:(i) one or more pathogen and/or tumor antigens or antigenic determinants, (ii) alarmins, (iii) DC binding molecules (e.g. ligands of DC uptake receptors). In one aspect, the peptide/polypeptides of the conjugate described herein may be fused/linked to each other and/or to a nucleic acid binding peptide (e.g. cationic peptides, protamine, HIV-tat, Arginine- or Histidine-rich sequence, LL-37, Nuclear localizing peptide).

In one embodiment, a composition of the invention comprises one or more targeting moiety (T) which binds a target molecules or component of a normal immune cell or tissue, such as antigen presentic cells or dendritic cells (APC/DC-targeting moiety).

In one embodiment, the targeting moiety binds a dendritic cell uptake receptor, such as DEC-205.

In one embodiment, the invention comprises a conjugate comprising an antibody or other moiety targeting an antigen presenting cell (APC)/Dendritic cell (DC), such as a DC uptake receptor, and a nucleic acid molecule which encodes a gene of interest.

In one embodiment, the invention comprises a conjugate of an APC/DC-targeting moiety and a nucleic acid molecule, wherein the nucleic acid molecule encodes one or more products (e.g. nucleic acids such as RNA, peptides, polypeptides, fusion peptides) and is capable of stimulating an immune response. In one embodiment, the nucleic acid molecule includes one or more pathogen associated molecular pattern (PAMP) or other immunostimulatory motif. In another embodiment, the nucleic acid molecule encodes one or more products that stimulate an immune response. In a related embodiment, the nucleic acid molecule includes one or more pathogen associated molecular pattern (PAMP) or other immunostimulatory motif, and encodes one or more products that stimulates an immune response.

In one embodiment, the invention comprises a conjugate of an APC/DC-targeting moiety, such as an antibody to DEC-205, and one or more nucleic acid molecules, wherein the nucleic acid molecule includes one or more pathogen associated molecular pattern (PAMP) and encodes one or more antigens or antigenic determinants derived from one or more pathogen(s), microorganism(s) or virus(es)(T or B cell epitopes). In a related embodiment, the targeting moiety-nucleic acid conjugate(s) described herein further comprises one or more PAMP and/or one or more DAMP/Alarm in(s).

In one embodiment, the invention comprises a conjugate of an APC/DC-targeting moiety, one or more pathogen associated molecular pattern (PAMP), and one or more nucleic acid molecule encoding one or more antigens or antigenic determinants derived from one or more pathogen(s), microorganism(s) or virus(es)(T or B cell epitopes). In a related embodiment, the targeting moiety-nucleic acid conjugate(s) described herein further comprises one or more DAMP/Alarmin(s).

In one embodiment, the invention comprises a conjugate of an APC/DC-targeting moiety, one or more damage associated molecular pattern (DAMP) or alarmin, and one or more nucleic acid molecule encoding one or more antigens or antigenic determinants derived from one or more pathogen(s), microorganism(s) or virus(es)(T or B cell epitopes).

In one embodiment, the invention comprises a conjugate of an APC/DC-targeting moiety and one or more nucleic acid molecule(s) encoding one or more antigens or antigenic determinants derived from one or more pathogen(s), microorganism(s) or virus(es)(T or B cell epitopes), and encoding one or more immunostimulatory molecules, such as molecules that recruit, bind, activate, mature and/or proliferate an antigen presenting cell or dendritic cell or other immune cell (such as T cells, B cells, NK cells) and molecules that counteract immune suppression (e.g. immunostimulatory cytokines, chemokines, costimulatory molecules, growth factors). In a related embodiment, the nucleic acid molecule encodes one or more pathogen antigens/antigenic determinants as fusion proteins. In a related embodiment, the targeting moiety-nucleic acid conjugate(s) described herein further comprises one or more PAMP and/or one or more DAMP/Alarmin(s). In one aspect, the conjugate further includes one or more peptides that include one or more pathogen-derived antigens or antigenic determinants.

In one embodiment, the invention comprises a conjugate of an APC/DC-targeting moiety and one or more nucleic acid molecules encoding one or more tumor antigens and encoding one or more of the following: (i) one or more antigens or antigenic determinants derived from one or more pathogen(s), microorganism(s) or virus(es)(e.g. CD4+ T cell epitopes), (ii) one or more immunostimulatory molecules, such as molecules that recruit, bind, activate, mature and/or proliferate an antigen presenting cell or dendritic cell or other immune cell (such as T cells, B cells, NK cells) and molecules that counteract immune suppression (e.g. immunostimulatory cytokines, chemokines, costimulatory molecules, growth factors). In a related embodiment, the nucleic acid molecule encodes one or more tumor antigens as fusion proteins with an antigen or antigenic determinant derived from one or more pathogen(s), microorganism(s) or virus(es)(CD4+ T cell epitope). In another example, the fusion partner is an alarmin. In a related embodiment, the targeting moiety-nucleic acid conjugate(s) described herein further comprises one or more PAMP and/or one or more DAMP/Alarmin(s). In one aspect, the conjugate further includes one or more peptides that include one or more pathogen-derived or tumor antigens or antigenic determinants.

In one embodiment, the invention comprises a conjugate of an APC/DC-targeting moiety, one or more pathogen associated molecular pattern (PAMP) and/or one or more alarmins, and one or more antigenic peptides that include one or more tumor antigens and/or antigens or antigenic determinants derived from one or more pathogen(s), microorganism(s) or virus(es)(T or B cell epitopes). In one embodiment the antigenic peptide is fused to or incorporated within the targeting moiety. In another aspect, the antigenic peptide is fused to an alarmin (e.g. LL-37).

In one embodiment, the invention comprises a conjugate of an APC/DC-targeting moiety, one or more nucleic acid molecules, and one or more antigenic peptides, wherein the nucleic acid molecule includes one or more pathogen associated molecular pattern (PAMP) and the antigenic peptides includes tumor antigens and/or antigens or antigenic determinants derived from one or more pathogen(s), microorganism(s) or virus(es)(T or B cell epitopes). In one embodiment the antigenic peptide is fused to or incorporated within the targeting moiety. In one related embodiment of the conjugate, the antigenic peptide is fused to a nucleic acid binding peptide (e.g. cationic peptides, NLS, Tat, Protamine, His6, Arg9, LL-37). In another aspect, the antigenic peptide is fused to a peptide motif targeting a DC uptake receptor. In one aspect, the antigenic peptide is fused to or incorporated within the targeting moiety. In another aspect, the antigenic peptide is fused to an alarmin.

In one embodiment, the invention comprises a conjugate or fusion protein incorporating a DC targeting peptide, antigenic peptide, and nucleic acid binding peptide (alarmin, e.g LL-37), wherein said protein is covalently or non-covalently linked to a nucleic acid molecule (coding or non-coding). In one aspect, the nucleic acid molecule includes one or more PAMP. In another aspect, the nucleic acid molecule further encodes one or more of the following: (i) one or more tumor antigens or antigenic determinants derived from one or more pathogen(s), microorganism(s) or virus(es), (ii) one or more immunostimulatory molecules, such as molecules that recruit, bind, activate, mature and/or proliferate an antigen presenting cell or dendritic cell or other immune cell (such as T cells, B cells, NK cells) and molecules that counteract immune suppression (e.g. immunostimulatory cytokines, chemokines, costimulatory molecules, growth factors).

In one embodiment, the invention comprises a conjugate comprising an immune complex of a fusion antigenic peptide/protein and antibody, wherein the fusion peptide/protein incorporates the antigenic peptide and a specific tag peptide that binds the said antibody. In one aspect of the conjugate, the fusion peptide/protein in the immune complex further includes a nucleic acid binding peptide (e.g. cationic peptides, protamine, HIV-tat, Arginine- or Histidine-rich sequence, LL-37, Nuclear localizing peptide). In another aspect of the conjugate, the fusion peptide in the immune complex further includes an alarmin (e.g. LL-37). In another aspect of the conjugate, the fusion peptide in the immune complex further incorporates a peptide that binds a DC uptake receptor. In another embodiment, a conjugate comprises an immunostimulatory nucleic acid molecule that is linked to either the antibody or the fusion peptide antigen, wherein the nucleic acid molecule includes one or more PAMP. In another aspect, the nucleic acid molecule further encodes one or more of the following: (i) one or more tumor antigens or antigenic determinants derived from one or more pathogen(s), microorganism(s) or virus(es), (ii) one or more immunostimulatory molecules.

Exemplary methods and compositions according to this invention are described in detail.

### BRIEF DESCRIPTION OF THE DRAWINGS

FIG. 1 illustrates nucleotide (DNA/RNA)-conjugated antibodies.

FIG. 2 illustrates nucleotide (DNA/RNA)-conjugated tumor targeted peptides (SEQ ID NO:6).

FIG. 3 illustrates the mechanism(s) of action of a nucleic acid-antibody conjugate (INAS=Immunostimulatory Nucleic Acid Sequence).

FIG. 4 illustrates the method of covalent conjugation of DNA or RNA (INAS) to antibodies/polypeptides/peptides.

Step 1. The 3′-phosphate group of oligonucleotide (e.g. CpG DNA) is conjugated with the amine group of the antibody using the carbodiimide cross-linker EDC;

Step 2. The EDC activated oligonucleotide interacts with Imidazole to form an active intermediate for conjugation;

Step 3. The active nucleotide intermediate forms a covalent bond with the targeted antibody (such as anti-EGFR or anti-HER2);

Step 4. The imidazole and the unconjugated nucleotide residues are removed by passage through a 10 kD cut off column plus PBS washing.

FIG. 5 shows immunoblots demonstrating DNA- or RNA-conjugated anti-EGFR antibody and anti-HER2 antibody.

Anti-human EGFR Antibody-DNA conjugate (DNA=SEQ ID: 1)

Anti-human HER2Antibody-DNA conjugate (DNA=SEQ ID: 1)

Anti-EGFR antibody-RNA conjugate (EGFR antibody-SVM274)

FIG. 6 is an immunoblot demonstrating the inhibition of EGFR phosphorylation (Tyr 1068) by either anti-EGFR antibody (EGFR Ab) or DNA-conjugated anti-EGFR antibody (EGFR Ab-DNA SEQ ID NO: 1 or EGFR Ab-DNA SEQ ID NO:2).

FIG. 7 is a showing of FACS analysis, which demonstrates the maturation of dendritic cells by DNA-conjugated anti-EGFR antibody (EGFR Ab-DNA SEQ ID NO: 1) but not with EGFR antibody.

FIG. 8 shows bar graphs demonstrating the effects of DNA-conjugated antibodies on the expression of Interferon-γ (IFN-γ) and Apo2L/TRAIL in PBMCs. A) shows the quantification of IFN-γ (pg/ml) by ELISA in supernatants of PBMCs treated with either anti-EGFR antibody (anti-EGFR Ab) 5 μg/ml, anti-human HER2 antibody (anti-HER2Ab) 5 μg/ml, DNA (ODN-SEQ ID NO: 1) 5 μg/ml, anti-EGFR Ab-DNA 5 μg/ml, anti-HER2Ab-DNA 5 μg/ml, or left untreated (control). B) shows the quantification of Apo2L/TRAIL (pg/ml) by ELISA in supernatants of PBMCs treated with either anti-EGFR antibody (anti-EGFR Ab) 5 g/ml, anti-human HER2 antibody (anti-HER2 Ab) 5 μg/ml, DNA (ODN-SEQ ID NO:1) 5 μg/ml, anti-EGFR Ab-DNA 5 μg/ml, anti-HER2Ab-DNA 5 μg/ml, or left untreated (control).

FIG. 9 is a showing of flow cytometry analysis of the expansion of CD56+PBMCs following treatment with EGFR antibody-DNA conjugate (EGFR Ab-DNA SEQ ID NO:1) but not with EGFR antibody (control).

FIG. 10 shows a table demonstrating increased expression of MHC molecules (DR; class II) in PBMCs following treatment with EGFR antibody-nucleotide conjugates (EGFR-DNA or EGFR-RNA).

FIG. 11 shows a table demonstrating induction of Apo2L/TRAIL in EGFR-expressing tumor cells (MDA-MB468) in response to treatment with EGFR antibody-DNA conjugates (EGFR Ab-DNA SEQ ID NO: 1 or EGFR Ab-DNA SEQ ID NO:2) and in HER2/neu-expressing tumor cells (SKBr-3) in response to treatment with HER2 antibody-DNA conjugates (HER2Ab-DNA SEQ ID NO: 1 or HER2Ab-DNA SEQ ID NO:2).

FIG. 12 shows a photomicrograph demonstrating the induction of direct death (with cell hyperfusion) of EGFR-expressing human colon cancer cells (HT29 cells) in response to treatment with EGFR antibody-DNA conjugates (EGFR Ab-DNA SEQ ID NO:1 or EGFR Ab-DNA SEQ ID NO:2).

FIG. 13 shows a cell culture plate demonstrating the induction of direct death (with loss of colony formation) of EGFR-expressing human colon cancer cells (HT29 cells) in response to treatment with EGFR antibody-DNA conjugate (EGFR Ab-DNA SEQ ID NO:1) but not with either EGFR antibody or unconjugated nucleic acid (DNA SEQ ID NO:1).

FIG. 14 shows a photomicrograph demonstrating the induction of direct death of EGFR-expressing human breast cancer cells (MCF-7 or MDA-MB468 cells) in response to treatment with EGFR antibody-DNA conjugates (EGFR Ab-DNA SEQ ID NO:1).

FIG. 15 shows a cell culture plate demonstrating the induction of direct death (with loss of colony formation) of EGFR-expressing human breast cancer cells (MCF-7 cells) in response to treatment with EGFR antibody-DNA conjugate [EGFR Ab-DNA 1 (SEQ ID NO:1) or EGFR Ab-DNA 2 (SEQ ID NO:2)] but not with either EGFR antibody or unconjugated nucleic acid (DNA SEQ ID NO:1 or DNA SEQ ID NO:2).

FIG. 16 shows a photomicrograph demonstrating the induction of direct death (with cell hyperfusion) of HER2/neu-expressing human breast cancer cells (MCF-7 and SKBr-3 cells) in response to treatment with HER2 antibody-DNA conjugates HER2Ab-DNA 1 (SEQ ID NO:1) or HER2Ab-DNA 2 (SEQ ID NO:2). Analysis of four hyperfused coalescent cell bodies demonstrate non-viable cells (stained with trypan-blue) and interspersed cell fragments.

FIG. 17 shows a photomicrograph demonstrating the induction of direct death (with cell hyperfusion) of Neu-expressing murine breast cancer cells in response to treatment with Neu antibody-DNA conjugates Neu Ab-DNA 1 (SEQ ID NO: 1) or Neu Ab-DNA 2 (SEQ ID NO:2).

FIG. 18 shows a graph demonstrating the induction of HT-29 tumor cell death by either anti-EGFR antibody or anti-EGFR antibody-DNA conjugate (EGFR Ab-DNA SEQ ID NO:1) as a function of PBMC:tumor cell ratio (A) or as a function of time (B).

FIG. 19 shows the inhibition of EGFR-expressing HT-29 tumor growth following administration of DNA-conjugated anti-EGFR antibody (EGFR Ab-DNA SEQ ID NO:1) compared with treatment with either EGFR antibody alone, DNA alone (DNA SEQ ID NO:1), or the combination of unconjugated antibody and nucleic acid.

FIG. 20 shows a graph demonstrating the inhibition of growth and reduction of volume of syngeneic Neu+ tumors in FVB mice in response to treatment with Neu antibody-DNA conjugates [Neu Ab-DNA SEQ ID NO:1] compared with treatment with either Neu antibody alone or DNA alone (DNA SEQ ID NO:1).

FIGS. 21A and 21B are graphs showing the inhibition of growth of tumors in (neu-N)-transgenic mice in response to intratumoral or systemic administration of DNA-conjugated anti-neu antibody: (A) tumor volume in untreated control mice. (B) tumor volume in Neu antibody-DNA conjugate-treated mice [Neu Ab-DNA SEQ ID NO: 1].

FIG. 22 illustrates Binding of Histidine (His)-tagged Protective Antigen (PA) of Bacillus Anthracis with an oligonucleotide.

FIG. 23 illustrates Triple Helix formation between an oligonucleotide and a plasmid.

FIG. 24. Illustrates plasmid delivery and gene expression by Anti-EGFR Antibody-HIV Tat peptide complex

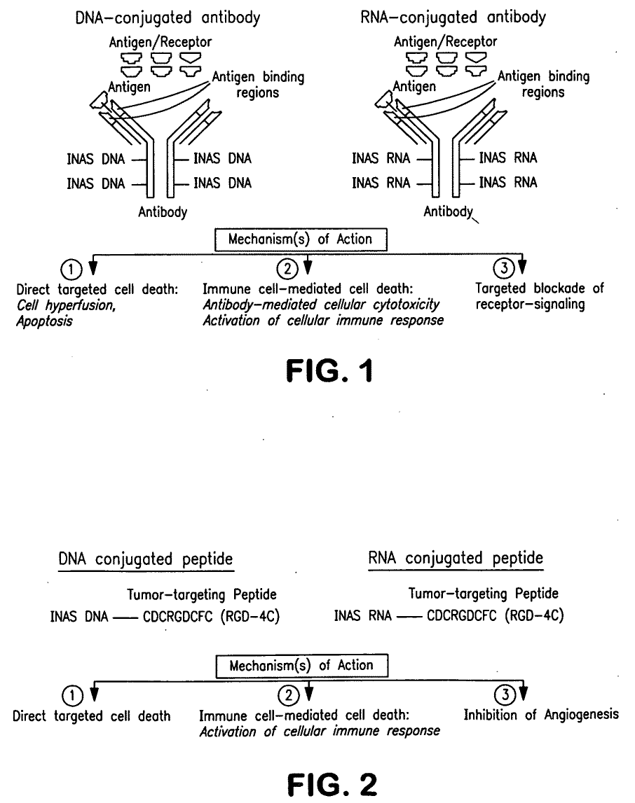

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

As used herein “immune effector cells” include T cells, NK cells, B cells, monocytes, macrophages, and dendritic cells (DC).

As used herein “a tumor targeting peptide” includes polymers containing fewer than 100 amino acids, where the polymer specifically binds to a cellular component of a tumor cell, tumor vasculature, and/or a component of a tumor microenvironment.

As used herein, “neoplasm,” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, melanoma, sarcoma, liver, kidney, lung, testicular, breast, ovarian, pancreatic, brain, head and neck, melanoma, leukemia, lymphoma cancer, and the like.

A used herein “subject,” including grammatical variations thereof, means a human or vertebrate animal including a dog, cat, horse, cow, pig, sheep, goat, chicken, monkey, rat, and mouse.

As used herein “conjugation,” including grammatical variations thereof, means directly or indirectly linking, coupling, binding and the like of the foreign DNA or RNA with target-specific antibodies and/or peptides and/or tumor targeting moieties, either chemically, electrostatically, non-covalently, or by other techniques. For example, an isolated antibody-nucleic acid conjugate or peptide-nucleic acid conjugate as presently disclosed would fall under this definition.

An “immunostimulatory nucleic acid sequence” (INAS) refers to a nucleic acid molecule that is a pathogen-associated molecular pattern (PAMP) or other motif that can activate immune cells, including, but not limited to, double stranded DNA (ds DNA), single stranded DNA (ss DNA), CpG DNA (CpG), herpes simplex virus (HSV) DNA, double stranded RNA (dsRNA), and single stranded RNA (ssRNA). In a related aspect, the INAS may be a coding or non-coding sequence. As illustrative examples, an INAS may be DNA (SEQ ID NO:1 or SEQ ID NO:2) or RNA (see below).

The term “therapeutically effective amount” means the amount of the subject compound that will elicit the biological or medical response of a tissue, system, animal or human that is being sought by the researcher, veterinarian, medical doctor or other clinician.

The term “composition,” as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention in a therapeutically effective amount to the individual in need of treatment. Administration can be intratumoral or systemic (intravenous) administration. Furthermore, in conjunction with vaccination of recipient with pathogen antigen vaccine (e.g. tetanus toxoid). In addition, in conjunction with agent to deplete or inactivate regulatory T cells (e.g. cyclophosphamide) or myeloid suppressor cells (e.g. gemcitabine). In a further example, Ex vivo treatment of immune cells and tumor cells for generation of tumor reactive or pathogen antigen reactive immune cells—for adoptive cellular immunotherapy. Administration can be intradermal or subcutaneous. Furthermore, administration can be in combination with one or more additional therapeutic agents deplete or inactivate regulatory T cells (cyclophosphamide) or myeloid suppressor cells (e.g. gemcitabine). The pharmaceutical compositions of the invention identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., cancer, pathogenic infectious agents, associated conditions thereof). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

In general, compositions and methods of the invention involve a therapeutic or diagnostic compound comprising a targeting moiety specific for a target cell and an active agent which enhances an immune response against the target cell. As further described herein, targeting moieties are specific for molecules or components of a cancer or tumor, of an infectious agent or of a normal cell. Furthermore, an active agent includes nucleic acids, peptides or combinations thereof.

In a first aspect of the invention, products and processes of the invention are directed to a composition comprising a targeting moiety and an one, two, three or more active agents.

In one embodiment, a composition of the invention comprises a targeting moiety coupled to an active agent. In another embodiment, a composition comprises a targeting moiety, and at least two active agent, which include a non-coding nucleic acid molecule and a peptide or polypeptide. In a further embodiment, the at least two active agents include a non-coding nucleic acid molecule and a coding nucleic acid molecule (e.g., plasmid or minicircle). In yet a further embodiment, the at least two active agents include a non-coding or coding nucleic acid molecule, and an antigenic peptide or polypeptide. For simplified illustration, compositions of the invention can be covered by the following formula: T-A₁ or T-A₁-A₂, where T=targeting moiety; A¹ is either a nucleic acid molecule or peptide or polypeptide or lipopeptide; and A₂ is either a nucleic acid molecule or peptide or polypeptide or lipopeptide. Furthermore, the nucleic acid molecule can be a coding or non-coding sequence as further described herein. In further embodiments, A¹ can be coupled (directly or indirectly) to an additional component including a nucleic acid molecule, a peptide, a polypeptide, or lipopeptide. Alternatively, in further embodiments an active agent is a component for packaging and/or delivery of a nucleic acid molecule

For example, in some embodiments of the invention, T=aptamer, peptide or antibody targeting a component of a tumor cell, normal cell or infectious agent, A₁=a immunostimulatory non-coding nucleic acid molecule; and A₂=an peptide or polypeptide which is antigenic to a subject (e.g., animal to whom the composition is administered). In another embodiment, a composition of the invention comprises T-A₁.

### I. Targeting Moiety

The targeting moiety (e.g., antibody) facilitates delivery of conjugated biologically active agent (e.g., nucleic acid) to the target cell (e.g. via receptor-mediated endocytosis of antibodies binding target cell receptors).

For example, the targeting moiety facilitates delivery of the biologically active agent(s) (e.g., INAS) and immunogenic apoptotic material from antibody-bound tumor targets to immune cells via interactions between their Fc and Fc receptors (on immune cells); this promotes internalization of nucleic acid via endocytosis and activation of endosomal pattern recognition receptors (e.g. Toll-like receptors).

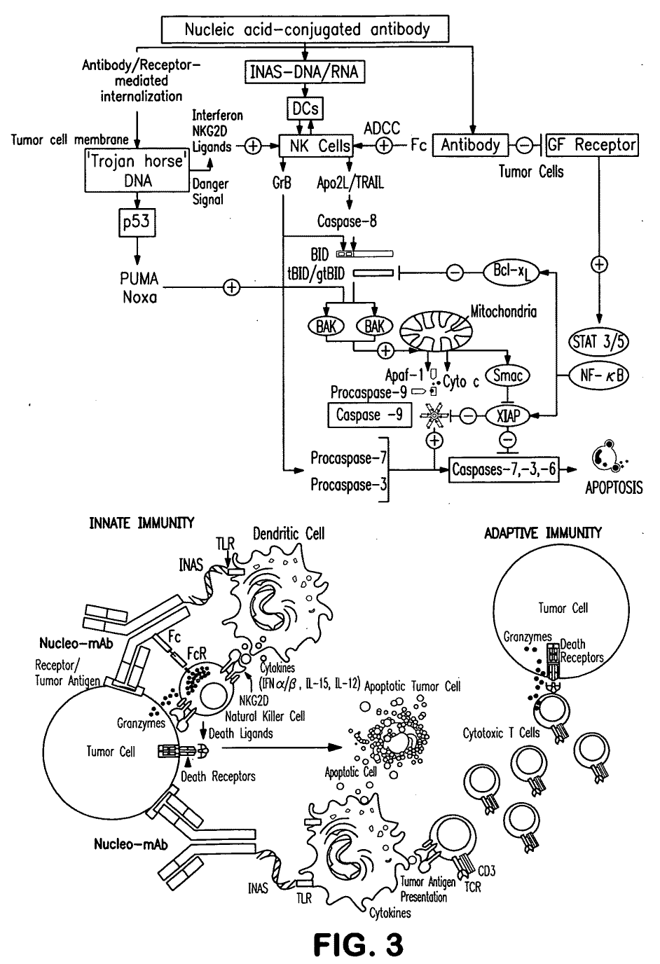

For example, the introduction of immunostimulatory DNA-conjugated or RNA-conjugated antibodies/peptides activates death signaling in targeted cells (e.g., neoplastic cells) (FIG. 3). While not being bound by theory, and in contrast to the effects of genotoxic chemotherapeutic agents, use of DNA-conjugated or RNA-conjugated antibodies/peptides enables the activation of death signaling in targeted cells without corresponding effects on normal tissues that do not express the targeted molecule or express significantly lower levels of the molecule compared to neoplastic cells.

In one aspect of the invention, the targeting moiety-biologically active agent conjugate functions to induce an immune response exclusive of the sequence of the biologically active agent. In various embodiments, a conjugate of the invention is able to promote death of target cells while simultaneously inducing direct or indirect activation of the innate and adaptive immune system. For example, the intracellular recognition of INAS-antibody conjugates serves to activate the production of cytokines/costimulatory molecules/alarmins/damage-associated molecular patterns (endogenous danger signals) by target cells, promote the direct and immune-mediated death of target cells, facilitate the uptake of apoptotic cells (carrying nucleic acid) by antigen presenting cells, and activate the immune system to generate antitumor responses against cross-presented tumor antigens (FIG. 3). These antibody-nucleic acid immune complexes can activate endosomal TLR-mediated or TLR-independent immune responses following engulfment of apoptotic tumor cells by macrophages and dendritic cells. This can induce autoimmune responses directed at antigens derived from antibody-bound apoptotic tumor cells.

As used herein, “targeting moiety” (or moieties) refers to a molecule(s) that has the ability to localize and bind to a molecule present on a normal cell/tissue and/or cancer cell/tumor in a subject. In other words, compositions of the invention comprising such a targeting moiety can bind to a ligand (directly or indirectly), which is present on a cell. Furthermore, targeting moeity refers to a molecule(s) that has the ability to localize to and bind a target molecule present on a normal cell/tissue and/or cancer cell/tumor or other molecule. In other words, compositions of the invention comprising such a targeting moiety can bind to a targeted cell or molecule (directly or indirectly). The targeting moieties of the invention contemplated for use with the biologically active agents include antibody, polypeptides, peptides, aptamers, other ligands, or any combination thereof, that can bind a component of the target cell or molecule.

In one embodiment, a targeting moeity binds a tumor cell(s) or can bind in the vicinity of a tumor cell(s) (e.g., tumor vasculature or tumor microenvironment) following administration to the subject. The targeting moiety may bind to a receptor or ligand on the surface of the cancer cell or may bind to an intracellular target of cancer cell provided that the target is accessible to the molecule. Accessibility to intracellular cancer cell targets may arise in cancer cells that have a compromised plasma membrane such as cells which are undergoing apoptosis, necrosis, and the like. Some cancer targeting molecules can bind intracellular portions of a cell that does not have a compromised plasma membrane.

In another aspect of the invention, a targeting moiety is selected which is specific for a non-cancerous cells or tissue. For example, a targeting moiety can be specific for a molecule present normally on a particular cell or tissue. Furthermore, in some embodiments, the same molecule can be present on normal and cancer cells. Various cellular components and molecules are known. For example, if a targeting moiety is specific for EGFR, the resulting conjugate of the invention can target cancer cells expressing EGFR as well as normal skin epidermal cells expressing EGFR. Therefore, in some embodiments, a conjugate of the invention can operate by two separate mechanisms (targeting cancer and non-cancer cells), as further discussed herein. In yet further embodiment, a conjugate of the invention comprises a targeting moiety which is specific for a component or molecule of an infectious agent.

In various aspects of the invention disclosed herein a conjugate of the invention comprises a targeting moiety which can bind/target a cellular component, such as a tumor antigen, a bacterial antigen, a viral antigen, a mycoplasm antigen, a fungal antigen, a prion antigen, an antigen from a parasite. As used herein, a cellular component, antigen or molecule can each be used to mean, a desired target for a targeting moiety. For example, in various embodiments, a targeting moiety is specific for or binds to a component, which includes but is not limited to, epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family; discoidin domain receptor (DDR) family; RET receptor family; KLG receptor family; RYK receptor family; MuSK receptor family; Transforming growth factor α (TGF-α) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), β-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: β-Dgalactose 2-α-Lfucosyltransferase (LDLR/FUT) fusion protein, HLA-A2. arginine to isoleucine exchange at residue 170 of the α-helix of the α2-domain in the HLA-A2 gene (HLA-A*201-R170I), HLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class I, NFYC, OGT, OS-9, pml-RARalpha fusion protein, PRDX5, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGE-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-1, LAGE, LAGE-1, CTL-recognized antigen on melanoma (CAMEL), MAGE-A1 (MAGE-1), MAGE-A2, MAGE-A3, MAGE-A4, MAGE-A5, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pmel17 (SILV), tyrosinase (TYR), TRP-1, HAGE, NA-88, NY-ESO-1, NY-ESO-1/LAGE-2, SAGE, Sp17, SSX-1,2,3,4, TRP2-INT2, carcino-embryonic antigen (CEA), Kallikrein 4, mammaglobin-A, OA1, prostate specific antigen (PSA), TRP-1/gp75, TRP-2, adipophilin, interferon inducible protein absent in melanoma 2 (AIM-2), BING-4, CPSF, cyclin D1, epithelial cell adhesion molecule (Ep-CAM), EphA3, fibroblast growth factor-5 (FGF-5), glycoprotein 250 (gp250), EGFR (ERBB1), HER-2/neu (ERBB2), interleukin 13 receptor α2 chain (IL13Ralpha2), IL-6 receptor, intestinal carboxyl esterase (iCE), alpha-feto protein (AFP), M-CSF, mdm-2, MUC1, p53 (TP53), PBF, PRAME, PSMA, RAGE-1, RNF43, RU2AS, SOX10, STEAP1, survivin (BIRC5), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA661, LDHC, MORC, SGY-1, SPO11, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), β-human chorionic gonadotropin, 1-2 microglobulin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins—LMP1, LMP2; others), Hepatitis B or C virus proteins, and HIV proteins. A conjugate can further comprise the foregoing as a peptide/polypeptide and/or encoding the same.

As noted herein, in various embodiments, a compound of the invention comprises a targeting moiety which binds a component (e.g., antigen) of an infectious agent, where such a compound is coupled to a biologically active agent, and wherein such a compound induces an immunostimulatory response (either directly/indirectly) in a subject. In general, such an infectious agent can be any pathogen including without any limitation bacteria, yeast, fungi, virus, eukaryotic parasites, etc. In various embodiments, compounds of the invention comprise a targeting moiety directed to a component present on a pathogen/infectious agent, which include but are not limited to Retroviridae (e.g. human immunodeficiency viruses, such as HIV-1 (also referred to as HTLV-III, LAV or HTLV-III/LAV, or HIV-III); and other isolates, such as HIV-LP); Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Rous sarcoma virus (RSV), avian leukemia virus (ALV), and avian myeloblastosis virus (AMV)) and C-type group B (including feline leukemia virus (FeLV), gibbon ape leukemia virus (GALV), spleen necrosis virus (SNV), reticuloendotheliosis virus (RV) and simian sarcoma virus (SSV)), D-type retroviruses include Mason-Pfizer monkey virus (MPMV) and simian retrovirus type 1 (SRV-1), the complex retroviruses including the subgroups of lentiviruses, T-cell leukemia viruses and the foamy viruses, lentiviruses including HIV-1, HIV-2, SIV, Visna virus, feline immunodeficiency virus (FIV), and equine infectious anemia virus (EIAV), simian T-cell leukemia virus (STLV), and bovine leukemia virus (BLV), the foamy viruses including human foamy virus (HFV), simian foamy virus (SFV) and bovine foamy virus (BFV), Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), Mycobacterium (Mycobacterium tuberculosis, M. bovis, M. avium-intracellulare, M. leprae), Pneumococcus, Streptococcus, Staphylcococcus, Diphtheria, Listeria, Erysipelothrix, Anthrax, Tetanus, Clostridium, Mixed Anaerobes, Neisseria, Salmonella, Shigella, Hemophilus, Escherichia coli, Klebsiella, Enterobacter, Serratia, Pseudomonas, Bordatella, Francisella tularensis, Yersinia, Vibrio cholerae, Bartonella, Legionella, Spirochaetes (Treponema, Leptospira, Borrelia), Fungi, Actinomyces, Rickettsia, Mycoplasma, Chlamydia, Protozoa (including Entamoeba, Plasmodium, Leishmania, Trypanosoma, Toxoplasma, Pneumocystis, Babasia, Giardia, Cryptosporidium, Trichomonas), Helminths (Trichinella, Wucheraria, Onchocerca, Schistosoma, Nematodes, Cestodes, Trematodes). Additional examples of antigens which can be targets for compositions of the invention are known, such as those disclosed in US Application No. 2007/0066554. In a further aspect of the invention, a conjugate can comprise an antigen or cellular component as described herein, but in addition to a targeting moiety and an immunostimulatory nucleic acid molecule. As further described herein below, a composition of the invention can comprise a targeting moiety, an immunostimulatory nucleic acid or nucleic acid coding a polypeptide or peptide of interest, and a peptide or polypeptide (antigen) associated with an infectious agent. A conjugate can further comprise the foregoing as a peptide/polypeptide and/or encoding the same. Furthermore, for DNA vaccination, a coding sequence delivered and expressed in a tumor cell as well as in DCs to provide enhanced immune response.

Each of the foregoing and subsequent lists is illustrative, and is not intended to be limiting.

In various embodiments, a compound of the invention comprising a targeting moiety to an infectious agent as described herein, and a biologically active agent which is an immunostimulatory nucleic acid or protein molecule. In further embodiments, such immunostimulatory biologically active agents comprise one or more nucleic acid or protein molecules corresponding to SEQ ID NO: 56 to 228. Furthermore, this sequences can be comprised in a conjugate in order to express the polypeptides in a tumor cell or DC to enhance the immune response. In yet further embodiments, a compound (e.g., conjugate) of the invention comprises two or more of the same or different biologically active agents.

Targeting moieties can be specific for particular antigens particular to various types of infectious agents. For example, influenza virus belongs to the genus orthomyxovirus in the family of Orthomyxoviridae. ssRNA enveloped viruses with a helical symmetry. Enveloped particles 80-120 nm in diameter. The RNA is closely associated with the nucleoprotein (NP) to form a helical structure. The genome is segmented, with 8 RNA fragments (7 for influenza C). There are 4 principle antigens present, the hemagglutinin (H), neuraminidase (N), nucleoprotein (NP), and the matrix (M) proteins. The NP is a type-specific antigen which occurs in 3 forms, A, B and C, which provides the basis for the classification of human and non-human influenza viruses. The matrix protein (M protein) surrounds the nucleocapsid and makes up 35-45% of the particle mass. Furthermore, 2 surface glycoproteins are seen on the surface as rod-shaped projections. The haemagglutinin (H) is made up of 2 subunits, H1 and H2. Haemagglutinin mediates the attachment of the virus to the cellular receptor. Neuraminidase molecules are present in lesser quantities in the envelope. The antigenic differences of the hemagglutinin and the neuraminidase antigens of influenza A viruses provide the basis of their classification into subtypes. e.g., A/Hong Kong/1/68 (H3N2) signifies an influenza A virus isolated from a patient in 1968, and of subtype H3N2, as well as specific targeting components. A conjugate can further comprise the foregoing as a peptide/polypeptide and/or encoding the same. Furthermore, for DNA vaccination, a coding sequence delivered and expressed in a tumor cell as well as in DCs to provide enhanced immune response.

Thus, in various embodiments, the compounds of the invention comprise a targeting moiety and a biologically active agent, which induce an immune response targeting an infectious agent. For example, targeting moieties can be specific for influenza virus type A for any H×Ny where x is 1-9 and y is 1-16, or any combination of xy thereof. For example, in one embodiment, a compound of the invention comprises a targeting moiety which binds to an antigen or fusion peptide comprising an antigen, e.g., influenza A subtype H1N5.

In one embodiment, a targeting moiety specific for an infectious agent component recognizes an epitope. As used herein, the term “epitope” refers to portions of a polypeptide having antigenic or immunogenic activity in an animal, preferably a mammal, and most preferably in a human. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998 4002 (1983)). The term “antigenic epitope,” as used herein, is defined as a portion of a protein to which an antibody can immunospecifically bind its antigen as determined by any method well known in the art. Immunospecific binding excludes non specific binding but does not necessarily exclude cross reactivity with other antigens. Antigenic epitopes need not necessarily be immunogenic. Antigenic epitopes can also be T-cell epitopes, in which case they can be bound immunospecifically by a T-cell receptor within the context of an MHC molecule. An epitope can comprise 3 amino acids in a spatial conformation which is unique to the epitope. Generally, an epitope consists of at least about 5 such amino acids, and more usually, consists of at least about 8-10 such amino acids. If the epitope is an organic molecule, it may be as small as Nitrophenyl.

Targeting moieties of the conjugates of the invention can be specific for known antigens associated with infectious agents. See <fda.gov/cber/products/testkits.htm> (listing various antigens to which commercially available antibodies/assays are available, including HIV, HBV, HTLV). Furthermore, additional examples of target components are disclosed in US Patent Application Publications 20070172881 (fungal); 20070166319 (HPV); 20060252132 (influenza variants); 20060115497 (Mycobacterium); U.S. Pat. No. 5,378,805 (HTLV); 20060099219 (HPV): 20070154883 (Rubella); U.S. Pat. No. 7,060,283 (Epstein Barr virus); U.S. Pat. No. 7,232,566 (HIV); U.S. Pat. No. 7,205,101 (HIV); and U.S. Pat. No. 6,878,816 (Borrelia). A conjugate can further comprise the foregoing as a peptide/polypeptide and/or encoding the same. Furthermore, for DNA vaccination, a coding sequence delivered and expressed in a tumor cell as well as in DCs to provide enhanced immune response.

A. Antibodies

In one embodiment, a composition of the invention comprises a targeting moiety, which is a polypeptide associated (e.g., conjugated) to a biologically active agent (e.g., immune response inducing nucleic acid molecule, nucleic acid molecule encoding a desired peptide or polypeptide, a peptide and antigen). In certain embodiments, an antibody is coupled with two, three or four of the same type or different types of biologically active agents. For example, in some embodiments, a composition of the invention comprises a targeting moiety coupled to a non-coding immunostimuatory nucleic acid molecule and a immunostimulatory peptide, polypeptide or PNA.

In some embodiments, a composition of the invention comprises a targeting moiety coupled to a tag (e.g., histadine tag). In another embodiment, a composition comprises a targeting moiety, a nucleic acid molecule and a tag (e.g., biotin/avidin). In further embodiments, an antibody can bind a tag on a fusion protein, which includes an antigenic peptide or polypeptide.

In one embodiment, the polypeptide molecule of the conjugate is an immunoglobulin. As used herein, the term “immunoglobulin” includes natural or artificial mono- or polyvalent antibodies including, but not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA, and IgY), class (e.g., IgG1, IgG2, IgG3, IgG4, IgA1, and IgA2) or subclass of immunoglobulin molecule.

A conjugate of the invention through its antibody targeting moiety will bind a cellular component of a tumor cell, tumor vasculature or tumor microenvironment, thereby promoting apoptosis of targeted cells via inhibition of survival signals (e.g., growth factor or cytokine or hormone receptor antagonists), activation of death signals, and/or immune-mediated cytotoxicity, such as through antibody dependent cellular cytotoxicity. Such conjugates can function through several mechanisms to prevent, reduce or eliminate tumor cells, such as to facilitate delivery of conjugated INAS to the tumor target, such as through receptor-mediated endocytosis of antibodies binding target cell receptors; facilitate delivery of INAS and immunogenic apoptotic material from antibody-bound tumor targets to immune cells via interactions between their Fc and Fc receptors (on immune cells); this promotes internalization of INAS via endocytosis and activation of endosomal pattern recognition receptors (e.g. Toll-like receptors); or such conjugates can recruit, bind, and/or activate immune cells (e.g. NK cells, monocytes/macrophages, dendritic cells, T cells, B cells) via interactions between their Fc and Fc receptors (on immune cells) and via the conjugated INAS. Moreover, in some instances one or more of the foregoing pathways may operate upon administration of one or more conjugate of the invention.

Antibodies of the invention include antibody fragments that include, but are not limited to, Fab, Fab′ and F(ab′)2, Fd, single-chain Fvs (scFv), single-chain antibodies, disulfide-linked Fvs (sdfv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. Also included in the invention are Fc fragments, antigen-Fc fusion proteins, and Fc-targeting moiety conjugates or fusion products (Fc-peptide, Fc-aptamer). The antibodies of the invention may be from any animal origin including birds and mammals. In one aspect, the antibodies are human, murine (e.g., mouse and rat), donkey, sheep, rabbit, goat, guinea pig, camel, horse, or chicken. Further, such antibodies may be humanized versions of animal antibodies. The antibodies of the invention may be monospecific, bispecific, trispecific, or of greater multispecificity.

The antibodies of the invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further, antibodies and antibody-like binding proteins may be made by phage display. Furthermore, antibodies can be produced in plants, as known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example; in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al (1975) Nature 256:495, or may be made by recombinant DNA methods (see, U.S. Pat. No. 4,816,567). The “monoclonal antibodies” may also be isolated from phage antibody libraries using the techniques described in Clackson et al (1991) Nature, 352:624-628; Marks et al (1991) J. Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape etc) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death. Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

“Antibody fragments” comprise a portion of an intact antibody, e.g. comprising the antigen-binding or variable region thereof. Examples of antibody fragments include Fab, Fab′, F(ab′)2, and Fv fragments; Fc fragments or Fc-fusion products; diabodies; linear antibodies; single-chain antibody molecules; and multispecific antibodies formed from antibody fragment(s).

An “intact” antibody is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof or any other modified Fc (e.g. glycosylation or other engineered Fc).

The intact antibody may have one or more “effector functions” which refer to those biological activities attributable to the Fc region (a native sequence Fc region or amino acid sequence variant Fc region or any other modified Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, Δ., ε, γ, and μ respectively. The subunit structures and three-dimensional configurations of different classes of immunoglobulins are well known.

In various embodiments, an antibody/targeting moiety recruits, binds, and/or activates immune cells (e.g. NK cells, monocytes/macrophages, dendritic cells) via interactions between Fc (in antibodies) and Fc receptors (on immune cells) and via the conjugated INAS for antibody/peptide/ligand or other targeting moiety. Examples of antibodies which can be incorporated into compositions and methods of the invention include but are not limited to antibodies such as cetuximab (chimeric monoclonal antibody to epidermal growth factor receptor EGFR), panitumumab (anti-EGFR), nimotuzumab (anti-EGFR), B8, Rituximab (chimeric murine/human anti-CD2O MAb); Herceptin, trastuzumab (anti-Her2 hMAb); Panorex™ (17-1A) (murine monoclonal antibody); Panorex @ (17-1A) (chimeric murine monoclonal antibody); IDEC-Y2B8 (murine, anti-CD20 MAb); BEC2 (anti-idiotypic MAb, mimics the GD epitope) (with BCG); Oncolym (Lym-1 monoclonal antibody); SMART M195 Ab, humanized 13′ I LYM-1 (Oncolym), Ovarex (B43.13, anti-idiotypic mouse MAb); MDX-210 (humanized anti-HER-2 bispecific antibody); 3622W94 MAb that binds to EGP40 (17-1A) pancarcinoma antigen on adenocarcinomas; Anti-VEGF, RhuMAb (Avastin; inhibits angiogenesis); Zenapax (SMART Anti-Tac (IL-2 receptor); SMART M195 Ab, humanized Ab, humanized); MDX-210 (humanized anti-HER-2 bispecific antibody); MDX-447 (humanized anti-EGF receptor bispecific antibody); NovoMAb-G2 (pancarcinoma specific Ab); TNT (chimeric MAb to histone antigens); TNT (chimeric MAb to histone antigens); Gliomab-H (Monoclonals—Humanized Abs); GNI-250 Mab; EMD-72000 (chimeric-EGF antagonist); LymphoCide (humanized LL2 antibody); and MDX-260 bispecific, targets GD-2, ANA Ab, SMART IDIO Ab, SMART ABL 364 Ab or ImmuRAIT-CEA. As illustrated by the forgoing list, it is conventional to make antibodies to a particular target epitope.

B. Aptamers

In one aspect of the invention, the targeting moiety is an aptamer molecule that is linked to an immunostimulatory sequence. For example, in some embodiments, the aptamer is comprised of nucleic acids that function as a targeting moiety, which are coupled to or further comprise one or more immunostimulatory nucleic acids. In various embodiments, a composition of the invention comprises an aptamer that is specific for a molecule on a tumor cell, tumor vasculature, and/or a tumor microenvironment. In addition, such compositions comprise a biologically active agent (e.g., nucleic acids or peptides). However, it should be made clear that the aptamer itself can comprise of a biologically active sequence, in addition to the targeting module (sequence), wherein the biologically active sequence can induce an immune response to the target cell. In other words, such an aptamer molecule is a dual use composition of the invention. In some embodiments, a composition of the invention comprises conjugation of an aptamer to an antibody, wherein the aptamer and the antibody are specific for binding to separate molecules on a tumor cell, tumor vasculature, tumor microenvironment, and/or immune cells.

The term “aptamer” includes DNA, RNA or peptides that are selected based on specific binding properties to a particular molecule. For example, an aptamer(s) can be selected for binding a particular gene or gene product in a tumor cell, tumor vasculature, tumor microenvironment, and/or an immune cell, as disclosed herein, where selection is made by methods known in the art and familiar to one of skill in the art. Subsequently, said aptamer(s) can be administered to a subject to modulate or regulate an immune response.

Some aptamers having affinity to a specific protein, DNA, amino acid and nucleotides have been described (e.g., K. Y. Wang, et al., Biochemistry 32:1899-1904 (1993); Pitner et al., U.S. Pat. No. 5,691,145; Gold, et al., Ann. Rev. Biochem. 64:763-797 (1995); Szostak et al., U.S. Pat. No. 5,631,146). High affinity and high specificity binding aptamers have been derived from combinatorial libraries (supra, Gold, et al.). Aptamers may have high affinities, with equilibrium dissociation constants ranging from micromolar to sub-nanomolar depending on the selection used, aptamers may also exhibit high selectivity, for example, showing a thousand fold discrimination between 7-methylg and g (Haller and Sarnow, Proc. Natl. Acad. Sci. USA 94:8521-8526 (1997)) or between D and L-tryptophan (supra, Gold et al.).

According to yet another aspect of the invention, there is provided the use of a compound or aptamer as defined above for the manufacture of a product for the diagnosis, detection and/or imaging and/or a medicament for the prevention and/or treatment of a disease or condition selected from an immune disorder, inflammatory disease, infectious disease, and neoplastic disease/cancer, including, but not limited to head and neck cancers, aero-digestive cancers, gastro-intestinal cancers, esophageal cancers, stomach/gastric cancers, pancreatic cancers, hepato-biliary/liver cancers, colorectal cancers, anal cancers, small intestine cancers, genito-urinary cancers, urologic cancers, renal/kidney cancers, ureter cancers, testicular cancers, urethra/penis cancers, gynecologic cancers, ovarian/fallopian tube cancers, peritoneal cancers, uterine/endometrial cancers, cervical/vagina/vulva cancers, gestational trophoblastic disease, prostate cancers, bone cancers, sarcoma (soft tissue/bone), lung cancers, mesothelioma, mediastinum cancers, breast cancers, central nervous system cancers, brain cancers, melanoma, hematologic malignancies, leukemia, lymphoma (Hodgkin's Disease and Non-Hodgkin's lymphoma), plasma cell neoplasms, myeloma, myelodysplastic syndrome, endocrine tumors, skin cancers, melanoma, thyroid cancers, parathyroid cancers, adrenal, pancreatic endocrine cancers, carcinoid, multiple endocrine neoplasia, AIDS-related malignancies, cancer of unknown primary site, and various childhood cancers.

According to another aspect of the invention, there is provided a kit for the prevention, treatment, diagnosis, detection and/or imaging of a disease or condition selected from an immune disorder, inflammatory disease, infectious disease, and neoplastic disease/cancer, comprising a compound, aptamer or composition of the invention.

Therefore, for various embodiments of the invention, one or more aptamer is selected based on the particular molecule targeted (e.g., aptamer targeting EGFR or other cancer markers). Standard procedures for in vitro selection are known, such as selex experiments, described at Science 249 (4968) 505-510 (1990), and Nature (London), 346 (6287) 818-822 (1990) which can be followed throughout, or with modifications and improvements known in the art. For example, fragments of target sequence are bound to a hi trap column (nhs activated) (selection column, provided by Pharmacia biotech) according to manufacturer instructions. The column forms a covalent bond with compounds having a primary amino group, such as a terminal amino group of a polypeptide. The pools of DNA templates (the library) are added to the chromatography column and let interact with the target peptide for approximately 1-hour at room temperature. The column is washed to remove any unbound aptamers and the bound aptamers are eluted with elution buffer (3M sodium thiocyanite). The eluted samples are then desalted with a nap-10 column (provided by Pharmacia biotech) and finally eluted in sterile water in an eppendorf. These are subsequently freeze-dried and polymerase chain reaction (“pcr”) reagents are added to the dry oligonucleotides to prepare them for the pcr, which is performed for 99 cycles with an annealing temperature of 56.degree. C. After the pcr procedure the DNA generated from this amplification is added to the chromatography column and used for the next selection round. These successive rounds of selection and amplification are carried out for 10 times. The final product achieved was a pcr product of about 100 μl.

After 10 rounds of selection and amplification, the pool is cloned to screen for DNA molecules with affinity for the desired target molecule (e.g., EGFR) (ta topo cloning kit, Invitrogen, UK). Individual clones are characterised using a general pcr protocol, with annealing temperature of 48.degree. C., for 35 cycles using m13 primers, and visualized on a 2.5% agarose gel. The positive clones are later grown in lb media in the presence of ampicillin and isolated using a standard plasmid DNA isolation kit (Quiagen, UK). The pool is further sequenced using standard ird-800 radioactive method (sequitherm excel ii, epicentre technologies, Madison, USA).

As such aptamers that are specific for a target molecule (e.g., cancer markers, such as EGFR) are selected. Such a target can be bound to a support in the identification of an aptamer as described previously. For example, a target peptide are immobilised onto functionalised sepharose beads in a chromatography column. Binding aptamers are thus retained in the column with non-binding or weakly binding aptamers being washed off. The strongly binding aptamers may then be removed for amplification by PCR. The column selection/amplification steps can be repeated to distinguish the most strongly binding aptamer(s). It is to be appreciated that a different population of aptamers will be present at each successive cycle, and that a large population is present initially. The entire process can be repeated, for example, for ten successive rounds of selection and amplification, to effect affinity maturation through competitive binding. The resulting final aptamer(s) can be cloned and sequenced and successful aptamer(s) of high affinity and specificity identified. Other numbers of selection/amplification cycles could be used.

The strongly-binding aptamers of the invention may be used in a large number of ways. For example, they may be used in the treatment and/or prevention of diseases or conditions where expression of the target molecule occurs. They may also be used in the diagnosis or detection of such diseases and conditions, for example by in vitro or in vivo methods or tests. In particular, the aptamers of the invention may be used to direct other agents to the proximity of the target. Thus, an aptamer may be bound to an agent which kills or damages cells and/or which is detectable to locate concentrations of the target either in vitro or in vivo. In various embodiments, an aptamer targeting a tumor/cancer cell or tumor vasculature, or a component of a tumor microenvironment is conjugated to one or more immunostimulatory sequences. In other embodiments, the tumor targeting aptamer may itself comprise of one or more immunostimulatory nucleic acid sequences (immunostimulatory aptamer). In one aspect, an immunostimulatory aptamer may be conjugated to an antibody, wherein the aptamer and/or the antibody can bind different components of a tumor cell/tumor vasculature/tumor microenvironment or an immune cell (e.g. macrophage or dendritic cell or others). This can allow bi-specific or multi-specific targeting of different components of a tumor cell while simultaneously activating immune responses against the target cell.

For example, the carboxylate group of the methionine arm or on the porphyrin may be used as the point of attachment to a targeting aptamer. This group allows the use of a peptide coupling methodology to attach the complex via an amino group on the aptamer. As such aptamers carrying a therapeutic moiety for tumor therapy may be produced (e.g., carrying immunostimulatory sequences or radioisotopes, etc.). Such coupling methodologies are attractive as they proceed under mild conditions and allow multiple complexes to be loaded onto a single aptamer. In this way, higher local concentrations of the one or more therapeutic moiety can be achieved at the site of the tumor. The porphyrin ligands used in the labelling protocol described above are obtained commercially or synthesised using established methods such as those described in tetrahedron, 1997, 53, 6755-6790.

Therefore, in various embodiments, aptamers may be linked to labeling moieties. For example, depending on the label used, labelling of the aptamer complexes can be verified using a range of physical techniques such as absorption spectroscopy, mass spectrometry, and in the case of fluorescent labels such as rhodamine and fluorescein, by fluorescence spectroscopy, and by relaxometry for MRI active labels.

The aptamer labelling may be carried out using standard peptide coupling protocols. For example, 0.01 mmol (0.004 g) of compound 11 or 0.01 mmol (0.009 g) porphyrin is dissolved in 0.5 cm.sup.3 water and 0.5 cm.sup.3 dmf. 0.002 g edci is added to the solution, which is stirred at room temperature for 15 min. 1 equivalent of the aptamer in 1 cm³ water is added and the reaction is allowed to proceed for 1 hour. The sample is applied onto a gel filtration column (nap-10) and the conjugate is eluted with 12 cm.sup.3 PBS (phosphate buffer saline). 1 cm³ fractions are collected, and the fractions containing the conjugate are combined.

Radiolabelled aptamers may be prepared for targeting purposes. In order to evaluate the efficacy of aptamers as therapeutic or diagnostic agents, the ligand would be loaded with the radionuclide as it comes off the generator and then coupled to the aptamer and administered immediately. Alternatively, the ligand may be first coupled to the aptamer and then only loaded with the radionuclide prior to administration. Monitoring under a gamma-camera after each administration and during the course of a treatment will provide evidence of the efficacy of the aptamer as a diagnostic- and therapeutic reagent.

It is to be appreciated the methodology of the invention is not limited to DNA aptamers. It is also applicable to other types of oligonucleotides, such as RNA, pyranosyl RNA (pRNA) and oligonucleotides comprising modified moieties, such as unnatural bases or modified natural bases. Therefore, in some embodiments, the aptamer molecule is comprised of DNA, RNA, pRNA along with a therapeutic moiety.

In another aspect of the invention, aptamers provides multivalent functionalized aptamer molecule which can be linked to one or more therapeutic moieties and/or one or more labeling moieties. A functionalised aptamer may have one attached ligand, however, it is possible to attach multiple ligands to an aptamer and/or attach multiple aptamers to a ligand. A unit comprising five ligands and four aptamers is schematically shown below: amino modified aptamers with modification at both the 3′ and the 5′ end are used. For example, four aptamer recognition units can be involved, which are attached via peptide bonds to the four carboxy groups of dota using a standard peptide coupling reaction with starting materials of excess aptamers (.gtoreq.4:1 of aptamer to dota) to allow for coupling to all available coupling sites. Mag3 (or any other ligand, such as ligand 9 or other commercial ligands) is then coupled to the other end of the aptamer, resulting in a four-aptamer complex carrying effectively 5 ligands loaded with targeting and/or therapeutic moieties (e.g., immunostimulatory nucleic acids, antibodies, immunostimulatory molecules, cytotoxic agents, and/or radionuclides).

A multivalent approach increases the amount or robustness of the therapeutic effect that may be delivered to the cell target. Furthermore, such an approach can also increase stability of the aptamer-therapeutic moiety molecule (e.g., resistance to nucleases) and increase the half-life of the aptamer, allowing it to remain active in the body. Furthermore, multivalency increases the size of the aptamer therapeutic. For example, by linking four aptamers together, the molecule is effectively increased in size (about 40 kda in total, instead of 10 kda for each individual unit), thus limiting its clearance from the system and offering additional useful time in circulation. The circulation time of such modified aptamers may be several hours, matching or surpassing the half-life of the relevant radionuclide.

As should be evident from the foregoing description, the aptamers of the invention, or variations thereon, may be connected to another compound for various uses, such as therapy or diagnosis. An aptamer may be joined to a ligand, such as those disclosed herein, by, for example, ionic or covalent bonds, or by other ways such as hydrogen bonding. The aptamer may thus guide the ligand to the target. The aptamer is preferably directly connected to the ligand. More specifically, the aptamer may be bound to the ligand without the use of a peptide tether. An aptamer may be joined to a ligand or other agent by a pendant moiety such as an amino or hydroxyl group. Several other agents may be attached to the same aptamer, and several aptamers may be attached to the same agent. The aptamers could be linked to ligands such as mag2 (mercaptoacetyl diglycerine), mag3 (mercaptoacetyl triglycerine), hynic (hydrazinonicotinic acid), n.sub.4-chelators, hydrazino-type chelators and thiol-containing chelators. In particular, dota and related cyclen derived ligands are suitable for functionalising aptamers. Also, the aptamer could be linked to fluorescent or phosphorescent groups and MRI agents. Examples include fluorescein, rhodamine, biotin, cyanine, acridine, digoxigenin-11-dutp, and lanthanides.

C. Peptides

In some aspects of the invention the targeting moiety for delivery of a biologically active agent is a peptide. For example, an INAS can be conjugated to a peptide which can bind with a component of a cancer or tumor cells. Therefore, such conjugates of the invention comprise peptide targeting moieties which binds to a cellular component of a tumor cell, tumor vasculature, and/or a component of a tumor microenvironment. In some embodiments, targeting moiety peptides can be an antagonist or agonist of an integrin. Integrins, which comprise an alpha and a beta subunit, include numerous types including: α₁β₂, α₂β₁, α₃β₁, α₄β₁, α₅β₁, α₆β₁, α₇β₁, α₈β₁, α₉β₁, α₆β₄, α₄β₇, α_{D}β₂, α_{D}β₂, α_{L}β₂, α_{M}β₂, αᵥβ₃, αᵥβ₅, αᵥβ₆, αᵥβ₈, αₓβ₂, α_{IIb}β₃, α_{IELb}β₇, and the like.

In one embodiment, the targeting moiety is αᵥβ₃. Integrin αᵥβ₃ is expressed on a variety of cells and has been shown to mediate several biologically relevant processes, including adhesion of osteoclasts to bone matrix, migration of vascular smooth muscle cells, and angiogenesis. Suitable targeting molecules for integrins include RGD peptides or peptidomimetics as well as non-RGD peptides or peptidomimetics (see, e.g., U.S. Pat. Nos. 5,767,071 and 5,780,426) for other integrins such as α₄.β₁ (VLA-4), α₄.β₇ (see, e.g., U.S. Pat. No. 6,365,619; Chang et al., Bioorganic & Medicinal Chem Lett, 12:159-163 (2002); Lin et al., Bioorganic & Medicinal Chem Lett, 12:133-136 (2002)), and the like.

In particular embodiments of the invention, targeting moiety peptides may be derived from phage display or other sources, and include but are not limited to, αvβ1 integrin (CRRETAWAC (SEQ ID NO:5)), αvβ3 integrin (CDCRGDCFC (SEQ ID NO:6)/RGD-4C; RGDWXE (SEQ ID NO:7)), αvβ5 integrin (TRGDTF (SEQ ID NO:8)), αvβ6 (RGDLxxL (SEQ ID NO:9) or xxDLxxL (SEQ ID NO: 10)), αIIβ3 (SRGDM (SEQ ID NO:11)), annexin V mimic for αvβ5 (VVISYSMPD (SEQ ID NO: 12)), E-selectin (IELLQAR (SEQ ID NO:13)), Endothelial cell mitochondria (CNGRC-GG-(KLAKLAK)2 (SEQ ID NO:14)), Ephrin-A2 and Ephrin-A4 (CVSNPRWKC (SEQ ID NO:15), CHVLWSTRC (SEQ ID NO:16)), Fibronectin (CWDDGWLC (SEQ ID NO:17)), ICAM-I or von Willebrand factor (CPCFLLGCC (SEQ ID NO:18)/LLG4C), lamin-1 (DFKLFAVY (SEQ ID NO:19)), P-selectin (EWVDV (SEQ ID NO:20)), MMP-9:integrin complex (D/E)(D/E)(G/L)W (SEQ ID NO:21), MMP-9 and MMP-2 (gelatinases) (CTTHWGFTLC (SEQ ID NO:22)), Type I cadherin on endothelium (N-Ac-CHAVC-NH2), Flt-1 region of VEGF NxxEIExYxxWxxxxxY(SEQ ID NO:23), KDR region of VEGF (HTMYYHHYQHHL (SEQ ID NO:24), ATWLPPR (SEQ ID NO:25)), VEGF receptor (WHSDMEWWYLLG (SEQ ID NO:26), RRKRRR (SEQ ID NO:27), Aminopeptidase N/CD13 (NGR), NG2 proteolgycan (TAASGVRSMH (SEQ ID NO:28), LTLRWVGLMS (SEQ ID NO:29)), Adrenal gland derived peptide (LMLPRAD (SEQ ID NO:30)), Adipose Tissue derived peptide (CKGGRAKDC SEQ ID NO:31)), Brain derived peptide (SR1), Brain endothelium derived peptide (CLSSRLDAC (SEQ ID NO:32)), Glioma cell derived peptide (VGLPEHTQ (SEQ ID NO:33)), Neuroblastoma derived peptide (VPWMEPAYQRFL (SEQ ID NO:34)), Bone Marrow derived peptide (GGG, GFS, LWS), Breast cancer (HER2/neu) derived peptide (LTVxPWx (SEQ ID NO:35), LTVxPWY (SEQ ID NO:36), HER2Ab/Trastuzumab mimotope—LLGPYELWELSH (SEQ ID NO:37)), Colon derived peptide (RPMC (SEQ ID NO:38)), Intestine derived peptide (YSGKWGW (SEQ ID NO:39)), Head and Neck Squamous Cell Cancer derived peptide (TSPLNIHNGQKL (SEQ ID NO:40)), Lung vasculature derived peptide (CGFELETC (SEQ ID NO:41)), Coronary artery endothelia derived peptide (NSVRDL(G/S) (SEQ ID NO:42), NSVSSx(S/A) (SEQ ID NO:43)), Lymphatic Vessel derived peptide (CGNKRTRGC (SEQ ID NO:44)/Lyp-1), Multiple Organ derived peptide (GVL, EGRx (SEQ ID NO:45), xFG(GNV) (SEQ ID NO:46)), Pancreatic Islet derived peptide (CVSSNPRWKC (SEQ ID NO:47), CHVLWSTRC (SEQ ID NO:48)), Pancreas derived peptide (SWCEPGWCR (SEQ ID NO:49)), Prostate derived peptide (AGG, DPRATPGS (SEQ ID NO:50), SMSIARL (SEQ ID NO:51), CGRRAGGSC (SEQ ID NO:52), GVL), Retina derived peptide (RDV, CSCFRDVCC (SEQ ID NO:53)), Teratogen ligand derived peptide (TPKTSVT (SEQ ID NO:54)), and Uterus derived peptide (GLSGGRS (SEQ ID NO:55)).

In one aspect, an αᵥβ₃ peptide can have the sequence characteristics of either the natural ligand of αᵥβ₃ or αᵥβ₃ itself at the region involved in αᵥβ₃-ligand interaction. In one aspect, an αᵥβ₃ peptide contains the RGD tripeptide and corresponds in sequence to the natural ligand in the RGD-containing region.

In one aspect, RGD-containing peptides have a sequence corresponding to the amino acid residue sequence of the RGD-containing region of a natural ligand of αᵥβ₃ such as fibrinogen, vitronectin, von Willebrand factor, laminin, thrombospondin, and the like ligands. The sequence of these αᵥβ₃ ligands are well known. Thus, an αᵥβ₃ peptide can be derived from any of the natural ligands.

In another aspect, an αᵥβ₃ peptide preferentially inhibits αᵥβ₃ binding to its natural ligand(s) when compared to other integrins. The identification of αᵥβ₃ peptides having selectivity for αᵥβ₃ can readily be identified in a typical inhibition of binding assay, such as the ELISA assay.

A peptide of the present invention typically comprises no more than about 100 amino acid residues, preferably no more than about 60 residues, more preferably no more than about 30 residues. Peptides of the invention can be linear or cyclic.

It should be understood that a subject peptide need not be identical to the amino acid residue sequence of an αᵥβ₃ natural ligand. Exemplary sequences include: CDCRGDCFC (SEQ ID NO:36) and GGCDGRCG (SEQ ID NO:4).

A peptide of the invention includes any analog, fragment or chemical derivative of a peptide whose amino acid residue sequence is shown herein. Therefore, a present peptide can be subject to various changes, substitutions, insertions, and deletions where such changes provide for certain advantages in its use. In this regard, an αᵥβ₃ peptide of this invention corresponds to, rather than is identical to, the sequence of a recited peptide where one or more changes are made and it retains the ability to function as an αᵥβ₃ peptide in one or more of the assays.

The term “analog” includes any peptide having an amino acid residue sequence substantially identical to a sequence specifically shown herein in which one or more residues have been conservatively substituted with a functionally similar residue and which displays the αᵥβ₃ activity as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The term “fragment” refers to any subject polypeptide having an amino acid residue sequence shorter than that of a polypeptide whose amino acid residue sequence is disclosed herein.

As used herein “a tumor targeting peptide” includes polymers containing fewer than 100 amino acids, where the polymer specifically binds to a cellular component of a tumor cell, tumor vasculature, and/or a component of a tumor microenvironment.

A peptide of the present invention can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis. Appropriate protective groups usable in such synthesis are described in the above texts and in J. F. W. McOmie, “Protective Groups in Organic Chemistry”, Plenum Press, New York, 1973.

### II. ACTIVE AGENTS

As described herein, compositions of the invention comprise a targeting moiety specific to a molecule present on a target cell coupled to a therapeutic agent. More particularly, such therapeutic agents are biologically active agents which induce an immune response to the target cell. Therefore, in some embodiments methods of use of compositions of the invention include preventing or treating cancer, such as to prevent proliferation of, elimination or reduction of tumor cells and/or tumor growth. In further embodiments, methods of use of compositions of the invention include preventing or treating diseases associated with infectious agents.

A. Nucleic Acid Molecules

As disclosed herein, a nucleic acid molecule comprises one or more of the following: double strand DNA (ds DNA), single strand DNA (ssDNA), multistrand DNA, double strand RNA (ds RNA), single strand RNA (ssRNA), multistrand RNA, DNA-RNA hybrid (single strand or multistrand), peptide nucleic acid (PNA), PNA-DNA hybrid (single or multistrand), PNA-RNA hybrid (single or multistrand), locked nucleic acids (LNA), LNA-DNA hybrid (single or multistrand), LNA-RNA hybrid (single or multistrand). In one embodiment, the nucleic acid molecule encodes one or more products (e.g. nucleic acids such as RNA, peptides, polypeptides, fusion peptides). In one embodiment, the nucleic acid molecule includes one or more immunostimulatory nucleic acid sequences (INAS) that can activate immune cells.

1. Immunostimulatory Nucleic Acid Molecules

In some embodiments, the therapeutic agent is an immunostimulatory DNA-conjugated or RNA-conjugated antibody or other targeting moiety that simultaneously activates the immune system, recruits immune effector cells to the targeted cells, and sensitizes tumor cells to immunologic cytotoxicity (e.g., by simultaneous blockade of growth factor-mediated signaling). The immune effector cells cooperate with direct DNA- or RNA-induced death signaling to induce apoptosis of tumor cells. Also, the tumor antigens released by apoptotic tumor cells, for example, are presented by dendritic cells (DCs) to generate long lasting adaptive antitumor immune responses. Therefore, selective activation of intracellular death signaling and immunologic elimination of targeted tumor cells can be achieved without toxicity to normal cells.

In one aspect, the therapeutic agent is a nucleotide-conjugated antibody or nucleotide-conjugated targeting moiety that induces direct death of targeted tumor cells via mechanisms that are independent of their immunostimulatory effects. Treatment of EGFR-expressing cancer cells with DNA-conjugated anti-EGFR antibodies or HER2/neu-expressing cancer cells with DNA-conjugated anti-HER2/neu antibodies results in direct target receptor-specific death in the absence of PBMCs. The deregulated cell-cell fusion of targeted cells in response to treatment with nucleotide-conjugated antibodies results in the formation of coalesced (hybrid or multinucleated) cells with a limited lifespan and impaired replicating ability. This novel form of targeted cell death (cell hyperfusion) is not observed in response to treatment with unconjugated parent antibodies (anti-EGFR or anti-HER2/neu antibodies) or free DNA. Examples of antibody-conjugated nucleotide sequences that induce direct cell death (* represents phosphorothioate bonds, rest are phosphodiester): 5′G*G*GGACGACGTCGTG-G*G*G*G*G*G 3′ (SEQ ID NO: 1); 5′G*G*GGGAGCATGCTGG*G*G*G*G*G 3′ (SEQ ID NO:2). Cell hyperfusion may be observed by methods which assay for cell survival/proliferation including, but not limited to phase contrast microscopy, trypan blue exclusion, crystal violet staining, detection of coalesced cell bodies and/or detection of formation of multinucleate cell bodies.

In one aspect, DNA-conjugated or RNA-conjugated polypeptides/peptides or tumor-targeting moieties simultaneously activate antitumor immune responses in the milieu of the tumor cells and inhibit tumor angiogenesis. In a related aspect, polypeptides/peptides targeting the tumor cell, tumor vasculature, or tumor microenvironment aid in the delivery of immunostimulatory DNA/RNA to the tumor, and also inhibit tumor angiogenesis.

In one embodiment, a targeting moiety is linked to a nucleic acid sequence that comprises a pathogen-associated molecular pattern (PAMP) or other sequence which directly or indirectly induces activation, maturation, proliferation, and/or survival of immune cells. Such immune cells include but are not limited to Dendritic Cells, T lymphocytes, Natural Killer Cells, B lymphocytes, Monocytes, or Macrophages. Furthermore, such nucleic acid sequences can activate innate or adaptive immunity, such as through ligation of endosomally expressed receptors, including members of the Toll-like receptor (TLR-) and nucleotide-binding oligomerization domain (NOD)-gene families, and/or through TLR-independent immune cell stimulation, including detection by Retinoic-acid-inducible protein I (RIG-I) and MDA-5, and/or through target cell responses, such as expression or release of endogenous immunostimulatory molecules, including alarmins, cytokines, chemokines, costimulatory molecules, and/or through immune danger signals from damaged or dying target cells. In various embodiments, the biologically active agent coupled to a targeting moiety are agonists of TLR, including but not limited to TLR3, TLR7/8 and TLR9.

In various embodiments of the invention, one or more targeting moiety is coupled to one or more biologically active agent(s) that comprise nucleic acid molecule(s). For example, the active agent may be one or more immunostimulatory nucleic acid sequences (INAS). In one embodiment, one or more of the nucleic acid sequences may comprise a pathogen-associated molecular pattern (PAMP) or other sequence which directly induces and/or promotes Toll-like receptor (TLR)-dependent or TLR-independent activation, proliferation and/or survival of immune cells. In another embodiment, the active agent may comprise stable/stabilized nucleic acid sequence(s) that induces activation/proliferation/survival of immune cells via cellular responses to undigested nucleotides that escape lysosomal degradation. In another embodiment, the nucleic acid sequences may comprise a structure or sequence that is recognized as a danger signal or damage-associated molecular pattern (DAMP) which triggers cellular responses that induce or promote activation, proliferation, and/or survival of immune cells. In yet another embodiment, such nucleic acid sequences are coding or non-coding sequences, which promote target cell death (activates death signaling responses and/or inhibits survival gene expression) and secondary immune activation triggered by immunostimulatory molecules from stressed, damaged or dying/apoptotic target cells. In another embodiment, the nucleic acid molecule functions as an immunostimulatory molecule by virtue of its secondary structure.

As should be evident based on the disclosure throughout, an INAS may be selected from the following: ssDNA, ds DNA, antisense DNA, oligodeoxynucleotides, ds RNA, ss RNA, siRNA, shRNA, miRNA, oligoribonucleotides, ribozymes, plasmids, DNA/RNA hybrids, or aptamers.

In various embodiments, a composition of the invention comprises a targeting moiety as described herein coupled to one or more nucleic acid sequences that comprise a pathogen-associated molecular pattern (PAMP) or other sequence which induces and/or promotes Toll-like receptor (TLR)-dependent or TLR-independent activation, proliferation and/or survival of immune cells.

Pathogen associated molecular patterns (PAMPs) are motifs from pathogens or damaged host cells, such as nucleic acids, that are recognized by the immune system via receptors that include members of the Toll-like receptor (TLR)— and nucleotide-binding oligomerization domain (NOD)-gene families. Nucleic acid sequences [double stranded (ds) RNA, single stranded (ss) RNA, ds DNA and ss DNA] activate the innate or adaptive immune system via their recognition/engagement by specific TLRs expressed in macrophages, monocytes, dendritic cells, and other antigen-presenting cells (APCs). In macrophages, and dendritic cells, TLRs that recognize nucleic acids are expressed in endosomes. These include TLR3, TLR7/8, and TLR9, which sense ds RNA, ss RNA, and DNA, respectively. Efficient translocation of nucleic acid ligands to intracellular endosomes (such as via antibody-mediated receptor-mediated endocytosis) induces TLR-activation and immunostimulation.

In various embodiments, a composition of the invention comprises a targeting moiety as described herein coupled to a TLR agonist. TLRs are activated by naturally occurring molecules that are released from microbial sources; synthetic molecules based on those of microbial products; small molecules with no obvious structural relationship to naturally occurring ligands; and endogenous ligands of host origin.

In one embodiment, a biologically active agent coupled to a targeting moiety (e.g., antibody specific for EGFR) is an INAS, which may be any sequence that comprises a PAMP or TLR agonist. INAS may comprise any nucleic acid sequence with a structure or chemistry that is capable of eliciting TLR activation (TLR agonist) and/or stimulation of immune responses. TLRs include any TLR, including but not limited to TLR1 to TLR11. INAS may comprise any DNA or RNA with a sequence or structure that is capable of TLR-activation and/or immunostimulation when introduced into macrophages, monocytes, and/or dendritic cells via conjugation to a targeting moiety. Conjugation of nucleic acids to antibodies facilitates their endosomal delivery to immune cells (via antibody-mediated Fc receptor-mediated endocytosis), and increases their ability to activate the immune system. It is notable that DNA or RNA sequences that do not strictly conform to specific or canonical immunostimulatory motifs are also rendered capable of TLR-activation and/or immunostimulation when introduced into macrophages or dendritic cells via antibody-conjugation.

In some embodiments, an attenuated or inactivated (live or killed) immunostimulatory pathogen carrying INAS, PAMP, or TLR agonist (such as bacteria or virus) is targeted to a tumor via expression or conjugation to a tumor targeting moiety (e.g., antibody, peptide, aptamer).

In various embodiments, an INAS contemplated for use in the compositions and methods of the invention is a genomic nucleic acid sequence (DNA or RNA) derived from bacterial or viral pathogens. In another embodiment, the INAS is a synthetic DNA or RNA “mimic” (e.g., derivatives and analogues) corresponding to a portion of a pathogen's or organism's genome. Exemplary nonlimiting sequences include bacterial DNA or RNA (e.g., attenuated mycobacteria bacillus Calmette Guerin DNA; Bacillus Anthracis; Brucella; Salmonella; Shigella), and viral DNA or RNA (e.g., Flaviviridae, Paramyxoviridae, Orthomyxoviridae, Rhabdoviridae; Herpes simplex virus type 1 or 2 DNA; Reovirus ds RNA; Influenza virus ss RNA; Avian Influenza; Norovirus; HIV-1 ss RNA; HIV gag mRNA).

In various embodiments, such active agents (INAS or TLR agonists) contemplated for use in the compositions and methods of the invention include but are not limited to agonists of TLR3, TLR7, TLR8 which can be in the form of double-stranded RNA (ds RNA); Single-stranded (ss) RNA; short interfering RNA (siRNA); Short hairpin RNA (sh RNA). Such agonists can be natural or synthetic RNA of different sequences and lengths which can activate TLR3, TLR7, and/or TLR8, and activate dendritic cells (DCs) and/or other immune cells.

In various embodiments, the immunostimulatory activity of INAS (in vitro transcribed RNA or chemically synthesized oligoribonucleotides) may be increased by one or more of the following specifications: Absence of methylated nucleosides (including 5-methylcytidine, N6-methyladenosine, N7-methylguanosine 5-methyluridine, 2′-O-methylated nucleosides); absence of modification of U residues (including 2-thiouridine or pseudouridine); absence of 3′ poly(A) tails; absence of 5′ terminal cap structure; presence of 5′ triphosphate moiety; sequences of a minimal length of 19 bases; or resistance to nucleases (e.g phosphorothiate internucleotide linkages). Exemplary nonlimiting sequences include e.g., 5′ pUGGAUCCGGCUUUG AGAUCUU (SEQ ID NO:56); 5′ppGGGAGACAGGGGUGUCCGCCAUUUCCAGGUU (SEQ ID NO:57); or 5′ pppGGGAGACAGGCUAUAACUCACAUAAUGUAUU (SEQ ID NO:58).

In further embodiments, such active agents (INAS or TLR agonists) are TLR3 agonists, including but not limited to dsRNA, Polyinosinic-polycytidylic acid (Poly I:C); long ds RNA (>30 bases); siRNA duplexes.

In yet other embodiments, such active agents (INAS or TLR agonists) are TLR7 or TLR8 agonists, which include but are not limited to, single-stranded (ss) RNAs; Double stranded (ds) RNAs; Short interfering RNA (siRNA); Short hairpin RNA (sh RNA); RNA with immunostimulatory sequences/motifs.

In various other embodiments, the biologically active agent(s) coupled to a targeting moiety includes but are not limited to synthetic RNAs with 5′-UGUGU-3′ (SEQ ID NO:60) or 5′-UGU-3′ (SEQ ID NO:61) motif(s) located on either strand of siRNA duplex or ds RNA or ss RNA or shRNA. Exemplary sequences include but are not limited to the following RNAs: 5′-CUACACAAAUCAGCGAUUU(SEQ ID NO:) (SEQ ID NO:62); 3′-GAUGUGUUUAGUCGCUAAA(SEQ ID NO:5′-63) UUGAUGUGUUUAGUCGCUA (SEQ ID NO:64); 3′-AACUACACAAAUCA GCGAU(SEQ ID NO:65); 5′-GAUUAUGUCCGGUUAUGUA(SEQ ID NO:66); 3′-CUAAUACAG GCCAAUACAU(SEQ ID NO:67); 5′-AUGUAUUGGCCUGUAUUAG(SEQ ID NO:68); 3′-UACAUAACCGGACAUAAUC(SEQ ID NO:69); 5′-GGUCGGAAUCGAAGGUUUA(SEQ ID NO:70); 3′-CCAGCCUUAGCUUCCAAAU(SEQ ID NO:71); 5′-GGUCGGAGCUAAAG GUUUA(SEQ ID NO:72); 3′-CCAGCCUCGAUUUCCAAAU(SEQ ID NO:73); 5′-CAGCUUUGUGUGAGCGUAU(SEQ ID NO:74); 3′-GUCGAAACACACUCGCAUA(SEQ ID NO:75).

In various other embodiments, the biologically active agent(s) coupled to a targeting moiety includes but are not limited to synthetic RNAs with 5′-GUCCUUCAA-3′ (SEQ ID NO:76) motif(s) located on either strand of an siRNA duplex or single strand RNA or short hairpin (sh) RNA. In some embodiments, such agents are have a minimum length of RNA=19 bases and are TLR9-independent. Exemplary sequences for such active agents include: 5′-AGCUUAACCU GUCCUUCAAdTdT-3′ (SEQ ID NO:78); 5′-UUGAAGGACAGGUUA AGCUdTdT-3′ (SEQ ID NO:79); 5′-ACCUGUCCUUCAAUUACCAdTdT-3′ (SEQ ID NO:80); 5′-UGGUAAUUG AAGGACAGGUdTdT-3′ (SEQ ID NO:81); 5′-AAAAAAAACUGUCCUUCAA (SEQ ID NO:82); 5′-AAAAAAAAAUGUCCUUCAA (SEQ ID NO:83); 5′-AAAAAAAAAAGUCCUUCAA (SEQ ID NO:84); 5′-UGUCCUUCAAUGUCCUUCAA (SEQ ID NO:85); 5′-AGCUUAACCU GUCCUUCAA (SEQ ID NO:86); or 5′-AGCUUAACCU GUCCUUCAACUACACAAA UUGAAGGACAGGUUAAGCU (SEQ ID NO:87).

In further embodiments, such active agents are GU- or U-rich requences. Exemplary sequences for such active agents include but not limited to: (G+U)-rich single stranded RNA (GU dinucleotides); Poly (U)-rich ssRNA 5′-UUUUUUUUUUUUUUUU (SEQ ID NO:59);

In further embodiments, such active agents are: Imidazole quinolines (e.g. imiquimod, resiquimod); Guanosine nucleotides and analogs (e.g. loxoribine; ′7-Thia-8-oxo-guanosine; 7-deazaguanosine; 7-allyl-8-oxoguanosine).

In further embodiments, such active agents are RNA sequences with repetitive elements, simple repeats, and contiguous repetition or “runs” of one base (adenine, thymine, guanine, cytosine, uracil, inosinic acid or xanthylic acid) e.g. poly(A), poly(C), poly(G), poly(U), poly(X), poly(I).

In other embodiments of the invention, the biologically active agents are TLR9 agonists, such as single stranded DNA (ss DNA) or double stranded DNA (ds DNA), bacterial DNA, Viral DNA, or plasmid DNA. In one example, such agonists comprise Herpes simplex virus type-1 DNA.

In other embodiments, such TLR9 agonist active agents are oligodeoxynucleotides with CpG (i.e., “CpG DNA” or DNA containing a cytosine followed by guanosine and linked by a phosphate bond), such as oligodeoxynucleotides with CpG motifs [TCGTT or TCGTA or TCGACGX or TCGATCG] (methylated or unmethylated). Examples of such immunostimulatory nucleic acid sequences include CpG A: Phosphorothioate(*) mixed backbone; Single CpG motif (hexameric purine-pyrimidine-CG-purine-pyrimidine); CpG flanking regions form a palindrome (self-complementary bases that have the potential to form a stem-loop structure); Poly-G tail at 3′ end (can interact to form ODN clusters). (e.g., G*G*TGCATCGATGCAG*G*G*G*G*G (SEQ ID NO:101)); C G B: Phosphorothioate backbone; multiple CpG motifs; TCG (e.g., TCGTCGTTTTTCGGTCGTTTT (SEQ ID NO: 102)); CpG C: Phosphorothioate backbone; Multiple CpG motifs; TCG dimer at 5′ end; CpG motif imbedded in a central palindrome (e.g., TCGTCGTITTCGGCGCGCGCCG (SEQ ID NO:103)); Other CpG compounds: 5′-TCGXCGX and 5′-TCGXTCG (X=any nucleotide).

In other embodiments, such active agents are presented as multiple copies with free 5′ ends having a phosphorothioate backbone with or without hydrophilic spacers (e.g., 5′TCGACGT (branched, with spacers); or 5′TCGATCG (branched, with spacers)).

In one embodiment, the invention provides an immunostimulatory nucleic acid sequence containing a CpG motif represented by the formula:

5′N₁X₁CGX₂N₂3′

where at least one nucleotide separates consecutive CpGs; X₁ is adenine, guanine, or thymine; X₂ is cytosine or thymine; N is any nucleotide and N₁+N₂ is from about 0-26 bases with the proviso that N₁ and N₂ do not contain a CCGG quadmer or more than one CCG or CGG trimer; and the nucleic acid sequence is from about 8-30 bases in length.

In another embodiment, the invention provides an isolated immunostimulatory nucleic acid sequence containing a CpG motif represented by the formula:

5′N₁X₁X₂CGX₃X₄N₂3′

where at least one nucleotide separates consecutive CpGs; X₁X₂ include GpT, GpG, GpA, ApT and ApA; X₃X₄ include TpT or CpT; N is any nucleotide and N₁+N₂ is from about 0-26 bases with the proviso that N₁ and N₂ do not contain a CCGG quadmer or more than one CCG or CGG trimer; and the nucleic acid sequence is from about 8-30 bases in length.

In a related aspect, the immunostimulatory nucleic acid sequences of the invention include X₁X₂ selected from GpT, GpG, GpA and ApA and X₃X₄ is selected from TpT, CpT and GpT. For facilitating uptake into cells, CpG containing immunostimulatory nucleic acid molecules may be in the range of 8 to 30 bases in length. However, nucleic acids of any size (even many kb long) are immunostimulatory if sufficient immunostimulatory motifs are present, since such larger nucleic acids are degraded into oligonucleotides inside of cells. In another aspect, synthetic oligonucleotides do not include a CGG quadmer or more than one CCG or CGG trimer at or near the 5′ and/or 3′ terminals and/or the consensus mitogenic CpG motif is not a palindrome. Prolonged immunostimulation can be obtained using stabilized oligonucleotides, where the oligonucleotide incorporates a phosphate backbone modification. For example, the modification is a phosphorothioate or phosphorodithioate modification. More particularly, the phosphate backbone modification occurs at the 5′ end of the nucleic acid for example, at the first two nucleotides of the 5′ end of the nucleic acid. Further, the phosphate backbone modification may occur at the 3′ end of the nucleic acid for example, at the last five nucleotides of the 3′ end of the nucleic acid.

In one aspect, the CpG DNA is in the range of between 8 to 30 bases in size when it is an oligonucleotide. Alternatively, CpG dinucleotides can be produced on a large scale in plasmids, which after being administered to a subject are degraded into oligonucleotides. In another aspect, nucleic acid molecules have a relatively high stimulation index with regard to B cell, monocyte and/or natural killer cell responses (e.g., cytokine, proliferative, lytic, or other responses).

Exemplary CpG DNA sequence: 5′ G*G*GGACGACGTCGTGG*G*G*G*G*G 3′ (SEQ ID NO:1)-Phosphorothioate(*) mixed backbone.

In some embodiments, conjugates of the invention have immunostimulatory nucleic acid sequences (INAS) that comprise RNA with unmethylated CpG motifs (CpG RNA), such as oligoribonucleotides with phosphorothiate (PS) backbone, unmethylated CpG motif, and 3′poly G tail (e.g., CpG ORN). Such sequences can function directly activate monocytes to produce IL-12, and indirectly stimulate NK cells to produce IFN-γ. Exemplary CpG ORN sequences include, 5′-GGUGCAUCGAUGCAGGGGGG (SEQ ID NO:115); 5′-GGUGCUUCGUUGCAGGGGGG (SEQ ID NO:116); 5′-GGUGCUUCGAUGCAGGGGGG (SEQ ID NO:117); or 5′-GGUGCUACGU UGCAGGGGGG (SEQ ID NO:118).

In some embodiments, conjugates of the invention have biologically active agents comprising synthetic oligodeoxynucleotides that do not contain unmethylated CpG. Examples of such immunostimulatory nucleic acid sequences include the following: ss DNA lacking canonical CpG motifs (GC inversion or methylated cytosines) can also activate TLR-9 (following endosomal translocation via receptor-mediated endocytosis); self-complementary polynucleotide, poly-(dG,dC); DNA with low content of non-methylated CpG sequences; and non-CpG ODN with phosphorothioate (PS*) backbone (PS-ODN). It is notable that PS-ODN lacking CpG motifs can induce monocytes to differentiate into a DC phenotype expressing high levels of CD83, CD86, CD40, and HLA-DR and low levels of CD14, and secrete CCL3 and CCL4 β-chemokines in a CpG-independent fashion. For example, in some embodiments, such a TLR9 agonist is T*G*C*T*G*C*T*T*T*T*G*T*G*C*T*T*T*T*G*T*G*C*T*T (SEQ ID NO: 108) or T*C*C*T*C*C*T*T*T*T*G*T*C*C*T*T*T*T*G*T*C*C*T*T (SEQ ID NO: 109).

In some embodiments, conjugates of the invention have biologically active agents comprising oligodeoxyribonucleotides with the immunostimulatory motif-PyN(T/A)(T/C/G)(T/C/G)(T/G)GT, wherein, Py=C/T; N=any deoxyribonucleotide; At least two positions within parentheses are Ts; At least 20 or more nucleotides; single stranded; Flanking sequence—5′XX Motif XXXX-3. Exemplary sequences include but are not limited to 5′-TCATCATTTTGT CATTTTGTCATT (SEQ ID NO:119); 5′-TCATTATTTTGTTATTTTGTCATT(SEQ ID NO: 120); 5′-TCATCCTTTTGT CCTTTTGTCATT (SEQ ID NO:121); 5′-TCATCTTTTTGT CTTTTTGTCATT (SEQ ID NO: 122); 5′-TCATCAATTTGT CAATFTTGTCATT (SEQ ID NO: 123); 5′-TCATCATCTTGT CATCTTGTCATT (SEQ ID NO: 124); 5′-TCATCATGTTGT CATGTTGTCATT (SEQ ID NO:125); 5′-TCATCATTCTGT CATTCTGTCATT (SEQ ID NO:126); 5′-TCATCATTGTGT CATTGTGTCATT (SEQ ID NO:127); 5′-TCATCATTTGGT CATTTGGTCATT (SEQ ID NO: 128); 5′-TCATTTTTTTGT TTTTTTGTCATT (SEQ ID NO:129); 5′-TCATTGTTTTGT TGTTTTGTCATT (SEQ ID NO:130); 5′-TCATTCTTTTGT TCTTTTGTCATT (SEQ ID NO:131).

In some embodiments, conjugates of the invention comprise nucleic acid sequences that induce TLR-independent immune stimulation via Retinoic-acid-inducible protein 1 (RIG-1) and MDA-5. Detection of pathogen-derived nucleic acids involves two cytosolic helicases, Retinoic-acid-inducible protein 1 (RIG-1) and MDA-5, which are essential for effective antiviral defense. RIG-1 recognizes a specific set of RNA viruses (Flaviviridae, Paramyxoviridae, Orthomyxoviridae, and Rhabdoviridae), whereas MDA-5 is responsible for defense against another set of RNA viruses (Picornaviridae). The structural basis for the distinction of viral RNA from abundant self RNA in the cytoplasm of virally infected cells involves (RIG-1)-mediated detection of the 5′-triphosphate end of RNA generated by viral polymerases. Detection of 5′-triphosphate RNA is abrogated by capping of the 5′-triphosphate end or by nucleoside modification of RNA, both occurring during posttranscriptional RNA processing in eukaryotes. Genomic RNA prepared from a negative-strand RNA virus and RNA prepared from virus-infected cells (but not from noninfected cells) can trigger a potent interferon-α-response. Furthermore, recognition of triphosphate RNA by RIG-1 induces an interferon response in DCs, monocytes, other eukaryotic cells. As such the response is not limited to immune cells.

Therefore, in various embodiments, INAS may comprise a RNA sequence with a molecular signature that is recognized by RIG-1: uncapped 5′-triphosphate RNA (now termed 3pRNA); absence of 5′ terminal cap structure (7-methyl guanosine cap); and absence of uridine modification (pseudouridine or 2-thiouridine or 2′-O-methylated UTP).

In other embodiments, conjugates of the invention comprise nucleic acid (DNA or RNA) vaccines encoding a viral polymerase (producing uncapped 5′-triphosphate in the cytosol), such as, but not limited to, the following: positive strand RNA viruses of the family of Flaviviridae; segmented NSV (VSV, Flu); non-segmented NSV, including Paramyxoviruses and Rhabdoviruses.

In other embodiments, conjugates of the invention comprise RNA (5′-triphosphate) with a minimal length of 19 bases (wherein no specific sequence motif is required and can be single stranded or double stranded), such as the following examples of in vitro transcribed RNA: 5′-pppAGCWUAACCUGUCCUUCAA-3′ (SEQ ID NO:110);
5′-pppGGGGCUGACCCUGAAGUUCAUCUU-3′ (SEQ ID NO: 111);
5′-pppGGGGAU GAAC UUCAGGGUCAGCUU-3′ (SEQ ID NO: 112);
5′-pppGGGGCUGACCCUGAAGUUCAUCUU-3′ (SEQ ID NO: 113)
3′-UUCGACUGGGACUUCAAGUAGGGGppp-5′ (SEQ ID NO:114).

In yet further embodiments, conjugates of the invention comprise in vitro transcribed triphosphate RNA via a cytosolically expressed T7 RNA polymerase; in vitro-generated dsRNA fragments of viral genomic sequences (e.g., Newcastle disease virus); genomic RNA or in vitro generated RNA from an RNA virus (e.g., Flaviviridae, Paramyxoviridae, Orthomyxoviridae, and Rhabdoviridae).

In yet further embodiments, conjugates of the invention comprise INAS which can be long double-stranded RNA, short ds RNA (such as siRNA) or short ds RNA with blunt ends.

In yet further embodiments, an INAS may comprise a RNA sequence with a molecular signature that is recognized by MDA-5, such as long double-stranded RNA or Poly(I:C).

In various embodiments, the biologically active agent(s) are stabilized nucleic acid sequence(s) that induces activation/proliferation/survival of immune cells via cellular responses to undigested nucleotides that escape lysosomal degradation.

Macrophages engulf apoptotic dying cells that are generated during programmed cell death and digest DNA by lysosomal DNase. Endogenous DNA that escapes lysosomal degradation in macrophages and dendritic cells triggers a Toll-like receptor-independent gene induction program that results in production of type I interferons and other cytokines/chemokines that activate the innate immune system. The introduction of endogenous DNA-immunoglobulin complexes into macrophages or dendritic cells activates immune cells and triggers autoimmunity independently of known TLRs or TLR signaling molecules (TLR9, TLR3, TLR1-2, TLR5-8; MyD88, TRIF adaptor). Mice or humans with deficiencies in DNase or defects in clearance of apoptotic cells develop autoimmunity. Cross-reactivity against autoantigens associated with apoptotic debris containing nucleic acid-macromolecules can drive systemic autoimmunity.

The conjugation of tumor targeting antibody to INAS can induce autoimmune responses against tumor cells by inducing apoptosis of tumor cells, enhancing the uptake/internalization of bound apoptotic bodies by macrophages/dendritic cells (via Fc-FcR interactions), and promoting the activation of immune cells (via the nuclease resistant INAS and/or undigested nucleic acids from damaged/dying/apoptotic tumor cells).

In some embodiments, conjugates of the invention comprise INAS which may be any stable/stabilized nucleic acid sequence (ss DNA, ds DNA, ss RNA) that can mimic the TLR-dependent or TLR-independent activation of immune cells by apoptotic DNA. For example, such biologically active agents can include an immunostimulatory nucleic acid sequence derived from nucleic acid-containing macromolecules (nucleosomes) within apoptotic bodies; an immunostimulatory nucleic acid sequence that is generated in response to cellular distress and DNA damage; a nucleic acid sequence that can activate immune cells when introduced into macrophages or dendritic cells as a conjugate with an antibody or as an immune complex (e.g. DNA-immunoglobulin); a stable/stabilized nucleic acid sequence recognized as a natural danger signal which triggers cellular responses that activate the immune system.

In some embodiments, ss RNA sequences within small nuclear ribonucleoprotein particles (snRNPs) associated with apoptotic bodies are utilized as the biologically active agents.

Exemplary sequences for such active agents include, but are not limited to U snRNA sequences (or derivatives): 5′-GGACUGCGUUCGCGCUUUCC-3′ (SEQ ID NO:88); 5′-GGCUUAUCCAUUGCACUCCGGA-3′ (SEQ ID NO:89); 5′-ACGAAGGUGGUUUUCCCAG-3′ (SEQ ID NO:90); 5′-UUUGUGGUAGUGGGGGACUG-3′ (SEQ ID NO:91); 5′-GUAGUGUUUGUGGGGGACUG-3′ (SEQ ID NO:92); 5′-GUAGUGGGGGACUGUUWGUG-3′ (SEQ ID NO:93); 5′-GGACUGCGUUGUGGCUUUCC-3′ (SEQ ID NO:94); 5′-GAUACUUACCUG-3′ (SEQ ID NO:95); 5′-AAUJUGUGG-3′ (SEQ ID NO:96); 5′-AAUUUUUGA-3′ (SEQ ID NO:97); Nucleic acid sequences fitting the following formula: 5′-RAUxGR-3′ (SEQ ID NO:98) (R=purine G/A; x=3-6). Further exemplary sequences for such active agents include but not limited to RNA sequences in Ro Ribonucleoproteins (Ro RNPs), including hY1-5 RNA sequences (or derivatives): 5′-GACUAGCUUGCUGUUU-3′ (SEQ ID NO:99); 5′-GACUAGCCUUU-3′ (SEQ ID NO:100).

In another embodiment, the nucleic acid sequences may comprise a structure or sequence that is recognized as a danger signal or damage-associated molecular pattern (DAMP) which triggers cellular responses that induce or promote activation, proliferation, and/or survival of immune cells.

The conjugation of INAS to a targeting moiety (antibody, ligand, peptide, other) that binds a molecule on target cells enables introduction of INAS into target cells (via receptor-mediated endocytosis, electroporation, other mechanism). INAS may comprise a nucleic acid sequence recognized as a danger signal or DAMP which triggers target cellular responses that secondarily activate the immune system. Recognition of intracellular nucleotides (INAS) as a danger signal or DAMP induces immune cell activation via upregulation and/or release of cytokines/chemokines/costimulatory molecules (e.g. Interferons. NKG2D ligands) in target cells, upregulation and/or release of immunostimulatory intracellular proteins/endogenous molecules by stressed/damaged/dying target cells (e.g. alarmins), and/or secondary ingestion of immunostimulatory material from dying or dead (apoptotic) target cells (with non-degradable INAS) by macrophages/dendritic cells.

In various embodiments, a composition of the invention comprises a targeting moiety and a single-stranded (ss) DNA and double stranded (ds) DNA or RNA (INAS) which results in activation of one or more of the following cellular responses: DNA damage or stress responses in eukaryotic cells [such as, via activation of the ataxia telangiectasia mutant (ATM) kinase, Chk2, p53, and DNA-phosphatidylinositol 3 kinase (PK)], including inhibition of target cell proliferation (via activation of cell cycle checkpoints) and/or induction of target cell apoptosis (via activation of intrinsic death signaling); TLR-dependent or TLR-independent production and/or release of type I Interferons, other cytokines/chemokines/costimulatory molecules (e.g. NKG2D ligands) via activation of transcription factors and kinases (such as retinoic acid inducible gene 1, IKK, TBK1, IRFs, NF-κB, p53, Chk2); upregulation and/or release of immunostimulatory intracellular proteins/endogenous molecules by stressed/damaged/dying target cells (e.g. PAMPs, DAMPs, alarmins).

Furthermore, administration of conjugates of the invention can induce stress responses in target cells (tumor cells or cells in the tumor microenvironment) which result in maturation, activation, proliferation, and/or survival of immune cells [such as via increased expression and/or release ligands, cytokines, chemokines and or costimulatory signals for immune cells and/or endogenous danger signals. For example, in some embodiments, administration results in release of alarmins-defensins, cathelicidins, high mobility group Box protein 1 (HMGB1), S100 proteins, Hepatoma derived growth factor (HDGF), eosinophil derived neurotoxin (EDN), heat shock proteins, IL-1α, uric acid, Galectins, Thymosins, Nucleolin, Annexins, any hydrophobic protein part (Hyppo), or other defense effectors.

The immune system responds to antigens perceived to be associated with a dangerous situation such as infection. Danger signals act by stimulating dendritic cells to mature so that they can present foreign antigens and stimulate T lymphocytes. For example, multicellular animals detect pathogens via a set of receptors that recognize pathogen-associated molecular patterns (PAMPs). Dying mammalian cells have also been found to release danger signals (Danger associated molecular patterns) which promote immune responses to antigens associated with injured cells. Tissue/cell damage is recognized via receptor-mediated detection of intracellular proteins/endogenous molecules released by the dying/dead cells (termed “Alarmin(s)”). Alarmins represent a group of structurally diverse multifunctional host proteins that are rapidly released following pathogen challenge and/or cell death are able to both recruit and activate antigen-presenting cells. These potent immunostimulants, including defensins, cathelicidins, eosinophil-derived neurotoxin, and high-mobility group box protein 1, serve as early warning signals to activate innate and adaptive immune systems. Alarmins include intracellular components which signal/activate an immune response.

Alarmins can engage TLRs, IL-IR, RAGE, or other receptors. Effector cells of innate and adaptive immunity can secrete alarmins via nonclassical pathways and often do so when they are activated by PAMPs or other alarmins. Endogenous alarmins and exogenous PAMPs therefore convey a similar message and elicit similar responses; they can be considered subgroups of a larger set, the damage-associated molecular patterns (DAMPs). PAMPs and alarmins can synergistically reinforce activation of immune cells. Additional Alarmins are known further disclosed below (infra, under Peptides).

In various embodiments, a conjugate of the invention comprises a targeting moiety coupled to one or more stable/stabilized nucleic acid sequence(s) recognized as a danger signal or DAMP that triggers target cellular responses leading to immune cell activation. Exemplary sequences include ss DNA (No CpG sequence requirement; TLR-independent): 5′-AAG AGG TGG TGG AGG AGG TGG TGG AGG AGG TGG AGG-3′ (SEQ ID NO:132); 5′-TTG AAT TCC TAG TIT CCC AGA TAC AGT-3′ (SEQ ID NO:133); 5′-TCG GTA ACG GG-3′ (SEQ ID NO: 134); 5′-TTA GGG TTA GGG TTA GGG-3′ (SEQ ID NO:135); 5′-CGTTA-3′ (SEQ ID NO:136); 5′-GCCACTGC-3′ (SEQ ID NO:137); 5′-GCAGTGGC-3′ (SEQ ID NO:138).

In further embodiments, such active agents include human Telomeric DNA sequences—(TTAGGG)n repeats; Poly-G motifs; double stranded B-form DNA (TLR-independent; No CpG sequence requirement); linearized plasmid DNA; circular DNA with a large gap; single stranded circular phagemid, ds RNA or ss RNA.

The upregulation and/or release of endogenous danger signals associated with cellular damage/stress promotes DC recruitment, antigen uptake, maturation, and antigen presentation, and co-stimulation/priming of anti-tumor T cells. Therefore, in various embodiments of the invention, one or more targeting moiety is coupled to one or more biologically active agents including INAS and additional active agents such as DAMPs and/or Alarmins.

In yet another embodiment, a conjugate of the invention comprises a targeting moiety coupled to active agents such as coding or non-coding nucleic acid sequence(s) that promote target cell death and secondary immune activation triggered by immunostimulatory molecules from stressed, damaged or dying/apoptotic target cells.

For example, such active agents include a stable/stabilized coding or non-coding nucleic acid sequence that activates death signaling responses that result in apoptosis and secondary immune activation triggered by immunogenic apoptotic material; a stable/stabilized coding or non-coding nucleic acid sequence that promotes target cell death (apoptosis) via inhibition of survival gene expression and secondary immune activation triggered by immunogenic apoptotic material.

In another aspect of the invention, a Nucleic acids, can form secondary structures. These secondary structure are generally divided into helices (contiguous base pairs), and various kinds of loops (unpaired nucleotides surrounded by helices). The stem-loop structure in which a base-paired helix ends in a short unpaired loop is extremely common and is a building block for larger structural motifs such as cloverleaf structures, which are four-helix junctions. Internal loops (a short series of unpaired bases in a longer paired helix) and bulges (regions in which one strand of a helix has “extra” inserted bases with no counterparts in the opposite strand) are also frequent.

For example stem-loop intramolecular base pairing is a pattern that can occur in a nucleic acid molecule. The structure is also known as a hairpin or hairpin loop, which occurs when two regions of the same molecule, usually palindromic in nucleotide sequence, base-pair to form a double helix that ends in an unpaired loop.

The formation of a stem-loop structure is dependent on the stability of the resulting helix and loop regions. Thus, the first prerequisite is the presence of a sequence that can fold back on itself to form a paired double helix. The stability of this helix is determined by its length, the number of mismatches or bulges it contains (a small number are tolerable, especially in a long helix), and the base composition of the paired region. Pairings between guanine and cytosine have three hydrogen bonds and are more stable compared to adenine-thymine pairings, which have only two. Base stacking interactions, which align the pi orbitals of the bases' aromatic rings in a favorable orientation, can promote helix formation.

The stability of the loop also influences the formation of the stem-loop structure. “Loops” that are less than three bases long are sterically impossible and do not form. Exemplary loop length can be about 4-8 bases long.

For example a palindromic DNA sequence

| | |
|---|---|
| - - - CCTGCXXXXXXXGCAGG - - - | (SEQ ID NO:3) |

can form the following hairpin structure

```
- - - C G - - -
            C G
            T A
            G C
            C G
            X X
            X X
            X X
              X
```

Naturally occurring secondary structures, such as repetitive extragenic palindromic (REP) sequences, have been observed to stimulate the immune system. Magnusson et al. The Journal of Immunology, 2007, 179: 31-35. The term “REP sequences” encompasses repetitive and palindromic sequences with a length between 21 and 65 bases. REP sequences have been detected in the extragenic space of some bacterial genomes constituting >0.5% of the total extragenic space. These sequences are present in many Gram-negative bacteria and play important roles in DNA physiology and genomic plasticity. Strong immunostimulatory ODNs comprising motifs, such as REPs, can be used in the present invention because they have an appropriate length, and are palindromic. REPs palindromicity allows one to envisage possible stem-loop secondary structures that they could adopt. DNA secondary or tertiary structures could endow REPs with higher stability and DNase resistance. Furthermore, REPs have two additional advantageous features for being a target of immune recognition of bacteria: abundance and conservation. ODNs comprising REPs from Gram-negative human pathogens such as E. coli, S. enterica typhi, N. meningitidis, and P. aeruginosa produce innate immune system stimulation, which is mediated by TLR9 receptors. Magnusson et al. The Journal of Immunology, 2007, 179: 31-35. Detection by TLR9 is believed to be facilitated by the stable stem-loop secondary structures that REPs probably adopt. DNA tertiary structures, stable even under denaturing conditions may also stimulate IFN-α release.

In various embodiments, the targeting moiety-biologically active agent conjugates of the invention comprise a nucleic acid molecule which functions as an immunostimulatory molecule by virtue of its secondary structure. In one embodiment dsODNs with a natural phosphodiester backbone may be used to mimic secondary structures such as those seen in REPs. Thus, double-stranded phosphodiester oligonucleotides with the sequence of representative REPs from bacteria such as E. coli, S. enterica typhi, N. meningitidis, and P. aeruginosa may be used to activate production of the proinflammatory cytokines such as IFN-α. In another embodiment dsODNS with a synthetic backbone may be used. In yet another embodiment ssODNs may be used which form secondary and tertiary immunostimulatory structures. In various such embodiments, the targeting moiety is an antibody that is specific for a component present on a tumor cell. In other various such embodiments, the targeting moiety is an antibody which is specific for a component present on a pathogen (e.g., bacteria or virus).

As should be evident based on the disclosure throughout, one or more targeting moiety is coupled to one or more biologically active agent(s) that include one or more nucleic acid molecule(s)/sequence(s). In various embodiments, the active agent includes one or more nucleic acid sequences that induces activation, proliferation, and/or survival of immune cells (such as Dendritic Cells, T lymphocytes, Natural Killer Cells, B lymphocytes, Monocytes, Macrophages)(termed: Immunostimulatory Nucleic Acid Sequence(s)=INAS). INAS may comprise either: a pathogen-associated molecular pattern (PAMP) or other sequence/structure that directly induces TLR-dependent or TLR-independent activation/proliferation/survival of immune cells; and/or a stable or stabilized nucleic acid sequence/structure that induces activation/proliferation/survival of immune cells via cellular responses to undigested nucleotides that escape lysosomal degradation; a nucleic acid sequence/structure that is recognized as a natural danger signal or damage-associated molecular pattern (DAMP) which triggers cellular responses that activate the immune system; and/or a coding or non-coding nucleic acid sequence that promotes target cell death and secondary immune activation triggered by immunostimulatory molecules from stressed, damaged or dying/apoptotic target cells; and/or a nucleic acid molecule which functions as an immunostimulatory molecule by virtue of its secondary structure.

In another embodiment, the INAS may be conjugated to an antibody (or fragment), ligand, peptide, aptamer or other tumor targeting moiety. The entry of conjugates into either tumor targets or immune cells may be facilitated by any method, including receptor-mediated endocytosis or electroporation.

In one embodiment, a conjugate of the invention is a multivalent molecule either in the context of multiple targeting moieties to the same or different target cell component, as well as in the context of the one or more of the same or different biologically active agent. Thus, for example, in various embodiments of the invention through utilizing different combinations biologically active agents, a synergistic therapeutic effect results.

In various embodiments, the INAS conjugated to the antibody or targeting moiety may be a naked plasmid DNA or coding immunostimulatory nucleic acid sequence (DNA, RNA) that induces specific gene expression. In one embodiment, the coding nucleic acid is a minicircle.

Therefore, in one embodiment, administration of a composition comprising at least a targeting moiety and a nucleic acid molecule encoding a gene of interest, allows targeting of a target cell type (i.e., to which the targeting moiety is specific to a particular cell type (e.g., tumor cell or other cell), expression of a gene of interest, and simultaneous activation of immune responses against the target cell (antibody-mediated plasmid endocytosis and targeted expression of genes via intracellular circular non-replicating episomes: antibody-directed non-viral gene immunotherapy).

In various embodiments, antibody or targeting moiety against a target cell component (e.g. against HER2, EGFR, other) is conjugated to a plasmid vector selected from: a naked plasmid DNA; a plasmid replicon expressing a self-replicating RNA vector (replicase-based nucleic acid—DNA or RNA, such as an alphavirus replicon or a Sindbis virus replicon); plasmids encoding viral RNA polymerase; or plasmids encoding a gene of interest such as, a target/tumor antigen (DNA vaccine), an immunostimulatory molecule (cytokine, co-stimulatory molecule, or other immunostimulatory molecule e.g. endogenous danger signal, such as alarmins, a TLR agonist), a membrane bound Fc fragment or Fc Receptor (FcR)(e.g. CD32a), or a molecule that promotes target cell death (e.g. death receptors—TRAIL-receptors, Fas; death ligands—TRAIL, FasL). In various embodiments, such a tumor-targeted antibody or targeting moiety can be designed to target any target cell component disclosed herein (e.g., HER2, EGFR, etc.).

In some embodiments, the INAS conjugated to the antibody or targeting moiety may be an immunostimulatory nucleic acid that inhibits specific gene expression (siRNA or antisense or shRNA). This can allow bi-specific targeting of two components of a tumor cell while simultaneously activating immune responses against the target cell. In one embodiment, an antibody against a target cell component (e.g, HER2) is conjugated to siRNA silencing a survival gene or a ribozyme silencing the same. In further embodiments, such a tumor-targeted antibody is conjugated to siRNA or ribozyme silencing expression of an immunosuppressive molecule (e.g., indoleamine 2,3-dioxygenase (IDO)).

In one aspect, the INAS conjugated to the antibody may be an immunostimulatory aptamer (RNA or DNA) that can also bind a component of a tumor cell/tumor vasculature/tumor microenvironment or an immune cell (e.g. macrophage or dendritic cell or others). This can allow bi-specific targeting of two components of a tumor cell while simultaneously activating immune responses against the target cell.

Therefore in various embodiments, an tumor-targeted antibody is conjugated to INAS aptamer targeting another tumor antigen or receptor (e.g., the estrogen receptor; EGFR, any component disclosed herein); a tumor-targeted antibody conjugated to INAS aptamer targeting a dendritic cell (DC) receptor; a tumor-targeted antibody is conjugated to INAS aptamer targeting death receptor (e.g., TRAIL-Receptors or CD95/Fas); or an tumor-targeted antibody against death receptor conjugated to INAS aptamer targeting a tumor antigen or receptor (e.g., HER-2); in yet another embodiment, conjugation of INAS to estrogen receptor (ER) binding molecules (such as tamoxifen).

In any of the foregoing embodiments, and subsequent embodiments disclosed herein, the tumor-targeted antibody can be designed to target a tumor antigen or tumor associated antigen (i.e., cellular components described herein, such as HER2, EGFR, etc.).

In another embodiment, the INAS is conjugated to an antibody that binds one or more tumor antigen(s)/epitope(s) or antigen(s) from a pathogen. The immune complex comprising the antigen(s) and antibody-INAS can be used to generate immune responses against specific tumor antigens or pathogen-derived antigens.

In another embodiment, the INAS is conjugated to an antibody that is directed against a component of an immune cell (DC or other). This INAS-antibody conjugate may be secondarily conjugated to one or more tumor antigen(s)/epitope or antigen(s) from a pathogen. The antigen-antibody-INAS immune complex can be used to generate immune responses against specific tumor antigens or pathogen-derived antigens. For example, an active agent can comprise INAS and antigen conjugated to an antibody against a DC antigen uptake receptor.

In another embodiment, INAS and antigen are conjugated to an antibody that targets an immune cell antigen or receptor (e.g., against CD40, CD28, etc.).

In a further embodiment, an INAS is conjugated to an antibody against an immune cell antigen or receptor (e.g., CD40, T cell antiens, such as CD3, CD4, etc.). Examples for such INAS include siRNA for silencing expression of a specific molecule such as GATA-3, IDO, etc.).

In some embodiments, the INAS is conjugated to an Fc protein or antigen-Fc fusion protein, wherein the antigen is a tumor antigen or pathogen-derived epitope. The INAS-Fc conjugate or INAS-antigen-Fc conjugate can be used to generate immune responses against specific tumor antigens or pathogen-derived antigens.

In another embodiment, a bi-specific antibody binds a specific tumor antigen (anti-tumor antibody) as well as immunostimulatory nucleic acids (INAS-DNA or RNA)(anti-INAS antibody). These nucleic acid containing immune complexes (bound to INAS and apoptotic cells) can activate endosomal TLR-mediated or TLR-independent immune responses following engulfment by macrophages and dendritic cells. This can induce autoimmune responses directed against antigens derived from antibody-bound apoptotic tumor cells (patient-specific tumor DNA vaccines).

In another embodiment, a immunostimulatory nucleic acid sequence (INAS) is conjugated to a bi-specific antibody which binds a specific tumor antigen as well as a death receptor that activates death signaling upon engagement by the antibody. The bi-specific antibody induces apoptosis of the targeted tumor cells, and the apoptotic cells (containing immune complexes bound to INAS) can activate endosomal TLR-mediated or TLR-independent immune responses following engulfment by macrophages and dendritic cells. This can induce autoimmune responses directed against antigens derived from antibody-bound apoptotic tumor cells (patient-specific tumor DNA vaccines).

In another embodiment, a immunostimulatory nucleic acid sequence (INAS) is conjugated to a bi-specific antibody which binds a specific tumor antigen as well as an immune cell, such as a dendritic cell. The bi-specific antibody induces apoptosis of the targeted tumor cells, and the apoptotic cells (containing immune complexes bound to INAS) can activate endosomal TLR-mediated or TLR-independent immune responses following engulfment by macrophages and dendritic cells. This can induce autoimmune responses directed against antigens derived from antibody-bound apoptotic tumor cells (patient-specific tumor DNA vaccines).

In another embodiment, the conjugate of the invention (e.g., antibody-INAS or targeting moiety-INAS conjugate) is designed to enable the combined detection of dual pathogen-associated molecular patterns, e.g., dsRNA and DNA, to mimic definitive viral recognition, resulting in an enhanced innate immune response that could be used for tumor vaccination or immunotherapy. In one embodiment, a conjugate comprises a plasmid CpG DNA encoding viral RNA polymerase or RNA replicon. In another embodiment, a conjugate comprises an antibody conjugated with DNA-RNA hybrid INAS (DNA+RNA).

In another embodiment, the conjugate of the invention (e.g., targeting moiety-INAS or antibody-INAS conjugate) may also be secondarily conjugated/linked to another INAS (DNA or RNA) or INAS-independent immunostimulatory molecule such as another PAMP, Damage-associated molecular pattern (DAMP), Toll-like receptor ligand, TLR-independent immunostimulatory ligand, or immunostimulatory danger signal, including, but not limited to the following: TLR ligands: (naturally occurring, synthetic analogues, or fully synthetic small molecules); TLR1 (such as triacyl lipopeptides); TLR2 (such as lipoproteins/lipopeptides, peptidoglycan, lipoteichoic acid, lipoarabinomannan, atypical lipopolysaccharide, Di- and triacyl lipopeptides, HSP70); TLR3 (INAS, such as ds RNA, Polyinosinic-polycytidylic acid, other agonists); TLR4 [such as lipopolysaccharide, taxol, HSP60 (Chlamydia pneumoniae), LPS/lipid A mimetics, such as monophosphoryl lipid A, synthetic lipd A, E5564, Ribi529, Oligosaccharides of hyaluronic acid, hyaluronan (HA)); TLR5 (such as bacterial flagellin, discontinuous 13-amino acid peptide; TLR6 (such as diacyl lipopeptides); TLR7 (INAS, such as ss RNA, oligonucleotides, loxoribine, resiquimod, imiquimod, other agonists); TLR8 (INAS, such as ssRNA, other agonists); TLR9 (INAS, such as bacterial or viral DNA, CpG oligodeoxynucleotides, Non-CpG DNA, other agonists); Immunostimulatory Danger signals including, but not limited to Alarmins, such as defensins, cathelicidins, high mobility group Box protein 1 (HMGB1), S100 proteins, Hepatoma derived growth factor (HDGF), eosinophil derived neurotoxin (EDN), heat shock proteins (including hsp70, hsp90, gp96 eHsp such as Hsp72, others), IL-10, uric acid, Galectins, Thymosins, Nucleolin, Annexins, or any hydrophobic protein part (Hyppo).

In various embodiments, INAS may be a DNA or RNA or DNA/RNA hybrid sequence derived from any of the following categories: Pathogen-derived nucleic acids including immunostimulatory pathogens/organisms (attenuated or live or killed); genomic DNA or RNA sequences derived from pathogens/organisms; synthetic DNA or RNA “mimics” (e.g., derivatives and analogues) corresponding to a portion of a pathogen's or organism's genome.

2. Nucleic Acid Encoding Genes of Interest

In another aspect of the invention, compositions and methods are provided comprise a targeting moiety coupled to a linear or circular nucleic acid molecule encoding one or more polypeptide of interest. Therefore, in some embodiments, the nucleic acid molecule expresses (i.e., transcription and/or translation) a gene of interest. Examples of such coding nucleic acid molecules include but are not limited to viral vectors, plasmids, minicircles, linear and circular dsDNA. In one embodiment, a composition of the invention comprises a targeting moiety as described herein coupled to an active agent, which is a nucleic acid molecule encoding a peptide or polypeptide of interest. Polypeptides encoded and expressed in this fashion include tumor and infectious agent antigens disclosed herein and known in the art, which will enhance or simulate a subject's immune response. Thus, a targeting moiety targets a particular cell or tissue and effectively delivers a nucleic acid molecule encoding a desired product which is immunostimulatory.

Such a mechanism can be used to provide vaccination against a particular disease or infectious agent, as well as providing a method for enhancing or increasing an immune response. Expression vectors are widely used and known, and can be adapted for use with compositions and methods of the invention. Examples are provided in U.S. Pat. Nos. 7,049,098, 6,143,530, 7,384,744, 7,279,568, 7,262,014, 6,977,296 and 6,692,750; and U.S. patent application publication nos. 2008/0145376; 2006/0281703; 2006/0211117; 2004/0214329 and 2004/0209836.

Plasmids. In various embodiments, vaccination can be mediated by several types of DNA constructs. For example, in one embodiment a conjugate of the invention comprises whole circular plasmid DNAs to deliver genes of interest. These circular double stranded DNA constructs are derived from bacteria and contain not only the gene of interest along with a mammalian specific promoter and terminator but also elements needed for replication and maintenance in bacterial cells (including the origin of replication and antibiotic resistance cassette). Examples of such expression vectors are known and can be applied in the context of the present invention.

Minicircles. As discussed herein, in one embodiment, a conjugate of the invention comprises a DNA minicircle, which can be used for encoding and expression of desired genes of interest. Minicircles have emerged in an effort to improve both the expression of the genes of interest as well as the overall safety of DNA vaccines. Minicircies are formed from the recombination of plasmid DNA into two parts, the minicircle and the miniplasmid. After recombination the minicircle contains only the essential elements of DNA vaccines, namely the mammalian specific promoter, genes of interest and terminator. The minicircle may also contain other sequences, such as the recombination site, but can be configured to contain as little DNA as possible. The miniplasmid contains all of the other plasmid replication, maintenance and bacterial derived sequences that are usually unnecessary or unwanted in DNA vaccines. One example of a minicircle vaccine is that of Chen et. al. (Minicircle DNA vectors devoid of bacterial DNA result in persistent and high-level transgene expression in vivo, Molecular Therapy 8 (3), 2003; Improved production and purification of minicircle DNA vector free of plasmid bacterial sequences and capable of persistent transgene expression in vivo. Human Gene Therapy (16) p 126-131, 2005). This minicircle system has four key components. The first two consist of the DNA coding sequence for the φC31 recombinase and its recognition sequence (repeated twice in the construct). During production in bacteria expression of the φC31 is induced and results in the recombination of the parent plasmid into the minicircle (containing the DNA vaccine portion) and the miniplasmid. The second two key components consist of the DNA coding sequence for the sequence specific restriction endonuclease I-SceI and its recognition sequence encoded in the plasmid backbone. After recombination the miniplasmid is cleaved and linearized by I-SceI and degraded by the endogenous bacterial endonucleases. The minicircle is then purified by standard plasmid purification processes.

In yet another embodiment a conjugate of the invention comprises a linear DNA construct which encodes a gene of interest. In these constructs polymerase chain reaction (PCR) is used to amplify a DNA vaccine coding construct (i.e., promoter, antigen, terminator). The amplified construct is usually engineered to be resistant to cellular nucleases to prevent degradation upon in vivo use. For example Johansson et. al. (PCR-generated linear DNA fragments utilized as a hantavirus DNA vaccine, Vaccine 20 p. 3379-3388, 2002) used phosphorothioate-modified PCR primers to amplify their DNA vaccine construct in order to prevent exonuclease degradation upon vaccination.

In yet another embodiment, a conjugate of the invention comprises a minimalistic, immunologically defined gene expression (MIDGE). MIDGE is a minimal-size gene transfer unit containing the expression cassette, including promoter, gene, and RNA-stabilizing sequence, flanked by two short hairpin oligonucleotide sequences. The resulting vector is a small, linear, covalently closed, dumbbell-shaped molecule. DNA not encoding the desired gene is reduced to a minimum.

In a further embodiment, a conjugate comprises nucleic acid modifications which allow hybridization of two different nucleic acid molecules. For example, dsDNA (circular plasmid/minicircle or linear DNA) is modified to incorporate a nucleotide sequence that hybridizes and binds with an oligonucleotide in a site specific manner. Therefore, if a targeting moiety is coupled to an oligonculeotide, the oligonucleotide can in turn link to a expression vector (e.g., dsDNA). In an alternative embodiment, if a targeting moiety of the invention is coupled to an expression vector, the expression vector can inturn link to an oligonucleotide. In either case, the oligonucleotide can be pre-selected based on its properties as a PAMP, DAMP, TLR agonist, or Alarmin.

a) Expression Regulatory Sequences

In further embodiments, expression of desired gene of interest is effected by a nucleic acid molecule comprising a “promoter” which is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence (i.e., ORF) to control transcriptional initiation and/or expression of that sequence. A promoter may or may not be used in conjunction with an “enhancer,” which refers to a cis-acting regulatory sequence involved in the transcriptional activation of a nucleic acid sequence.

Certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well. However, in certain embodiments a promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence.

Naturally, it will be important to employ a promoter and/or enhancer that effectively directs the expression of the DNA segment in the organelle, cell, tissue and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment. In various embodiments, the human cytomegalovirus (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. One well known inducible system that would be useful is the Tet-Off™ or Tet-On™ system (Clontech, Palo Alto, Calif.) originally developed by Gossen and Bujard (Gossen and Bujard, 1992; Gossen et al., 1995). This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The tetracycline operator sequence to which the tetracycline repressor binds, and the tetracycline repressor protein. The gene of interest is cloned into a expression element behind a promoter that has tetracycline-responsive elements present in it. A second plasmid contains a regulatory element called the tetracycline-controlled transactivator, which is composed, in the Tet-Off™ system, of the VP16 domain from the herpes simplex virus and the wild-type tertracycline repressor. Thus in the absence of doxycycline, transcription is constitutively on. In the Tet-On™ system, the tetracycline repressor is not wild type and in the presence of doxycycline activates transcription. For gene therapy vector production, the Tet-Off™ system would be preferable so that the producer cells could be grown in the presence of tetracycline or doxycycline and prevent expression of a potentially toxic transgene, but when the vector is introduced to the patient, the gene expression would be constitutively on.

In some circumstances, it is desirable to regulate expression of a transgene in a gene therapy vector. For example, different viral promoters with varying strengths of activity are utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that are used depending on the desired effect include SV40, RSV LTR, HIV-1 and HIV-2 LTR, adenovirus promoters such as from the E1A, E2A, or MLP region, AAV LTR, HSV-TK, and avian sarcoma virus. Similarly tissue specific promoters are used to effect transcription in specific tissues or cells so as to reduce potential toxicity or undesirable effects to non-targeted tissues. For example, promoters such as the PSA associated promoter or prostate-specific glandular kallikrein.

In certain indications, it is desirable to activate transcription at specific times after administration of the gene therapy vector. This is done with such promoters as those that are hormone or cytokine regulatable. Cytokine and inflammatory protein responsive promoters that can be used include K and T kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitrypsin, lipoprotein lipase (Zechner et al., 1988), angiotensinogen (Ron et al., 1991), fibrinogen, c-jun (inducible by phorbol esters, TNF-alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-1 anti-chymotrypsin.

b) Enhancers

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Any promoter/enhancer combination (as per the Eukaryotic Promoter Data Base EPDB) can be used to drive expression of the gene. Eukaryotic cells can support cytoplasmic transcription from certain bacterial promoters if the appropriate bacterial polymerase is provided, either as part of the delivery complex or as an additional genetic expression construct.

c) Polyadenylation Signals

Where a cDNA insert is employed, one will typically desire to include a polyadenylation signal to effect proper polyadenylation of the gene transcript. The nature of the polyadenylation signal is not believed to be crucial to the successful practice of the invention, and any such sequence is employed such as human or bovine growth hormone and SV40 polyadenylation signals. Also contemplated as an element of the expression cassette is a terminator. These elements can serve to enhance message levels and to minimize read through from the cassette into other sequences.

d) Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be in-frame with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements is used to create multigene, or polycistronic messages. IRES elements are able to bypass the ribosome-scanning model of 5′ methylated cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

The promoter may be heterologous or endogenous. For example, a polynucleotide promoter sequence is selected from the group consisting a constitutive promoter (i.e., simian virus 40 (SV40) early promoter, a mouse mammary tumor virus promoter, a human immunodeficiency virus long terminal repeat promoter, a Moloney virus promoter, an avian leukemia virus promoter, an Epstein-Barr virus immediate early promoter, a Rous sarcoma virus promoter, a human action promoter, a human myosin promoter, a human hemoglobin promoter, cytomegalovirus (CMV) promoter, an EF1-alpha promoter, and a human muscle creatine promoter) an inducible promoter (i.e., metallothionein promoter, a glucocorticoid promoter, a progesterone promoter, and a tetracycline promoter) and a tissue specific promoter (i.e., dendritic cell (i.e., CD11c), PSA associated promoter or prostate-specific glandular kallikrein). Additional examples of various promoter elements which can be incorporated into the compositions and methods of the invention are known, such as those disclosed on various regulatory sequence databases: Tissue Specific Promoter Database available at tiprod.cbi.pku.edu.cn:8080/index.html; Eukaryotic Promoter Databse available at http://www.epd.isb-sib.ch/; Database of Orthologous Promoters http://doop.abc.hu.

Such promoters can be selected based on the target cell or tissue to which a composition of the invention is delivered in order to provide expression of a desired gene product. Furthermore, another level of selectivity in targeting comprises utilizing a targeting moiety that is selective for a desired cell or tissue type. For example, in such an embodiment, a composition comprises a targeting moiety that is specific for a cell type, and further comprises a nucleic acid molecule encoding a desired antigen and where expression is under control of a promoter specific for the cell-type.

In yet an alternative embodiment, a composition comprises two different targeting moities, where one is cell-type specific and the other is disease specific (e.g., targets tumor antigens or antigens associated with an infectious agent). Therefore, a general formula for such a composition could be illustrated as T1-T2-A1-A2 or a variation thereof, where T1=targeting moiety one and T2=targeting moiety two. Furthermore, such compositions can comprise one or more non-coding immunostimulatory nucleic acid molecules, one or more antigenic peptides, and one or more nucleic acid molecules encoding an antigenic polypeptide or costimulatory polypeptide.

B. Peptides-Co-Stimulatory

As indicated herein, in various embodiments, a composition of the invention comprises nucleic acid molecules which are immunostimulatory. In another aspect of the invention, compositions of the invention comprise a polypeptide or a nucleic acid which encodes a polypeptide which are stimulate a subject's immune response.

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism (Janeway et al., 1989; Medzhitov et al., 1997).

The receptors of the innate immune system that recognize PAMPs are called Pattern Recognition Receptors (PRRs) (Janeway et al., 1989; Medzhitov et al., 1997). These receptors vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly (Medzhitov et al., 1997; Fearon et al., 1996).

Cellular PRRs are expressed on effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, as discussed below. This latter function allows efficient mobilization of effector forces to combat the invaders.

The primary function of dendritic cells (DCs) is to acquire antigen in the peripheral tissues, travel to secondary lymphoid tissue, and present antigen to effector T cells of the immune system (Banchereau, et al., 2000; Banchereau, et al., 1998). As DCs carry out their crucial role in the immune response, they undergo maturational changes allowing them to perform the appropriate function for each environment (Termeer, C. C. et al., 2000). During DC maturation, antigen uptake potential is lost, the surface density of major histocompatibility complex (MHC) class I and class II molecules increases by 10-100 fold, and CD40, costimulatory and adhesion molecule expression also greatly increases (Lanzavecchia, A. et al., 2000). In addition, other genetic alterations permit the DCs to home to the T cell-rich paracortex of draining lymph nodes and to express T-cell chemokines that attract naive and memory T cells and prime antigen-specific naive TH0 cells (Adema, G. J. et al., 1997). During this stage, mature DCs present antigen via their MHC II molecules to CD4+ T helper cells, inducing the upregulation of T cell CD40 ligand (CD40L) that, in turn, engages the DC CD40 receptor. This DC:T cell interaction induces rapid expression of additional DC molecules that are crucial for the initiation of a potent CD8+ cytotoxic T lymphocyte (CTL) response, including further upregulation of MHC I and II molecules, adhesion molecules, costimulatory molecules (e.g., B7.1, B7.2), cytokines (e.g., IL-12) and anti-apoptotic proteins (e.g., Bcl-2) (Anderson, D. M., et al., 1997; Caux, C., et al., 1997; Ohshima, Y., et al., 1997; Sallusto, F., et al., 1998). CD8+ T cells exit lymph nodes, reenter circulation and home to the original site of inflammation to destroy pathogens or malignant cells.

One key parameter influencing the function of DCs is the CD40 receptor, serving as the “on switch” for DCs (Bennett, S. R. et al., 1998; Clark, S. R. et al., 2000; Fernandez, N. C., et al., 1999; Ridge, J. P. et al., 1998; Schoenberger, S. P., et al., 1998). CD40 is a 48-kDa transmembrane member of the TNF receptor superfamily (Mcwhirter, S. M., et al., 1999). CD40-CD40L interaction induces CD40 trimerization, necessary for initiating signaling cascades involving TNF receptor associated factors (TRAFs) (Ni, C. Z., et al., 2000; Pullen, S. S. et al., 1999). CD40 uses these signaling molecules to activate several transcription factors in DCs, including NF.kappa.B, AP-1, STAT3, and p38MAPK (McWhirter, S. M., et al., 1999).

Co-stimulatory polypeptides include any molecule or polypeptide that activates the NFκB pathway, Akt pathway, and/or p38 pathway. The DC activation system is based upon utilizing a recombinant signaling molecule fused to a ligand-binding domains (i.e., a small molecule binding domain) in which the co-stimulatory polypeptide is activated and/or regulated with a ligand resulting in oligomerization (i.e., a lipid-permeable, organic, dimerizing drug). Other systems that may be used to crosslink or oligomerization of co-stimulatory polypeptides include antibodies, natural ligands, and/or artificial cross-reacting or synthetic ligands. Yet further, other dimerization systems contemplated include the coumermycin/DNA gyrase B system.

Co-stimulatory polypeptides that can be used in the present invention include those that activate NFκB and other variable signaling cascades for example the p38 pathway and/or Akt pathway. Such co-stimulatory polypeptides include, but are not limited to Pattern Recognition Receptors, C-reactive protein receptors (i.e., Nod1, Nod2, PtX3-R), TNF receptors (i.e., CD40, RANK/TRANCE-R, OX40, 4-1BB), and HSP receptors (Lox-1 and CD-91).

As described herein, PRRs include, but are not limited to endocytic pattern-recognition receptors (i.e., mannose receptors, scavenger receptors (i.e., Mac-1, LRP, peptidoglycan, techoic acids, toxins, CD11c/CR4)); external signal pattern-recognition receptors (Toll-like receptors (TLR1, TLR2, TLR3, TLR4, TLR5, TLR6, TLR7, TLR8, TLR9, TLR10, TLR11), peptidoglycan recognition protein, (PGRPs bind bacterial peptidoglycan, and CD14); and internal signal pattern-recognition receptors (i.e., NOD-receptors 1 & 2).

In yet a further embodiment, a composition of the invention comprises a targeting moiety, and at least a nucleic acid sequence which encodes one or more co-stimulatory polypeptides. The co-stimulatory polypeptide(s) can be expressed in addition to or in place of an antigenic polypeptide. For example, in one embodiment, a immunoconjugate comprises a targeting moiety, an immunostimulatory nucleic acid (e.g., PAMP), and a expressable nucleic acid encoding one or more (e.g., two or three) co-stimulatory polypeptide. In an additional embodiment, the immunoconjugate comprises an antigenic peptide or polypeptide, or an additional nucleic acid molecule encoding an antigenic peptide or polypeptide.

The co-stimulatory polypeptide includes, but is not limited to Pattern Recognition Receptors, C-reactive protein receptors (i.e., Nod1, Nod2, PtX3-R), TNF receptor (i.e., CD40, RANK/TRANCE-R, OX40, 4-1 BB), and HSP receptors (Lox-1 and CD-91). More specifically, the co-stimulatory polypeptide is a CD40 cytoplasmic domain.

Therefore, in various embodiments of the invention, a composition comprising a targeting moiety, and at least one co-stimulatory polypeptide or a nucleic acid molecule encoding a co-stimulatory polypeptide. Such co-stimulatory polypeptide molecules are capable of amplifying the T-cell-mediate response by upregulating dendritic cell expression of antigen presentation molecules. Co-stimulatory proteins that are contemplated in the present invention include, for example, but are not limited to the members of tumor necrosis factor (TNF) family (i.e., CD40, RANK/TRANCE-R, OX40, 4-1B), Toll-like receptors, C-reactive protein receptors, Pattern Recognition Receptors, and HSP receptors. In one embodiment, composition of the invention comprise a nucleic acid molecule expressing the cytoplasmic domains from these co-stimulatory polypeptides. The cytoplasmic domain from one of the various co-stimulatory polypeptides, including mutants thereof, where the recognition sequence involved in initiating transcription associated with the cytoplasmic domain is known or a gene responsive to such sequence is known. Additional examples of co-stimulatory polypeptides which can be used within the context of the invention herein are known in the art, such as disclosed in U.S. Pat. Nos. 7,404,950; 6,891,030; 6,803,192; and 7,074,590, and U.S. patent application nos. 2007/0172947; 20060269566 and 2005/0084913.

C. Antimicrobial Peptide (Alarmins)

In another embodiment, a conjugate of the invention is linked to or comprises a sequence which encodes one or more antimicrobial peptide. The antimicrobial peptide according to the present invention is a peptide capable of killing a microbial organism or inhibiting its growth. The antimicrobial activities of the antimicrobial peptides of the present invention include, without limitation, antibacterial, antiviral, or antifungal activities. Antimicrobial peptides include various classes of peptides, e.g., peptides originally isolated from plants as well as animals. In animals, antimicrobial peptides are usually expressed by various cells including neutrophils and epithelial cells. In mammals including human, antimicrobial peptides are usually found on the surface of the tongue, trachea, and upper intestine. Naturally occurring antimicrobial peptides are generally amphipathic molecules that contain fewer than 100 amino acids. Many of these peptides generally have a net positive charge (i.e., cationic) and most form helical structures.

In one embodiment, the antimicrobial peptide according to the present invention comprises about 2 to about 100 amino acids, from about 5 to about 50, or from about 7 to about 20. In one preferred embodiment, the targeting peptide has a length of 5, 6, 7, 8, 9, 10, 11, 12, 13, 14, 16, 17, 18, 19, 20, 21, 22, 23, 24, 25, 26, 27, 28, 29, or 30 amino acids.

In another embodiment, the antimicrobial peptide has the antimicrobial activity with a minimum inhibitory concentration (MIC) of no more than about 40 μM, no more than about 30 μM, no more than 20 μM, or no more than 10 μM.

In another embodiment, the antimicrobial peptide contains one or more antimicrobial peptides including, without limitation, alexomycin, andropin, apidaecin, bacteriocin, .beta.-pleated sheet bacteriocin, bactenecin, buforin, cathelicidin, alpha.-helical clavanin, cecropin, dodecapeptide, defensin, .beta.-defensin, .alpha.-defensin, gaegurin, histatin, indolicidin, magainin, melittin, nisin, novispirin G10, protegrin, ranalexin, tachyplesin, and derivatives thereof.

Among these known antimicrobial peptides, tachyplesins are known to have antifungal and antibacterial activities. Andropin, apidaecin, bactencin, clavanin, dodecappeptide, defensin, and indolicidin are antimicrobial peptides having antibacterial activities. Buforin, nisin and cecropin peptides have been demonstrated to have antimicrobial effects on Escherichia. coli, Shigella disenteriae, Salmonella typhimurium, Streptococcus pneumoniae, Staphylococcus aureus, and Pseudomonas aeroginosa. Magainin and ranalexin peptides have been demonstrated to have antimicrobial effects on the same organisms, and in addition have such effects on Candida albicans, Cryptococcus neoformans, Candida krusei, and Helicobacter pylori. Magainin has also been demonstrated to have antimicrobial effects on herpes simplex virus. Alexomycin peptides have been demonstrated to have antimicrobial effects on Campylobacter jejuni, Moraxella catarrhalis and Haemophilus influenzae while defensin and .beta.-pleated sheet defensin peptides have been shown to have antimicrobial effects on Streptococcus pneumoneae. Histatin peptides and the derivatives thereof are another class of antimicrobial peptides, which have antifungal and antibacterial activities against a variety of organisms including Streptococcus mutans (MacKay, B. J. et al., Infect. Immun. 44:695-701 (1984); Xu, et al., J. Dent. Res. 69:239 (1990)).

In one embodiment, the antimicrobial peptide of the present invention contains one or more antimicrobial peptides from a class of histadine peptides and the derivatives thereof. Additional examples are provide in U.S. patent application publication no. US20080170991

In another embodiment, the antimicrobial peptide of the present invention contains one or more antimicrobial peptides from a class of protegrins and the derivatives thereof. For example, the antimicrobial peptide of the present invention contains protegrin PG-1.

Protegrin peptides are described in U.S. Pat. Nos. 5,693,486, 5,708,145, 5,804,558, 5,994,306, and 6,159,936, all of which are incorporated herein by reference.

The antimicrobial peptide according to the present invention can be produced by any suitable method known to one skilled in the art by itself or in combination with a targeting peptide and a linker peptide. For example, the antimicrobial peptides can be chemically synthesized via a synthesizer or recombinantly made using an expression system, e.g., a bacterial, yeast, or eukaryotic cell expression system. In the chemical synthesis, the antimicrobial peptide can be made by L-amino acid enantiomers or D-amino acid enantiomers.

In one embodiment, a conjugate of the invention comprises an antimicrobial peptideLL-37-cathelicidin-derived antimicrobial peptide: Alarmin

Antimicrobial peptides play an important role in the innate host defense of multicellular organisms against microbial intruders. A common characteristic among antimicrobial peptides is the ability to adopt an amphipathic conformation where clusters of hydrophobic and cationic amino acids are spatially organized in discrete sections of the molecule. The defensins and the cathelicidins are the two major families of antimicrobial peptides in mammals. Cathelicidins consist of a highly conserved N-terminal cathelin domain and a more diverse antimicrobial C terminus. LL-37, a 37-amino acid peptide with two N-terminal leucines, is the only known human cathelin-associated antimicrobial peptide. The precursor of LL-37, hCAP-18, and its mouse homolog, CRAMP, are primarily expressed in bone marrow cells but are also broadly expressed in nonmyeloid tissues, including epididymis, spermatids, and epithelial cells of a number of organs. Importantly, expression of LL-37 is induced upon infectious or inflammatory stimuli, both in keratinocytes and in epithelial cells at other sites. LL-37 induces bacterial cell lysis, neutralizes bacterial endotoxin and has chemoattractive effects on leukocytes. LL-37 represents an alarmin and TLR agonist that is capable of activating dendritic cells. LL-37 protects plasmid DNA against serum nuclease degradation and efficiently targets DNA to the nuclear compartment of mammalian cells. LL-37-DNA complexes enter mammalian cells via endocytosis that involves noncaveolar lipid raft domains as well as cell surface proteoglycans.

Preparation of complexes of Antibody-DNA conjugate and LL37: The LL-37 peptide (LLGDFFRKSKEKIGKEFKRIVQRIKDFLRNLVPRTES-C-amide) (SEQ ID NO:232) is synthesized, and the peptide sequence confirmed by reverse phase high pressure liquid chromatography and mass spectrometry. To form LL-37-DNA complexes, DNA (10 μg/ml) and LL-37 (5-100 μg/ml) are mixed by inversion and incubated for 30 min at room temperature. Alternatively, LL-37 may be covalently coupled to the antibody or incorporated in the antibody/targeting ligand as a fusion protein.

In some embodiments, a conjugate comprises a histidine-rich amphipathic antimicrobial peptide. Synthetic cationic amphipathic peptides containing a variable number of histidine residues may also be complexed with the antibody-DNA conjugates of the invention. The transfection efficiency depends on the number and positioning of histidine residues in the peptide as well as on the pH at which the in-plane to transmembrane transition takes place. Endosomal acidification is also required. These peptides maintain a high level of antibacterial activity even when complexed to DNA. Examples include amphipathic peptides that are rich in alanine and leucine residues with various numbers of lysine and histidine residues. Whereas the lysines at both ends of the peptides assist DNA condensation, the histidine residues favor endosomal escape of the DNA (11). Examples of peptide sequences include:

| | |
|---|---|
| KKALLALALHHLAHLALHLALALKKA; | (SEQ ID NO:233) |
| KKALLALALHHLAHLAHHLALALKKA; | (SEQ ID NO:234) |
| or | |
| KKALLALALHHLALLAHHLALALKKA-NH_{2.} | (SEQ ID NO:235) |

An illustrative method for forming a peptide-DNA complexes, peptide (4-6 ug/1 ug DNA) and DNA (each diluted in 100 μl of 150 mM NaCl) are mixed and incubated for 20 min at room temperature. Alternatively, the peptide may be covalently coupled to the antibody or incorporated in the antibody/targeting ligand as a fusion protein.

Other peptide for use in the context of the present invention include polybasic antimicrobial peptides, such as multifunctional peptides that bind DNA and destabilize membranes. In addition, such peptides include polybasic “membrane-penetrating peptides”: HIV-1 transactivator (Tat)—YGRKKRRQRRRPPQC (SEQ ID NO:236); Antennapedia protein of Drosophila—RQIKIWFQNRRMKWKK (SEQ ID NO:237); Herpes simplex VP22; or Polylysine. These peptides mediate DNA internalization via PG-dependent and nonclathrin-mediated endocytosis

In further embodiment, peptides include antimicrobial peptides such as KALA, ppTG20, and Vpr52-96. KALA and ppTG20 combine a positively charged lysine or arginine stretch required for DNA binding and an amphipathic membrane-destabilizing domain deriving from the fusogenic peptides GALA and JTS-1. These transfecting peptides have a strong propensity for an α-helical conformation that positions the lysines or arginines on one face of the helix.

In yet a further embodiment, a conjugate of the invention is linked to protamine sulfate. For example, the antibody-DNA conjugate is linked to nucleic acid binding protein or fragment of protamine (amino acids 8-29), which nucleates sperm DNA. Alternatively, the peptide may be covalently coupled to the antibody or incorporated in the antibody/targeting ligand as a fusion protein. Furthermore, other polycations (e.g., Polyethyleneimine (PEI)) or cationic liposomes (e.g., DOTAP) are known in the art and can be used in the context of the conjugates of the invention.

In yet further embodiments, a conjugate of the invention comprises such peptides described and a PAMP (such as a TLR agonist—listed in specifications) or DAMP (such as an alarmin—listed in specifications) (e.g., linked to an antibody-DNA conjugate as described herein).

D. Permeabilizing Peptides

In some embodiments, a composition (conjugate) comprises one or more permeabilzing peptides. Such peptides can be coupled to a conjugate of the invention using conventional coupling methods and those disclosed herein. Efficient transfer of proteins or nucleic acids across cellular membranes is one of the major problems in cell biology. To deliver the functional domain of a selected protein from the outside to the inside of intact cells, a carrier is needed. Cell Permeable Peptides, also known as Protein Transduction Domains (PTDs), are carriers with small peptide domains that can freely cross cell membranes. Several PTDs have been identified that allow a fused protein to efficiently cross cell membranes in a process known as protein transduction. Studies have demonstrated that a TAT peptide derived from the HIV TAT protein has the ability to transduce peptides or proteins into various cells. PTDs have been utilized in anticancer strategy, for example, a cell permeable Bcl-2 binding peptide, cpm1285, shows activity in slowing human myeloid leukemia growth in mice. Cell-permeable phosphopeptides, such as FGFR730pY, which mimics receptor binding sites for specific SH2 domain-containing proteins are potential tools for cancer research and cell signaling mechanism studies.

Examples of peptides which can be incorporated into the compositions and methods of the invention include but are not limited to, (Arg)₉, TAMRA-labeled, (Arg)9 FAM-labeled, [Cys58]105Y, Cell Penetrating Peptide, 1-antitrypsin (358-374)105Y, alpha1-antitrypsin (359-374), Aminopeptidase N Ligand (CD13), NGR peptide, Aminopeptidase N Ligand (CD13), NGR peptide, Antennapedia Leader Peptide (CT), Antennapedia Peptide, acid, Antennapedia Peptide, amide, Anti-BetaGamma (MPS-Phosducin—like protein C terminus), Anti-BetaGamma (MPS-Phosducin—like protein C terminus), Biotin-TAT (47-57), Buforin, Chimeric Rabies Virus Glycoprotein Fragment (RVG-9R), Cys(Npys) Antennapedia Peptide, amide, Cys(Npys)-(Arg)₉, Cys(Npys)-(D-Arg)₉, Cys(Npys)-TAT (47-57), Cys(Npys)-TAT (47-57), FAM-labeled, Cys-TAT (47-57), FITC-LC-Antennapedia Peptide, FITC-LC-MTS, FITC -LC-TAT (47-57), Lipid Membrane Translocating Peptide, Lipid Membrane Translocating Peptide, D-isomer, Mastoparan, Mastoparan X, MEK1 Derived Peptide Inhibitor 1, MEK1 Derived Peptide Inhibitor 1, Membrane-Permeable Sequence, MPS, MPG, HIV related, MPS-Gαi2, MPS-Gαi3, Myristol, NGR Peptide 1,2,3,4, Nuclear Localiation Signal Peptide, Pep-1: Chariot (Non-Covalent Delivery of Peptides and Proteins), Rabies Virus Matrix Protein Fragment (RV-MAT), Stearyl-MEK-1 Derived Peptide Inhibitor 1, amide, SynB1, TAT (47-57), TAT (47-57) GGG-Cys(Npys), TAT (47-57), FAM-labeled, TAT (47-57), TAMRA-labeled, TAT (47-57)-Lys(TAMRA), Tat (48-57), Tat-C (48-57), Tat-NR2Bct, TAT-NSF222 Fusion Peptide, TAT-NSF222scr Fusion Polypeptide, scrambled, TAT-NSF700 Fusion Peptide, TAT-NSF700scr, TAT-NSF81 scr Fusion Polypeptide, scrambeled, Transdermal Peptide, or Transportan. Furthermore, these peptides can be used for nucleic acid binding.

### III. COMPOSITIONS

A. Tumor Targeted Compositions

In another aspect of the invention, compositions and methods are provided which allow prophylactic or treatment of a disease condition described herein. In one embodiment, a composition of the invention provides a means for vaccination of an animal.

In one embodiment, a composition of the invention comprises one or more targeting moiety (T) which binds a target molecules or component of a cancer or tumor (tumor-targeting moiety). The targeted molecule may be a component of a tumor cells, tumor vasculature, or tumor microenvironment.

In one embodiment, the invention comprises a conjugate of a tumor-targeting moiety, such as an antibody, and a nucleic acid molecule, wherein the nucleic acid molecule encodes one or more products (e.g. nucleic acids such as RNA, peptides, polypeptides, fusion peptides) and is capable of stimulating an immune response. In one embodiment, the nucleic acid molecule includes one or more pathogen associated molecular pattern (PAMP) or other immunostimulatory motif. In another embodiment, the nucleic acid molecule encodes one or more products that stimulate an immune response. In a related embodiment, the nucleic acid molecule includes one or more pathogen associated molecular pattern (PAMP) or other immunostimulatory motif, and encodes one or more products that stimulates an immune response.

In a related embodiment, the nucleic acid molecule of the tumor-targeted conjugate encodes one or more antigens or antigenic determinants which can be processed and presented for recognition by T cells and/or B cells. The encoded antigenic determinants include one or more of each of the following: CD4⁺ T cell epitopes, CD8⁺ T cell epitopes, B cell epitopes. In one embodiment, the nucleic acid molecule encodes one or more antigens or antigenic determinants derived from one or more pathogen(s), microorganism(s) or virus(es). For example, the nucleic acid encodes sequences derived from tetanus toxin to provide CD4⁺ T-cell help [e.g. Tetanus derived T_{H} activating sequences: fragment C (FrC), FrC domain DOM1, or the promiscuous MHC class II-binding peptide p30]. In a related embodiment, the nucleic acid encodes one or more antigens or antigenic determinants derived from a microbial vaccine or other non-self source (e.g. Pseudomonas aeruginosa exotoxin, green fluorescent protein, plant viral coat proteins).

In a related embodiment, the invention comprises a conjugate of a tumor-targeting moiety, such as an antibody, one or more pathogen associated molecular pattern (PAMP) and/or nucleic acid molecule(s) encoding one or more antigens or antigenic determinants derived from one or more pathogen(s), microorganism(s) or virus(es)(T or B cell epitopes). In a related embodiment, the conjugate comprises a tumor targeting moiety and one or more PAMP(s). In another related embodiment, the conjugate comprises a tumor targeting moiety and one or more nucleic acid molecule(s) encoding one or more antigens or antigenic determinants derived from one or more pathogen(s), microorganism(s) or virus(es)(T or B cell epitopes). In another related embodiment, the conjugate comprises a tumor targeting moiety, one or more PAMP(s), and one or more nucleic acid molecule(s) encoding one or more antigens or antigenic determinants derived from one or more pathogen(s), microorganism(s) or virus(es)(T or B cell epitopes).

In one embodiment, the invention comprises a conjugate of a tumor-targeting moiety, such as an antibody, one or more damage associated molecular pattern (DAMP) or alarmin(s), and one or more nucleic acid molecule(s) encoding one or more antigens or antigenic determinants derived from one or more pathogen(s), microorganism(s) or virus(es)(T or B cell epitopes). In a related embodiment, the conjugate comprises a tumor targeting moiety and one or more DAMP/Alarmin(s). In another related embodiment, the conjugate comprises a tumor targeting moiety and one or more nucleic acid molecule(s) encoding one or more antigens or antigenic determinants derived from one or more pathogen(s), microorganism(s) or virus(es)(T or B cell epitopes). In another related embodiment, the conjugate comprises a tumor targeting moiety, one or more DAMP/Alarmin(s), and one or more nucleic acid molecule(s) encoding one or more antigens or antigenic determinants derived from one or more pathogen(s), microorganism(s) or virus(es)(T or B cell epitopes).

In one embodiment, the invention comprises a conjugate of a tumor-targeting moiety, such as an antibody, and one or more nucleic acid molecule(s) encoding one or more of the following: (i) one or more antigens or antigenic determinants derived from one or more pathogen(s), microorganism(s) or virus(es)(T or B cell epitopes), (ii) one or more pathogen associated molecular pattern (PAMP), (iii) one or more damage associated molecular patterns (DAMP)/alarmin(s), (iv) one or more immunostimulatory molecules, including molecules that recruit, bind, activate, mature and/or proliferate an antigen presenting cell or dendritic cell or other immune cell (such as T cells, B cells, NK cells) and molecules that counteract immune suppression (e.g. ligands/antibodies for DC uptake receptors, immunostimulatory cytokines, chemokines, costimulatory molecules, growth factors). In a related embodiment, the nucleic acid molecule additionally encodes one or more tumor antigens/antigenic determinants or tumor antigen-containing fusion proteins. In one aspect, the fusion partner of the tumor antigen facilitates antigen uptake by DCs, immune recognition, and/or immune activation. In another example, the fusion partner includes a molecule targeting a DC uptake receptor. In another example, the fusion partner is an antigen or antigenic determinant derived from one or more pathogen(s), microorganism(s) or virus(es). In another example, the fusion partner is an alarmin. In a related embodiment, the targeting moiety-nucleic acid conjugate(s) described herein further comprises one or more PAMP and/or one or more DAMP/Alarmin(s).

In one embodiment, the invention comprises a conjugate of a tumor-targeting moiety, such as an antibody, and one or more nucleic acid molecule(s) encoding one or more RNA molecules that can interfere with expression of one or more target cell genes [e.g. short interfering RNA (siRNA), short hairpin RNA (shRNA)]. In another embodiment, the nucleic acid molecule of the conjugate encodes one or more immunostimulatory RNA molecules.

In one embodiment, the invention comprises a conjugate of a tumor-targeting moiety, such as an antibody, and one or more nucleic acid molecule(s) encoding a molecule that induces death of the target cell.

In each of the targeting moiety-nucleic acid conjugates described herein, the nucleic acid molecule encodes one or more gene of interest under control of a transcription promoter that is functionally active in the desired cell. In one embodiment, tissue or tumor cell selective promoters are used for targeted expression in the desired cell type.

In one embodiment, each of the tumor targeting moiety-nucleic acid conjugates described herein is linked to one or more components for packaging and/or delivery of a nucleic acid molecule or conjugate. For example, these molecules include cationic peptide, cell permeabilizing peptide, DC targeting peptide, nucleic acid binding molecule, nuclear localization peptide, cationic liposome, lipophilic moiety, nanoparticle.

In one embodiment, the invention comprises a conjugate of a tumor-targeting moiety, such as an antibody, one or more nucleic acid molecule(s), and one or more peptide/polypeptide/lipopeptide(s). In one embodiment, the nucleic acid molecule incorporates one or more pathogen associated molecular pattern (PAMP) or other immunostimulatory motif, and/or encodes one or more products that stimulate an immune response, as described herein. In various related embodiments, the peptide/polypeptide/lipopeptide(s) include one or more of the following: (i) one or more antigens or antigenic determinants derived from one or more pathogen(s), microorganism(s) or virus(es)(e.g. CD4+ T cell epitopes), (ii) alarmins, (iii) DC binding molecules (e.g. ligands of DC uptake receptors). In one aspect, the peptide/polypeptides of the conjugate described herein may be fused/linked to each other and/or to a nucleic acid binding peptide or cell permeabilizing peptide [e.g. cationic peptides, protamine, HIV-tat, Arginine- or Histidine-rich sequence, LL-37).

In one embodiment, the invention comprises a conjugate of a tumor-targeting moiety, such as an antibody or aptamer, and one or more of the following: (a) one or more pathogen associated molecular pattern (PAMP), (b) one or more of the following peptide/polypeptide/lipopeptide(s):(i) one or more antigens or antigenic determinants derived from one or more pathogen(s), microorganism(s) or virus(es)(e.g. CD4+ T cell epitopes), (ii) alarmins, (iii) DC binding molecules (e.g. ligands of DC uptake receptors). In one aspect, the peptide/polypeptides of the conjugate described herein may be fused/linked to each other and/or to a nucleic acid binding peptide [e.g. cationic peptides, protamine, HIV-tat, Arginine- or Histidine-rich sequence, LL-37). In one aspect, the conjugate includes an immunostimulatory nucleic acid.

In one embodiment, the invention comprises a conjugate of a targeting moiety, such as an antibody, and a nucleic acid molecule which is an aptamer. In one embodiment the antibody and nucleic acid aptamer bind to different targets on the same cell type or different cell types. In one embodiment, the conjugate comprises an antibody targeting a tumor cell surface receptor (EGFR) and an aptamer targeting prostate specific membrane antigen (PSMA), thereby targeting both proteins in prostate cancer cells. In one embodiment, the nucleic acid molecule comprises the aptamer and one or more of the following: (i) PAMP or other immunostimulatory nucleic acid, (ii) DNA encoding one or more products that stimulate an immune response, as described herein.

While not intending to be limited to any one mechanism of action, one mechanism by which conjugates of the invention can operate is as follows. (1) The antibody-DNA conjugate binds the targeted molecule, such as a cell surface antigen or receptor on the tumor cell. (2) Binding of the conjugate to the tumor cell results in receptor-mediated endocytosis and facilitates cellular entry of the nucleic acid molecule. (3) Cellular entry enables promoter-driven expression of the gene of interest encoded by the nucleic acid molecule; and (4) Expression of the specified genes of interest in the targeted tumor cell triggers the following effects: (a) Expression of one or more encoded pathogen or pathogen-derived antigens or antigenic determinants (T or B cell epitopes); (b) Presentation of pathogen antigen-derived epitopes in tumor cells (and DCs) in the context of Major Histocompatibility Complex (MHC) molecules for recognition by T cells (CD4+ or CD8+) or B cells; (c) Antibodies recognizing pathogen antigen-derived B cell epitopes bind and promote antibody-dependent cellular cytotoxicity of tumor cells presenting these epitopes (via Fc-Fc receptor interactions); these antibodies may pre-exist in the recipient via prior exposure to the pathogen antigen vaccine or are generated following conjugate administration; (d) T cells recognizing pathogen antigen-derived T cell epitopes provide CD4+ T cell help (to DCs and CD8+ T cells) and CD8+ T-cell mediated cytotoxicity of tumor cells presenting these epitopes; these T cells may pre-exist via prior exposure to the pathogen antigen vaccine or are generated following conjugate administration or delivered via adoptive transfer of ex vivo activated/expanded antigen-reactive T cells.

Furthermore, phagocytosis of antibody coated tumor cells (opsonized cells) by dendritic cells (DCs) facilitate cross-presentation of pathogen-derived and tumor associated antigens in the context of MHC molecules (via Fc-Fc receptor interactions). In addition, antigen presenting cells (DCs) are activated by (a) Pathogen associated molecular patterns (in the nucleic acid molecule of the conjugate); (b) Damage associated molecular patterns (endogenous alarmins produced by dying tumor cells); (c) CD4+ T helper cells recognizing pathogen-derived CD4+ T cell epitopes. Therefore, activation of CD4+ T helper (T_{H}) cells and CD8+ T cells recognizing cross-presented pathogen antigen- or tumor antigen-derived epitopes results in antigen spreading. In addition, activated T cells induce cytotoxicty of tumor cells expressing pathogen-derived T cell epitopes as well as tumor cells expressing endogenous tumor antigen epitopes.

In addition, expression of the following classes of encoded immunostimulatory molecules may enhance recruitment, proliferation, survival and/or activation of DCs and/or T cells that recognize pathogen antigen- or tumor antigen epitopes on tumor cells: (1) Immunostimulatory cytokines (e.g. Interferons, IL-12, IL-15, GM-CSF); (2) T cell co-stimulatory molecules; (3) DC recruitment or activating molecules (PAMPs, DAMPs, alarmins)

Also, expression of the following classes of encoded molecules that induce death of targeted tumor cells, with production of immunostimulatory DAMPs, may enhance recruitment, proliferation, survival and/or activation of DCs and/or T cells that recognize pathogen antigen- or tumor antigen epitopes on tumor cells: (1) si RNA to silence survival genes of interest; (2) direct cytocidal or death signaling proteins; and (3) proteins encoded by suicide genes.

In one embodiment, a conjugate comprises a tumor-targeted antibody and DNA plasmid/minicircle encoding a pathogen antigen-derived gene. For example, an antibody targets the human Epidermal growth factor receptor cell surface receptor on tumor cells (anti-EGFR); or an antibody targets the human HER2/neu receptor cell surface receptor on tumor cells (anti-HER2/neu).

In another embodiment, a conjugate comprises a tumor-targeted aptamer and DNA plasmid/minicircle encoding a pathogen antigen-derived gene. For example, an aptamer targeting a cell surface molecule (prostate specific membrane antigen (PSMA) on tumor cells (PSMA RNA aptamer).

In another embodiments, a conjugate comprises a tumor-targeted peptide and DNA minicircle encoding a pathogen antigen-derived gene. Examples of such tumor targeted Peptide are known and disclosed herein (e.g., RGD peptide).

DNA Vaccine design and rationale: CD4+ T helper (T_{H}) cells are vital for the induction and maintenance of immune responses. T_{H} cells are required for priming and secondary expansion of CD8+ T cells and providing help to B cells for antibody production. Since autologous tumor antigens are incapable of inducing significant T_{H} responses, the tumor targeted DNA conjugate vaccines of the invention incorporate encoded pathogen-derived sequences, such as from tetanus toxin or Pseudomonas aeruginosa exotoxin, so that T_{H} cells from the existing anti-microbial repertoire can help mount CD8+ T cell and/or B cell responses against tumor antigens derived from the immunoconjugate-targeted tumor cell and/or antigens co-encoded/fused within the same plasmid or minicircle. DNA vaccines can also provide T-cell help by incorporating other non-self antigens such as green fluorescent protein, plant viral coat proteins, or immune targeting molecules (alone or co-expression with tumor antigens or as fusion partners).

The conjugation of DNA vaccines incorporating pathogen-derived sequences to tumor targeted moieties results in the expression of these antigenic determinants in the targeted tumor cell as well as the indirect transfer of antigenic material (pathogen-derived and endogenous tumor cells/antigens) to APCs that have phagocytosed the targeted tumor cells (cross-presentation). A proportion of the antibody-DNA vaccine may also be directly taken up and presented by APCs (via antibody Fc interactions with Fc receptors on APC FcR). Such cross-presentation and direct presentation of pathogen- and tumor-derived antigens can provide effective T-cell help and result in the following immune responses: (1) Induction of pathogen antigen- and tumor antigen-specific antibodies: The antibody-DNA conjugate of the invention enables expression of pathogen antigen (e.g. Tetanus toxin derived fragment C-FRC) in the targeted tumor cells as well as cross-presentation of FrC and tumor antigens by DCs (from apoptotic tumor cells and/or co-encoded/fused tumor antigens in the vaccine). (FrC)-specific T_{H} cells stimulated by DC are able to prime and boost B cells to produce antibodies against FrC peptide or tumor cell antigens (via CD40-CD40 ligand interaction and cytokine production). The expression of FrC antigenic determinants in tumor cells also renders them susceptible to ADCC by either anti-FrC antibodies or anti-tumor antibodies, thereby reinforcing the cross-presentation of these antigens by DC that have phagocytosed the opsonized or apoptotic tumor cells; (2) Induction of tumor-reactive cytotoxic T cells: The antibody-DNA vaccine encoding microbial antigens or other non-self antigens may be used to initiate and amplify CD8+ T lymphocyte (CTL) immune responses against a range of otherwise weak tumor antigens. (FrC)-specific T_{H} cells license DCs cross-presenting both FrC and tumor antigens to prime and boost CD8+ T cell responses against weak tumor antigens. Since immunodominant pathogen-derived peptides can restrict responses to sub-dominant tumor-derived epitopes, the pathogen-derived antigen encoded by the DNA vaccine may be minimized to contain epitopes required to provide CD4+ T cell help (such as a single domain of FrC-DOM1, or promiscuous MHC class II binding peptides, such as tetanus toxin p30).

These immune responses are facilitated and reinforced by the ability of the immunoconjugate of this invention to simultaneously activate DC via one or more of the following:(1) PAMPs that are incorporated in the conjugate (such as immunostimulatory nucleic acids); (2) Damage associated molecular patterns (DAMPs) that are included in the conjugate (e.g. alarmins, such as LL-37 cathelicidin); (3) Endogenous PAMPs or DAMPs produced via expression of the encoded genes or in response to cellular stress and damage; (4) Other endogenous immunostimulatory molecules that are produced via expression of the encoded genes or as a bystander effect of activating immune responses in the tumor cell milieu.

Also, expression of the following classes of encoded molecules that induce death of targeted tumor cells, with production of immunostimulatory DAMPs, may enhance recruitment, proliferation, survival and/or activation of DCs and/or T cells that recognize pathogen antigen- or tumor antigen epitopes on tumor cells: (1) si RNA to silence survival genes of interest; (2) direct cytocidal or death signaling proteins; and (3) proteins encoded by suicide genes.

In one embodiment, a conjugate comprises a tumor-targeted antibody and DNA plasmid/minicircle encoding a pathogen antigen-derived gene. For example, an antibody targets the human Epidermal growth factor receptor cell surface receptor on tumor cells (anti-EGFR); or an antibody targets the human HER2/neu receptor cell surface receptor on tumor cells (anti-HER2/neu).

In another embodiment, a conjugate comprises a tumor-targeted aptamer and DNA plasmid/minicircle encoding a pathogen antigen-derived gene. For example, an aptamer targeting a cell surface molecule (prostate specific membrane antigen (PSMA) on tumor cells (PSMA RNA aptamer).

In another embodiments, a conjugate comprises a tumor-targeted peptide and DNA minicircle encoding a pathogen antigen-derived gene. Examples of such tumor targeted Peptide are known and disclosed herein (e.g., RGD peptide).

The following provides an illustrative method for producing a Tumor Targeting moiety-DNA vaccine conjugate: (1) DNA minicircle vaccines encoding pathogen-derived genes (a) DNA minicircle encoding Bacillus anthracis Protective Antigen (PA); (b) the DNA sequence for B. anthracis Protective Antigen (PA) was codon optimized for efficient expression in mammalian cells (DNA 2.0); (c) DNA minicircle for Clostridium Tetani (tetanus) toxin derived gene fragment (e.g. Tetanus toxin Fragment C-FrC, or DOM1). For example, the DNA sequence for Clostridium Tetani (tetanus) toxin derived gene fragment (Tetanus Fragment C or DOM1) was codon optimized for efficient expression in mammalian cells (DNA 2.0).

DNA Vaccine design and rationale: CD4+ T helper (T_{H}) cells are vital for the induction and maintenance of immune responses. T_{H} cells are required for priming and secondary expansion of CD8+ T cells and providing help to B cells for antibody production. Since autologous tumor antigens are incapable of inducing significant T_{H} responses, the tumor targeted DNA conjugate vaccines of the invention incorporate encoded pathogen-derived sequences, such as from tetanus toxin or Pseudomonas aeruginosa exotoxin, so that T_{H} cells from the existing anti-microbial repertoire can help mount CD8+ T cell and/or B cell responses against tumor antigens derived from the immunoconjugate-targeted tumor cell and/or antigens co-encoded/fused within the same plasmid or minicircle. DNA vaccines can also provide T-cell help by incorporating other non-self antigens such as green fluorescent protein, plant viral coat proteins, or immune targeting molecules (alone or co-expression with tumor antigens or as fusion partners).

The conjugation of DNA vaccines incorporating pathogen-derived sequences to tumor targeted moieties results in the expression of these antigenic determinants in the targeted tumor cell as well as the indirect transfer of antigenic material (pathogen-derived and endogenous tumor cells/antigens) to APCs that have phagocytosed the targeted tumor cells (cross-presentation). A proportion of the antibody-DNA vaccine may also be directly taken up and presented by APCs (via antibody Fc interactions with Fc receptors on APC FcR). Such cross-presentation and direct presentation of pathogen- and tumor-derived antigens can provide effective T-cell help and result in the following immune responses: (1) Induction of pathogen antigen- and tumor antigen-specific antibodies: The antibody-DNA conjugate of the invention enables expression of pathogen antigen (e.g. Tetanus toxin derived fragment C-FRC) in the targeted tumor cells as well as cross-presentation of FrC and tumor antigens by DCs (from apoptotic tumor cells and/or co-encoded/fused tumor antigens in the vaccine). (FrC)-specific T_{H} cells stimulated by DC are able to prime and boost B cells to produce antibodies against FrC peptide or tumor cell antigens (via CD40-CD40 ligand interaction and cytokine production). The expression of FrC antigenic determinants in tumor cells also renders them susceptible to ADCC by either anti-FrC antibodies or anti-tumor antibodies, thereby reinforcing the cross-presentation of these antigens by DC that have phagocytosed the opsonized or apoptotic tumor cells; (2) Induction of tumor-reactive cytotoxic T cells: The antibody-DNA vaccine encoding microbial antigens or other non-self antigens may be used to initiate and amplify CD8+ T lymphocyte (CTL) immune responses against a range of otherwise weak tumor antigens. (FrC)-specific T_{H} cells license DCs cross-presenting both FrC and tumor antigens to prime and boost CD8+ T cell responses against weak tumor antigens. Since immunodominant pathogen-derived peptides can restrict responses to sub-dominant tumor-derived epitopes, the pathogen-derived antigen encoded by the DNA vaccine may be minimized to contain epitopes required to provide CD4+ T cell help (such as a single domain of FrC-DOM1, or promiscuous MHC class II binding peptides, such as tetanus toxin p30).

These immune responses are facilitated and reinforced by the ability of the immunoconjugate of this invention to simultaneously activate DC via one or more of the following: (1) PAMPs that are incorporated in the conjugate (such as immunostimulatory nucleic acids); (2) Damage associated molecular patterns (DAMPs) that are included in the conjugate (e.g. alarmins, such as LL-37 cathelicidin); (3) Endogenous PAMPs or DAMPs produced via expression of the encoded genes or in response to cellular stress and damage; (4) Other endogenous immunostimulatory molecules that are produced via expression of the encoded genes or as a bystander effect of activating immune responses in the tumor cell milieu.

In one embodiment, a formulation of DNA plasmid/minicircle vaccine is utilized in a conjugate of the invention. The specific codon optimized pathogen-derived DNA sequence (either PA or Tetanus fragment C/DOM1) and the DNA sequences at the repeat binding sites 1 and 2, found on the GeneGrip plasmid series are cloned into an intermediate mammalian expression vector containing a CMVie promoter and SV40 terminator vector. After sequence confirmation the entire expression cassette (CMV promoter, antigen, SV40, oligonucleotide binding motif) is PCR amplified with PCR primers containing either SpeI (5′ end) or ApaI (3′ end) restriction endonuclease site specific tails. The PCR product is then digested with SpeI and ApaI and ligated into the SpeI and ApaI sites of the p2 φC31 minicircle vector. The construct, p2φC31-PA is then transformed into E. coli NM522 cells and tested for recombination capability. E. coli containing the plasmid are grown and then recombination is induced by the addition of arabinose (0.25% final concentration). An aliquot of culture is taken before (time 0) and after (60 and 120 minutes) induction and subjected to miniprep plasmid isolation. The resulting plasmid prep is subjected to electrophoresis to determine if the mother plasmid had recombined into the miniplasmid and minicircle. The recombination is successful as determined by the presence of a minicircle band on the gel. The backbone plasmid band (miniplasmid) is also present, but its intensity decreased over time (indicating that the I-SceI enzyme cuts the plasmid backbone and it is being degraded by the cellular endonucleases).

Conjugation of DNA minicircle vaccine with tumor targeting moiety. The conjugation of the specific DNA vaccines to tumor-targeting moieties described in this invention provides a multifactorial improvement of antitumor efficacy: (1) Provides targeted delivery, retention, and receptor-mediated internalization of the DNA vaccine to tumor cells. Expression of encoded pathogen-derived antigens in tumor cells allows pathogen antigen-reactive antibodies to opsonize tumor cells, thereby increasing ADCC and Fc-mediated cross-presentation of pathogen- and endogenous tumor antigens by DCs; (2) Antibody-DNA conjugate coated tumor cells enhance activation of DCs that have phagocytosed tumor cells via conjugate-derived exogenous and cell-derived endogenous immunostimulatory PAMPs and DAMPs, thereby facilitating activation of CD4+ T helper cells and CD8+ cytotoxic T cells against tumor cells. DC-NK cell cross-talk further amplifies ADCC and complement-mediated lysis of antibody-conjugate coated tumor cells; (3) Intracellular delivery of immunostimulatory molecules of the conjugate (Immunostimulatory nucleic acids, PAMPs) into the tumor cell via antibody/receptor-mediated endocytosis results in cellular responses leading to upregulation of MHC molecules and presentation of tumor-derived antigens for recognition of tumor cells by B and T cells; (4) Antibody-conjugates targeting a tumor growth factor receptor block receptor-mediated tumor cell survival and growth signals, thereby improving susceptibility to CTL-mediated cytotoxicity; and (5) Antibody-DNA vaccines enable cross-presentation of conjugate-bound apoptotic tumor cells to DCs, thereby inducing bystander stimulation of memory T cells against a range of endogenous tumor-derived antigens (antigen spreading). This is preferable to DNA vaccines delivering or expressing specific chosen tumor peptides, whose efficacy may be limited by escape of variant tumor cells that do not express the selected antigens.

The foregoing is illustrative and not a limiting process, for the formation of a conjugate of a tumor targeting antibody and a minicircle DNA vaccine, wherein both moieties are directly coupled in a sequence, site, and orientation specific manner with a controlled number of plasmid/minicircle DNA copies attached to each antibody, thereby allowing maintenance of the key functional properties of the antibody as well as tumor targeted expression of the DNA vaccine. The selection of the specific tumor targeting antibody and the composition of the encoded pathogen antigen gene in the DNA minicircle are designed to optimize the synergistic functional components of the conjugate for antitumor therapy. Another key function enabled by this invention is the expression of the encoded pathogen antigenic determinants in the targeted tumor cell and tumor milieu, and the specific immune responses triggered by this enablement. These features distinguish the specific tumor antibody-DNA vaccine conjugates of this invention from other DNA vaccines and delivery platforms, such as particle-mediated delivery, gene gun, viral or bacterial vectors, or electroporation.

In one method to synthesize the antibody-plasmid/minicircle DNA conjugate, a linear ss oligonucleotide [LNA/DNA ODNs containing either a (CT)n or a (GA)n repeat motif complementary to the corresponding ds DNA sequence in the double stranded plasmid or minicircle DNA] is bound to the supercoiled, double-stranded minicircle DNA.

(SEQ ID NO:238)

LNA ODN (5′-NH₂-GAGG-CTCTCTCTCTCTC-3′)
Hybrid LNA-DNA with immunostimulatory CpG DNA
phosphorothioate backbone:

(SEQ ID NO:239)

5′ tccatgacgttcctgacgttt CTCTCTCTCTCTC-GGAG-NH₂-3′

(SEQ ID NO:240)

5′ cggcggataaccgcgagcggttattcgccctacgg CTCTCTCTCTC
TC-GGAG-NH₂-3′
(repetitive extragenic palindromic REP sequence;
P. Aeruginosa)

(SEQ ID NO:241)

5′ gggggacgatcgtcggggg CTCTCTCTCTCTC-GGAG-NH₂-3′
(A class CpG ODN)

For example, a minicircle DNA is incubated with LNA ODN or hybrid LNA-DNA ODN with a CpG DNA phosphorothioate backbone in 10 mM phosphate buffer, 1 mM EDTA, pH 5.8 for 16 h at 37° C., at a maximum of 4- to 40-fold molar excess of ODN to ODN-binding sites in the plasmid. Heterobifunctional reagents containing an amine reactive NHS ester on one end and a sulfhydryl reactive maleimide group on the other end are used to produce antibody-DNA conjugates, as described (Ref. Bioconjugate techniques, Hermanson, G. T., Academic Press, 1996, pages 456-527).

The antibody-plasmid/minicircle conjugate may incorporate a described cationic peptide, such as the alarmin LL-37, which can promote protection of the DNA from nucleases, facilitate cellular entry, and/or enhance DC activation.

Analysis of the effects of Targeting moiety-DNA vaccine conjugate can be performed as follows: (1) Receptor-mediated endocytosis in target tumor cell (e.g. EGFR+ or HER2+ cells); (2) Expression of gene of interest in target tumor cell—Pathogen antigen-derived epitopes (B or T cell antigen determinants) presented by MHC molecules; (3) Phagocytosis of opsonized tumor cell by APC/DC: activation of DCs by TLR agonists, PAMPs; presentation of pathogen antigen CD4+ T cell and B cell epitopes; and cross-presentation of tumor associated antigens; (4) Activation of pathogen antigen-reactive CD4+ T helper cells; help to DCs cross-presenting tumor antigens; help to B cells for generation of pathogen antigen-reactive antibodies; and help for activation and survival of pathogen antigen- or tumor-reactive CD8+ T cells; (5) Cytolysis of tumor cells: ADCC (pathogen antigen-reactive antibodies); CD8+ T-cell mediated cytotoxicity (pathogen antigen-reactive T cells); and CD8+ T cell mediated cytotoxicty (tumor antigen reactive CD8+ T cells—via antigen spreading).

B. Skin Targeted Composition

In one embodiment, a composition of the invention comprises one or more targeting moiety (T) which binds a target molecules or component of a normal cell or tissue, such as keratinocytes in skin (tissue-targeting moiety). In one embodiment, the targeting moiety binds a cell surface molecule or receptor on keratinocytes, such as the epidermal growth factor receptor (EGFR).

In one embodiment, the invention comprises a conjugate of a tissue-targeting moiety, such as an antibody to EGFR, and a nucleic acid molecule, wherein the nucleic acid molecule encodes one or more products (e.g. nucleic acids such as RNA, peptides, polypeptides, fusion peptides) and is capable of stimulating an immune response. In one embodiment, the nucleic acid molecule includes one or more pathogen associated molecular pattern (PAMP) or other immunostimulatory motif. In another embodiment, the nucleic acid molecule encodes one or more products that stimulate an immune response. In a related embodiment, the nucleic acid molecule includes one or more pathogen associated molecular pattern (AMP) or other immunostimulatory motif, and encodes one or more products that stimulates an immune response.

In one embodiment, the invention comprises a conjugate of a tissue-targeting moiety, such as an antibody to EGFR, and a nucleic acid molecule, wherein the nucleic acid molecule includes one or more pathogen associated molecular pattern (PAMP) and encodes one or more antigens or antigenic determinants derived from one or more pathogen(s), microorganism(s) or virus(es)(T or B cell epitopes).

In one embodiment, the invention comprises a conjugate of a tissue-targeting moiety, such as an antibody to EGFR, one or more pathogen associated molecular pattern (PAMP), and nucleic acid molecule encoding one or more antigens or antigenic determinants derived from one or more pathogen(s), microorganism(s) or virus(es)(T or B cell epitopes).

In one embodiment, the invention comprises a conjugate of a tissue-targeting moiety, such as an antibody to EGFR, one or more damage associated molecular pattern (DAMP) or alarmin, and a nucleic acid molecule encoding one or more antigens or antigenic determinants derived from one or more pathogen(s), microorganism(s) or virus(es)(T or B cell epitopes).

In one embodiment, the invention comprises a conjugate of a a tissue-targeting moiety, such as an antibody to EGFR, one or more nucleic acid molecule(s) encoding one or more antigens or antigenic determinants derived from one or more pathogen(s), microorganism(s) or virus(es)(T or B cell epitopes), and encoding none, one, or more of the following: (i) one or more pathogen associated molecular pattern (PAMP), (ii) one or more damage associated molecular patterns (DAMP)/alarmin(s), (iii) one or more immunostimulatory molecules, including molecules that recruit, bind, activate, mature and/or proliferate an antigen presenting cell or dendritic cell or other immune cell (such as T cells, B cells, NK cells) and molecules that counteract immune suppression (e.g. ligands/antibodies for DC uptake receptors, immunostimulatory cytokines, chemokines, costimulatory molecules, growth factors). In a related embodiment, the nucleic acid molecule encodes one or more pathogen antigens/antigenic determinants as fusion proteins. In one aspect, the fusion partner of the antigen facilitates antigen uptake by DCs, immune recognition, and/or immune activation. In another aspect, the fusion partner includes a molecule targeting a DC uptake receptor. In another aspect, the fusion partner is an alarmin. In a related embodiment, the targeting moiety-nucleic acid conjugate(s) described herein further comprises one or more PAMP and/or one or more DAMP/Alarmin(s).

In one embodiment, the invention comprises a conjugate of a tissue-targeting moiety, such as an antibody to EGFR, one or more nucleic acid molecule(s) encoding one or more tumor antigens/antigenic determinants and encoding one or more of the following:

(i) one or more antigens or antigenic determinants derived from one or more pathogen(s), microorganism(s) or virus(es)(e.g. CD4+ T cell epitopes), (ii) one or more pathogen associated molecular pattern (PAMP), (ii) one or more damage associated molecular patterns (DAMP)/alarmin(s), (iii) one or more immunostimulatory molecules, including molecules that recruit, bind, activate, mature and/or proliferate an antigen presenting cell or dendritic cell or other immune cell (such as T cells, B cells, NK cells) and molecules that counteract immune suppression (e.g. ligands/antibodies for DC uptake receptors, immunostimulatory cytokines, chemokines, costimulatory molecules, growth factors). In a related embodiment, the nucleic acid molecule encodes one or more tumor antigen-containing fusion proteins. In one aspect, the fusion partner of the tumor antigen facilitates antigen uptake by DCs, immune recognition, and/or immune activation. In another example, the fusion partner includes a molecule targeting a DC uptake receptor. In another example, the fusion partner is an antigen or antigenic determinant derived from one or more pathogen(s), microorganism(s) or virus(es)(CD4+ T cell epitope). In another example, the fusion partner is an alarmin. In a related embodiment, the targeting moiety-nucleic acid conjugate(s) described herein further comprises one or more PAMP and/or one or more DAMP/Alarmin(s).

In one embodiment, the invention comprises a conjugate of a tissue-targeting moiety, such as an antibody to EGFR, one or more pathogen associated molecular pattern (PAMP) and/or alarmin, and an antigenic peptide/polypeptide that includes one or more of the following: (i) one or more antigens or antigenic determinants derived from one or more pathogen(s), microorganism(s) or virus(es), (ii) one or more tumor antigens or antigenic determinants. In one aspect of the conjugate, the tumor or pathogen-derived antigen or antigenic determinant is linked or fused to an alarmin (e.g. LL 37).

In another embodiment, the invention comprises a conjugate of an antibody or other moiety targeting a skin cell surface receptor (e.g. EGFR), one or more pathogen associated molecular pattern (PAMP), and nucleic acid molecule incorporating a gene encoding one or more pathogen or pathogen-derived antigens or antigenic determinants (T or B cell epitopes). For example, a conjugate of the invention comprises a Targeting moiety+any PAMP+plasmid/minicircle DNA coding pathogen antigen.

In another embodiment, a conjugate comprises an antibody or other moiety targeting a skin cell surface receptor (e.g. EGFR), one or more damage associated molecular pattern (DAMP) or alarmin, and a nucleic acid molecule incorporating a gene encoding one or more pathogen or pathogen-derived antigens or antigenic determinants (T or B cell epitopes). For example, a conjugate comprises a Targeting moiety+any DAMP/Alarmin+plasmid/minicircle DNA coding pathogen antigen.

In yet another embodiments, a conjugate comprises an antibody or other moiety targeting a skin cell surface receptor (e.g. EGFR), and a nucleic acid molecule incorporating a gene encoding one or more of the following: pathogen or pathogen-derived antigens or antigenic determinants (T or B cell epitopes), pathogen associated molecular pattern (PAMP), damage associated molecular patterns (DAMPs), alarmin. For example, a conjugate comprises a Targeting moiety+DNA encoding pathogen or pathogen-derived antigens or antigenic determinants; or a conjugate comprises Targeting moiety+DNA encoding pathogen or pathogen-derived antigens or antigenic determinants and one or more PAMP, DAMP, alarmin.

In another embodiment, a conjugate comprises an antibody or other moiety targeting a skin cell surface receptor (e.g. EGFR), a nucleic acid molecule incorporating a gene encoding one or more tumor antigens and one or more of the following: pathogen or pathogen-derived antigens or antigenic determinants (T or B cell epitopes), pathogen associated molecular pattern (PAMP), damage associated molecular patterns (DAMPs), alarmin. For example, a conjugate comprises a Targeting moiety+DNA encoding tumor antigen+pathogen or pathogen-derived antigens or antigenic determinants, DAMP, alarmin.

In one embodiment, the invention comprises a conjugate comprising an antibody or other moiety targeting a skin cell surface receptor (e.g. EGFR) and a nucleic acid molecule, wherein the nucleic acid molecule incorporates one or more pathogen associated molecular pattern (PAMP) and a gene encoding one or more pathogen or pathogen-derived antigens or antigenic determinants (T or B cell epitopes).

In yet another embodiment, the invention comprises a conjugate of an antibody or other moiety targeting a skin cell surface receptor (e.g. EGFR), one or more pathogen associated molecular pattern (PAMP)/alarmin and nucleic acid molecule incorporating a gene encoding one or more pathogen or pathogen-derived or tumor antigens or antigenic determinants (T or B cell epitopes). For example, a conjugate comprises a Targeting moiety+any PAMP/alarmin+plasmid/minicircle DNA coding tumor antigen; or a conjugate comprises Targeting moiety+any PAMP/alarmin+plasmid/minicircle DNA coding tumor antigen and pathogen antigen.

While not intending to be limited to any one mechanism of action, the following is one mode of action for a conjugate is of the invention: (a) EGFR receptor-mediated binding of minicircle/plasmid DNA to target skin cell (keratinocyte) and retention/immobilization of DNA in skin; (b) Receptor-mediated endocytosis in keratinocyte and expression of minicircle encoded gene of interest in target—e.g. Plasmodium epitopes (CSP-1 antigen derived B or T cell antigen determinants) presented by MHC molecules; (c) Phagocytosis of conjugate-opsonized keratinocyte by APC/DC in skin (Langerhans cells): (i) Antibody Fc-DC Fc receptor interaction-mediated presentation of DNA encoded pathogen antigen or tumor antigen epitopes (T cell and B cell epitopes)-indirect antigen cross-presentation; (ii) Uptake of minicircle—expression of gene of interest in APC (T cell and B cell epitopes)—direct presentation; (iii) Activation of DCs by TLR agonists, PAMPs, DAMPs, alarmins (conjugate-derived and endogenous); (iv) Activation of antigen-reactive T cells and B cells recognizing pathogen antigen- or tumor antigen derived epitopes (e.g. multiple CSP-1 epitopes).

In one embodiment, a conjugates comprises an EGFR-targeted moiety and a DNA plasmid/minicircle encoding a pathogen antigen-derived gene. In another embodiment, a conjugate an antibody targeting the human Epidermal growth factor receptor on keratinocytes (anti-EGFR Ab: e.g. cetuximab, nimotuzumab, panitumumab) and a DNA minicircle encoding a pathogen antigen-derived gene. In yet a further embodiment, a conjugate of an Aptamer targeting the human Epidermal growth factor receptor on keratinocytes (anti-EGFR DNA or RNA aptamer) and a DNA minicircle encoding a pathogen antigen-derived gene. In addition, the targeting moiety can be EGFR-targeted peptide and DNA minicircle encoding a pathogen antigen-derived gene.

Examples of DNA plasmid and minicircle encoded pathogen antigen-derived gene are provided herein. In one embodiment, the encoded antigen is circumsporozoite protein (CSP-1) from plasmodium (malaria antigen). In a further embodiment, such a conjugate can be administered to provide DNA vaccination with malaria CSP-p28 construct. The malarial circumsporozoite protein (CSP) is the major surface protein of the sporozoite and has been shown to confer protection mouse models of malaria. Bergmann-Leitner et. al. (C3d-defined complement receptor-binding peptide p28 conjugated to circumsporozoite protein provides protection against Plasmodium berghei. Vaccine 25 (45), 2007) demonstrated that a DNA vaccine encoding CSP along with three copies of the C3d complement receptor binding peptide p28 induced protection against challenge in a mouse model of P. berghei infection. This vaccine is directly conjugated to an EGFR antibody to form a conjugate contained herein. As such, conjugates of this type target keratinocytes, and the encoded antigen-p28 fusion proteins can target DC uptake receptors.

In further embodiments, the encoded antigen is a Merozoite antigens from plasmodium; Bacillus anthracis Protective Antigen (PA); Mycobacterium tuberculosis antigens; Shigella IpaB and IpaC; Influenza Virus antigens or a combination thereof. Expansive lists of pathogenic antigens are known in the art and such antigens can readily be used in the context of the present invention.

In another aspect of the invention, a conjugates of an EGFR-targeted moiety and a DNA plasmid/minicircle encoding one or more tumor antigens or tumor associated antigens.

In one embodiment, a conjugate comprises an antibody targeting the human Epidermal growth factor receptor on keratinocytes (anti-EGFR Ab: e.g. cetuximab, nimotuzumab, panitumumab) and a DNA minicircle encoding tumor antigens or tumor associated antigens. In further embodiments, the targeting moiety can be any variation disclosed herein (e.g, aptamer, peptide).

Expansive lists of tumor antigen or tumor associated antigens are known in the art and such antigens can be used in the context of the present invention. Some non-limiting examples of such antigens include cancer-testis antigens, such as MAGE-1, BAGE, GAGE-1, NY-ESO-1; Lineage specific antigens: e.g. Melanocyte antigens (tyrosinase, MART-1, gp100); Tumor-specific altered gene products (amplified, aberrantly expressed, overexpressed, or mutated genes, splice variants, gene fusion products): e.g., HER2/neu, p53, Ras genes—KRAS2, HRAS, NRAS, Mucin-1, beta catenin, MUM1, CDK4, BCR-ABL fusion products, surviving, TERT, CEA, AFP, N-acetylglucosaminyltransferase V; Immunoglobulin idiotypes in B-cell malignancies; Viral oncoantigens; e.g. HPV E6 and E7 antigens from Human Papilloma Virus, EBV LMP1 and LMP2, just to name a few. In one further embodiment, one or more tumor antigens may be encoded in the DNA minicircle downstream or as fusion partners of pathogen-derived antigenic determinants (such as tetanus FrC or DOM1) to provide CD4+ T cell help (as noted for tumor targeting conjugates above).

An illustrative method of making such a conjugate is as follow: isolate a DNA plasmid/minicircle encoding Bacillus anthracis Protective Antigen (PA) using conventional techniques for minicircle isolation; optimize the DNA sequence for B. anthracis Protective Antigen (PA) for efficient expression in mammalian cells (DNA 2.0), using codon optimization. In another embodiment, the DNA plasmid/minicricle encodes Cricumsporozoite protein (CSP-1) and is also codon optimized for expression in mammalian cells. Furthermore, expression can be regulated using tissue/cell-specific promoters known in the art and disclosed herein.

DNA Vaccine design and rationale: The conjugation of DNA vaccines incorporating pathogen- or tumor antigen-derived sequences to EGFR targeted moieties results in the expression of these antigenic determinants in the targeted keratinocyte as well as the indirect transfer of antigenic material (pathogen- or tumor antigen-derived antigens) to APCs that have phagocytosed the targeted keratinocytes (cross-presentation; facilitated via antibody Fc interactions with Fc receptors on APC FcR). A proportion of the antibody-DNA vaccine may also be directly taken up and expressed by APCs. Such cross-presentation and direct presentation of pathogen- or tumor-derived antigens can provide effective T-cell help and result in the following immune responses:

Induction of pathogen antigen- and tumor antigen-specific antibodies: The antibody-DNA conjugate of the invention enables expression of pathogen antigen in the targeted keratinocytes as well as cross-presentation of pathogen or tumor antigens by DCs (from phagocytosed opsonized keratinocytes and/or co-encoded/fused antigens in the vaccine). Antibody-DNA conjugates enhance activation of DCs presenting these antigens via conjugate-derived exogenous and cell-derived endogenous immunostimulatory PAMPs and DAMPs, thereby facilitating activation of antigen reactive CD4+ T helper cells and CD8+ cytotoxic T cells. Pathogen antigen-specific T_{H} cells stimulated by DC are able to prime and boost B cells to produce antibodies against cross-presented antigens (via CD40-CD40 ligand interaction and cytokine production).

Induction of pathogen antigen- or tumor-reactive cytotoxic T cells: The antibody-DNA vaccine encoding microbial antigens or other non-self antigens may be used to initiate and amplify CD8+ T lymphocyte (CTL) immune responses against a range of otherwise weak tumor antigens. For example, Tetanus FrC-specific T_{H} cells license DCs cross-presenting both FrC and tumor antigens to prime and boost CD8+ T cell responses against weak tumor antigens. Since immunodominant pathogen-derived peptides can restrict responses to sub-dominant tumor-derived epitopes, the pathogen-derived antigen co-encoded by antitumor DNA vaccine may be minimized to contain epitopes required to provide CD4+ T cell help (such as a single domain of FrC-DOM1, or promiscuous MHC class II binding peptides, such as tetanus toxin p30).

Formulation of DNA plasmid/minicircle vaccine: The specific codon optimized pathogen-derived DNA sequence (DNA minicircle encoding either PA or CSP), with or without three copies of the C3d complement receptor region p28), and the DNA sequences at the repeat binding sites 1 and 2, found on the GeneGrip plasmid series are cloned into an intermediate mammalian expression vector containing a CMVie promoter and SV40 terminator vector. After sequence confirmation the entire expression cassette (CMV promoter, antigen, SV40, oligonucleotide binding motif) is PCR amplified with PCR primers containing either SpeI (5′ end) or ApaI (3′ end) restriction endonuclease site specific tails. The PCR product is then digested with SpeI and ApaI and ligated into the SpeI and ApaI sites of the p2φC31 minicircle vector. The construct, p2φC31-PA is then transformed into E. coli NM522 cells and tested for recombination capability. E. coli containing the plasmid are grown and then recombination is induced by the addition of arabinose (0.25% final concentration). An aliquot of culture is taken before (time 0) and after (60 and 120 minutes) induction and subjected to miniprep plasmid isolation. The resulting plasmid prep is subjected to electrophoresis to determine if the mother plasmid had recombined into the miniplasmid and minicircle. The recombination is successful as determined by the presence of a minicircle band on the gel. The backbone plasmid band (miniplasmid) is also present, but its intensity decreased over time (indicating that the I-SceI enzyme cuts the plasmid backbone and it is being degraded by the cellular endonucleases).

Conjugation of DNA plasmid/minicircle vaccine with EGFR targeting moiety. The conjugates of DNA vaccines/EGFR-targeting moieties described in this invention provide a multifactorial improvement of immunologic efficacy: (1) Enables targeted delivery, retention, and receptor-mediated internalization of the DNA vaccine to keratinocytes and expression of encoded pathogen- or tumor-derived antigens in keratinocytes; (2) Phagocytosis of conjugate opsonized keratinocytes facilitates Fc-mediated cross-presentation of pathogen- and tumor antigens by DCs as well as direct expression and presentation of the conjugate encoded genes in DCs; (3) Antibody-DNA conjugate coated tumor cells enhance activation of DCs via conjugate-derived exogenous and cell-derived endogenous immunostimulatory PAMPs and DAMPs, thereby facilitating activation of CD4+ T helper cells and B cell and CD8+ cytotoxic T cells reacting against presented antigens.

In one embodiment, a conjugate of the invention comprises an oligonucleotide which is used to couple the conjugate to a minicircle. Such an oligonucleotide can comprise a linear ss oligonucleotide [LNA/DNA ODNs containing either a (CT)n or a (GA)ₙ repeat motif complementary to the corresponding ds DNA sequence in the double stranded plasmid or minicircle DNA] is bound to the supercoiled, double-stranded minicircle DNA. Examples of such oligonucleotides include but are not limited to LNA ODN (5′—NH₂-GAGG-CTCTCTCTCTCTC-3′) (SEQ ID NO:238); Hybrid LNA-DNA ODN with a CpG DNA phosphorothioate backbone: 5′tccatgacgttcctgacgttt CTCTCTCTCTCTC-GGAG-NH₂-3′ (SEQ ID NO:239); 5′cggcggataaccgcgagcggttattcgccctacgg CTCTCTCTCTCTC-GGAG-NH₂-3′ (SEQ ID NO:240) (repetitive extragenic palindromic —REP sequence; P. Aeruginosa); or 5′ gggggacgatcgtcggggg CTCTCTCTCTCTC-GGAG-NH₂-3′ (SEQ ID NO:241) (A class CpG ODN).

For example, a Minicircle DNA is incubated with LNA ODN or hybrid LNA-DNA ODN with a CpG DNA phosphorothioate backbone in 10 mM phosphate buffer, 1 mM EDTA, pH 5.8 for 16 h at 37° C., at a maximum of 4- to 40-fold molar excess of ODN to ODN-binding sites in the plasmid. Heterobifunctional reagents containing an amine reactive NHS ester on one end and a sulfhydryl reactive maleimide group on the other end are used to produce antibody-DNA conjugates, as described (Ref. Bioconjugate techniques, Hermanson, G. T., Academic Press, 1996, pages 456-527).

In a further embodiment, the antibody-plasmid/minicircle conjugate may incorporate a described cationic peptide, such as the alarmin LL-37, which can promote protection of the DNA from nucleases, facilitate cellular entry, and/or enhance DC activation.

Effects of Targeting moiety-DNA vaccine conjugate can be analyzed as follows: (a) EGFR-mediated endocytosis in target cell (e.g. keratinocytes); (b) Expression of gene of interest in keratinocytes—Pathogen antigen-derived or tumor antigen epitopes (B or T cell antigen determinants) presented by MHC molecules; (c) Phagocytosis of opsonized keratinocytes by APC/DC: (i) activation of DCs by conjugate-derived PAMPs, DAMPs; (ii) presentation of pathogen antigen CD4+ T cell and B cell epitopes; (iii) cross-presentation of tumor associated antigens; (d) Activation of pathogen antigen-reactive CD4+ T helper cells; (i) provide help to DCs cross-presenting tumor antigens; (ii) provide help to B cells for generation of pathogen antigen-reactive antibodies; (iii) provide help for activation and survival of pathogen antigen- or tumor-reactive CD8+ T cells.

C. APC/DC Targeting Compositions

In one embodiment, the invention comprises a conjugate of a tissue-targeting moiety, such as an antibody to EGFR, one or more a nucleic acid molecule(s), and one or more peptide/polypeptide. In one embodiment, the nucleic acid molecule incorporates one or more pathogen associated molecular pattern (AMP) or other immunostimulatory motif, and/or encodes one or more products that stimulate an antigen-specific immune response, as described herein. In various embodiments of the conjugate, the peptide/polypeptide includes one or more of the following:(i) one or more pathogen and/or tumor antigens or antigenic determinants, (ii) alarmins, (iii) DC binding molecules (e.g. ligands of DC uptake receptors). In one aspect, the peptide/polypeptides of the conjugate described herein may be fused/linked to each other and/or to a nucleic acid binding peptide (e.g. cationic peptides, protamine, HIV-tat, Arginine- or Histidine-rich sequence, LL-37, Nuclear localizing peptide).

In one embodiment, a composition of the invention comprises one or more targeting moiety (T) which binds a target molecules or component of a normal immune cell or tissue, such as antigen presentic cells or dendritic cells (APC/IDC-targeting moiety).

In one embodiment, the targeting moiety binds a dendritic cell uptake receptor, such as DEC-205.

In one embodiment, the invention comprises a conjugate comprising an antibody or other moiety targeting an antigen presenting cell (APC)/Dendritic cell (DC), such as a DC uptake receptor, and a nucleic acid molecule which encodes a gene of interest.

In one embodiment, the invention comprises a conjugate of an APC/DC-targeting moiety and a nucleic acid molecule, wherein the nucleic acid molecule encodes one or more products (e.g. nucleic acids such as RNA, peptides, polypeptides, fusion peptides) and is capable of stimulating an immune response. In one embodiment, the nucleic acid molecule includes one or more pathogen associated molecular pattern (PAMP) or other immunostimulatory motif. In another embodiment, the nucleic acid molecule encodes one or more products that stimulate an immune response. In a related embodiment, the nucleic acid molecule includes one or more pathogen associated molecular pattern (PAMP) or other immunostimulatory motif, and encodes one or more products that stimulates an immune response.

In one embodiment, the invention comprises a conjugate of an APC/DC-targeting moiety, such as an antibody to DEC-205, and one or more nucleic acid molecules, wherein the nucleic acid molecule includes one or more pathogen associated molecular pattern (PAMP) and encodes one or more antigens or antigenic determinants derived from one or more pathogen(s), microorganism(s) or virus(es)(T or B cell epitopes). In a related embodiment, the targeting moiety-nucleic acid conjugate(s) described herein further comprises one or more PAMP and/or one or more DAMP/Alarmin(s).

In one embodiment, the invention comprises a conjugate of an APC/DC-targeting moiety, one or more pathogen associated molecular pattern (PAMP), and one or more nucleic acid molecule encoding one or more antigens or antigenic determinants derived from one or more pathogen(s), microorganism(s) or virus(es)(T or B cell epitopes). In a related embodiment, the targeting moiety-nucleic acid conjugate(s) described herein further comprises one or more DAMP/Alarmin(s).

In one embodiment, the invention comprises a conjugate of an APC/DC-targeting moiety, one or more damage associated molecular pattern (DAMP) or alarmin, and one or more nucleic acid molecule encoding one or more antigens or antigenic determinants derived from one or more pathogen(s), microorganism(s) or virus(es)(T or B cell epitopes).

In one embodiment, the invention comprises a conjugate of an APC/DC-targeting moiety and one or more nucleic acid molecule(s) encoding one or more antigens or antigenic determinants derived from one or more pathogen(s), microorganism(s) or virus(es)(T or B cell epitopes), and encoding one or more immunostimulatory molecules, such as molecules that recruit, bind, activate, mature and/or proliferate an antigen presenting cell or dendritic cell or other immune cell (such as T cells, B cells, NK cells) and molecules that counteract immune suppression (e.g. immunostimulatory cytokines, chemokines, costimulatory molecules, growth factors). In a related embodiment, the nucleic acid molecule encodes one or more pathogen antigens/antigenic determinants as fusion proteins. In a related embodiment, the targeting moiety-nucleic acid conjugate(s) described herein further comprises one or more PAMP and/or one or more DAMP/Alarmin(s). In one aspect, the conjugate further includes one or more peptides that include one or more pathogen-derived antigens or antigenic determinants.

In one embodiment, the invention comprises a conjugate of an APC/DC-targeting moiety and one or more nucleic acid molecules encoding one or more tumor antigens and encoding one or more of the following: (i) one or more antigens or antigenic determinants derived from one or more pathogen(s), microorganism(s) or virus(es)(e.g. CD4+ T cell epitopes), (ii) one or more immunostimulatory molecules, such as molecules that recruit, bind, activate, mature and/or proliferate an antigen presenting cell or dendritic cell or other immune cell (such as T cells, B cells, NK cells) and molecules that counteract immune suppression (e.g. immunostimulatory cytokines, chemokines, costimulatory molecules, growth factors). In a related embodiment, the nucleic acid molecule encodes one or more tumor antigens as fusion proteins with an antigen or antigenic determinant derived from one or more pathogen(s), microorganism(s) or virus(es)(CD4+ T cell epitope). In another example, the fusion partner is an alarmin. In a related embodiment, the targeting moiety-nucleic acid conjugate(s) described herein further comprises one or more PAMP and/or one or more DAMP/Alarmin(s). In one aspect, the conjugate further includes one or more peptides that include one or more pathogen-derived or tumor antigens or antigenic determinants.

In one embodiment, the invention comprises a conjugate of an APC/DC-targeting moiety, one or more pathogen associated molecular pattern (PAMP) and/or one or more alarmins, and one or more antigenic peptides that include one or more tumor antigens and/or antigens or antigenic determinants derived from one or more pathogen(s), microorganism(s) or virus(es)(T or B cell epitopes). In one embodiment the antigenic peptide is fused to or incorporated within the targeting moiety. In another aspect, the antigenic peptide is fused to an alarmin (e.g. LL-37).

In one embodiment, the invention comprises a conjugate of an APC/DC-targeting moiety, one or more nucleic acid molecules, and one or more antigenic peptides, wherein the nucleic acid molecule includes one or more pathogen associated molecular pattern (PAMP) and the antigenic peptides includes tumor antigens and/or antigens or antigenic determinants derived from one or more pathogen(s), microorganism(s) or virus(es)(T or B cell epitopes). In one embodiment the antigenic peptide is fused to or incorporated within the targeting moiety. In one related embodiment of the conjugate, the antigenic peptide is fused to a nucleic acid binding peptide (e.g. cationic peptides, NLS, Tat, Protamine, His6, Arg9, LL-37). In another aspect, the antigenic peptide is fused to a peptide motif targeting a DC uptake receptor. In one aspect, the antigenic peptide is fused to or incorporated within the targeting moiety. In another aspect, the antigenic peptide is fused to an alarmin.

One non-limiting example of a mechanism of action involving DCs is as follows. Dendritic cells have a range of uptake receptors for efficient and specific capture of antigens by absorptive endocytosis. DCs process the captured antigens and present them primarily as peptide-major histocompatibility complex (MHC) molecule complexes to effect the specific activation of T cells. This process requires activation and maturation of DCs in response to environmental stimuli, such as by recognition of pattern associated molecular patterns (PAMPs), or endogenous stimuli, such as alarmins. The conjugates of the invention enable both antigen gene expression (for antigen presentation) and DC activation/maturation (by coupled or encoded PAMPs/DAMPs) to occur simultaneously, thereby enhancing the ability to activate antigen specific immune cells in vivo or ex vivo.

Therefore, a conjugate is a multifunctional molecule with the following mechanisms of action: (a) DC Receptor-mediated uptake/endocytosis in dendritic cell; (b) Expression of gene of interest in DC-tumor or pathogen epitopes or fusion products (antigen derived B or T cell antigen determinants) presented by MHC molecules; (c) Presentation of T or B cell epitopes and simultaneous activation of APC/DC: (i) activation of TLRs by encoded or linked PAMPs, DAMPs/alarmins; (ii) presentation of T cell and B cell epitopes; and (iii) Activation of antigen-reactive T cells and B cells recognizing antigen epitopes

In various embodiments, a DC targeting moieties may include an antibody, aptamer, peptide, or ligand that targets a DC uptake receptors, such as the following: C-type lectin like receptors: DC-SIGN (Dendritic cell-specific ICAM-3-grabbing nonintegrin), MMR (MRC1)(macrophage mannose receptor), DEC-205 (LY75)(ligated by anti-DEC-205 antibody), BDCA-2 (blood dendritic cell antigen)(C type lectin superfamily CLECSF11), Langerin or Dectin-1; Fc receptors: (ligated by immune complexes and opsonized cells), FcgRI (CD32), FcgRII (CD64); Integrins: (ligated by apoptotic cells and opsonized antigens), aVb5, aMb2 (CD11b/CD18, complement receptor 3-CR3), or aXb2 (CD11c/CD18, complement receptor 4-CR4); Scavenger receptors: (ligated by apoptotic cells and heat shock protein (hsp)-peptide complexes), CD36, LOX-1 low density lipoprotein, oxidized, receptor-1(OLR1); or CD91, aquaporins. For example, Antigen uptake via DEC-205, Fcg receptors, aVb5 integrin, CD36, LOX-1, and CD91 have all been associated with cross-presentation.

DC targeting moieties are known and can be utilized in the context of the present invention. In one embodiment, the DC targeting moeity is anti-DEC205: DEC-205 (NLDC-145) which is an endocytic receptor expressed at high levels in DCs.

An antibody can be prepared using convention techniques. DC targeting peptide (e.g. p28). The C3d-defined complement receptor-binding peptide p28 is used to prepare a DNA-antibody conjugate of the invention.

DNA vaccines used for synthesis of the conjugate may include linear or circular plasmids, minicircle DNA, or MIDGE. The specific gene encoded by the DNA vaccine is selected from the following: Pathogen antigen-derived gene encoded by DNA plasmid or minicircle; Circumsporozoite protein (CSP-1) or merozoite proteins from plasmodium (malaria antigen): parasite; Bacillus anthracis Protective Antigen (PA): Gram positive bacteria; Mycobacterium tuberculosis antigens: Mycobacteria; Shigella IpaB and IpaC: Gram negative bacteria; Influenza Virus antigens: Virus.

Tumor antigens and tumor associated antigens encoded by DNA plasmid or minicircle (complete list in specifications); Cancer-testis antigens; e.g. MAGE-1, BAGE, GAGE-1, NY-ESO-1; Lineage specific antigens; e.g. Melanocyte antigens (tyrosinase, MART-1, gp100); Tumor-specific altered gene products (amplified, aberrantly expressed, overexpressed, or mutated genes, splice variants, gene fusion products) e.g. HER2/neu, p53, Ras genes—KRAS2, HRAS, NRAS, Mucin-1, beta catenin, MUM1, CDK4, BCR-ABL fusion products, surviving, TERT, CEA, AFP, N-acetylglucosaminyltransferase V; Immunoglobulin idiotypes in B-cell malignancies; Viral oncoantigens; e.g. HPV E6 and E7 antigens from Human Papilloma Virus, EBV LMP1 and LMP2. In a further embodiment, a tumor antigens may be encoded in the DNA minicircle downstream or as fusion partners of pathogen-derived antigenic determinants (such as tetanus FrC or DOM1) to provide CD4+ T cell help (as noted for tumor targeting conjugates above).

In another embodiment, a method of identifying a nucleic acid conjugate which induces immune cell activation/maturation and target cell death is disclosed including contacting one or more cells in vitro with a test nucleic acid conjugate containing an antibody or peptide or targeting moiety that specifically binds to a cellular component of a tumor cell, tumor vasculature, and/or a component of a tumor microenvironment, where the antibody or peptide or targeting moiety is conjugated to a nucleic acid comprising one or more immunostimulatory nucleic acid sequences (INAS), and where one or more of the nucleic acid sequences include a pathogen-associated molecular pattern (PAMP) or other motif that can activate immune cells, and determining induction of a marker or a phenotypic change in the one or more cells in the presence or absence of immune cells, where the determined induction or change in the presence of the test antibody/peptide-nucleic acid conjugate is indicative of immune cell activation/maturation, modulation of target cell signaling, and target cell death.

In another aspect, the antibody-nucleic acid conjugate is further conjugated with an antigen derived from an infectious microbe or pathogenic microorganism including viruses, bacteria, mycobacteria, spirochetes, fungi, rickettsia, mycoplasma, chlamydia, protozoan and metazoan parasites, or helminth.

### IV. METHODS

In various aspects of the invention, a composition of the invention is administered to a subject in need thereof to prevent or treat a disease condition. In various embodiments, the composition of the invention is selected based on its targeting moiety and the active agents. As described herein above, a formula T-A₁-A₂ or a variation thereof is used based on the particular disease sought to be treated or prevented.

For example, if the disease condition is pancreatic cancer, an immunoconjugate is selected to comprise a targeting moiety selective for a tumor antigen and/or a pancreatic cell component, one or more immunostimulatory nucleic acid molecule (e.g., PAMP, DAMP, Alarmin, and alternatively a antigenic polypeptide. In another example, the immunoconjugate can further comprise a nucleic acid molecule (e.g., minicircle coupled to the targeting moiety) which encodes an antigenic polypeptide, a co-stimulatory polypeptide, or both.

In various embodiments, the nucleic acid sequences comprising the conjugate may be stable/stabilized (to resist nucleases or lysosomal degradation) to facilitate their delivery and recognition by the immune system.

A “stable” or “stabilized nucleic acid molecule” shall mean a nucleic acid molecule that is relatively resistant to in vivo degradation (e.g., via an exo- or endo-nuclease). Stabilization can be a function of length or secondary structure. For shorter immunostimulatory nucleic acid molecules, secondary structure can stabilize and increase their effect. For example, if the 3′ end of a nucleic acid molecule has self-complementarily to an upstream region, so that it can fold back and form a sort of stem loop structure, then the nucleic acid molecule becomes stabilized and therefore exhibits more activity.

In one aspect, stabilized nucleic acid molecules of the instant invention have a modified backbone. For use in immune stimulation, stabilized nucleic acid molecules may include phosphorothioate (i.e., at least one of the phosphate oxygens of the nucleic acid molecules is replaced by sulfur) or phosphorodithioate modified nucleic acid molecules. More particularly, the phosphate backbone modification occurs at the 5′ end of the nucleic acid for example, at the first two nucleotides of the 5′ end of the nucleic acid. Further, the phosphate backbone modification may occur at the 3′ end of the nucleic acid for example, at the last five nucleotides of the 3′ end of the nucleic acid. In addition to stabilizing nucleic acid molecules, as reported further herein, phosphorothioate-modified nucleic acid molecules (including phosphorodithioate-modified) can increase the extent of immune stimulation of the nucleic acid molecule.

Other stabilized nucleic acid molecules include: nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated. Nucleic acid molecules which contain a diol, such as tetraethylenglycol or hexaethyleneglycol, at either or both termini have also been shown to be substantially resistant to nuclease degradation. In one aspect, the nucleic acid molecules contain peptide bonds (i.e., peptide nucleic acids: PNAs).

Additional methods of stabilizing nucleic acids for in vivo which can be used with compositions and methods of the instant invention are known, such as disclosed in U.S. Pat. Nos. 7,223,741; 7,220,549; 6,239,116; 6,379,930; 6,406,705; 6,218,371; 6,429,199; 6,55,206; 6,271,206; U.S. Patent Application Publication NOs: 20070161590; 20070135372; 20070078104; 20070065467; 20070037767; 20060240093; 20060211639; 20060172966; 20060008910; and 20050191342.

### Coupling

In various embodiments of the invention, one or more components comprised in a composition of the invention are coupled together via a covalent or non-covalent linkage. Various convention methods of coupling nucleic acid molecules to other nucleic acid molecules, nucleic acid molecules to peptides or polypeptides, and peptides/polypeptides to other peptides/polypeptides are known in the art. Non-covalent coupling can be through hydrogen bonding, ionic interactions, Van der Waals interactions, and hydrophobic bonds

Furthermore, various methods are known which employ a variety of chemistries for covalent coupling of active agents. Such agents may include targeting moieties such as antibodies, polypeptides and nucleic acids, as well as other substances to direct the active agents to selected target cells. For example, active agents have been conjugated to various particulate carriers and have been encapsulated into liposomes, micelles and nanoparticles where they are protected from serum degradation.

For example, conjugation of plasmid/minicircle bound-oligonucleotide (3′ or 5′ end) can be effected to a targeting moiety, such as an antibody. Heterobifunctional reagents containing an amine reactive NHS ester on one end and a sulthydryl reactive maleimide group on the other end are used to produce antibody-DNA conjugates. Cross-linking reagents possessing these functional groups can be used to synthesize conjugates (eg. SMCC or sulfo-SMCC). This allows activation of either DNA or antibody via the amine reactive NHS ester end, resulting in a maleimide-activated intermediate. The intermediate species is purified away from excess cross-linker and reaction byproducts before mixing with the second molecule to be conjugated. The multistep nature of this process limits polymerization of the conjugated proteins and provides control over the extent and sites of cross-linking. In protocols involving DNA activation by SMCC and subsequent conjugation with the antibody molecule, the antibody is prepared for coupling to the maleimide groups on the DNA by introduction of sulfhydryl groups via the following options: (a) the disulfide residues in the hinge region of the IgG structure may be reduced with either 2-mercaptoethylamine or dithiothreitol (DTT) to expose free sulfhydryl groups; (b) a thiolation reagent may be used to modify the intact antibody to contain sulfhydryl groups (e.g. SATA and Traut's reagent; 2-Iminothiolane) (Ref. Bioconjugate techniques, Hermanson, G. T., Academic Press, 1996, pages 456-527).

Activation of DNA with NHS Ester-Maleimide Cross-linkers: The triple helix with the oligonucleotide DNA carrying a terminal amine is treated with sulfo-SMCC to yield maleimide-DNA which is then purified away from excess cross-linker by column chromatography. The maleimide activated DNA may be used immediately to conjugate the antibody or freeze-dried for later use.

In another example, conjugation of maleimide-activated DNA to reduced or thiolated antibodies: The antibody is reduced with MEA or DTT in the presence of EDTA to prevent reoxidation of the sulfhydryls by metal catalysis. The reduced IgG is purified by column chromatography. For thiolation of antibodies, antibody is reacted with a thiolating agent (e.g. 2-Iminothiolane or SATA)(molar excess of 10-50× over antibody) for 30 minutes at 37° C. or 1 h at room temperature. The thiolated antibody is purified by column chromatography. The reduced or thiolated antibody fraction is mixed with the maleimide-activated DNA at the desired DNA-to-antibody ratio (eg. 4:1 to 15:1 molar ratio) and incubated 30-60 minutes at 37° C. or 2 h at room temperature or overnight at 4° C. The conjugate is purified away from the unconjugated DNA by affinity chromatography, as described. The conjugate is frozen, lyophilized, or sterile filtered and kept at 4° C. Other methods are provided in the art: (Ref. Bioconjugate techniques, Hermanson, G. T., Academic Press, 1996, pages 456-527).

In additional embodiments, a conjugate of the invention comprises Formulation of conjugate is produced using attachment of an auxillary molecules that protects DNA from nuclease degradation and facilitates cellular entry

In some embodiments, a targeting moiety, e.g., an intact antibody, an antibody fragment (e.g. Fab, etc.), a single chain antibody, is chemically conjugated to the immunostimulatory molecule (e.g., nucleic acid and/or peptide/polypeptide) directly or through a linker. A linker can be a short stretch (e.g., 3 to 15, to 25 amino acids or nucleic acid bases). Examples of linkers which can be used in the context of the present invention are disclosed in US Patent application publication no. 2007/0003514.

In one embodiment, a targeting moiety of the present invention is cross-linked to one or more components. For example, an antibody may be coupled to avidin and the other to biotin. Such antibodies can, for example, target immune system cells to unwanted cells (see for instance U.S. Pat. No. 4,676,980). Suitable peptide cross-linking agents and techniques are well known in the art, and examples of such agents and techniques are disclosed in for instance U.S. Pat. No. 4,676,980.

Furthermore, means of chemically conjugating molecules are well known to those of skill. The procedure for attaching an immunostimulatory molecule to an antibody will vary according to the chemical structure of the agent. Polypeptides typically contain variety of functional groups; e.g., carboxylic acid (COOH) or free amine (—NH₂) groups, that are available for reaction with a suitable functional group on an effector molecule to bind the effector thereto.

In addition, a targeting moiety may be chemically modified by covalent conjugation to a polymer to for instance increase their circulating half-life. Exemplary polymers, and methods to attach them to peptides, are illustrated in for instance U.S. Pat. No. 4,766,106, U.S. Pat. No. 4,179,337, U.S. Pat. No. 4,495,285 and U.S. Pat. No. 4,609,546. Additional illustrative polymers include polyoxyethylated polyols and polyethylene glycol (PEG) (e.g., a PEG with a molecular weight of between about 1,000 and about 40,000, such as between about 2000 and about 20,000, e.g., about 3,000-12,000). A targeting moiety may also be conjugated with any suitable type of chemical group, such as a methyl or ethyl group, or a carbohydrate group. These and other suitable conjugated groups may be used to improve the biological characteristics of a targeting moiety, such as an antibody or functional fragment thereof, e.g., to increase serum half-life, solubility, and/or tissue binding.

Antibody derivatives may be produced by chemically conjugating, protein, or other agent/moiety/compound to (a) the N-terminal side or C-terminal side of the Antibody or subunit thereof (e.g., an anti-CD38 antibody H chain, L chain, or anti-CD38 specific/selective fragment thereof) an appropriate substituent group or side chain or (b) a sugar chain in the Antibody (see, e.g., Antibody Engineering Handbook, edited by Osamu Kanemitsu, published by Chijin Shokan (1994)). Derivatives may also be generated by conjugation at internal residues or sugars, where appropriate.

Antibodies may also be derivatized with a detection agents, for instance fluorescent compounds, including fluorescein, fluorescein isothiocyanate, rhodamine, 5-dimethylamine-1-napthalenesulfonyl chloride, lanthanide phosphors, and the like. Additional examples of suitable fluorescent labels include a ¹²⁵Eu label, an isothiocyanate label, a phycoerythrin label, a phycocyanin label, an allophycocyanin label, an o-phthaldehyde label, a fluorescamine label, etc. Examples of chemiluminescent labels include luminal labels, isoluminal labels, aromatic acridinium ester labels, imidazole labels, acridinium salt labels, oxalate ester labels, a luciferin labels, luciferase labels, aequorin labels, etc.

In one embodiment, an antibody derivative comprises a conjugated nucleic acid or nucleic acid-associated molecule. As provided herein, a nucleic acid molecule can be a coding nucleic acid, a non-coding nucleic acid, or a combination of coding and non-coding nucleic acid sequences. In one embodiment, the noncoding sequences are immunostimulatory in and of themselves.

Alternatively, an antibody and/or immunostimulatory component(s) can be derivatized to expose or attach additional reactive functional groups. The derivatization can involve attachment of any of a number of linker molecules such as those available from Pierce Chemical Company, Rockford Ill. Furthermore, suitable crosslinkers for use in the context of the invention include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are also available from Pierce Chemical Company.

A “linker”, as used herein, is a molecule that is used to join the antibody to the immunostimulatory component(s) comprising a nucleic acid molecule and/or a polypeptide or peptide. The linker is typically capable of forming covalent bonds to both the antibody and to the immunostimulatory active agent. Suitable linkers are well known to those of skill in the art and include, but are not limited to, straight or branched-chain carbon linkers, heterocyclic carbon linkers, or peptide linkers. Where the antibody and the immunostimulatory molecule are polypeptides, the linkers can be joined to the constituent amino acids through their side groups (e.g., through a disulfide linkage to cysteine). However, one embodiment, the linkers will be joined to the alpha carbon amino and carboxyl groups of the terminal amino acids.

In some embodiments, a linker can provide one or more cleavage sites. Therefore, a conjugate of the invention can comprise cleavable or non-cleavable linkers. For the instant invention, biocleavable linkages are defined as types of specific chemical moieties or groups that can be used within the compositions to covalently couple or cross-link components such as nucleic acids, intercalators, active agents, targeting moieties, amphiphilic molecules and polymers described herein. Some suitable examples are disclosed for use in oral delivery by V. R. Sinha, et al, Europ. J Pharmaceutical Sci. 18, 3-18 (2003) and references therein. Biocleavable linkages or bonds are distinguishable by their structure and function.

Cleavable Peptide Linkages. Another preferred category of biocleavable linkages is biocleavable peptides or polypeptides from 2 to 100 residues in length, preferably from 3 to 20 residues in length. These are defined as certain natural or synthetic polypeptides that contain certain amino acid sequences (i.e. are usually hydrophobic) that are cleaved by specific enzymes such as cathepsins, found primarily inside the cell (intracellular enzymes). Using the convention of starting with the amino or “N” terminus on the left and the carboxyl or “C” terminus on the right, some examples are: any peptides that contain the paired amino acids Phe-Leu, Leu-Phe or Phe-Phe, such as Gly-Phe-Leu-Gly (GFLG) (SEQ ID NO:242) and other combinations. Preferred examples (among others) include leucine enkephalin derivatives and any cathepsin cleavable peptide linkage sequences disclosed by J. J. Peterson, et al, in Bioconj. Chem., Vol. 10, 553-557, (1999), and references therein and in U.S. patent application Ser. No. 10/923,112 that are incorporated herein by reference.

Another preferred type of biocleavable linkage is any “hindered” or “protected” disulfide bond that sterically inhibits attack from thiolate ions or other cleavage mechanisms. Examples of (but not limited to) such protected disulfide bonds are found in the coupling agents: S-4-succinimidyl-oxycarbonyl-.alpha.-methyl benzyl thiosulfate (SMBT) and 4-succinimidyloxycarbonyl-.alpha.-methyl-.alpha.-(2-pyridyldithio) toluene (SMPT). Another useful coupling agent resistant to reduction is SPDB disclosed by Worrell, et al., Anticancer Drug Design 1:179-188 (1986). Also included are certain aryldithio thioimidates, substituted with a methyl or phenyl group adjacent to the disulfide, which include ethyl S-acetyl 3-mercaptobutyrothioimidate (M-AMPT) and 3-(4-carboxyamido phenyldithio) proprionthioimidate (CDPT), disclosed by S. Arpicco, et al., Bioconj. Chem. 8 (3):327-337 (1997).

Many procedures and linker molecules for attachment of various compounds to proteins such as antibodies are known (see, e.g., European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075).

A bifunctional linker or trifunctional linker having one functional group reactive with a group on each component of the chimeric moiety, can be used to form the desired immunoconjugate. Alternatively, in certain embodiments derivatization can involve chemical treatment of the antibody, e.g., glycol cleavage of a sugar moiety of a glycoprotein antibody with periodate to generate free aldehyde groups. The free aldehyde groups on the antibody can be reacted with free amine or hydrazine groups on, e.g., a linker bind the polypeptide (see, e.g., U.S. Pat. No. 4,671,958). Procedures for generation of free sulfhydryl groups on polypeptide, such as antibodies or antibody fragments, are also known (see, e.g., U.S. Pat. No. 4,659,839).

In another embodiment, coupling is between a double stranded nucleic acid molecule and a single stranded nucleic acid. In alternative embodiments, either the single strand or double strand can be coupled to the targeting moiety. In one embodiment, a targeting moiety is linked to a nucleic acid molecule which couples (e.g., is conjugated) to another nucleic acid molecule to form a triplex nucleic acid molecule. Furthermore, triplex nucleic acid molecules can themselves further interact with either double-stranded or single-stranded nucleic acid, i.e., forming quadraplex and quantaplex nucleic acid molecules. In one embodiment, a triplex is formed, in which three strands of DNA form a complex dependant on both Watson-Crick and Hoogsteen base-pairing. Triplex molecules can bind target regions with high affinity and specificity. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566 and 5,962,426.

In one embodiment, a composition of the invention comprises a nucleic acid molecule which is immunostimulatory and which forms a triplex with a nucleic acid molecule which encodes one or more tumor antigens. In a further embodiment, the nucleic acid encoding one or more tumor antigens, further encodes or alternatively encodes one or more antigen associated with a pathogen. In yet another embodiment, the nucleic acid encoding such polypeptides, is a minicircle DNA. Minicircle expression vectors are known and can be used within the context of the present invention, including those disclosed in U.S. Pat. Nos. 6,143,530, 6,825,012 and 7,018,833.

In yet another method, coupling of an antibody to a active agent (e.g., nucleic acid molecule) is effected through photoaffinity. Antibodies contain one or more photoaffinity sites which provide for the selective site-specific attachment of photoaffinity compounds thereto. In particular, it has been discovered that antibodies comprise one or more sites having high affinity for purines, azido-purines and other similar heterocyclic organic compounds, and specifically ATP- or GTP-analogs. Furthermore, other photoaffinity binding sites may further be identified, e.g., by reaction of antibodies with non-purine containing photoaffinity compounds, e.g., pyrimidine derivatives such as photoactive analogs of dUTP, including 5-azido-2′-deoxyuridine 5′-triphosphate (5-N.sub.3 dUTP).

The purine or azidopurine nucleotide affinity site will hereinafter be referred to as the “purine ring binding” or simply the “PRB” domain or site. The PRB site on antibody molecules was discovered after it was found by the present inventors that photoaffinity compounds, in particular purine or azidopurine photoaffinity compounds readily attach to antibodies and antibody fragments by a photoactivated chemical reaction which occurs under mild, physiological conditions. Specifically antibodies comprise one or more PRB sites which exhibit such a high affinity for purines and azidopurine photoaffinity analogs, that reaction of antibodies with purine and azidopurine photoaffinity analogs under mild, physiological conditions, and more particularly after only a single 2-5 minute photolysis results in nearly 100% photoattachment.

As described in U.S. Pat. No. 5,693,764, photoaffinity provides for the effective photoinsertion of a nucleotide or nucleoside photoaffinity compound, preferably a purine, azidopurine or similar heterocyclic base containing photoaffinity analog, and most preferably an ATP- or GTP-analog photoaffinity compound, into an antibody molecule, which does not result in substantial loss of antigen binding.

Suitable methods for attaching nucleotide photoaffinity analogs to proteins are described, e.g., in Potter & Haley, Meth. in Enzymol., 91:613-633, (1983); Owens & Haley, J. Biol. Chem., 259:14843-148 48, (1987); Atherton et al, Biol. of Reprod., 32:155-171, (1985); Khatoon et al, Ann. of Neurology, 26:210-219, (1989); King et al, J. Biol. Chem., 269:10210-10218, (1989); Dholakia et al, J. Biol. Chem., 264:20638-20642, (1989); Campbell et al, Proc. Natl. Acad. Sci., 87:1243-1246, (1990); and Kim et al, J. Biol. Chem., 265:3636-3641, (1990), which references are incorporated by reference in their entirety herein.

Any antibody or antibody containing composition which effectively binds nucleotide or nucleoside photoaffinity compounds is within the scope of the present invention. This includes by way of example, polyclonal and monoclonal antibodies, recombinant antibodies, chimeric antibodies, bispecific antibodies, single chain antibodies, antibodies from different species (e.g., mouse, goat, rabbit, human, rat, bovine, etc.), anti-idiotypic antibodies, antibodies of different isotype (IgG, IgM, IgE, IgA, etc.), as well as fragments and derivatives thereof. (e.g., (Fab)₂ fragments.)

As an example, a nucleotide sequence included in plasmid and minicircle DNA can be produced per the following specifications:

a. ds DNA sequence capable of hybridizing and binding with a oligonucleotide

b. Specific sequence is preferably fully complementary to oligonucleotide used for formation of a triple helix

c. Sequence incorporated at site that does not affect promoter-directed expression of the gene of interest

d. Sequence may be 3-50 base pairs in length; preferably >10 base pairs

e. Example sequences may preferably be a homopurine (Pu)-homopyrimidine (Py) ds DNA: a region in the plasmid of repeating sequences, based upon (CT)ₙ with complementary repeat (GA)ₙ on the opposite strand. e.g. 5′CTCTCTCTCTCTCTC 3′ (SEQ ID NO:243)
1) 3′ GAGAGAGAGAGAGAG 5′
2) a region in the plasmid of repeating sequences, based upon (CCTT)n, with complementary strand (GGAA)n e.g. 5′ CCTTCCTTCCTTCC 3′ (SEQ ID NO:244)
   (1) 3′ GGAAGGAAGGAAGG 3′
      a region in the plasmid of repeating sequences, based upon (CTT)n, with complementary strand (GAA)n
      e.g. 5′CTT CTT CTT CTT CTT CTT 3′(SEQ ID NO:245)
a. 3′ GAAGAA GAA GAA GAA GAA 5′
   a region in the plasmid of repeating sequences, based upon (CCT)n, with complementary strand (GGA)n
   e.g. 5′CCT CCT CCT CCT CCT CCT 3′(SEQ ID NO:246)
b. 3′ GGAGGA GGAGGA GGA GGA 5′ any other homopurine-homopyrimidine sequence
   e.g. 5′ TCT CCT CCT TT 3′ (SEQ ID NO:247) 3′ AGA GGA GGA AA 5′

In some embodiments, guanine-rich DNAs can assemble to form four-stranded structures, which are based on stacks of square-planar arrays of G-quartets (1-4). The G-quartets consist of four guanines that are linked by Hoogsteen type base pairing. Monovalent cations are selectively bound in the central cavity between the G-quartets, and these structures are specifically stabilized by potassium; sodium produces less stable complexes, whereas lithium inhibits assembly (5,6). G-quadruplexes can be formed by the intermolecular association of four DNA strands (5,7,8), by the dimerization of sequences that contain two G-tracts (9,10) or by the intramolecular folding of one strand containing at least four G-tracts (11-15). In particular, telomeric sequences consist of highly repeated G-rich sequences such as (GGGTTA)ₙ in humans and other higher organisms, (GGGGTT)ₙ in Tetrahymena, and (GGGGTTTT)ₙ in Oxytrichia. Quadruplexes have also been implicated in the control regions of some oncogenes, especially c-myc (16,17), immunoglobulin switch regions (3), the retinblastoma susceptibility gene (18), the FMR-1 gene (19), the chicken 13-globin gene (20), and the insulin gene (21). In addition, several synthetic aptamers are known to be based around a G-quadruplex platform including those targeted to HIV-integrase (22) and thrombin (12). Molecules containing G-quartets can self-associate by forming non-Watson-Crick, guanine-guanine base-paired, intramolecular structures. These structures form below 40° C. at moderate ionic strength and neutral pH and behave like hairpin duplexes. It has previously been shown that addition of a terminal T (3′ end or 5′ end) stabilizes quadruplex structures (37), an effect which is caused by the additional base stacking with possibly some pairing with the terminal G-quartet (38).

For example, a sequence for forming can be: 5′ TGGGGT 3′
(3) 3′ TGGGGT 5′

In one embodiment, a method for incorporating specified nucleotide sequences is provided (including target cell active promoter sequence, gene of interest, and oligonucleotide binding sequence) in plasmid or minicircle DNA, as follows. The DNA sequence for the gene of interest is first codon optimized for efficient expression in mammalian cells (DNA 2.0). The chosen sequences (target cell specific promoter, gene of interest, oligonucleotide binding motif) are cloned into an intermediate mammalian expression vector containing a CMVie promoter and SV40 terminator vector. [e.g. The plasmid pGL3 Basic (Promega) with the CMV immediate early promoter driving gene expression]. After sequence confirmation the entire expression cassette (promoter, gene of interest, SV40 terminator, oligonucleotide binding motif) is PCR amplified with PCR primers containing either SpeI (5′ end) or ApaI (3′ end) restriction endonuclease site specific tails. The PCR product is then digested with SpeI and ApaI and ligated into the SpeI and ApaI sites of the p2 φC31 minicircle vector. The construct, p2φC31-Gene, is then transformed into E. coli NM522 cells and tested for recombination capability. E. coli containing the plasmid are grown and then recombination is induced by the addition of arabinose (0.25% final concentration). An aliquot of culture is taken before (time 0) and after (60 and 120 minutes) induction and subjected to miniprep plasmid isolation. The resulting plasmid prep is subjected to electrophoresis to determine if the mother plasmid had recombined into the miniplasmid and minicircle. Successful recombination is determined by the presence of a minicircle band on the gel. The decrease in the intensity of the backbone plasmid band (miniplasmid) over time indicates that the plasmid backbone is cut by I-SceI enzyme and degraded by the cellular endonucleases.

Plasmid DNA is prepared using the Qiagen MaxiPrep procedure or by the Qiagen Endofree Plasmid Maxi Kit and re-suspended in TE (10 mM Tris±HCl, 1 mM EDTA) pH 8.0 at 1 mg/ml. Plasmids are >95% supercoiled by agarose gel electrophoresis.

The molecular methods and cloning techniques, such as digestion with restriction enzymes, gel electrophoresis, transformation of E. Coli (types-methylation), nucleic acid precipitation, nucleic acid hybridization, and the like are described in the literature (Maniatis et al., T, E. F. Fritsch, and J. Sambrook, 1989. Molecular cloning: a laboratory manual, second edition. Cold Spring Harbor Laboratory Press, New York; Ausubel F. M., R. Brent, R. E. Kinston, D. D. Moore, J. A. Smith, J. G. Seidman and K. Struhl. 1987. Current protocols in molecular biology 1987-1988. John Willey and Sons, New York).

In some embodiments, plasmids are capable of site-specific binding of an oligonucleotide, such as DNA, LNA, PNA. Plasmids based upon the pGeneGrip series, expressing either luciferase (gWiz) or green fluorescent protein (GFP; pGGGFP) [GTS; Zelphati et al. (8)]. Within the transcriptional terminator of plasmids gWiz and pGGGFP, enabling site-specific binding without interfering with gene expression, is GeneGrip site 1, a region in the plasmid of repeating sequences, based upon (CT)n with complementary repeat (GA)n on the opposite strand. Site 2, which is located 5′ to the cytomegalovirus (CMV) promoter, is based upon (CCTT)ₙ, with complementary strand (GGAA)ₙ, and is found only in plasmids pGG2XGFP and pGG2XEMPTY, which additionally contain site 1 [GTS Catalogue 2002; Zephati et al. (8)]. Plasmid pGG2XEMPTY is derived from pGG2XGFP by deletion of the GFP gene. To construct plasmid pGG2XEMPTY, pGG2XGFP is digested with NheI and BamHI, and the remaining 5.1 kb plasmid fragment is gel purified, treated with Klenow DNA polymerase and re-circularized by ligation (33).

Olignucleotides can be produced used convention methods. For example, synthesis of linear single strand oligonucleotide for hybridization to plasmid/minicircle DNA. In some embodiments, the oligonucleotide is a linear strand of DNA, RNA, LNA, PNA or hybrid (DNA-LNA, DNA-PNA, RNA-LNA, RNA-PNA or the like) that includes a specific sequence that binds (and is preferably complementary) to a nucleotide sequence in the double stranded plasmid or minicircle DNA molecule.

Furthermore, an oligonucleotide sequence may bind to plasmid or minicircle DNA via Hoogsteen base-pair based formation of a triple helix by hybridization. Hoogsteen base pairing is more robust for PNAs containing pseudoisocytosine, not cytosine, residues, enabling Hoogsteen base pairing at high pH>5±6, whereas PNAs containing cytosine only bind at low pH<5±6 (30). The addition of certain amino acids improves the stability of ‘bis’ PNAs bound to DNA.

Alternatively, an olignucleotide can bind a plasmid/minicircle DNA via Watson-Crick based Strand invasion and strand displacement. For example, LNA ODNs are strand displacement agents of supercoiled plasmid DNA. Sequence-specific LNA ODN binding to plasmid DNA, at its cognate binding site, causes strand displacement of the unbound DNA strand. ‘bis’ PNA ODNs with the addition of a few, positively charged amino acids are also excellent strand displacement agents.

In addition, such nucleic acid molecules can form quadruplexes. For example, the oligonucleotide may include Guanine-rich nucleotides that can assemble to form four-stranded structures, which are based on stacks of square-planar arrays of G-quartets.

The oligonucleotide can contain the following bases: Thymidine (T)—to form base pairs with A and/or triplets with AT doublets of ds DNA; Cytosine or Protonated cytosine (C+)—to form base pairs with G and/or triplets with GC doublets of ds DNA; Adenine (A)—to form base pairs with T and/or triplets with AT doublets of ds DNA; Guanine (G)—to form base pairs with C and/or triplets with GC doublets of ds DNA; Uracil (U)—to form base pairs with A and/or triplets with AT doublets of ds DNA.

In further embodiments, the oligonucleotide may be composed of unmodified natural bases or chemically modified bases to increase its resistance to nucleases and/or improve affinity for its complementary ds DNA: Nuclease resistance—modification of backbone (methylphosphonates, phosphorothioates, phosphoamidate, etc.); 2′ 0 methyl modification; and/or improve binding to complementary ds DNA in plasmid/minicircle—e.g. methylation of cytosines (to form a stable triple helix at neutral pH).

In some embodiments, the length of an oligonucleotide may be between 3-50 bases, and the hybridizing region is preferably greater than 10, 11, 12, 13, 14, 15 16, 17, 18, 19 or 20 bases.

In one embodiment, ‘Hybrid’ oligonucleotides may consist of the hybridizing region (DNA, LNA, PNA) and an extension of any length (DNA, RNA, LNA, PNA) to add functionality (linker arm for attachment of targeting moiety, immunostimulatory sequence such as CpG motifs, additional binding motifs, sequences to enable circularization, and the like). For example, LNA (hybridization motif) extended to a phosphorothioate CpG ODN. Use of LNA to bind PTO CpG ODNs to plasmid encoding an antigen can lead to an immune adjuvant effect without inhibiting high-level antigen expression. Furthermore, a linker arm can be any sequence with bases that do not interfere with hybridization to the plasmid/minicircle DNA and enables coupling of the plasmid/minicircle to the antibody at a preferred distance (eg. Linker may contain 3-20 purine bases; GAGG).

In another embodiment, an oligonucleotide may conform to Padlock oligonucleotides for duplex DNA based on sequence-specific triple helix formation. An oligonucleotide may be circularized around double-stranded DNA via triple helix formation by binding into the DNA major groove at an oligopurine-oligopyrimidine sequence. After sequence-specific recognition of a double-stranded DNA target through triple helix formation, the ends of the triplex-forming oligonucleotide may be joined through the action of T4 DNA ligase, thus creating a circular DNA molecule catenated to the plasmid containing the target sequence. The labeling of the double-stranded DNA sequence has been carried out without any chemical or enzymatic modification of this sequence. These “padlock” oligonucleotides provide a tool to attach a noncovalent tag in an irreversible way to super-coiled plasmid or other double-stranded DNAs. [Ref. Padlock oligonucleotides for duplex DNA based on sequence-specific triple helix formation. Escude, C., T Garestier, C Helene. Proc. Natl. Acad. Sci. USA Vol. 96, pp. 10603-10607, September 1999, Biochemistry]

The oligonucleotide may be synthesized by any known technique (nucleic acid synthesizers, phosphoramidite chemistry). In some embodiments, an oligonucleotide may be functionalized with a 3′ and/or 5′ modification (amine, thiol, carboxyl, phosphate group, and the like) to enable covalent conjugation to the targeting moiety/antibody (carrying disulfide, maleimide, amine, carboxyl, ester, epoxide, or aldehyde) via disulfide, thioether, ester, amide, or amine linkage. Any other functionalization of the oligonucleotide may also be performed for conjugation to the targeting moiety/antibody via known bifunctional coupling reagents according to standard protocols.

Example sequences of oligonucleotide (corresponding to complementary DNA incorporated in plasmid/minicircle ds DNA):

(i) complementary to a region in the plasmid of repeating sequences, based upon (CT)ₙ with complementary repeat (GA)ₙ on the opposite strand.
e.g. Oligonucleotide=5′CTCTCTCTCTCTCTC 3′ (SEQ ID NO:243)
Plasmid/minicircle DNA; 5′CTCTCTCTCTCTCTC 3′ (SEQ ID NO:243)
i) 3′ GAGAGAGAGAGAGAG 5′

(ii) complementary to a region in the plasmid of repeating sequences, based upon (CCTT)n, with complementary strand (GGAA)n
e.g. Oligonucleotide=5′CCTTCCTTCCTTCC 3′ (SEQ ID NO:244)
Plasmid/minicircle DNA; 5′CCTTCCTTCCTTCC 3′ (SEQ ID NO:244)
2) 3′ GGAAGGAAGGAAGG 3′

(iii) complementary to a region in the plasmid of repeating sequences, based upon (CTT)n, with complementary strand (GAA)n
e.g. Oligonucleotide=5′CTT CTT CTT CTT CTT CTT 3′ (SEQ ID NO:245)
Plasmid/minicircle DNA; 5′CTT CTT CTT CTT CTT CTT 3′ (SEQ ID NO:245)
a) 3′ GAAGAA GAAGAA GAAGAA 5′

(iv) complementary to a region in the plasmid of repeating sequences, based upon (CCT)n, with complementary strand (GGA)n
e.g. Oligonucleotide=5′CCT CCT CCT CCT CCT CCT 3′ (SEQ ID NO:246)
Plasmid/minicircle DNA; 5′CCT CCT CCT CCT CCT CCT 3′ (SEQ ID NO:246)
b) 3′GGAGGA GGAGGA GGAGGA 5′

(v) complementary to any other homopurine-homopyrimidine sequence
e.g. Oligonucleotide=5′ TCT CCT CCT TT 3′ (SEQ ID NO:247)
Plasmid/minicircle DNA: 5′ TCT CCT CCT TT 3′ (SEQ ID NO:247)
3′ AGAGGA GGAAA5′

(vi) Guanine-rich nucleotides that can assemble to form four-stranded structures, which are based on stacks of square-planar arrays of G-quartets
e.g. Oligonucleotide=5′ TGGGGT 3′
ii. 3′TGGGGT 5′
Plasmid/minicircle DNA: 5′ TGGGGT 3′
1) 3′TGGGGT 5′

In some embodiment, an oligonculeotide is ss RNA oligonucleotide (corresponding to complementary ds DNA incorporated in plasmid/minicircle ds DNA). Illustrative sequences are as follows:

| | |
|---|---|
| i. | |
| 5′ CUCUCUCUCUCUCUC 3′ | (SEQ ID NO:248) |
| ii. | |
| 5′ CCUUCCUUCCUUCC 3′ | (SEQ ID NO:249) |
| iii. | |
| 5′ CUU CUU CUU CUU CUU CUU 3′ | (SEQ ID NO:250) |
| iv. | |
| 5′ CCU CCU CCU CCU CCU CCU 3′ | (SEQ IS NO:251) |
| v. | |
| 5′ UCU CCU CCU UU 3′ | (SEQ ID NO:252) |
| vi. | |
| 5′ UGGGGU3′ | |

Example sequences of LNA and PNA oligonucleotides (ODN-binding sites present on the GeneGrip plasmid series; LNA and PNA ODNs based upon DNA sequences at the repeat binding sites 1 and 2, found on the GeneGrip plasmid series (GTS). PNA/LNA ODNs containing either a (CT)n or a (GA)ₙ repeat motif are designed to bind to GeneGrip site 1; ODNs containing (CCTT)n and (GGAA)n are designed to bind to GeneGrip site 2.

| | |
|---|---|
| Description | Sequence |

| | |
|---|---|
| 13mer 100% LNA | 5′-NH2-CTCTCTCTCTCTC-3′ |
| | (SEQ ID NO:253) |
| 13mer 100% LNA | 5′-NH2-GAGAGAGAGAGAG-3′ |
| | (SEQ ID NO:254) |
| 17mer 50% LNA | 5′-NH2-CtCtCtCtCtCtCtCtC-3′ |
| | (SEQ ID NO:255) |
| 14mer 100% LNA | 5′-NH2-CCTTCCTTCCTTCC-3′ |
| | (SEQ ID NO:256) |
| 14mer 100% LNA | 5′-NH2-GGAAGGAAGGAAGG-3′ |
| | (SEQ ID NO:257) |
| 9mer ‘bis’ 50% | 5′-NH2-CtCtCtCtC-XXX-CtCtCtCtC-3′ |
| LNA,50% DNA | (SEQ ID NO:258) |
| 21mer DNA, | 5′-tccatgacgttcctgacgtttGAGAGAGAGAG |
| 13mer LNA | AG-3′ |
| | (SEQ ID NO:259) |
| 21mer DNA, | 5′-tccatgagcttcctgagtcttGAGAGAGAGAG |
| 13mer LNA | AG-3′ |
| | (SEQ ID NO:260) |
| GTS PNA 8mer | 5′O-O-TCTCTCTC-O-O-O-JTJTJTJT-CONH2 |
| ‘bis’ 100% PNA | (SEQ ID NO:77) |
| OsPNA13mer | 5′O-O-gCTCTCTCTCTCTC-O- |
| ‘bis’ 100% PNA | CTCTCTCTCTCTCk |
| | (SEQ ID NO:261) |
| OsPNA13mer | 5′O-O-gCTCTCTCTCTCTC-O-O-O- |
| ‘bis’ 100% PNA | CTCTCTCTCTCTCk |
| | (SEQ ID NO:262) |
| OsPNA13mer | 5′O-O-gCTCTCTCTCTCTCk |
| 100% PNA | (SEQ ID NO:263) |
| 35mer DNA REP, | 5′ cggcggataaccgcgagcggttattcgcccta |
| 13mer LNA | cgg-CTCTCTCTCTCTC-GGAG-NH₂-3′ |
| (repetitive | (SEQ ID NO:264) |
| extragenic | |
| palindromic- | |
| REP sequence; | |
| P. Aeruginosa) | |
| 19mer CpG A | 5′gggggacgatcgtcggggg- |
| DNA,13mer LNA | CTCTCTCTCTCTC-GGAG-NH₂-3′ |
| | (SEQ ID NO 265) |

LNA residues are bold upper case; DNA residues are bold lower case;
PTO residues are additionally italicised;
PNA and amino acid residues are italicised, normal text, with PNA bases upper case;
O = 8-amino-3,6-dioxaoctanoic acid linker; J = pseudoisocytosine; g = glycine; k = lysine; X = ‘PEG spacer’-9-O-dimethoxytrityl-triethyleneglycol, 1-[(2-cyanoethyl)-(N,N-diisopropyl)]-phosphoramidite, spacer phosphoramidate 9;
NH2 = 5′-amino-modifier C12 phosphoramidite spacer.

[Ref.: Use of locked nucleic acid oligonucleotides to add functionality to plasmid DNA. Kirsten M. L. Hertoghs, Jonathan H. Ellis and Ian R. Catchpole. Nucleic Acids Research, 2003, Vol. 31, No. 20 5817-5830]

In some embodiments, conjugates of the invention comprise oligonucleotides comprising padlock oligonucleotides. Example sequences of padlock oligonucleotides for circularization around a ds DNA: Oligonucleotide (A) containing a central triple helix-forming sequence connected by two Tₙ linkers to sequences that can form 10 base pairs each with a 20-mer oligonucleotide (B). The total length of the oligonucleotide (A) should enable binding to both the duplex target by forming a 15-base-triplet triple helix and to a 20-mer template (oligonucleotide B) by forming a 20-bp double helix. A phosphate group is added to the 5′ end of this oligonucleotide, as required for enzymatic circularization.

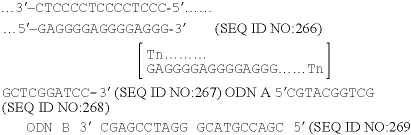

Therefore, any of the oligonucleotides disclosed herein can be used for hybridization of the linear oligonucleotide to a complementary nucleotide sequence in the double stranded plasmid or minicircle DNA. An illustrative method for binding of oligonucleotides to plasmid or minicircle DNA can comprise the following specifications: (i) Plasmid is incubated with PNA/LNA ODNs in 10 mM phosphate buffer, 1 mM EDTA, pH 5.8 for 16 h at 37° C., at a maximum of 4- to 40-fold molar excess of ODN to ODN-binding sites in the plasmid; (ii) For DNA+LNA ODNs binding to plasmid, the ODNs are pre-heated at 80° C. for 10 min and then plunged into ice, to disrupt any self-complementary interaction between the DNA and LNA bases within the ODN that might affect plasmid binding. Any additional binding of DNA ODNs to plasmid DNA+LNA complexes is at 37° C. for 45 min in 10 mM sodium phosphate pH 7.1, 1 mM EDTA at 4 mM DNA ODN; (iii) Annealing methodology for triple helix formation: (a) The DNA oligonucleotide is added to the plasmid/minicircle containing the complementary ds DNA nucleotide sequence in a buffer containing 0.2 M Sodium Acetate and 0.1 M Sodium Chloride; The mixture is incubated at 20° C. for 30 minutes. (b) Triplexes of duplex DNA and triplex-forming oligonucleotide are prepared in 50 mM sodium acetate pH 5.0, containing 150 mM NaCl. (c) For triple helix formation, the oligonucleotide (100 fmol) is incubated in 10 ml of 50 mM Tris HCl, pH 7.5, 10 mM MgCl2, 10 mM DT T, 1 mM ATP, 25 mg/ml BSA, in the presence of various amounts of double-stranded target. The samples are heated to 75° C., then cooled slowly to 45° C. The triple helix containing the plasmid/minicircle and the oligonucleotide is recovered by ethanol precipitation and centrifugation.

Furthermore, to visualize bound ODN, 2.5 mg of plasmid DNA is analysed by agarose±TAE gel electrophoresis without ethidium bromide (EtBr). High percentage (2%) gels are used to maximise separation of both plasmid-bound and free ODN. Any unbound ODNs are separated from plasmid and plasmid-bound ODN by gel exclusion chromatography using MicroSpin Sephacryl S400 HR columns.

In addition, restriction enzyme analysis can also be performed: Restriction enzyme digests of 2.5 mg of plasmid DNA are performed after overnight LNA or PNA ODN binding at 37° C. Plasmid gWiz is digested with BsaI and SphI, and plasmid pGG2XGFP is digested with NdeI. Samples are then analysed on 2% agarose±TAE gels without EtBr.

Confirmation of strand displacement by LNA or PNA binding to plasmid DNA: DNA sequencing reactions: Standard dsDNA sequencing is performed by ‘big dye’ PCR-based thermocycle sequencing using the fluorescent dideoxy terminator method, run on a PE-Biosystems Prism 3700 Capillary sequencer and visualised on an ABI 3700 DNA Analyser. To identify strand displacement from LNA or PNA ODNs binding to plasmid DNA, an ssDNA sequencing assay is performed based upon established methods demonstrating PNA or LNA ODN strand displacement.

An optimal DNA sequencing primer (RevGG2B, 22mer 100% DNA-5′(Cy5) ggaaggaagttaggaaggaagg-3′ (SEQ ID NO:270)) is designed and verified by good quality sequencing across the GeneGrip site 2 repeat region in pGG2XGFP by standard ‘big dye’ sequencing. A 25 mg aliquot of plasmid pGG2XGFP (0.024 mM) is bound with ODN LNA (low concentration: 0.5 mM) and unbound LNA ODN is removed. Plasmid pGG2XGFP with and without bound LNA is then subject to a modified ssDNA sequencing protocol using the AutoRead Sequencing Kit (Amersham Pharmacia Biotech) with Cy5-labelled RevGG2B DNA primer and T7 DNA polymerase. The dose of template plasmid DNA is varied from 1 to 3 mg and the annealing temperature reduced to either 37 or 42° C., but the annealing time is extended to 30 min to maximise sequence-specific binding of the DNA sequencing primer to any displaced ssDNA regions under conditions that should not disrupt the double-stranded nature of the plasmid. Sequencing reactions are then run on a Visible Genetics DNA Sequencer and modified using Chromas software. Using the known DNA sequence of the region, the DNA sequence obtained for plasmid with LNA bound is interpreted by eye. [Ref.: Use of locked nucleic acid oligonucleotides to add functionality to plasmid DNA. Kirsten M. L. Hertoghs, Jonathan H. Ellis and Ian R. Catchpole. Nucleic Acids Research, 2003, Vol. 31, No. 20 5817-5830

In some embodiments, oligonucleotide is circularized around the plasmid/minicircle. To circularize the plasmid-bound oligonucleotide, ligation reactions are carried out in buffer (50 mM Tris-HCl, pH 7.5, 10 mM MgCl2, 10 mM DTF, 1 mM ATP, 25 mg/ml BSA), by adding the template oligonucleotide (1 pmol) and 40 units of T4 DNA ligase, and incubating for 1 hr at 45° C. Ligase is heat inactivated for 15 min at 65° C. [Ref. Padlock oligonucleotides for duplex DNA based on sequence-specific triple helix formation. Escude, C., T Garestier, C Helene. Proc. Natl. Acad. Sci. USA Vol. 96, pp. 10603-10607, September 1999, Biochemistry

The foregoing means for coupling nucleic acids and polypeptides/peptides is merely illustrative and not limiting.

The methods of the present invention can be generally employed to link an INAS to a variety of amino acid polymers, including peptides and antibodies. Conjugation of biologically active agents with a targeting moiety (e.g., peptide, antibody, aptamer) may be accomplished by any conventional method, including: covalent or non-covalent conjugation, chemical conjugation, physical conjugation, conjugation via linkers (such as protamine, biotin-avidin binding, etc.). Furthermore, in some embodiments, a composition of the invention comprises a nucleic acid molecule, wherein the composition is associated with a polycation (e.g, protamine) or other agent conventionally used to condense or package nucleic acid molecules for delivery into a cell.

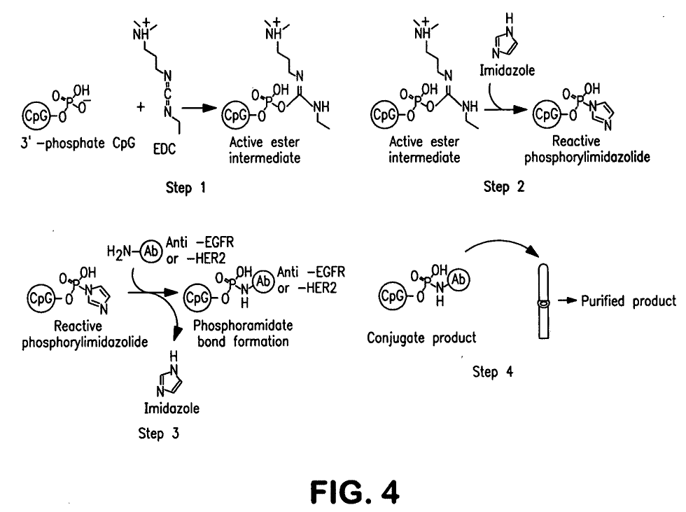

An exemplary method of conjugation is disclosed and shown in FIG. 4.

Additional methods for coupling or associating two or more components of a composition of the invention are conventional and include use of triplex, or quadraplex nucleic acid strand formation, Such methods include, but are not limited to, activation of a carboxylic acid moiety on a peptide or antibody by the addition of an activating agent. Activating agents include HATU (O-(7-azabenzotriazol-1-yl)-N,N,N′,N′-tetramethyluronium hexafluorophosphate); HBTU (O-benzotriazol-1-yl)-N,N,N′,N′-tetramethyluronium hexafluorophosphate); TBTU (2-(1H-benzotriazo-1-yl)-1-1,3,3-tetramethyluronium hexafluorophosphate); TFFH(N,N′,N″,N″-tetramethyluronium 2-fluoro-hexafluorophosphate); BOP (benzotriazol-1-yloxytris(dimethylamino)phosphonium hexafluorophosphate); PyBOP (benzotriazole-1-yl-oxy-tris-pyrrolidino-phosphonium hexafluorophosphate); EEDQ (2-ethoxy-1-ethoxycarbonyl-1,2-dihydro-quinoline); DCC (dicyclohexylcarbodiimide); DIPCDI (diisopropylcarbodiimide); HOBt (1-hydroxybenzotriazole); N-hydroxysuccinimide; MSNT (1-(mesitylene-2-sulfonyl)-3-nitro-1H-1,2,4-triazole); aryl sulfonyl halides, e.g. triisopropylbenzenesulfonyl chloride. Preferred activating agents are carbodiimides. In one aspect, activating agents are 1-ethyl-3-(3-dimethylaminopropyl) carbodiimide hydrochloride (EDC) and/or 1-cyclohexyl-3-(2-morpholinoethyl) carbodiimide (CDC).

The activated carboxylic acid moiety as described above reacts with the nucleophilic moiety on the INAS, under conditions known to the skilled practitioner as sufficient to promote the reaction of the activated carboxylic acid moiety with the nucleophilic moiety. Under appropriate conditions, a relatively low pH is maintained, i.e., a pH less than about 6.5. Under traditional methods (i.e., at higher pH levels) it is believed that the activated carboxylic acid and/or the activating agent hydrolyze quickly, reducing the efficiency of the conjugation reaction.

The biologically active agents of the invention can be coupled to targeting moieties of the invention through conventional methods. For example, for immunostimulatory nucleic acid molecules (INAS) of the present invention, the INAS may be coupled with a peptide or polypeptide in a number of ways including, but not limited to, conjugation (linkage). The polynucleotide portion can be coupled with the peptide or polypeptide portion of a conjugate involving covalent and/or non-covalent interactions. Generally, an INAS and peptide or polypeptide are linked in a manner that allows enhanced or facilitated uptake of the conjugate by a tumor or targeted cell.

The link between the peptide or polypeptide and INAS can be made at the 3′ or 5′ end of the INAS, or at a suitably modified base at an internal position in the INAS. If the peptide or polypeptide contains a suitable reactive group (e.g., an N-hydroxysuccinimide ester) it can be reacted directly with the N⁴ amino group of cytosine residues. Depending on the number and location of cytosine residues in the INAS, specific coupling at one or more residues can be achieved.

The methods of the present invention can be used to prepare a variety of conjugates. In one aspect, conjugates of the present invention include, but are not limited to, DNA-antibody conjugates, DNA-peptide conjugates, RNA-antibody conjugates, and RNA-peptide conjugates.

Following the conjugation reaction, the conjugate can be isolated by a variety of methods familiar to those skilled in the art. For example, the reaction mixture can be applied to a column chromatography system and separated by size-exclusion. Furthermore, the entry of conjugates (e.g., targeting moiety-INAS conjugate) into either tumor targets or immune cells may be facilitated by any method, including receptor-mediated endocytosis or electroporation.

B. Screening

Another aspect of the invention is directed to method of screening for biologically active agents to determine if such test agents are immunostimulatory. In general such screening methods provide a means for determining which agents and to what level such agents are immunostimulatory. Such agents can be any nucleic acid molecule, peptide or polypeptide which are coupled to a targeting moiety of the invention, which are described herein (e.g., antibody, aptamer, peptide). In various embodiments of the invention, a targeting moiety and a biologically active agent can be directly conjugated, coupled through any convention method, or coupled via a linker which can be a peptide or nucleic acid linker.

For example, markers can be screened before/after administration of a test agent to determine DNA damage or cell stress. For example, DNA double stranded breaks may occur and can be assayed. Cells react to DSBs by mounting a range of responses, including the activation of DNA repair mechanisms and the triggering of checkpoint events whose primary function is to halt or slow cell cycle progression until the DNA damage has been removed (Shiloh, Y. Nature Reviews Cancer 3, 155-68 (2003), Nyberg, K. A. et al Annu Rev Genet. 36, 617-56 (2002), Khanna & Jackson Nat. Genet. 27 247-254 (2001)).

For example, cells can be assayed for increased activity of ATM or ATR kinases. Treatment of human cells with IR leads to the rapid activation of the DNA-damage transducer protein kinases ATM and ATR. These kinases then phosphorylate and activate a series of downstream targets, including the effector protein kinases CHK1 and CHK2, and the checkpoint mediator proteins 53BP1 and MDC1. In addition, ATM and ATR phosphorylate the histone variant H2AX on Ser-139; this response can be detected within a minute of IR exposure and eventually extends over a large domain of chromatin flanking the site of DNA damage. This evolutionarily conserved response can be triggered by as little as one DNA DSB (Chen, H. T. et al. Science 290, 1962-1964 (2000)) and is widely recognized as a specific and unequivocal marker for the in vivo generation of this type of damage. The phosphorylation of histone H2AX then facilitates the recruitment to sites of DNA damage of a series of checkpoint and DNA repair factors, including 53BP1, MDC1, the MRE11/RAD50/NBS1 complex and the phosphorylated form of the structural maintenance of chromosomes 1 (SMC1) protein. The formation of these foci at sites of DNA DSBs is characteristic feature of the checkpoint response (Goldberg, M. et al. Nature 421, 952-6 (2003)). The foregoing is but one example of the various markers that can be screened in methods of assaying one or more biologically active agent using the compounds and methods of the instant invention.

For example, in methods of screening a test agent for effects on a cell (e.g., apoptosis inducing agent) a checkpoint response polypeptide can be assayed (e.g., immunochemistry or PCR for expression/protein activity). Such polypeptides are active in mediating the activation of a cell cycle checkpoint in response to DNA damage, in particular double strand breaks i.e. a polypeptide which is component of the DNA damage checkpoint response pathway. Suitable polypeptides include ATM, ATR, ATRIP, CHK1, CHK2, BRCA1, NBS1, RAD50, MRE11, CDC25C, 14-3-3.sigma., CDK2/cyclin E, CDK2/cyclin B153BP1, MDC1, histone variant H2AX, SMC1, RAD17, RAD1, RAD9, HUS1 and MRC 1. The DNA damage checkpoint response as described herein includes both ATM and ATR dependent signalling pathways.

The phosphorylation of a DNA damage checkpoint pathway polypeptide may be indicative of its activated state. Activity may also therefore be determined by determining the phosphorylation of a DNA damage checkpoint pathway polypeptide. DNA damage checkpoint pathway polypeptides which are activated by phosphorylation include ATRIP, CHK1, CHK2, BRCA1, NBS1, RAD50, MRE11, CDC25C, 14-3-3.sigma., CDK2/cyclin E, CDK2/cyclin B1 53BP1, MDC1, histone variant H2AX, SMC1, RAD17, RAD1, RAD9, HUS1 and MRC1.

The nucleic acid and protein sequences of various components of the DNA damage checkpoint pathway in humans and yeast are available from the GenBank database, under the following accession numbers: Human ATM (Nucleic acid coding sequence (CDS): W82828, protein sequence: AAB65827, Human CHK1 (CDS: AF016582, protein: AAC51736), Human CHK2 (CDS: NM.sub.—007194, protein: 096017), NBS1 (CDS: AF3169124, protein: BAA28616), Human RAD50 (CDS: 5032016, protein: NP.sub.—005723), MRE11 (CDS: U37359, protein: AAC78721), BRCA1 (CDS: U14680, protein: A58881), ATR, (CDS: NM.sub.—001184, protein: NP.sub.—001175) ATRIP (CDS: AF451323, protein: AAL38042.1), CDC25C (CDS: NM.sub.—001790, protein: NP 001781.1), 53BP1 (CDS: NM.sub.—005657, protein: NP.sub.—005648), MDC1 (CDS: NM.sub.—014641 protein: NP.sub.—055456), histone variant H2AX (CDS: NM.sub.—002105, protein: NP.sub.—002096), SMC1 (CDS: NM.sub.—006306, protein: NP.sub.—006297), RAD17 (CDS: NM.sub.—133338, protein: NP.sub.—579916), RAD1 (CDS: NM.sub.—002853, protein: NP.sub.—002844), RAD9 (CDS: NM.sub.—004584, protein: NP.sub.—004575), HUS1 (CDS: NM.sub.—148959, protein: NP.sub.—683762) and NMRCI (CDS: NM.sub.—002438, protein: NP.sub.—002429).

Furthermore, screening methods of the invention can comprise assaying activity of immune stimulatory compounds. For example, immunostimulatory activity may arise from the stimulation of Interferons, IL-12, NKG2D ligands, IL-15, and IL-2 by dendritic cells. This leads to the stimulation of NK cells to produce IFN-.gamma. and induces the development of CD⁴+ Th1 cells. The induced Th1 cells then produce IFN-.gamma. and IL-2. The IL-2 then enhances further proliferation of Th1 cells and the differentiation of antigen (e.g. tumour and pathogen)-specific CD8+ T cells. The IL-2 and IFN also stimulates the cytolytic activity of NK cells of the innate immune system.

In other embodiments of the assay methods described herein, an immunostimulatory response in cells or animals is determined by assaying the response of immune cells to contact with one or more test compounds. Thus, pro-inflammatory or immunestimulatory factors can be assayed. For example, it is known that IL-12 is the primary mediator of type-1 immunity (the Th1 response). It induces natural killer (NK) cells to produce IFN-γ as part of the innate immune response and promotes the expansion of CD4+ Th1 cells and cytotoxic CD8+ cells which produce IFNγ. It therefore increases T-cell invasion of tumours as well as the susceptibility of tumour cells to T-cell invasion.

Thus, if a test compound is assayed using a method of the invention and is determined to be a stimulator of cytokine secretion, for example, it is determined to be immunostimulatory. Particularly preferred are compounds which induce, potentiate, activate or stimulate the release one or more cytokines (for example Th1 cytokines, e.g. IFN, IL-12 and/or IL-2, optionally together with one or more other cytokines) in vitro. Such an immunomodulatory activity of a test compound is particularly important in certain medical applications. For example, increased production of IFNs and IL-12 may overcome the suppression of innate and cellular immunities observed in immune escape by cancer cells.

Furthermore, cytokine stimulation exhibited may be dependent, in whole or in part, on the presence of co-stimulatory agents. Such co-stimulatory agents may include, for example, agents that stimulate the innate immune system, including Toll-like receptor (TLR) ligands.

In various embodiments of the invention, the methods for screening a test agent for immunostimulatory activity comprise contacting a cell with a conjugate of the invention (including multivalent conjugates) to determine whether the biologically active agent. In any of such embodiments, the biologically active agent are administered to cells and a resulting readout provides information as to whether the test agent (e.g., nucleic acid molecule, peptide, polypeptide) results in cell stress (e.g., DNA damage), apoptosis, physical stress, cell hyperfusion, or increased expression of cell stress associated markers.

In another embodiment, a readout is provided by a marker present on the test conjugate (e.g., fluorescence or radioisotope marker), wherein the readout provides information as to whether the test conjugate is taken up by target cells (e.g., immune cells such as dendritic cells, macrophages), of whether the test agent induces immune cell activity (e.g., NK activity, co-stimulatory receptor expression; immune cell engagement such as through CD40, B7 family, CD86/CD83, MHC expression, cytokine release, pro-inflammatory response, etc.). Such markers for immune activity are known and can be measured using conventional techniques such as ELISA, immunochemistry (See, e.g., CURRENT PROTOCOLS IN IMMUNOLOGY (Coligan, John E. et. al., eds. 1999). See also, U.S. Patent Publication Nos. 20070155814, 20070135372, or 20070134261.

For example, cells (e.g., dendritic cell, tumor cell) can be contacted in culture with a compound comprising a targeting moiety which specifically binds a component present on such cells. The compound also comprise one or more test agent (e.g., nucleic acid or peptide) and one or more detectable labels (e.g., fluorescent or radiolabel). The cells can be examined under a microscope to determine if the tagged marker is observed in the cells (e.g. uptake) thus determining whether the test agent is capable of traversing the cell membrane (e.g., endocytosis).

In other embodiments, one or more test agent is administered to an non-human animal to determine the immunostimulatory effects. For example, a tumor transplanted into the flanks of a mouse using conventional techniques can be targeted by a test conjugate (e.g., with an antibody specific for a tumor cell antigen) and the tumor can be allowed to take, before administering the test conjugate systemically through the tail vein or directly by injecting into the tumor. Subsequently, markers for immunoactivation can be assessed to determine whether the test agent induces an immune response. Depending on the markers expressed, the screening methods of the invention can be used to determine whether a test agent is a PAMP, DAMP (e.g., LL37), alarmin inducing agent, a Toll-like receptor(TLR)-independent manner; a TLR-dependent activator (e.g., TLR3, 7, 8 or 9); an agent which activates death signaling or inhibits survival gene expression; or an agent which indirectly induces an immune response by causing cell stress/damage.

Test agents can be any nucleic acid molecule, including plasmid, ODN, RNA, DNA, ssRNA, ssDNA, dsDNA, RNA-DNA hybrid, PNA, peptide or polypeptide. In various embodiments, a multivalent compound comprising one or more test agents can be a administered, wherein such a compound also comprises a targeting moiety of the invention binding a specific target cell (e.g., in vitro or in vivo). For example, in the case of multivalent conjugates of the invention, two or more combination of different test agents can be screened to determine if a synergistic effect is observed. Furthermore, two or more compounds each comprising a targeting moiety to the same (or different) cell component can be used in the screening or therapeutic methods of the invention. In yet further embodiments, two or more compounds each comprising a targeting moiety the same or different comprises a test agent that is the same or different. For example, a first compound comprises targeting moiety a, while a second compound comprises targeting moiety b, while the first compound comprises a test agent x and the second compound comprises a test agent y. In other words, multiple test agents in various combinations of targeting moieties and test agents can be utilized in screening or therapeutic methods of the invention.

Of course, in further embodiments, the test conjugates can be screened along with one or more pharmaceutical compounds to determine the synergistic effect of such conjugates in combination with one or more such compounds in inducing an immunostimulatory response, to reduce or eliminate tumor cell growth or proliferation. As discussed above, where markers are used to “tag” a test conjugate, entry into the cell can be determined and/or measured.

In various embodiments, measurements of markers associated with immunostimulation can be made my conventional amplification (e.g., PCR, RT-PCR). Various commercially available reagents are available for RT-PCR, such as One-step RT-PCR reagents, including Qiagen One-Step RT-PCR Kit and Applied Biosystems TaqMan One-Step RT-PCR Master Mix Reagents kit. Such reagents can be used to determine the modulation of expression levels of marker genes associated with an immune response in control cells/animals versus cells/animals contacted with one or more test compounds described herein.

Furthermore, in some embodiments, a test agent may be a plasmid replicon (e.g., capable of expressing a peptide/protein encoded by a nucleic acid sequence). Thus, such a plasmid can express a “tagged” protein which is detectable and/or quantifiable.

Detectable labels (also referred to as markers) which can be coupled to compounds of the invention and utilized in cell culture or in vivo methods of the invention include but are not limited to include, chromophores, electrochemical moieties, enzymes, radioactive moieties, phosphorescent groups, fluorescent moieties, chemiluminescent moieties, or quantum dots, or more particularly, radiolabels, fluorophore-labels, quantum dot-labels, chromophore-labels, enzyme-labels, affinity ligand-labels, electromagnetic spin labels, heavy atom labels, probes labeled with nanoparticle light scattering labels or other nanoparticles, fluorescein isothiocyanate (FITC), TRITC, rhodamine, tetramethylrhodamine, R-phycoerythrin, Cy-3, Cy-5, Cy-7, Texas Red, Phar-Red, allophycocyanin (APC), epitope tags such as the FLAG or HA epitope, and enzyme tags such as alkaline phosphatase, horseradish peroxidase, I²-galactosidase, alkaline phosphatase, β-galactosidase, or acetylcholinesterase and hapten conjugates such as digoxigenin or dinitrophenyl, or members of a binding pair that are capable of forming complexes such as streptavidin/biotin, avidin/biotin or an antigen/antibody complex including, for example, rabbit IgG and anti-rabbit IgG; fluorophores such as umbelliferone, fluorescein, fluorescein isothiocyanate, rhodamine, tetramethyl rhodamine, eosin, green fluorescent protein, erythrosin, coumarin, methyl coumarin, pyrene, malachite green, stilbene, lucifer yellow, Cascade Blue, dichlorotriazinylamine fluorescein, dansyl chloride, phycoerythrin, fluorescent lanthanide complexes such as those including Europium and Terbium, Cy3, Cy5, molecular beacons and fluorescent derivatives thereof, a luminescent material such as luminol; light scattering or plasmon resonant materials such as gold or silver particles or quantum dots; or radioactive material include ¹⁴C, ¹²³I, ¹²⁴I, ¹²⁵I, ¹³¹I, Tc99m, ³⁵S or ³H intercalating dyes such as phenanthridines and acridines (e.g., ethidium bromide, propidium iodide, hexidium iodide, dihydroethidium, ethidium homodimer-1 and -2, ethidium monoazide, and ACMA); some minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine; cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red). See, e.g., Principles of Fluorescence Spectroscopy, Joseph R. Lakowicz (Editor), Plenum Pub Corp, 2nd edition (July 1999) and the 6^{th} Edition of the Molecular Probes Handbook by Richard P. Hoagland; See also, U.S. Pat. No. 6,207,392.

In one embodiment, a method of identifying a conjugate of the present invention which induces cell death, cell maturation, and/or NKG2D ligand dependent signaling is disclosed including, contacting one or more cells in vitro with a test conjugate containing an antibody that specifically binds to a cellular component of a tumor cell, tumor vasculature, and/or a component of a tumor microenvironment or an integrin derived peptide containing an RGD motif or a CDGRC motif, where the antibody or peptide is conjugated to a nucleic acid comprising one or more immunostimulatory nucleic acid sequences, and where one or more of the nucleic acid sequences comprise a pathogen-associated molecular pattern (PAMP) and determining induction of a marker or a phenotypic change in the one or more cells in the presence or absence of immune cells, where the determined induction or change in the presence of the test nucleic acid conjugate in one or more cells is indicative of cell death signaling, cell maturation, and/or NKG2D ligand dependent signaling. For example, if contacting causes (a) cells to fuse in the absence of immune cells, where the cells are tumor cells, (b) tumor cells to lyse in a mixture of PBMC cells and tumor cells, and (c) the induction of expression of one or more markers, which include, but are not limited to, CD86, IFN-γ, and/or Apo2L/TRAIL, where the cells are PBMC or dendritic cells (DC), the test conjugate is associated with the induction of cell death signaling, cell maturation, and/or NKG2D ligand dependent signaling.

Induction of expressed markers may be accomplished by cell sorting. Further, cells are obtained from the bone marrow of a non-fetal animal, including, but not limited to, human cells. Fetal cells may also be used.

Cell sorting may be by any method known in the art to sort cells, including sorting by fluorescent activated cell sorting (FACS) and Magnetic bead cell sorting (MACS). To sort cells by MACS, one labels cells with magnetic beads and passes the cells through a paramagnetic separation column. The separation column is placed in a strong permanent magnet, thereby creating a magnetic field within the column. Cells that are magnetically labeled are trapped in the column; cells that are not pass through. One then elutes the trapped cells from the column.

In one embodiment, an antibody-nucleic acid conjugate is disclosed including an antibody that specifically binds to a cellular component of a tumor cell, tumor vasculature, and/or a component of a tumor microenvironment. A tumor microenvironment may contain epithelial cells, basement membrane, fibroblasts, stromal cells, and/or myofibroblasts, which surround the tumor. In a further related aspect, such cells surrounding the tumor may express functional CLIC4. Further, the conjugate has a binding affinity of at least 1 nM to 20 nM, including that such conjugate triggers cell hyperfusion between tumor cells in vitro subsequent to binding of the cellular component of the tumor cells.

C. Treatment

In general the compositions and methods of the invention are directed to preventing or treating cancer or an infectious disease. In various aspects of the invention, the compositions of the invention comprising one or more targeting moiety coupled to one or more biologically active agent are administered to a cell to prevent, reduce or eliminate a neoplasm. In other aspects of the invention, the compositions of the invention comprising one or more targeting moiety coupled to one or more biologically active agent are administered to a cell to prevent, reduce or eliminate a disease or condition caused by an infectious agent. In some embodiments, compositions of the invention are administered alone, or in combination with other therapeutics to treat a subject suffering a neoplastic disease or infectious disease, which are described herein.

For example, in various embodiments, an antibody or functional fragment thereof, an polypeptide (e.g., antibody), aptamer or ligand which specifically targets such cellular components is administered to prevent or treat cancer, wherein such a composition comprises the targeting moiety as well as one or more biologically active components of the invention.

According to yet another aspect of the invention, there is provided the use of a compound (conjugate) comprising one or more targeting moiety coupled to one or more biologically active agent (as defined above) for the manufacture of a product for the diagnosis, detection and/or imaging, and/or a medicament for the prevention and/or treatment of a disease or condition. Such diseases or conditions include but are note limited to an immune disorder, inflammatory disease, infectious disease, and neoplastic disease/cancer, including, but not limited to head and neck cancers, aero-digestive cancers, gastro-intestinal cancers, esophageal cancers, stomach/gastric cancers, pancreatic cancers, hepato-biliary/liver cancers, colorectal cancers, anal cancers, small intestine cancers, genito-urinary cancers, urologic cancers, renal/kidney cancers, bladder, ureter cancers, testicular cancers, urethra/penis cancers, gynecologic cancers, ovarian/fallopian tube cancers, peritoneal cancers, uterine/endometrial cancers, cervical/vagina/vulva cancers, gestational trophoblastic disease, prostate cancers, bone cancers, sarcoma (soft tissue/bone), lung cancers (e.g., non-small cell lung, small-cell lung), mesothelioma, mediastinum cancers, breast cancers, central nervous system cancers, brain cancers, melanoma, hematologic malignancies, leukemia, lymphoma (Hodgkin's Disease and Non-Hodgkin's lymphoma), retinoblastoma, astrocytoma, glioblastoma, plasma cell neoplasms, myeloma, myelodysplastic syndrome, endocrine tumors, skin cancers, melanoma, thyroid cancers, parathyroid cancers, adrenal, pancreatic endocrine cancers, carcinoid, multiple endocrine neoplasia, AIDS-related malignancies, cancer of unknown primary site, and various childhood cancers. The cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a brain cancer cell, an ovarian cancer cell, a lymphatic cancer cell, a skin cancer cell, a brain cancer cell, a bone cancer cell, or a soft tissue cancer cell.

Examples of pathogens and infectious agents which cause disease are known and disclosed herein.

In one aspect, the conjugates of the present invention are used alone or in combination with other anticancer such as chemotherapeutic agents, ionizing radiation, hormonal therapy, cytokines, immunotherapy, cellular therapy, vaccines, monoclonal antibodies, antiangiogenic agents, targeted therapeutics (small molecule drugs), or biological therapies. For example, chemotherapeutic agents include, but are not limited to, antitumor alkylating agents such as Mustards (mechlorethamine HCl, melphalan, chlorambucil, cyclophosphamide, ifosfamide, busulfan), Nitrosoureas (BCNU/carmustine, CCNU/lomustine, MeCCNU/semustine, fotemustine, streptozotocin), Tetrazines (dacarbazine, mitozolomide, temozolomide), Aziridines (thiotepa, mitomycin C, AZQ/diaziquone), procarbazine HCl, hexamethylmelamine, adozelesin; cisplatin and its analogues, cisplatin, carboplatin, oxaliplatin; antimetabolites, methotrexate, other antifolates, 5-fluoropyrimidines (5-fluorouracil/5-FU), cytarabine, azacitidine, gemcitabine, 6-thiopurines (6-mercaptopurine, thioguanine), hydroxyurea; topoisomerase interactive agents epipodophyllotoxins (etoposide, teniposide), camptothecin analogues (topotecan HCl, irinotecan, 9-aminocamptothecin), anthracyclines and related compounds (doxorubicin HCl, liposomal doxorubicin, daunorubicin HCl, daunorubicin HCl citrate liposomal, epirubicin, idarubicin), mitoxantrone, losoxantrone, actinomycin-D, amsacrine, pyrazoloacridine; antimicotubule agents Vinca alkaloids (vindesine, vincristine, vinblastine, vinorelbine), the taxanes (paclitaxel, docetaxel), estramustine; fludarabine, 2-chlorodeoxyadenosine, 2′-deoxycoformycin, homoharringtonine, suramin, bleomycin, L-asparaginase, floxuridine, capecitabine, cladribine, leucovorin, pentostatin, retinoids (all-trans retinoic acid, 13-cis-retinoic acid, 9-cis-retinoic acid, isotretinoin, tretinoin), pamidronate, thalidomide, cyclosporine; hormonal therapies antiestrogens (tamoxifen, toremifene, medroxyprogesterone acetate, megestrol acetate), aromatase inhibitors (aminoglutethimide, letrozole/femara, anastrozole/arimidex, exemestane/aromasin, vorozole), gonadotropin-releasing hormone analogues, antiandrogens (flutamide, casodex), fluoxymeterone, diethylstilbestrol, octreotide, leuprolide acetate, zoladex; steroidal and non-steroidal anti-inflammatory agents (dexamethasone, prednisone); Monoclonal antibodies including, but not limited to, anti-HER2/neu antibody (herceptin/trastuzumab), anti-EGFR antibody (cetuximab/erbitux, ABX-EGF/panitumumab, nimotuzumab), anti-CD20 antibody (rituxan/rituximab, ibritumomab/Zevalin, tositumomab/Bexxar), anti-CD33 antibody (gemtuzumab/MyloTarg), alemtuzumab/Campath, bevacizumab/Avastin; and small molecule inhibitors.

In one aspect, the conjugates of the present invention are used in combination with adjunctive therapies designed to induce tumor cell death and/or inhibit tumor growth including, but not limited to chemotherapy, radiation, death ligands, antibodies, cryotherapy, radiofrequency ablation, toxins, electroporation, viral gene therapy, non-viral gene therapy, plasmids, vaccines, nanoparticles, aptamers, peptides/peptidomimetics, hormonal therapy, cytokines, bacteriotherapy, other cancer therapeutics.

In one aspect, conjugates of the present invention are used in combination with adjunctive therapies designed to break tolerance to tumor antigens/cells and/or amplify immune responses against tumor cells and/or increase immune-mediated death of tumor cells, such as: (a) allogeneic or autologous cellular therapy with one or more of the following: allogeneic or autologous T cells; allogeneic or autologous dendritic cells (DCs); allogeneic or autologous NK cells; and/or (b) vaccines (e.g., against tumor or pathogen); and/or (c) depletion or inactivation of T regulatory/suppressor cells (via antibody, e.g. anti-CD25; chemotherapy; modulation of polarization e.g. GATA3 inhibition; indoleamine 2,3-dioxygenase (IDO) inhibition; TLR agonists; or other methods); and/or (d) delivery or expression of cytokines or co-stimulatory molecules or other immunostimulatory agents that enhance immune response (flt-3 ligand, IL-12, GM-CSF, CD40L, B7-1, IL-2, TLR agonists, alarmins, PAMPs, DAMPs); and/or (e) administration of antibodies that enhance the immune response (e.g. anti-CTLA-4, anti-41BB, anti-CD28, anti-CD40, anti-B7 family); and/or (f) administration of antibodies against tumor cells, tumor vasculature, or the tumor microenvironment (e.g. antibodies targeting various tumor- or tumor-associated antigens or receptors; conjugated antibodies); and/or (g) administration of any agent which can modify tumor gene expression or target cell signaling including signal transduction inhibitors (STI), demethylating agents (e.g. azacytidine), histone deacetylase (HDAC) modulators.

In one aspect of the invention, one or more active agents (as defined above) are administered before, after or concurrent to administration of the targeting-therapeutic conjugates described herein. In such embodiments, the one or more active agents may increase tumor cell death, inhibit tumor growth, and/or enhance antitumor immune responses.

For example, in one embodiment, one or more active agent is an inhibitor of indoleamine 2,3-dioxygenase (IDO). Inhibitors of IDO can be on the enzymatic level, such as small molecule inhibitors that block the active site or bind the active site of the enzyme. Alternatively, inhibitors can function on the gene expression level, such as targeting with antisense, siRNA or ribozymes to reduce IDO activity. Therefore, in various embodiments, a therapeutic of the invention (e.g., antibody-INAS conjugate) is administered with any IDO inhibitor, whereby administration is sequential in any order or concurrent.

The extrahepatic enzyme indoleamine 2,3-dioxygenase (IDO) catalyzes tryptophan degradation in the first and rate-limiting step towards biosynthesis of the central metabolic co-factor nicotinamide adenine dinucleotide (NAD). IDO was implicated with an immunological role with the observation that IDO expression is stimulated by interferon-gamma and subsequently confirmed by the discovery of its physiological importance in protecting the fetus from maternal immunity. IDO, which is commonly elevated in tumors and draining lymph nodes, suppresses T cell immunity in the tumor microenvironment. In cancer, IDO activity may help promote acquired tolerance to tumor antigens. By creating peripheral tolerance to tumor antigens, IDO can undermine immune responses that thwart tumor cell survival in the context of an underlying inflammatory environment that facilitates tumor outgrowth. In preclinical studies, small molecule inhibitors of IDO compromise this mechanism of immunosuppression and strongly leverage the efficacy of a variety of classical chemotherapeutic agents, supporting the clinical development of IDO inhibitors as a therapeutic goal.

The IDO inhibitor 1-methyl-tryptophan is being developed for clinical trials. Hou et al. Cancer Res. 2007 Jan. 15; 67(2):792-801. As shown by Hou et al. the D isomer of 1-methyl-tryptophan specifically targeted the IDO gene because the antitumor effect of D-1-methyl-tryptophan was completely lost in mice with a disruption of the IDO gene (IDO-knockout mice). Therefore, in various embodiments, either the D or L isomer, preferrably the D-1-methyl-tryptophan is administered to effect IDO inhibition and to block host-mediated immunosuppression and enhance antitumor immunity in the setting of combined with therapeutics of the present inventions.

Furthermore, in other embodiments combination administration can further include targeting upstream activators of IDO activity so as to reduce or eliminate IDO activity by precluding activation of IDO expression. For example, IDO is induced by interferon (IFN)-γ-mediated effects of the signal transducer and activator of transcription 1α (STAT1α) and interferon regulatory factor (IRF)-1. The induction of IDO can also be mediated through an IFN-γ-independent mechanism, although the mechanism of induction has not been identified. Therefore, small molecule inhibitors, or knock-down nucleic acids targeting upstream activators of IDO expression provide additional targets for enhancing the anti-cancer effects of the compositions and methods of the present invention. In a related aspect, conjugates of a targeting moiety with immunostimulatory siRNA targeting IDO (INAS) may be used to enhance antitumor immunity.

Therefore, the compositions and methods of the invention can be utilized in combination with one or more other active agents, including small molecule inhibitors and as well compounds preventing IDO expression and/or activity. Such active agents are contemplated to be administered with therapeutic compositions and methods of the invention. Such active agents and methods of use thereof are known, such as disclosed in U.S. Patent Applications 20070173524, 20070105907, 20070099844, 20070077234, 20060292618, 20060110371, 20050186289 and 20040294623.

According to yet another aspect of the invention, there is provided the use of a conjugate comprising one or more targeting moiety coupled to one or more biologically active agent (as defined above) for the manufacture of a product for the diagnosis, detection and/or imaging and/or a medicament for the prevention and/or treatment of an infectious disease caused by an infection selected from the group consisting of a microbial infection, fungal infection, parasitic infection, bacterial infection and viral infection.

The present invention also provides pharmaceutical compositions comprising at least one compound capable of treating a disorder in an amount effective therefor, and a pharmaceutically acceptable vehicle or diluent. The compositions of the present invention may contain other therapeutic agents as described, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Pharmaceutical compositions employed as a component of invention articles of manufacture can be used in the form of a solid, a solution, an emulsion, a dispersion, a micelle, a liposome, and the like, where the resulting composition contains one or more of the compounds described above as an active ingredient, in admixture with an organic or inorganic carrier or excipient suitable for enteral or parenteral applications. Compounds employed for use as a component of invention articles of manufacture may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used.

Invention pharmaceutical compositions may be administered by any suitable means, for example, orally, such as in the form of tablets, capsules, granules or powders; sublingually; buccally; parenterally, such as by subcutaneous, intradermal, intravenous, intramuscular, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present conjugates may also be administered liposomally. In one aspect, the composition may be administered systemically, intratumorally, or peritumorally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The subjects treated in the above methods, in which cells targeted for modulation is desired, are mammals, including, but not limited to, cows, sheep, goats, horses, dogs, cats, guinea pigs, rats or other bovine, ovine, equine, canine, feline, rodent or murine species, and preferably a human being, male or female.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release

Formulations for oral use may also be presented as hard gelatin capsules where the active ingredient is mixed with an inert solid diluent, for example, calcium carbonate, calcium phosphate or kaolin, or as soft gelatin capsules where the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In the treatment of a subject where cells are targeted for modulation, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In one embodiment, an aliquot of blood is extracted from a mammalian subject, preferably a human, and the aliquot of blood is treated ex vivo with the conjugate of the present invention. The effect of the conjugate is to modulate the activity of immune effector cells in the blood which are contained in the aliquot. The modified aliquot is then re-introduced into the subject's body by any route suitable for vaccination.

In one aspect, a method is disclosed including removing immune cells from a subject, contacting the cells with the conjugate ex vivo, and reintroducing the cells into the subject.

In one aspect, the volume of the aliquot is up to about 400 ml, from about 0.1 to about 100 ml, from about 5 to about 15 ml, from about 8 to about 12 ml, or about 10 ml, along with an anticoagulant (e.g., 2 ml sodium citrate).

In one aspect, the subject undergoes a course of treatments, such individual treatments comprising removal of a blood aliquot, treatment thereof as described above and re-administration of the treated aliquot to the subject. A course of such treatments may comprise daily administration of treated blood aliquots for a number of consecutive days, or may comprise a first course of daily treatments for a designated period of time, followed by an interval and then one or more additional courses of daily treatments.

In a related aspect, the subject is given an initial course of treatments comprising the administration of 4 to 6 aliquots of treated blood. In another preferred embodiment, the subject is given an initial course of therapy comprising administration of from 2 to 4 aliquots of treated blood, with the administration of any pair of consecutive aliquots being either on consecutive days, or being separated by a rest period of from 1 to 21 days on which no aliquots are administered to the patient, the rest period separating one selected pair of consecutive aliquots being from about 3 to 15 days. In another related aspect, the dosage regimen of the initial course of treatments comprises a total of three aliquots, with the first and second aliquots being administered on consecutive days and a rest period of 11 days being provided between the administration of the second and third aliquots.

In a further related aspect, additional courses of treatments following the initial course of treatments. For example, subsequent courses of treatments are administered at least about three weeks after the end of the initial course of treatments. In one aspect, the subject receives a second course of treatment comprising the administration of one aliquot of treated blood every 30 days following the end of the initial course of treatments, for a period of 6 months.

It will be appreciated that the spacing between successive courses of treatments should be such that the positive effects of the treatment of the invention are maintained, and may be determined on the basis of the observed response of individual subjects.

The following examples are intended to illustrate but not limit the invention.

### EXAMPLES

### Example 1

### Generation of Conjugated Antibodies or Peptides

Conjugation of nucleic acid sequences (DNA or RNA) to anti-human EGFR antibody, Anti-human HER2 antibody, and Anti-murine neu antibody:

Antibodies:
(1) anti-human EGFR antibody (chimeric)
(2) anti-human HER2/neu antibody
(3) anti-murine neu antibody

DNA Sequences:
(1) Oligodeoxynucleotide (ODN)— (SEQ ID NO: 1)
5′ G*G*G GAC GAC GTC GTG G*G*G*G*G*G-3′phosphate
(*phosphorothioate bonds, rest are phosphodiester bonds)
Type=DNA-PS; Size=21; Epsilon 1/(mMcm)=208;

MW (g/mole)=6842 CpG A; Class=CpG A; 21.92 μM

Oligodeoxynucleotide (ODN)— (SEQ ID NO: 2)

5′ G*G*G GGA GCA TGC TGG*G*G*G*G*G-3′phosphate

(*phosphorothioate bonds, rest are phosphodiester bonds)

Type=DNA-PS; Size=20; Epsilon 1/(mMcm)=197.6;

MW (g/mole)=6553; Class=Non-CpG; 18.34 μM

Plasmid DNA

Plasmid DNA (an empty plasmid DNA vector) cut with DpnI+Hha into a size between 100 bp to 250 bp, denatured under 90 degrees C., and purified in phenol+chloroform as well as EtoH. The purified denatured plasmid DNA fragments were conjugated to the antibody as described below.

RNA Sequences:

| | |
|---|---|
| Oligoribonucleotide | (SEQ ID NO: 229) |

5′ phosphate GGG GAC GAC GUC GUG GGG GGG
(*phosphorothioate bonds - stable ss RNA)
siRNA

| | |
|---|---|
| 5′-UGUCCUUCAAUGUCCUUGAA | (SEQ ID NO: 85) |
| 5′-AAUUGUGUAAUGUCCUUCAA | (SEQ ID NO: 230) |

Tumor-targeting peptide sequences:

| | | |
|---|---|---|
| 1. | CDCRGDCFC (RGD-4C peptide); | (SEQ ID NO: 3) |
| (2) | GGCDGRCG | (SEQ ID NO: 4) |
| | CDGRC | (SEQ ID NO: 5) |

500 μl of antibody peptide solution was transferred into eppendorf tubes, to which 540 μl of 0.1M imidazole was added (i.e., 3M imidazole diluted in PBS to 0.1 M). 5 mg of 1-ethyl-3-[3-dimethylaminopropyl]oarbodiimide hydrochloride (EDC) was mixed with CpG DNA (ODN) in a separate tube, and immediately mixed with either antibody imidazole or peptide imidazole solution (Ab:ODN molar ratio=1:30.6).

The tubes were vortexed until the contents were dissolved, and the solution was briefly centrifuged. An additional 250 μl of 0.1 M imidazole was added subsequent to centrifugations, and the resulting solution was incubated at 50° C. for 2 hours.

The non-reacted EDC, its by-products, and imidazole was removed by CENTRICON® filtration (Millipore Corporation, Billerica, Mass.). The samples were then assayed by SDS-PAGE gels and mass spectrometry to determine conjugation of the nucleotide to the antibody and/or peptide. A protein assay was performed to quantify antibody or peptide concentration.

Method of conjugation of nucleic acids to antibody/targeting moieties (FIG. 4)

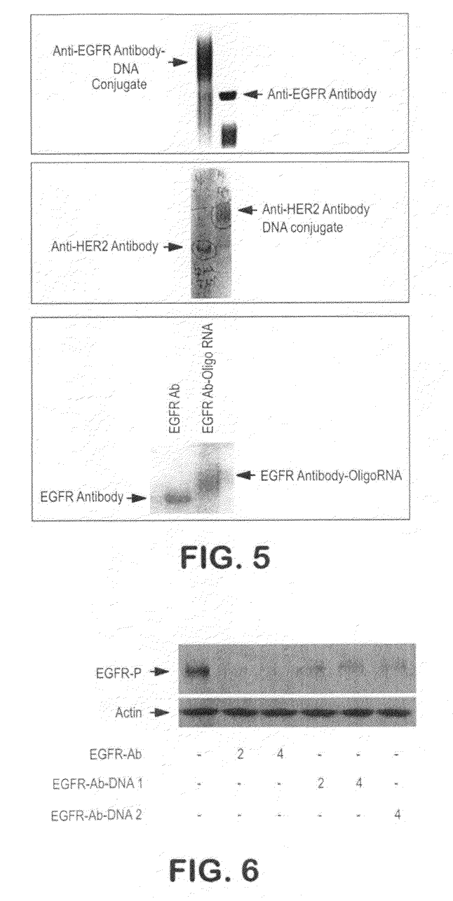

SDS-PAGE/immunoblotting demonstrated that the DNA- and RNA-conjugated monoclonal antibodies were in fact generated (FIG. 5).

### Example 2

### Inhibition of EGFR Activity by DNA-Conjugated Anti-EGFR Antibody

HT-29 colon carcinoma cells were cultured in 0.5% fetal bovine serum in the presence of either anti-EGFR antibody or DNA-conjugated anti-EGFR antibodies [anti-EGFR Ab-DNA 1 (SEQ ID NO:1) or anti-E3FR Ab-DNA 2 (SEQ ID NO:2), and then stimulated with EGF (5 ng/ml) for 20 minutes at 37° C. Cells were then washed with ice-cold PBS containing 1 mM sodium orthovanadate, and cell lysates were subjected to Western blot analysis using antibodies that detect phospho-specific EGFR (tyrosine 1068; Cell Signaling). Treatment of HT-29 cells with anti-EGFR antibody or DNA-conjugated anti-EGFR antibodies inhibited EGF-stimulated phosphorylation of EGFR (FIG. 6).

### Example 3

### Maturation of Dendritic Cells by DNA/RNA Conjugated Anti EGFR Antibody

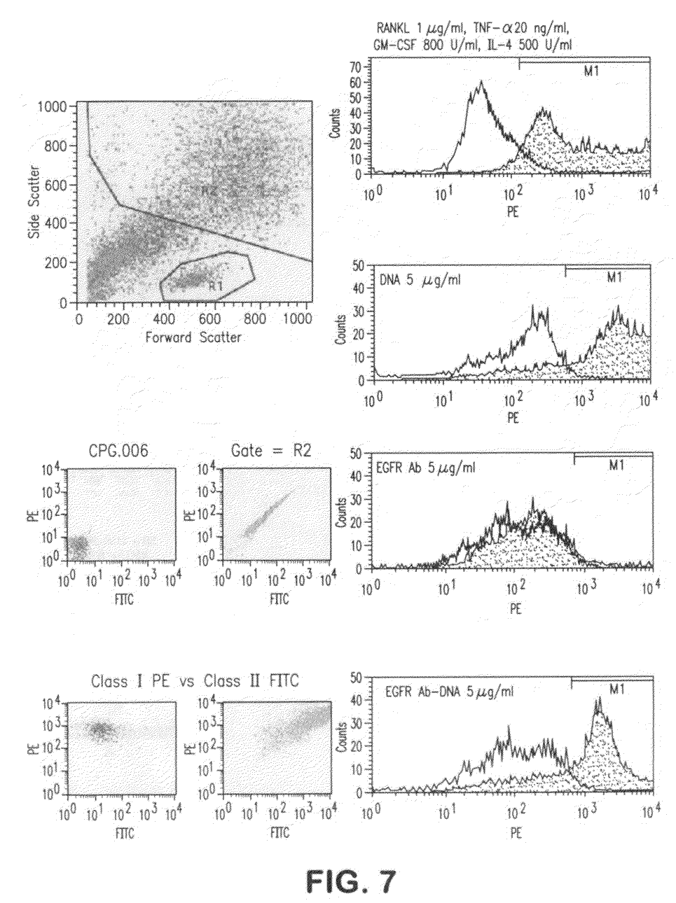

Human monocytes were isolated from bone marrow mononuclear cells and cultured for 6 days in AIM5 media (with 10% human AB serum) and either of the following: (1) combination of the following cytokines: RANKL 1 μg/ml+TNF-α 20 ng/ml+GM-CSF 800 U/ml+IL-4 500 U/ml; (2) oligodeoxynucleotide SEQ ID NO:1 (DNA)(5 μg/ml)(without cytokines; (3) DNA-conjugated anti-EGFR antibody (EGFR Ab-DNA)(5 μg/ml)(without cytokines). Cells were harvested on day 7 and stained with antibodies to MHC class I PE, MHC class II FITC, and CD86-PE. Maturation of dendritic cells (DCs) was assessed by flow cytometric analysis of increased cell surface expression of the maturation marker CD86. DNA-conjugated anti-EGFR antibody induced CD86 expression (i.e., maturation of DCs) that was similar to that observed in response to the cocktail of cytokines (FIG. 7). Analogous results were obtained with anti-EGFR Ab-DNA 2 (SEQ ID NO:2), anti-EGFR Ab-plasmid DNA, and anti-EGFR Ab-RNA conjugates.

### Example 4

### DNA-Conjugated Anti-EGFR Antibody or DNA-Conjugated Anti-HER2 Antibody Induce Expression of Cytokines [Interferon-γ (INF-γ) and Apo2L/TRAIL] by Human Peripheral Blood Mononuclear Cells (PBMCs)

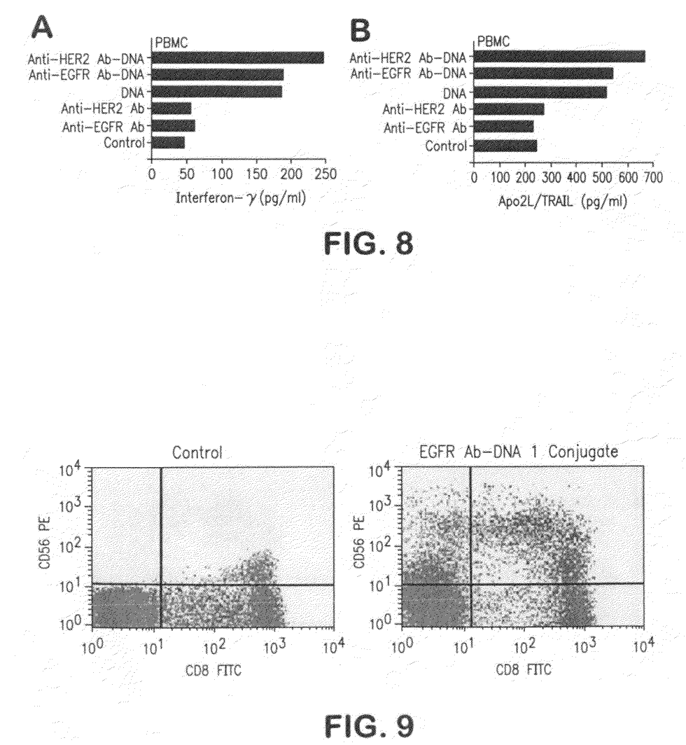

Human peripheral blood mononuclear cells (PBMCs) were treated with either anti-human EGFR antibody (anti-EGFR Ab) 5 μg/ml, anti-human HER2 antibody (anti-HER2 Ab) 5 μg/ml, oligodeoxynucleotide SEQ ID NO: 1 (DNA) 5 μg/ml, or DNA-conjugated antibodies [anti-EGFR antibody-DNA (anti-EGFR Ab-DNA) or anti-HER2 antibody-DNA (anti-HER2Ab-DNA) 5 μg/ml]. Levels of cytokines (INF-γ or Apo2L/TRAIL) in supernatants of PBMCs were assessed after 24 hours by ELISA (pg/ml). Treatment of PBMCs with either DNA (SEQ ID NO: 1) or DNA conjugated antibodies increased expression of soluble INF-γ or Apo2L/TRAIL in cell supernatants (FIG. 8). Analogous results were obtained with anti-EGFR Ab-DNA 2 (SEQ ID NO:2).

### Example 5

### Activation of Natural Killer Cells by DNA-Conjugated Anti-EGFR Antibody

Normal peripheral blood mononuclear cells (PBMCs)(Johns Hopkins leucopheresis Unit) were treated with either DNA-conjugated anti-EGFR antibody [anti-EGFR Ab-DNA 1 (SEQ ID NO: 1)] or EGFR Ab (Control) (4 μg/ml) for 3 d or left untreated. Cells were labeled with anti-CD56 phycoerythrin (CD56 PE) and anti-CD8 FITC (CD8 FITC) and then analyzed by flow cytometry. PBMCs showed increased numbers of CD56+ cells following stimulation with EGFR Ab-DNA 1 conjugate (FIG. 9).

### Example 6

### Increased MHC Expression by DNA- or RNA-Conjugated Anti-EGFR Antibody

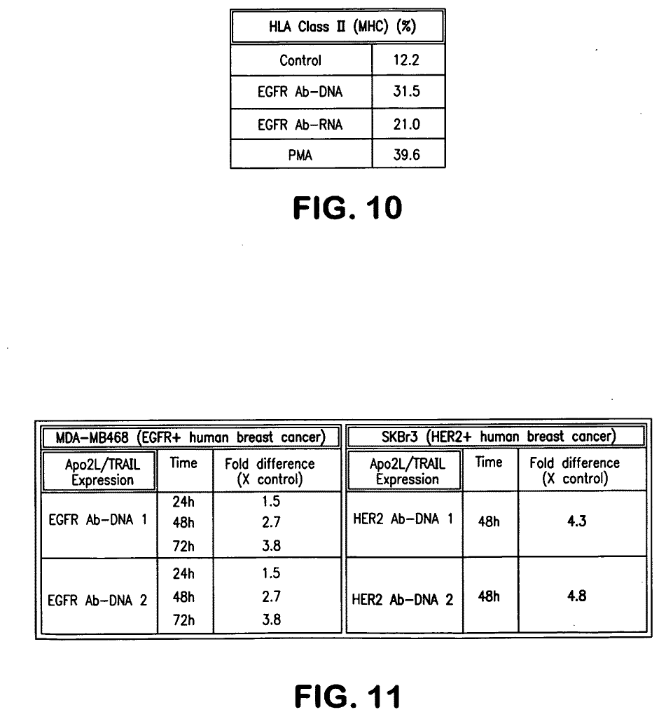

Normal peripheral blood mononuclear cells (PBMCs)(Johns Hopkins leucopheresis Unit) were treated with either DNA-conjugated anti-EGFR antibody [anti-EGFR Ab-plasmid DNA] or anti-EGFR Ab-RNA (SEQ ID NO:) or EGFR Ab (Control) (4 μg/ml) for 3 d or left untreated. Cells were labeled with anti-HLA class II (DR) and analyzed by flow cytometry. PBMCs showed increased percentage of DR⁺ cells following stimulation with EGFR Ab-plasmid DNA or EGFR Ab-RNA conjugates (FIG. 10).

### Example 7

### Induction of Apo2L/TRAIL in Tumor Cells in Response to DNA-Conjugated Anti-EGFR Antibody or DNA-Conjugated Anti-HER2 Antibody

EGFR-expressing MDA-MB468 cells were treated with EGFR antibody-DNA conjugates (EGFR Ab-DNA SEQ ID NO: 1 or EGFR Ab-DNA SEQ ID NO:2) or EGFR Ab (Control) (5 μg/ml) for 3 d. HER2-expressing SKBr-3 cells were treated with HER2 antibody-DNA conjugates (4ER2Ab-DNA SEQ ID NO: 1 or HER2Ab-DNA SEQ ID NO:2) or HER2Ab (Control) (5 μg/ml) for 3 d. Levels of Apo2L/TRAIL in cells was assessed after 24, 48, and 72 hours by quantitative PCR. Apo2L/TRAIL expression was induced in EGFR-expressing tumor cells (MDA-MB468) in response to treatment with EGFR antibody-DNA conjugates (EGFR Ab-DNA SEQ ID NO: 1 or EGFR Ab-DNA SEQ ID NO:2) and in HER2/neu-expressing tumor cells (SKBr-3) in response to treatment with HER2 antibody-DNA conjugates (HER2Ab-DNA SEQ ID NO: 1 or HER2Ab-DNA SEQ ID NO:2)(FIG. 11).

### Example 8

### DNA Conjugated Antibodies Directly Induce a Novel Form of Targeted Tumor Cell Death—Cell Hyperfusion—that is Not Observed in Response to Unconjugated Antibodies or Any Known Class of Anticancer Agents

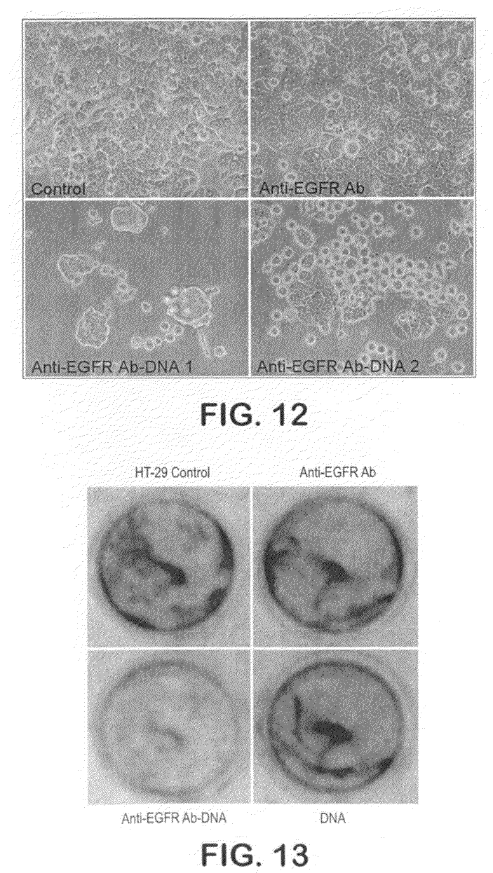

EGFR expressing human colon cancer cells (HT-29) were plated (5×10⁴ cells/ml) in the presence of either anti-EGFR antibody (anti-EGFR Ab) or EGFR antibody-DNA conjugates (EGFR Ab-DNA SEQ ID NO: 1 or EGFR Ab-DNA SEQ ID NO:2) or free oligodeoxynucleotide (DNA) (5 μg/ml). Cells were followed by phase-contrast and time lapse microscopy for 96 h. Treatment with either of the DNA-conjugated Anti-EGFR antibodies induced fusion of HT-29 cells and resulted in the formation of coalesced (hybrid or multinucleated) cells with a shorter lifespan and impaired replicating ability (hyperfusion) that was not observed with EGFR Ab or free DNA (FIG. 12). HT29 cell culture plates demonstrated the induction of direct death (with loss of colony formation) in response to treatment with either EGFR antibody-DNA conjugate but not with either EGFR antibody or unconjugated nucleic acid (FIG. 13).

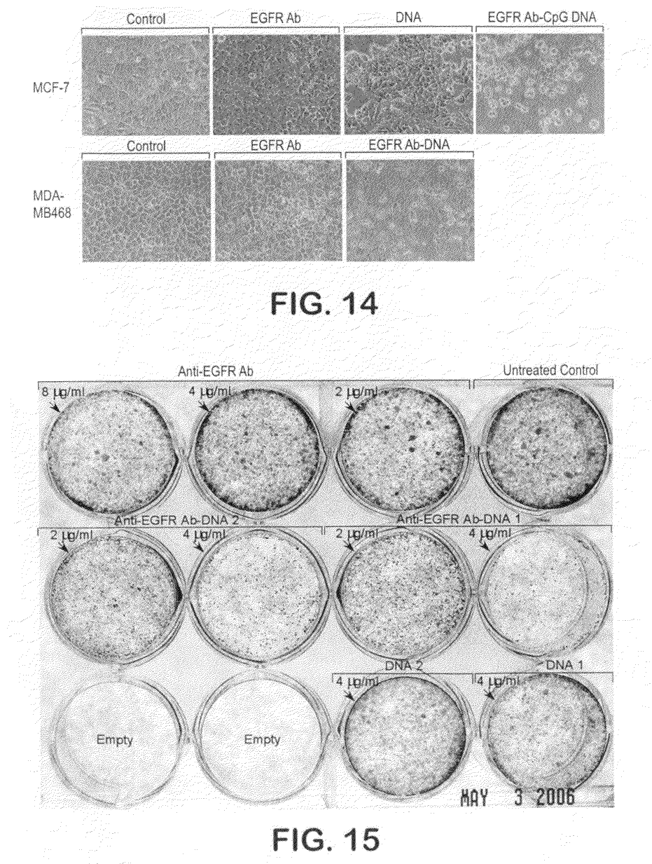

EGFR expressing human breast cancer cells (MCF-7 or MDA-MB-468) were plated (5×10⁴ cells/ml) in the presence of either anti-EGFR antibody (anti-EGFR Ab) (2-8 μg/ml) or DNA-conjugated anti-EGFR antibody (EGFR Ab-DNA SEQ ID NO: 1 or EGFR Ab-DNA SEQ ID NO:2) (2-4 μg/ml) or free oligodeoxynucleotide (DNA) (4 μg/ml). Treatment with either of the DNA-conjugated Anti-EGFR antibodies induced hyperfusion of breast cancer cells and formed coalesced cell-bodies with a shorter lifespan and replicating ability compared to cells that were treated with the parental (unconjugated) anti-EGFR antibody (FIG. 14). Cell culture plates demonstrated the induction of direct death (with loss of colony formation) in response to treatment with either of the EGFR antibody-DNA conjugates but not with either EGFR antibody or unconjugated nucleic acid (FIG. 15).

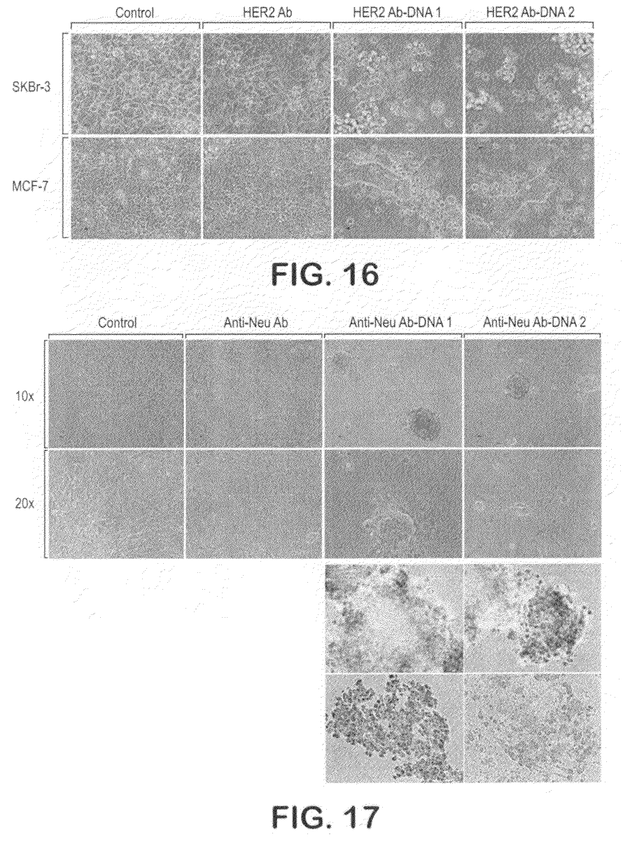

HER2/neu-expressing human breast cancer cells (SKBr or MCF-7) were plated (5×10⁴ cells/ml) in the presence of either anti-human HER2/neu antibody (anti-HER2/neu Ab) or DNA-conjugated anti-HER2/neu antibody (anti-HER2/neu Ab-DNA 1; SEQ ID NO: 1 or anti-HER2/neu Ab-DNA 2; SEQ ID:2)(5 μg/ml). Cell survival/proliferation was assessed by phase-contrast microscopy. Treatment with either of the DNA-conjugated Anti-HER2/neu antibodies induced hyperfusion of breast cancer cells and formed coalesced cell-bodies with a shorter lifespan and replicating abilities, which was not observed with cells treated by parental anti-HER2/neu antibody (FIG. 16).

Mouse neu-expressing breast cancer cells (NT2 cells) were plated (5×10⁴ cells/ml) in the presence of either anti-neu antibody (anti-neu Ab) or DNA conjugated anti-neu antibody (anti-neu Ab-DNA1; SEQ ID NO: 1)(5 μg/ml). Cell survival/proliferation was assessed by phase-contrast microscopy and trypan-blue dye exclusion assays. Treatment with DNA-conjugated anti-neu antibody induced hyperfusion of mouse neu-expressing breast cancer cells (NT2) and formed coalesced cell-bodies with reduced lifespan and replicating ability. Again, such hyperfusion and cell death was not induced by unconjugated antibody or DNA (FIG. 17).

### Example 9

### DNA-conjugated Anti-EGFR Antibody Induces Immune Cell-Mediated Lysis of EGFR-Expressing Tumor Cells

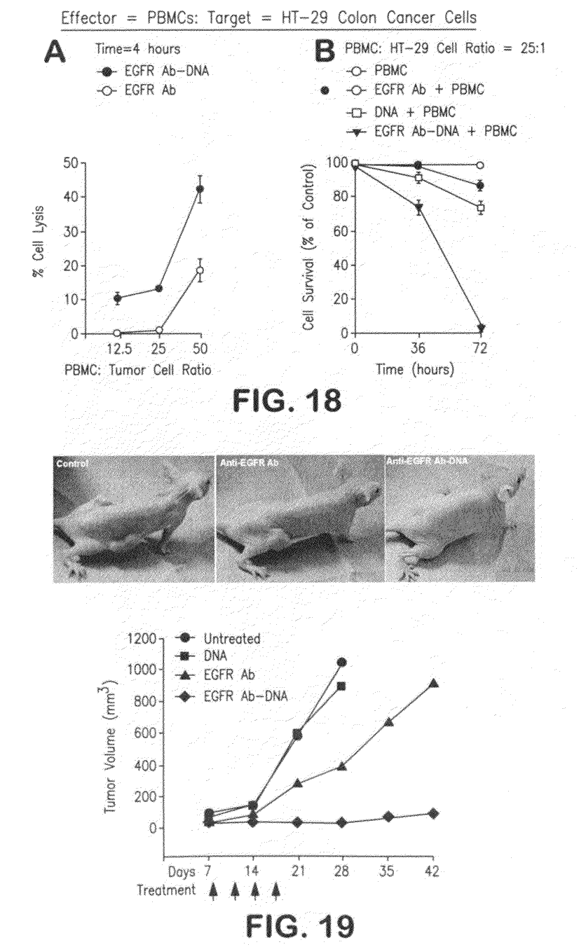

HT-29 colon carcinoma cells were labeled with ³H-thymidine (2.5 μCi/ml), trypsinized, washed with PBS, and treated with either EGFR-Ab or EGFR Ab-DNA 1 (SEQ ID NO: 1) or free DNA (4 μg/ml), were co-cultured in triplicate in 96-well plates (5×10³ cells/well) with PBMCs at varying E:T ratios at 37° C. for 4 h-72 h. Cells were harvested onto a filter paper and cell death/survival was quantified by percent specific ³H-thymidine release. Compared to EGFR-Ab, treatment with EGFR Ab-DNA resulted in more rapid death of HT-29 cells over 4 h (FIG. 18A). In contrast to treatment of HT-29 cells with either EGFR-Ab or DNA, culture of HT-29 cells with EGFR Ab-DNA resulted in elimination of HT-29 cells over 72 h (PBMC: tumor cell ratio=25) (FIG. 18B).

### Example 10

### DNA Conjugated Anti-EGFR Antibody Inhibits Growth of Human EGFR+Colon Cancer Xenografts in Nude Mice

BALB/c nude mice were injected subcutaneously with HT-29 human colon cancer cells (4×10⁶). Five days following tumor inoculation, mice were administered either anti-EGFR antibody or DNA-conjugated anti-EGFR antibody (EGFR Ab-DNA 1-SEQ ID NO: 1) (20 μg peri-tumoral twice weekly for three weeks), or left untreated. Analysis of tumor size and volume demonstrated marked inhibition of tumor growth following administration of EGFR Ab-DNA that was significantly greater than that of the unconjugated parent anti-EGFR antibody (FIG. 19A, 19B). In contrast to the transient effect of EGFR Ab, the inhibition of tumor growth in response to treatment with EGFR Ab-DNA was sustained for more than 12 months.

### Example 11

### DNA Conjugated Anti-Neu Antibody Inhibits Growth of Neu+ Tumors in Syngeneic FVB Mice and Spontaneous Tumors in HER2/Neu Transgenic Mice

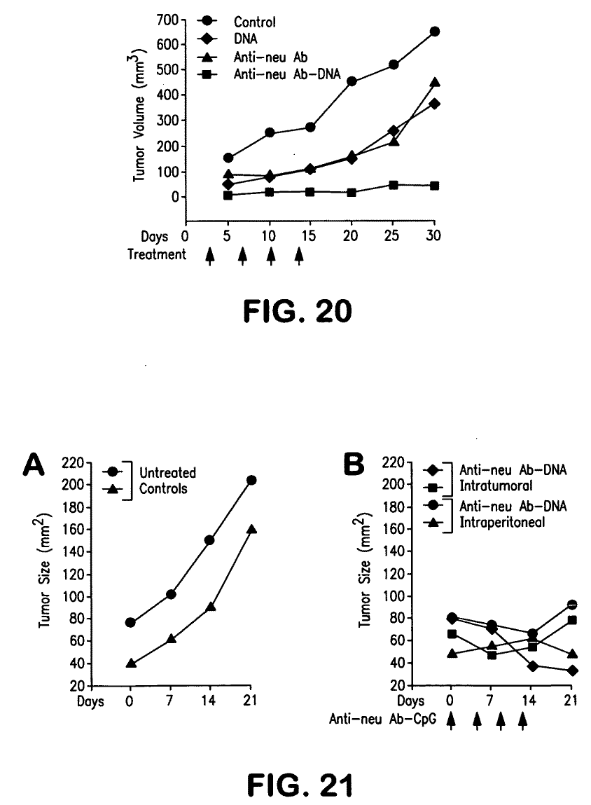

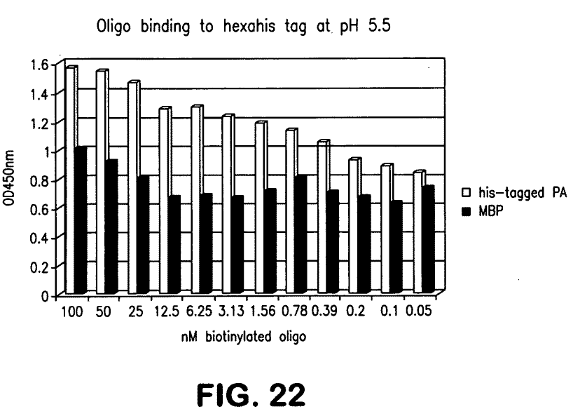

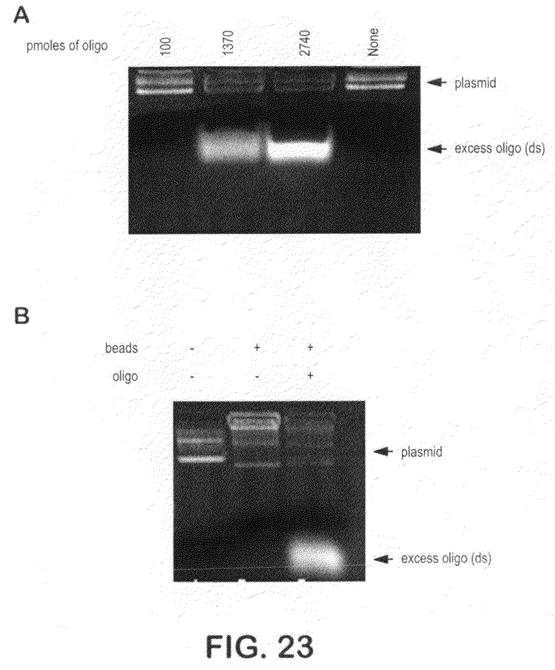

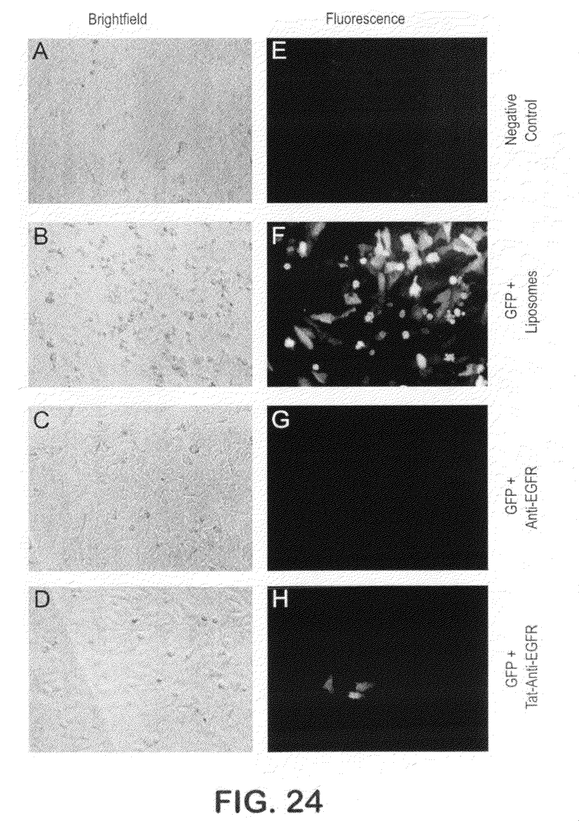

FVB mice were injected subcutaneously with NT2 neu+breast cancer cells (4×10⁶). Five days following tumor inoculation, mice were administered either anti-Neu antibody or DNA-conjugated anti-Neu antibody (Neu Ab-DNA 1-SEQ ID NO: 1) (20 μg peri-tumoral twice weekly for three weeks), or left untreated. Analysis of tumor size and volume demonstrated marked inhibition of tumor growth following administration of Neu Ab-DNA that was significantly greater than that of the unconjugated parent anti-Neu antibody or DNA (FIG. 20).

Neu (neu/N)-transgenic mice bearing spontaneous mammary carcinomas were administered DNA-conjugated anti-neu antibody (Neu Ab-DNA 1-SEQ ID NO: 1) (100 μg i.p. twice weekly for two weeks or 50 μg intratumoral twice weekly for two weeks), or left untreated. Analysis of tumor size and volume demonstrated marked inhibition of tumor growth and reduction of tumor volume following administration of DNA-conjugated anti-neu antibody. (FIGS. 21A and 21B).

Although the invention has been described with reference to the above examples, it will be understood that modifications and variations are encompassed within the spirit and scope of the invention. Accordingly, the invention is limited only by the following claims.

1. An isolated targeting moiety-biologically active agent conjugate comprising:
a targeting moiety that binds to a cellular component or specific molecule;
one or more nucleic acid molecule(s); and one or more antigenic peptide or one or more polypeptide.

2. The conjugate of claim 1, wherein the targeting moiety is selected from a group consisting of an antibody, a peptide, an aptamer, a ligand and a combination thereof.

3. The conjugate of claim 1, wherein said cellular component is a tumor antigen, tumor associated antigen, or tumor cell surface molecule.

4. The conjugate of claim 1, wherein said cellular component is a cell surface molecule present on a normal cell.

5. The conjugate of claim 1, wherein said cellular component is a molecule present on an immune cell.

6. The conjugate of claim 1, wherein said cellular component is an antigen or antigenic determinant of a pathogen or microorganism.

7. The conjugate of claim 1, wherein said component is a fusion protein comprising an antigen and a tag.

8. The conjugate of claim 1, wherein said nucleic acid molecule is selected from a group consisting of a double strand DNA (ds DNA), single strand DNA (ssDNA), multistrand DNA, double strand RNA (ds RNA), single strand RNA (ssRNA), multistrand RNA, DNA-RNA hybrids (single strand or multistrand), peptide nucleic acid (PNA), PNA-DNA hybrid (single or multistrand), PNA-RNA hybrid (single or multistrand), locked nucleic acids (LNA), LNA-DNA hybrid (single or multistrand), and LNA-RNA hybrid (single or multistrand).

9. The conjugate of claim 1, wherein said nucleic acid molecule includes a coding sequence which is transcribed and/or translated in a target cell.

10. The conjugate of claim 9, wherein said coding sequence is a DNA plasmid or DNA molecule derived from a plasmid.

11. The conjugate of claim 10, wherein said nucleic acid molecule comprises a circular double stranded DNA molecule generated from a plasmid by site-specific recombination, comprising a gene of interest operably linked to an cell-specific expression regulatory element, and wherein said DNA molecule does not contain either an origin of replication or optionally a marker gene.

12. The conjugate of claims 10 or 11, wherein said DNA molecule comprises a nucleotide sequence predetermined to hybridize with an oligonucleotide.

13. The conjugate of claim 12, wherein said oligonucleotide is configured to form multistrand nucleic with said DNA molecule.

14. The conjugate of claim 13, wherein said oligonucleotide is a linear single strand or double strand RNA.

15. The conjugate of claim 13, wherein said oligonucleotide is a linear single strand DNA or double strand DNA peptide nucleic acid (PNA), locked nucleic acid (LNA), hybrid DNA-LNA, DNA-PNA.

16. The conjugate of claims 14 or 15, wherein said targeting moiety is bound to said olignucleotide, and wherein said oligonucleotide is further bound to a DNA molecule.

17. The conjugate of claim 14, wherein said targeting moiety is an aptamer molecule.

18. The conjugate of claim 17, wherein said aptamer further comprises said oligonucleotide.

19. An isolated targeting-moiety-biologically active agent conjugate comprising: a targeting moiety that binds to a cellular component; and a nucleic acid molecule which encodes one or more product designed to enhance an immune response.

20. The conjugate of claims 1 or 19, wherein said nucleic acid molecule comprises a double stranded DNA which is capable of stimulating an immune response.

21. The conjugate of claims 1 or 19, wherein said nucleic acid molecule comprises one or more immunostimulatory molecules selected from a group that includes: PAMP.

22. The conjugate of claim 1 or 19, wherein said nucleic acid molecule comprises a sequence that encodes one or more antigenic determinants.

23. The conjugate of claim 22, wherein said antigenic determinants is selected from a CD4+ T cell epitope, a CD8+ T cell epitope, a B cell epitope and a combination thereof.

24. The conjugate of claim 23, wherein said antigenic determinants are from a pathogen or microorganism.

25. The conjugate of claim 24, wherein said antigenic determinant is derived from tetanus toxin, diptheria toxin, pertussis toxin, hepatitis surface antigen, or pDOM1.

26. The conjugate of claims 1 or 19, wherein said nucleic acid molecule comprise a double stranded DNA molecule that encodes and tumor antigen; and at least one CD4+ T cell epitope from a pathogen or microorganism.

27. The conjugate of claims 1 or 19, wherein said one or more product comprises a pathogen associated molecular pattern (PAMP), Alarmin and/or damage associated molecular pattern (DAMP).

28. The conjugate of claim 27, wherein said nucleic acid molecule further encodes one or more immunostimulatory cytokines.

29. The conjugate of claims 1 or 19, wherein said nucleic acid molecule further encodes one or more co-stimulatory polypeptides.

30. The conjugate of claims 1 or 19, wherein said nucleic acid molecule further encodes one or more molecules that recruit, bind, mature/proliferative or activate an antigen presenting cell or dendritic cell.

31. The conjugate of claims 1 or 19, wherein said nucleic acid molecule encodes one or more immunostimulatory RNA molecules.

32. The conjugate of claims 19, wherein said nucleic acid molecule encodes one or more RNA molecules that can interfere with expression of at least one gene.

33. The conjugate of claims 1 or 19, wherein said nucleic acid molecule encodes a molecule that induces death of a target cell.

34. The conjugate of claims 1 or 19, wherein said nucleic acid molecule encodes one or more gene of interest under control of a transcription promoter which is functionally active in a target cell.

35. The conjugate of claims 1 or 19, further comprising a cationic peptide, cationic liposome, lipophilic moiety or nanoparticle.

36. The conjugate of claims 1 or 19, further comprising an Alarmin.

37. The conjugate of claims 1 or 19, further comprising a cathelicidin-derived LL37 peptide.

38. The conjugate of claims 1 or 19, wherein the nucleic acid molecule is a multistrand strand nucleic acid helix, DNA, RNA, DNA-RNA hybrid, PNA-DNA hybrid, LNA-DNA hybrid, or LNA-RNA hybrid.

39. The conjugate of claims 1 or 19, wherein the nucleic acid molecule is a DNA, RNA, PNA or LNA.

40. The conjugate of claims 1, 19 or 27, wherein said conjugate is further linked to an antigen or antigenic determinant.

41. The conjugate of claim 40, wherein the antigen or antigenic determinant is fused to a cationic peptide.

42. The conjugate of claim 41, wherein said cationic peptide is selected from a group consisting of LL37, His6 and Arg9.

43. The conjugate of claims 5, 24 or 25, wherein said targeting moiety binds a tumor cell, tumor associated antigen, or tumor vasculature.

44. The conjugate of claims 1 or 19, wherein the targeting moiety is capable of binding a molecule present on a normal skin or muscle cell.

45. The conjugate of claims 1 or 19, wherein the targeting moiety is capable of binding EGFR.

46. The conjugate of claims 1 or 19, wherein the targeting moiety is capable of binding an antigen presenting cell or a dendritic cell.

47. The conjugate of claims 1 or 19, wherein the targeting moiety is capable of binding a DC antigen uptake receptor.

48. The conjugate of claims 47, where receptor is selected from a group consisting of C type leptin-like receptors, Fc receptors, integrins and scavenger receptors.

49. The conjugate of claims 1 or 19, wherein the receptor is selected from a group consisting of DEC205, Fcγ receptor, αVβ5, CD36, Lox1, and CD91.

50. The conjugate of claim 1 or 19, wherein the targeting moiety is capable of binding a tumor antigen, tumor associated antigen, or tumor cell surface molecule.

51. The conjugate of claims 1 or 19, wherein the targeting moiety is capable of binding a cationic peptide.

52. The conjugate of claim 40, wherein said targeting moiety is coupled to LL37, His6, or Arg9.

53. The conjugate of claims 1 or 19, wherein said nucleic acid molecule is a linear DNA or minicircle DNA.

54. The conjugate of claim 53, wherein said DNA encodes an antigenic determinant derived from a pathogen or microorganism.

55. The conjugate of claim 51, further comprising a non-coding nucleic acid molecule comprising a DAMP, or Alarmin.

56. The conjugate of claims 1, 19, or 53, wherein said nucleic acid encodes a tumor antigen.

57. The conjugate of claim 53, wherein said antigenic determinant is derived from a pathogen.

58. The conjugate of claim 53, wherein said nucleic acid further comprises a sequence that is a PAMP.

59. The conjugate of claim 51, wherein said minicircle encodes a fusion protein comprising a tumor antigen fused with antigen derived from a pathogen or microorganism.

61. The conjugate of claims 1 or 19, wherein said targeting moiety comprise is capable of binding EGFR.

62. A method for treating or preventing a neoplastic disorder comprising administering to a subject in need thereof a therapeutically effective amount of the conjugate of claims 1 or 19.

63. A method for treating or preventing an infectious disease in a subject in need thereof comprising administering to a subject in need thereof a therapeutically effective amount of the conjugate of claims 1 or 19.

64. A method for ex vivo activation of immune cells, comprising contacting an immune cell with a composition of claims 1 or 19.

65. The method of claim 64, further comprising administering a therapeutically effective amount of said immune cell to a subject in need thereof.

66. A method of treating a tumor comprising, administering a composition of claims 1 or 19, in combination with corresponding microbial vaccine, wherein said conjugate comprises a antigenic determinant from said microbe.
